(19) United States
(12) Patent Application Publication
(10) Pub. No.: US 2012/0238576 A1
(43) Pub. Date: Sep. 20, 2012

(54) Triazine Derivatives and their Therapeutical Applications

(75) Inventors: Chunlin Tao, Los Angeles, CA (US); Qinwei Wang, Alhambra, CA (US); Laxman Nallan, Alhambra, CA (US); Tulay Polat, Los Angeles, CA (US); Lukasz Koroniak, Poznan (PL); Neil Desai, Los Angeles, CA (US)

(73) Assignee: CALIFORNIA CAPITAL EQUITY, LLC, LOS ANGELES (CA)

(21) Appl. No.: 13/376,964

(22) PCT Filed: Jun. 7, 2010
(86) PCT No.: PCT/US10/37570

§ 371 Date: Jun. 11, 2012

Related U.S. Application Data
(60) Provisional application No. 61/185,052, filed on Jun. 8, 2009.

(51) Int. Cl. A61K 31/53; C07D 417/14; A61K 31/5377; C07D 403/14; C07D 403/12; A61P 35/00; A61P 9/10; A61P 25/00; A61P 9/04; A61P 19/02; A61P 37/00; A61P 27/02; A61P 29/00; A61P 37/06; A61P 17/02; A61P 7/10; A61P 11/00; C07D 413/14; A61P 9/00; C07D 417/12

(52) U.S. Cl. 514/236.2; 514/245; 544/209; 544/113; 544/212

(57) ABSTRACT

The present invention comprises inter alia compounds as shown in formula (I) or a pharmaceutically acceptable salt thereof.

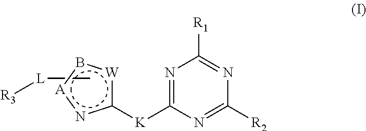

### CROSS-REFERENCE TO RELATED APPLICATIONS

This patent application claims the benefit of U.S. Provisional Patent Application No. 61/185,052, filed Jun. 8, 2009, which is incorporated by reference.

### FIELD OF THE INVENTION

The present invention relates generally to the use of compounds to treat a variety of disorders, diseases and pathologic conditions and more specifically to the use of triazine compounds to modulate protein kinases and for treating protein kinase-mediated diseases.

### BACKGROUND OF THE INVENTION

Protein kinases constitute a large family of structurally related enzymes that are responsible for the control of a variety of signal transduction processes within the cell. Protein kinases, containing a similar 250-300 amino acid catalytic domain, catalyze the phosphorylation of target protein substrates.

The kinases may be categorized into families by the substrates in the phosphorylate (e.g., protein-tyrosine, protein-serine/threonine, lipids, etc.). Tyrosine phosphorylation is a central event in the regulation of a variety of biological processes such as cell proliferation, migration, differentiation and survival. Several families of receptor and non-receptor tyrosine kinases control these events by catalyzing the transfer of phosphate from ATP to a tyrosine residue of specific cell protein targets. Sequence motifs have been identified that generally correspond to each of these kinase families [Hanks et al., FASEB J., (1995), 9, 576-596; Knighton et al., Science, (1991), 253, 407-414; Garcia-Bustos et al., EMBO J., (1994), 13:2352-2361). Examples of kinases in the protein kinase family include, without limitation, abl, Akt, bcr-abl, Blk, Brk, Btk, c-kit, c-Met, c-src, c-fms, CDK1, CDK2, CDK3, CDK4, CDK5, CDK6, CDK7, CDK8, CDK9, CDK10, cRaf1, CSF1R, CSK, EGFR, ErbB2, ErbB3, ErbB4, Erk, Fak, fes, FGFR1, FGFR2, FGFR3, FGFR4, FGFR5, Fgr, flt-1, Fps, Frk, Fyn, Hck, IGF-1R, INS-R, Jak, KDR, Lck, Lyn, MEK, p38, PDGFR, PIK, PKC, PYK2, ros, Tie, Tie-2, TRK, Yes, and Zap70.

Studies indicated that protein kinases play a central role in the regulation and maintenance of a wide variety of cellular processes and cellular function. For example, kinase activity acts as molecular switches regulating cell proliferation, activation, and/or differentiation. Uncontrolled or excessive kinase activity has been observed in many disease states including benign and malignant proliferation disorders as well as diseases resulting from inappropriate activation of the immune system (autoimmune disorders), allograft rejection, and graft vs host disease.

It is reported that many diseases are associated with abnormal cellular responses triggered by protein kinase-mediated events. These diseases include autoimmune diseases, inflammatory diseases, bone diseases, metabolic diseases, neurological and neurodegenerative diseases, cancer, cardiovascular diseases, allergies and asthma, Alzheimer's disease and hormone-related diseases. In addition, endothelial cell specific receptor PTKs, such as VEGF-2 and Tie-2, mediate the angiogenic process and are involved in supporting the progression of cancers and other diseases involving uncontrolled vascularization. Accordingly, there has been a substantial effort in medicinal chemistry to find protein kinase inhibitors that are effective as therapeutic agents.

One kinase family of particular interest is the Src family of kinases. Src kinase is involved in proliferation and migration responses in many cell types, cell activation, adhesion, motility, and survival, growth factor receptor signaling, and osteoclast activation (Biscardi et al., Adv. Cancer Res. (1999), 76, 61-119; Yeatman et al., Nat. Rev. Cancer (2004), 4, 470-480; Owens, D. W.; McLean et al., Mol. Biol. Cell (2000), 11, 51-64). Members of the Src family include the following eight kinases in mammals: Src, Fyn, Yes, Fgr, Lyn, Hck, Lck, and Blk (Bolen et al., Annu. Rev. Immunol, (1997), 15, 371). These are nonreceptor protein kinases that range in molecular mass from 52 to 62 kD. All are characterized by a common structural organization that is comprised of six distinct functional domains: Src homology domain 4 (SH4), a unique domain, SH3 domain, SH2 domain, a catalytic domain (SH1), and a C-terminal regulatory region (Brown et al., Biochim Biophys Acta (1996), 1287, 121-149; Tatosyan et al. Biochemistry (Moscow) 2000, 65, 49-58). SH4 domain contains the myristylation signals that guide the Src molecule to the cell membrane. This unique domain of Src proteins is responsible for their specific interaction with particular receptors and protein targets (Thomas et al., Annu Rev Cell Dev Biol (1997), 13, 513-609). The modulating regions, SH3 and SH2, control intra- as well as intermolecular interactions with protein substrates which affect Src catalytic activity, localization and association with protein targets (Pawson T., Nature (1995), 373, 573-580). The kinase domain, SH1, found in all proteins of the Src family, is responsible for the tyrosine kinase activity and has a central role in binding of substrates. The N-terminal half of Src kinase contains the site(s) for its tyrosine phosphorylation and regulates the catalytic activity of Src (Thomas et al., Annu Rev Cell Dev Biol (1997), 13: 513-609). v-Src differs from cellular Src (c-Src) on the basis of the structural differences in C-terminal region responsible for regulation of kinase activity.

The prototype member of the Src family protein tyrosine kinases was originally identified as the transforming protein (v-Src) of the oncogenic retrovirus, Rous sarcoma virus, RSV (Brugge et al., Nature (1977), 269, 346-348); Hamaguchi et al. (1995), Oncogene 10: 1037-1043). Viral v-Src is a mutated and activated version of a normal cellular protein (c-Src) with intrinsic tyrosine kinase activity (Collett et al., Proc Natl Acad Sci USA (1978), 75, 2021-2024). This kinase phosphorylates its protein substrates exclusively on tyrosyl residues (Hunter et al., Proc Natl Acad Sci USA (1980), 77, 1311-1315).

Investigations indicated that Src is a cytoplasmic protein tyrosine kinase, whose activation and recruitment to perimembranal signaling complexes has important implications for cellular fate. It has well-documented that Src protein levels and Src kinase activity are significantly elevated in human breast cancers (Muthuswamy et al., Oncogene, (1995), 11, 1801-1810); Wang et al., Oncogene (1999), 18, 1227-1237; Warmuth et al., Curr. Pharm. Des. (2003), 9, 2043-2059], colon cancers (Irby et al., Nat Genet (1999), 21, 187-190), pancreatic cancers (Lutz et al., Biochem Biophys Res Commun (1998), 243, 503-508], certain B-cell leukemias and lymphomas (Talamonti et al., J. Clin. Invest. (1993), 91, 53; Lutz et al., Biochem. Biophys. Res. (1998), 243, 503; Biscardi et al., Adv. Cancer Res. (1999), 76, 61; Lynch et al., Leukemia (1993), 7, 1416; Boschelli et al., Drugs of the Future (2000), 25(7), 717), gastrointestinal cancer (Cartwright et al., Proc. Natl. Acad. Sci. USA, (1990), 87, 558-562 and Mao et al., Oncogene, (1997), 15, 3083-3090), non-small cell lung cancers (NSCLCs) (Mazurenko et al., European Journal of Cancer, (1992), 28, 372-7), bladder cancer (Fanning et al., Cancer Research, (1992), 52, 1457-62), oesophageal cancer (Jankowski et al., Gut, (1992), 33, 1033-8), prostate and ovarian cancer (Wiener et al., Clin. Cancer Research, (1999), 5, 2164-70), melanoma and sarcoma (Bohlen et al., Oncogene, (1993), 8, 2025-2031; tatosyan et al., Biochemistry (Moscow) (2000), 65, 49-58). Furthermore, Src kinase modulates signal transduction through multiple oncogenic pathways, including EGFR, Her2/neu, PDGFR, FGFR, and VEGFR (Frame et al., Biochim. Biophys. Acta (2002), 1602, 114-130; Sakamoto et al., Jpn J Cancer Res, (2001), 92: 941-946).

Thus, it is anticipated that blocking signaling through the inhibition of the kinase activity of Src will be an effective means of modulating aberrant pathways that drive oncologic transformation of cells. Src kinase inhibitors may be useful anti-cancer agents (Abram et al., Exp. Cell Res., (2000), 254, 1). It is reported that inhibitors of src kinase had significant antiproliferative activity against cancer cell lines (M. M. Moasser et al., Cancer Res., (1999), 59, 6145; Tatosyan et al., Biochemistry (Moscow) (2000), 65, 49-58).) and inhibited the transformation of cells to an oncogenic phenotype (R. Karni et al., Oncogene (1999), 18, 4654). Furthermore, antisense Src expressed in ovarian and colon tumor cells has been shown to inhibit tumor growth (Wiener et al., Clin. Cancer Res., (1999), 5, 2164; Staley et al., Cell Growth Diff (1997), 8, 269). Src kinase inhibitors have also been reported to be effective in an animal model of cerebral ischemia (Paul et al. Nature Medicine, (2001), 7, 222), suggesting that Src kinase inhibitors may be effective at limiting brain damage following stroke. Suppression of arthritic bone destruction has been achieved by the overexpression of CSK in rheumatoid synoviocytes and osteoclasts (Takayanagi et al., J. Clin. Invest. (1999), 104, 137). CSK, or C-terminal Src kinase, phosphorylates and thereby inhibits Src catalytic activity. This implies that Src inhibition may prevent joint destruction that is characteristic in patients suffering from rheumatoid arthritis (Boschelli et al., Drugs of the Future (2000), 25(7), 717).

It is well documented that Src-family kinases are also important for signaling downstream of other immune cell receptors. Fyn, like Lck, is involved in TCR signaling in T cells (Appleby et al., Cell, (1992), 70, 751). Hck and Fgr are involved in Fcγ receptor signaling leading to neutrophil activation (Vicentini et al., J. Immunol. (2002), 168, 6446). Lyn and Src also participate in Fcγ receptor signaling leading to release of histamine and other allergic mediators (Turner, H. and Kinet, J-P Nature (1999), 402, B24). These-findings suggest that Src family kinase inhibitors may be useful in treating allergic diseases and asthma.

Other Src family kinases are also potential therapeutic targets. Lck plays a role in T-cell signaling. Mice that lack the Lck gene have a poor ability to develop thymocytes. The function of Lck as a positive activator of T-cell signaling suggests that Lck inhibitors may be useful for treating autoimmune disease such as rheumatoid arthritis (Molina et al., Nature, (1992), 357, 161).

Hck is a member of the Src protein-tyrosine kinase family and is expressed strongly in macrophages, an important HIV target cell and its inhibition in HIV-infected macrophages might slow disease progression (Ye et al., Biochemistry, (2004), 43 (50), 15775-15784).

Hck, Fgr and Lyn have been identified as important mediators of integrin signaling in myeloid leukocytes (Lowell et al., J Leukoc. Biol., (1999), 65, 313). Inhibition of these kinase mediators may therefore be useful for treating inflammation (Boschelli et al., Drugs of the Future (2000), 25(7), 717).

It is reported that Syk is a tyrosine kinase that plays a critical role in the cell degranulation and eosinophil activation and Syk kinase is implicated in various allergic disorders, in particular asthma (Taylor et al., Mol. Cell. Biol. (1995), 15, 4149).

BCR-ABL encodes the BCR-AEL protein, a constitutively active cytoplasmic tyrosine kinase present in 90% of all patients with chronic myelogenous leukemia (CML) and in 15-30% of adult patients with acute lymphoblastic leukemia (ALL). Numerous studies have demonstrated that the activity of BCR-ABL is required for the cancer causing ability of this chimeric protein.

Src kinases play a role in the replication of hepatitis B virus. The virally encoded transcription factor HBx activates Src in a step required for propagation of the virus (Klein et al., EMBO J. (1999), 18, 5019; Klein et al., Mol. Cell. Biol. (1997), 17, 6427). Some genetic and biochemical data clearly demonstrate that Src-family tyrosine kinases serve as a critical signal relay, via phosphorylation of c-Cbl, for fat accumulation, and provide potential new strategies for treating obesity (Sun et al., Biochemistry, (2005), 44 (44), 14455-14462). Since Src plays a role in additional signaling pathways, Src inhibitors are also being pursued for the treatment of other diseases including osteoporosis and stroke (Susva et al., Trends Pharmacol. Sci. (2000), 21, 489-495; Paul et al., Nat. Med. (2001), 7, 222-227).

It is also possible that inhibitors of the Src kinase activity are useful in the treatment of osteoporosis (Soriano et al., Cell (1991), 64, 693; Boyce et al. J. Clin. Invest (1992), 90, 1622; Owens et al., Mol. Biol. Cell (2000), 11, 51-64), T cell mediated inflammation (Anderson et al., Adv. Immunol. (1994), 56, 151; Goldman, F D et al. J. Clin. Invest. (1998), 102, 421), and cerebral ischemia (Paul et al. Nature Medicine (2001), 7, 222).

In addition, src family kinases participate in signal transduction in several cell types. For example, fyn, like Ick, is involved in T-cell activation. Hck and fgr are involved in Fe gamma receptor mediated oxidative burst of neutrophils. Src and lyn are believed to be important in Fc epsilon induced degranulation of mast cells, and so may play a role in asthma and other allergic diseases. The kinase lyn is known to be involved in the cellular response to DNA damage induced by UV light (Hiwasa et al., FEBS Lett. (1999), 444, 173) or ionizing radiation (Kumar et al., J Biol Chein, (1998), 273, 25654). Inhibitors of lyn kinase may thus be useful as potentiators in radiation therapy.

T cells play a pivotal role in the regulation of immune responses and are important for establishing immunity to pathogens. In addition, T cells are often activated during inflammatory autoimmune diseases, such as rheumatoid arthritis, inflammatory bowel disease, type I diabetes, multiple sclerosis, Sjogren's disease, myasthenia gravis, psoriasis, and lupus. T cell activation is also an important component of transplant rejection, allergic reactions, and asthma.

T cells are activated by specific antigens through the T cell receptor, which is expressed on the cell surface. This activation triggers a series of intracellular signaling cascades mediated by enzymes expressed within the cell (Kane et al. Current Opinion in Immunol. (2000), 12, 242). These cascades lead to gene regulation events that result in the production of cytokines, like interleukin-2 (IL-2). IL-2 is a necessary cytokine in T cell activation, leading to proliferation and amplification of specific immune responses.

Therefore, Src kinase and other kinase have become intriguing targets for drug discovery (Parang et al., Expert Opin. Ther. Pat. (2005), 15, 1183-1207; Parang et al., Curr. Opin. Drug Discovery Dev. (2004), 7, 630-638). Many classes of compounds have been disclosed to modulate or, more specifically, inhibit kinase activity for use to treat kinase-related conditions or other disorders. For example, U.S. Pat. No. 6,596,746 and the PCT WO 05/096784A2 disclosed benzotrianes as inhibitors of kinases; the PCT WO 01/81311 disclosed substituted benzoic acid amides for the inhibition of angiogenisis; U.S. Pat. No. 6,440,965, disclosed substituted pyrimidine derivatives in the treatment of neurodegenerative or neurological disorders; PCT WO 02/08205 reported the pyrimidine derivatives having neurotrophic activity; PCT WO 03/014111 disclosed arylpiperazines and arylpiperidines and their use as metalloproteinase inhibiting agents; PCT WO 03/024448 described compounds as inhibitors of histone deacetylase enzymatic activity; PCT WO 04/058776 disclosed compounds which possess anti-angiogenic activity. PCT WO 01/94341 and WO 02/16352 disclosed Src kinase inhibitors of quinazoline derivatives. PCT WO03/026666A1 and WO03/018021A1 disclosed pyrimidinyl derivatives as kinase inhibitors. U.S. Pat. No. 6,498,165 reported Src kinase inhibitor compounds of pyrimidine compounds. Peptides as Src Tyrosine Kinase Inhibitors is reported recently (Kumar et al., J. Med. Chem., (2006), 49 (11), 3395-3401). The quinolinecarbonitriles derivatives was reported to be potent dual Inhibitors of Src and Abl Kinases (Diane et al., J. Med. Chem., (2004), 47 (7), 1599-1601).

Although many inhibitors of kinases are known, there exists a need for new treatment options for conditions associated with protein kinases.

### BRIEF SUMMARY OF THE INVENTION

Accordingly, the present invention provides an antitumor agent comprising a triazine derivative as described in formula (I) or formula (II), pharmaceutically-acceptable formulations thereof, methods for making novel compounds and compositions for using the compounds. The compounds and compositions comprising the compounds of formula (I) or formula (II) have utility in treatment of a variety of diseases.

The combination therapy described herein may be provided by the preparation of the triazine derivative of formula (I) or formula (II) and the other therapeutic agent as separate pharmaceutical formulations followed by the administration thereof to a patient simultaneously, semi-simultaneously, separately or over regular intervals.

The present invention also provides methods for using certain chemical compounds such as kinase inhibitors in the treatment of various diseases, disorders, and pathologies, for example, cancer, and vascular disorders, such as myocardial infarction (MI), stroke, or ischemia. The triazine compounds described in this invention may block the enzymatic activity of some or many of the members of the Src family, in addition to blocking the activity of other receptor and non-receptor kinase. Such compounds may be beneficial for treatment of the diseases where disorders affect cell motility, adhesion, and cell cycle progression, and in addition, diseases with related hypoxic conditions, osteoporosis and conditions, which result from or are related to increases in vascular permeability, inflammation or respiratory distress, tumor growth, invasion, angiogenesis, metastases and apoptosis.

### DETAILED DESCRIPTION OF THE INVENTION

The present invention comprises compounds as shown in formula (I)

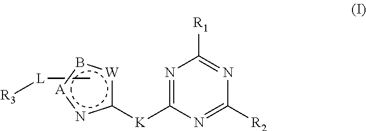

or a pharmaceutically acceptable salt thereof, wherein:

A, B, W is selected from S, O, NR₄, CR₄ or L-R₃;

R4 is independently selected from hydrogen or an optionally substituted C₁₋₄ aliphatic group.

R₁ represents hydrogen, halogen, hydroxy, amino, cyano, alkyl, cycloalkyl, alkenyl, alkynyl, alkylthio, aryl, arylalkyl, heterocyclic, heteroaryl, heterocycloalkyl, alkylsulfonyl, alkoxycarbonyl and alkylcarbonyl.

R₂ is selected from:

(i) amino, alkyl amino, aryl amino, heteroaryl amino;

(ii) C₁-C₆ alkyl, C₂-C₆ alkenyl, C₂-C₆ alkynyl;

(iii) heterocyclic, heteroaryl; and

(iv) groups of the formula (Ia):

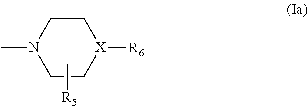

wherein:

R₅ represents hydrogen, C₁-C₄ alkyl, oxo;

X is CH, when R₆ is hydrogen; or X—R₆ is O; or X is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)amino-C₀-C₄alkyl, (4- to 7-membered heterocycle)C₀-C₄alkyl, C₁-C₆ alkylsulfonyl, mono- and di-(C₁-C₆ alkyl) sulfonamido, and mono- and di-(C₁-C₆alkyl)aminocarbonyl, each of which is substituted with from 0 to 4 substituents independently chosen from halogen, hydroxy, cyano, amino, —COOH and oxo;

L represents O, S, SO, CO, SO2, CO2, NR4, (CH₂)ₘ, m=0-3, CONR₄, NR₄CO, NR₄SO₂, SO₂NR₄, NR₄CO₂, NR₄COR₄, NR₄SO₂NR₄, NR₄NR₄, OCONR₄, C(R₄)₂CONR₄, NR₄COC(R₄), C(R₄)₂SO, C(R₄)₂SO₂, C(R₄)₂SO₂NR₄, C(R₄)₂NR₄, C(R₄)₂NR₄CO, C(R₄)₂NR₄CO₂, C(R₄)═NNR₄, C(R₄)═N—O, C(R₄)₂NR₄NR₄, C(R₄)₂NR₄SO₂NR₄, C(R₄)₂NR₄CONR₄, O(CH₂)ₚ, S(CH₂)ₚ, p=1-3, or (CH₂)_{q}O, or (CH₂)_{q}S, q=1-3.

R₃ is selected from:

(i) C₁-C₆ alkyl, C₂-C₆ alkenyl, C₂-C₆ alkynyl;

(ii) heterocyclic,

(iii) Ar.

Ar represents heteroaryl or aryl, each of which is substituted with from 0 to 4 substituents independently chosen from:
(1) halogen, hydroxy, amino, cyano, —COOH, —SO₂NH₂, oxo, nitro and alkoxycarbonyl; and
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- and di-(C₁-C₆alkyl)amino, C₁-C₆ alkylsulfonyl, mono- and di-(C₁-C₆alkyl) sulfonamido and mono- and di-(C₁-C₆alkyl)aminocarbonyl; phenylC₀-C₄alkyl and (4- to 7-membered heterocycle)-C₀-C₄alkyl, each of which is substituted with from 0 to 4 secondary substituents independently chosen from halogen, hydroxy, cyano, oxo, imino, C₁-C₄alkyl, C₁-C₄alkoxy and C₁-C₄haloalkyl.

K is selected from

i) absence;

ii) O, S, SO, SO₂;

iii) (CH₂)ₘ, m=0-3, O(CH₂)ₚ, p=1-3, (CH₂)_{q}O, q=1-3.

iv) NR_{S}

R₇ represents hydrogen, alkyl, cycloalkyl, alkenyl, alkynyl, alkylthio, aryl, arylalkyl.

The present invention also comprises compounds of formula (II)

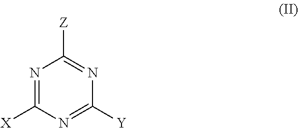

or a pharmaceutically acceptable salt thereof, wherein:

Y is selected from −OR⁴, —NR⁴R⁵, and -Q-R³;

Q is selected from cycloalkyl and heterocycloalkyl, each of which is optionally substituted with C₁-C₆ alkyl or oxo;

R³ is selected from H, C₁-C₆ alkyl, C₁-C₆ alkyl-R⁶, aryl, and heteroaryl, each of which is optionally substituted with C₁-C₆ alkyl, halo, trifluoromethyl, or oxo;

R⁴ and R⁵ are each independently selected from H, C₁-C₆ alkyl-R⁶, aryl, and heteroaryl;

R⁶ is selected from hydroxy, cyano, aryl, heteroaryl, cycloalkyl, heterocycloalkyl, —NH₂, mono(C₁-C₆)alkylamino, di(C₁-C₆)alkylamino, and C₁-C₆ alkoxy;

X is —NH—Ar¹—R¹;

Ar¹ is selected from aryl and heteroaryl, each of which is optionally substituted with C₁-C₆ alkyl or halo;

R¹ is selected from —(CH₂)ₙC(O)NHW, —CH₂C(O)NHAr¹, and —NH₂;

n=0, 1;

W is selected from C₁-C₆ alkyl, cycloalkyl, and —(CH₂)Ar¹;

Z is selected from H, C₁-C₆ alkyl, aryl, and heteroaryl.

The invention further comprises compounds of formula (II)

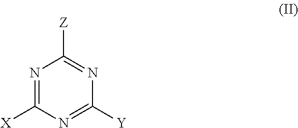

or a pharmaceutically-acceptable salt thereof, wherein:

Y is selected from —OR⁴, —NR⁴R⁵, and -Q-R³;

Q is selected from morpholinyl, piperazinyl and piperidinyl;

R³ is selected from H, C₁-C₆ alkyl, hydroxy(C₁-C₆)alkyl, cyano(C₁-C₆)alkyl, pyridinylmethyl, pyridinyl, phenyl, trifluoromethylphenyl, and oxo;

R⁴ and R⁵ are each independently selected from H, C₁-C₆ alkyl-R⁶, and phenyl;

R⁶ is selected from hydroxy, morpholinyl, di(C₁-C₆)alkylamino, imidazolyl, and C₁-C₆ alkoxy;

X is —NH—Ar¹—R¹;

Ar¹ is selected from thiazolyl, oxazolyl, oxadiazolyl, methyl-imidazolyl, pyrazolyl;

R¹ is selected from —(CH₂)ₙC(O)NHW and —NH₂;

n=0, 1;

W is selected from C₁-C₆ alkyl and —(CH₂)ₙPh optionally substituted with C₁-C₆ alkyl or halo;

Z is selected from H, C₁-C₆ alkyl, and phenyl.

The invention also comprises compounds of formula (II)

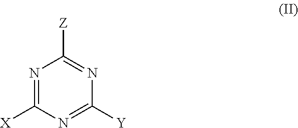

or a pharmaceutically-acceptable salt thereof, wherein:

Y is selected from —OR⁴, —NR⁴R⁵, and -Q-R³;

Q is selected from morpholinyl, piperazinyl and piperidinyl;

R³ is selected from H, C₁-C₆ alkyl, hydroxy(C₁-C₆)alkyl, cyano(C₁-C₆)alkyl, pyridinylmethyl, pyridinyl, phenyl, trifluoromethylphenyl, and oxo;

R⁴ and R⁵ are each independently selected from H, C₁-C₆ alkyl-R⁶, and phenyl;

R⁶ is selected from hydroxy, morpholinyl, di(C₁-C₆)alkylamino, imidazolyl, and C₁-C₆ alkoxy;

X is —NH—Ar¹—R¹;

Ar¹ is selected from thiazolyl, oxazolyl, oxadiazolyl, methyl-imidazolyl, pyrazolyl;

R¹ is selected from —(CH₂)ₙC(O)NHW, —CH₂C(O)NHAr², and —NH₂;

n=0, 1;

W is selected from C₁-C₆ alkyl, cycloalkyl, and —(CH₂)Ar²;

Ar² is phenyl, optionally substituted with C₁-C₆ alkyl or halo;

Z is selected from H, C₁-C₆ alkyl, and phenyl.

The following definitions refer to the various terms used above and throughout the disclosure.

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Certain compounds are described herein using a general formula that include, variables (e.g. X, Ar.). Unless otherwise specified, each variable within such a formula is defined independently of any other variable, and any variable that occurs more than one time in a formula is defined independently at each occurrence.

The term “halo” or “halogen” refers to fluorine, chlorine, bromine or iodine.

The term “alkyl” herein alone or as part of another group refers to a monovalent alkane (hydrocarbon) derived radical containing from 1 to 12 carbon atoms unless otherwise defined. Alkyl groups may be substituted at any available point of attachment. An alkyl group substituted with another alkyl group is also referred to as a “branched alkyl group”. Exemplary alkyl groups include methyl, ethyl, propyl, isopropyl, n-butyl, t-butyl, isobutyl, pentyl, hexyl, isohexyl, heptyl, dimethylpentyl, octyl, 2,2,4-trimethylpentyl, nonyl, decyl, undecyl, dodecyl, and the like. Exemplary substituents include but are not limited to one or more of the following groups: alkyl, aryl, halo (such as F, Cl, Br, I), haloalkyl (such as CCl₃ or CF₃), alkoxy, alkylthio, hydroxy, carboxy (—COOH), alkyloxycarbonyl (—C(O)R), alkylcarbonyloxy (—OCOR), amino (—NH₂), carbamoyl (—NHCOOR— or —OCONHR—), urea (—NHCONHR—) or thiol (—SH). In some preferred embodiments of the present invention, alkyl groups are substituted with, for example, amino, heterocycloalkyl, such as morpholine, piperazine, piperidine, azetidine, hydroxyl, methoxy, or heteroaryl groups such as pyrrolidine. “Alkyl” also includes cycloalkyl.

The term “cycloalkyl” herein alone or as part of another group refers to fully saturated and partially unsaturated hydrocarbon rings of 3 to 9, preferably 3 to 7 carbon atoms. The examples include cyclopropyl, cyclobutyl, cyclopentyl and cyclohexyl, and like. Further, a cycloalkyl may be substituted. A substituted cycloalkyl refers to such rings having one, two, or three substituents, selected from the group consisting of halo, alkyl, substituted alkyl, alkenyl, alkynyl, nitro, cyano, oxo (═O), hydroxy, alkoxy, thioalkyl, —CO2H, —C(═O)H, CO₂-alkyl, —C(═O)alkyl, keto, ═N—OH, ═N—O-alkyl, aryl, heteroaryl, heterocyclo, —NR′R″, —C(═O)NR′R″, —CO₂NR′R″, —C(═O)NR′R″, —NR′CO₂R″, —NR′C(═O)R″, —SO₂NR′R″, and —NR′SO₂R″, wherein each of R′ and R″ are independently selected from hydrogen, alkyl, substituted alkyl, and cycloalkyl, or R′ and R″ together form a heterocyclo or heteroaryl ring.

The term “alkenyl” herein alone or as part of another group refers to a hydrocarbon radical straight, branched or cyclic containing from 2 to 12 carbon atoms and at least one carbon to carbon double bond. Examples of such groups include the vinyl, allyl, 1-propenyl, isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, and like. Alkenyl groups may also be substituted at any available point of attachment. Exemplary substituents for alkenyl groups include those listed above for alkyl groups, and especially include C₃ to C₇ cycloalkyl groups such as cyclopropyl, cyclopentyl and cyclohexyl, which may be further substituted with, for example, amino, oxo, hydroxyl, etc.

The term “alkynyl” refers to straight or branched chain alkyne groups, which have one or more unsaturated carbon-carbon bonds, at least one of which is a triple bond. Alkynyl groups include C₂-C₈ alkynyl, C₂-C₆ alkynyl and C₂-C₄ alkynyl groups, which have from 2 to 8, 2 to 6 or 2 to 4 carbon atoms, respectively. Illustrative of the alkynyl group include ethenyl, propenyl, isopropenyl, butenyl, isobutenyl, pentenyl, and hexenyl. Alkynyl groups may also be substituted at any available point of attachment. Exemplary substituents for alkynyl groups include those listed above for alkyl groups such as amino, alkylamino, etc. The numbers in the subscript after the symbol “C” define the number of carbon atoms a particular group can contain.

The term “alkoxy” alone or as part of another group denotes an alkyl group as described above bonded through an oxygen linkage (—O—). Preferred alkoxy groups have from 1 to 8 carbon atoms. Examples of such groups include the methoxy, ethoxy, n-propoxy, isopropoxy, n-butoxy, isobutoxy, sec-butoxy, tert-butoxy, n-pentyloxy, isopentyloxy, n-hexyloxy, cyclohexyloxy, n-heptyloxy, n-octyloxy and 2-ethylhexyloxy.

The term “alkylthio” refers to an alkyl group as described above attached via a sulfur bridge. Preferred alkoxy and alkylthio groups are those in which an alkyl group is attached via the heteroatom bridge. Preferred alkylthio groups have from 1 to 8 carbon atoms. Examples of such groups include the methylthio, ethylthio, n-propythiol, n-butylthiol, and like.

The term “oxo,” as used herein, refers to a keto (C═O) group. An oxo group that is a substituent of a nonaromatic carbon atom results in a conversion of —CH₂— to —C(═O)—.

The term “alkoxycarbonyl” herein alone or as part of another group denotes an alkoxy group bonded through a carbonyl group. An alkoxycarbonyl radical is represented by the formula: —C(O)OR, where the R group is a straight or branched C₁-C₆ alkyl group, cycloalkyl, aryl, or heteroaryl.

The term “alkylcarbonyl” herein alone or as part of another group refers to an alkyl group bonded through a carbonyl group or —C(O)R.

The term “arylalkyl” herein alone or as part of another group denotes an aromatic ring bonded through an alkyl group (such as benzyl) as described above.

The term “aryl” herein alone or as part of another group refers to monocyclic or bicyclic aromatic rings, e.g. phenyl, substituted phenyl and the like, as well as groups which are fused, e.g., napthyl, phenanthrenyl and the like. An aryl group thus contains at least one ring having at least 6 atoms, with up to five such rings being present, containing up to 20 atoms therein, with alternating (resonating) double bonds between adjacent carbon atoms or suitable heteroatoms. Aryl groups may optionally be substituted with one or more groups including, but not limited to halogen such as I, Br, F, or Cl; alkyl, such as methyl, ethyl, propyl, alkoxy, such as methoxy or ethoxy, hydroxy, carboxy, carbamoyl, alkyloxycarbonyl, nitro, alkenyloxy, trifluoromethyl, amino, cycloalkyl, aryl, heteroaryl, cyano, alkyl S(O)ₘ (m=0, 1, 2), or thiol.

The term “aromatic” refers to a cyclically conjugated molecular entity with a stability, due to delocalization, significantly greater than that of a hypothetical localized structure, such as the Kekule structure.

The term “amino” herein alone or as part of another group refers to —NH₂. An “amino” may optionally be substituted with one or two substituents, which may be the same or different, such as alkyl, aryl, arylalkyl, alkenyl, alkynyl, heteroaryl, heteroarylalkyl, cycloheteroalkyl, cycloheteroalkylalkyl, cycloalkyl, cycloalkylalkyl, haloalkyl, hydroxyalkyl, alkoxyalkyl, thioalkyl, carbonyl or carboxyl. These substituents may be further substituted with a carboxylic acid, any of the alkyl or aryl substituents set out herein. In some embodiments, the amino groups are substituted with carboxyl or carbonyl to form N-acyl or N-carbamoyl derivatives.

The term “alkylsulfonyl” refers to groups of the formula (SO₂)-alkyl, in which the sulfur atom is the point of attachment. Preferably, alkylsulfonyl groups include C₁-C₆ alkylsulfonyl groups, which have from 1 to 6 carbon atoms. Methylsulfonyl is one representative alkylsulfonyl group.

The term “heteroatom” refers to any atom other than carbon, for example, N, O, or S.

The term “heteroaryl” herein alone or as part of another group refers to substituted and unsubstituted aromatic 5 or 6 membered monocyclic groups, 9 or 10 membered bicyclic groups, and 11 to 14 membered tricyclic groups which have at least one heteroatom (O, S or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom.

The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain zero, one, two or three substituents selected from the group consisting of halo, alkyl, substituted alkyl, alkenyl, alkynyl, aryl, nitro, cyano, hydroxy, alkoxy, thioalkyl, —CO₂H, —C(═O)H, —CO₂-alkyl, —C(═O)alkyl, phenyl, benzyl, phenylethyl, phenyloxy, phenylthio, cycloalkyl, substituted cycloalkyl, heterocyclo, heteroaryl, —C(═O)NR′R″, —CO₂NR′R″, —C(═O)NR′R″, —NR′CO₂R″, —NR′C(═O)R″, —SO₂NR′R″, and —NR′SO₂R″, wherein each of R′ and R″ is independently selected from hydrogen, alkyl, substituted alkyl, and cycloalkyl, or R′ and R″ together form a heterocyclo or heteroaryl ring.

Preferably monocyclic heteroaryl groups include pyrrolyl, pyrazolyl, pyrazolinyl, imidazolyl, oxazolyl, diazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like.

Preferably bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, dihydroisoindolyl, tetrahydroquinolinyl and the like.

Preferably tricyclic heteroaryl groups include carbazolyl, benzidolyl, phenanthrollinyl, acridinyl, phenanthridinyl, xanthenyl and the like.

The term “heterocycle” or “heterocycloalkyl” herein alone or as part of another group refers to a cycloalkyl group (nonaromatic) in which one of the carbon atoms in the ring is replaced by a heteroatom selected from O, S or N. The “heterocycle” has from 1 to 3 fused, pendant or spiro rings, at least one of which is a heterocyclic ring (i.e., one or more ring atoms is a heteroatom, with the remaining ring atoms being carbon). The heterocyclic ring may be optionally substituted which means that the heterocyclic ring may be substituted at one or more substitutable ring positions by one or more groups independently selected from alkyl (preferably lower alkyl), heterocycloalkyl, heteroaryl, alkoxy (preferably lower alkoxy), nitro, monoalkylamino (preferably a lower alkylamino), dialkylamino (preferably a alkylamino), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy; lower alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups. A heterocyclic group may generally be linked via any ring or substituent atom, provided that a stable compound results. N-linked heterocyclic groups are linked via a component nitrogen atom.

Typically, a heterocyclic ring comprises 1-4 heteroatoms; within certain embodiments each heterocyclic ring has 1 or 2 heteroatoms per ring. Each heterocyclic ring generally contains from 3 to 8 ring members (rings having from to 7 ring members are recited in certain embodiments), and heterocycles comprising fused, pendant or spiro rings typically contain from 9 to 14 ring members which consists of carbon atoms and contains one, two, or three heteroatoms selected from nitrogen, oxygen and/or sulfur.

Examples of “heterocycle” or “heterocycloalkyl groups include piperazine, piperidine, morpholine, thiomorpholine, pyrrolidine, imidazolidine and thiazolide.

The term “substituent,” as used herein, refers to a molecular moiety that is covalently bonded to an atom within a molecule of interest. For example, a “ring substituent” may be a moiety such as a halogen, alkyl group, haloalkyl group or other group discussed herein that is covalently bonded to an atom (preferably a carbon or nitrogen atom) that is a ring member.

The term “optionally substituted” as it refers that the aryl or heterocyclyl or other group may be substituted at one or more substitutable positions by one or more groups independently selected from alkyl (preferably lower alkyl), alkoxy (preferably lower alkoxy), nitro, monoalkylamino (preferably with one to six carbons), dialkylamino (preferably with one to six carbons), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy and lower alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups. Optional substitution is also indicated by the phrase “substituted with from 0 to X substituents,” where X is the maximum number of possible substituents. Certain optionally substituted groups are substituted with from 0 to 2, 3 or 4 independently selected substituents.

A dash (“-”) that is not between two letters or symbols is used to indicate a point of t attachment for a substituent. For example, —CONH2 is attached through the carbon atom.

A dashed cycle that locates inside of a heterocyle ring is used to indicate a conjugated system. The bonds between two atomes may be single bond or double bond.

The term “anticancer” agent includes any known agent that is useful for the treatment of cancer including, but is not limited, Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

The term “kinase” refers to any enzyme that catalyzes the addition of phosphate groups to a protein residue; for example, serine and threonine kineses catalyze the addition of phosphate groups to serine and threonine residues.

The terms “Src kinase,” “Src kinase family,” and “Src family” refer to the related homologs or analogs belonging to the mammalian family of Src kineses, including, for example, c-Src, Fyn, Yes and Lyn kineses and the hematopoietic-restricted kineses Hck, Fgr, Lck and Blk.

The term “therapeutically effective amount” refers to the amount of the compound or pharmaceutical composition that will elicit the biological or medical response of a tissue, system, animal or human that is being sought by the researcher, veterinarian, medical doctor or other clinician, e.g., restoration or maintenance of vasculostasis or prevention of the compromise or loss or vasculostasis; reduction of tumor burden; reduction of morbidity and/or mortality.

The term ‘pharmaceutically acceptable” refers to the fact that the carrier, diluent or excipient must be compatible with the other ingredients of the formulation and not deleterious to the recipient thereof.

The terms “administration of a compound” or “administering a compound” refer to the act of providing a compound of the invention or pharmaceutical composition to the subject in need of treatment.

The term “protected” refers that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al., Protective Groups in Organic Synthesis, John Wiley & Sons, New York (1999).

The term “pharmaceutically acceptable salt” of a compound recited herein is an acid or base salt that is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC— (CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol or acetonitrile, is preferred. It will be apparent that each compound of formula (I) or formula (II) may, but need not, be formulated as a hydrate, solvate or non-covalent complex. In addition, the various crystal forms and polymorphs are within the scope of the present invention. Also provided herein are prodrugs of the compounds of formula (I) or formula (II).

The term of “prodrug” refers a compound that may not fully satisfy the structural requirements of the compounds provided herein, but is modified in vivo, following administration to a patient, to produce a compound of formula (I) or formula (II), or other formula provided herein. For example, a prodrug may be an acylated derivative of a compound as provided herein. Prodrugs include compounds wherein hydroxy, amine or thiol groups are bonded to any group that, when administered to a mammalian subject, cleaves to form a free hydroxy, amino, or thiol group, respectively. Examples of prodrugs include, but are not limited to, acetate, formate and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

Groups that are “optionally substituted” are unsubstituted or are substituted by other than hydrogen at one or more available positions. Such optional substituents include, for example, hydroxy, halogen, cyano, nitro, C₁-C₆ alkyl, C₂-C₆ alkenyl, C₂-C₆ alkynyl, C₁-C₆ alkoxy, C₂-C₆ alkyl ether, C₃-C₆ alkanone, C₂-C₆ alkylthio, amino, mono- or di-(C₁-C₆ alkyl)amino, C₁-C₆ haloalkyl, —COOH, —CONH₂, mono- or di-(C₁-C₆ alkyl)aminocarbonyl, —SO₂NH₂, and/or mono or di(C₁-C₆ alkyl) sulfonamido, as well as carbocyclic and heterocyclic groups.

Optional substitution is also indicated by the phrase “substituted with from 0 to X substituents,” where X is the maximum number of possible substituents. Certain optionally substituted groups are substituted with from 0 to 2, 3 or 4 independently selected substituents.

Preferred R₁ groups of formula (I) are listed below:

—CH₃, —CH₂CH₃, —CH₂CH(CH₃)₂, Cyclopropanyl, Ph, —CH₂Ph, —CH₂PhOMe.

Preferred R₂ groups of formula (I) are listed below:

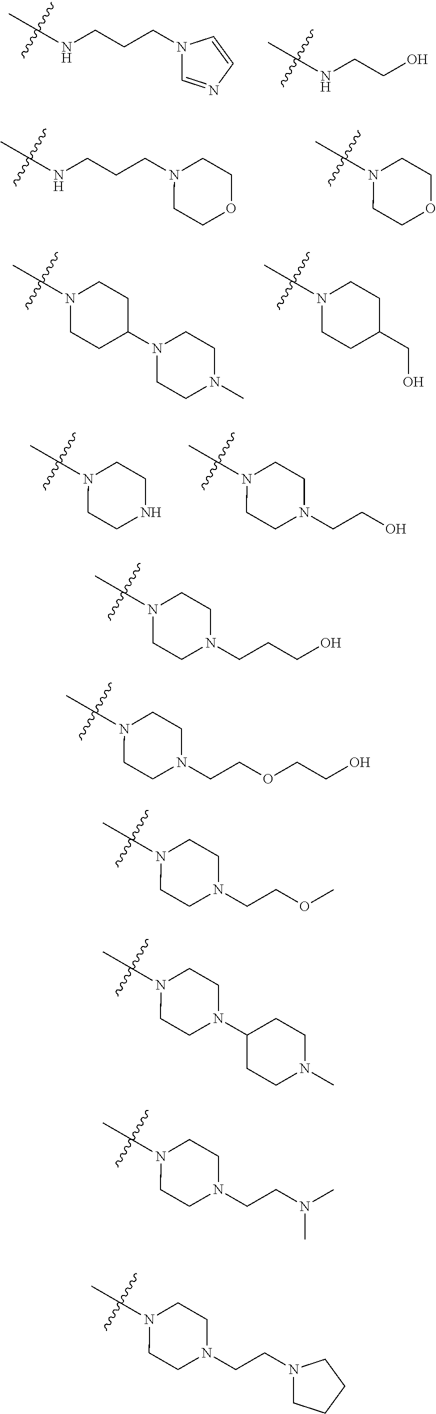

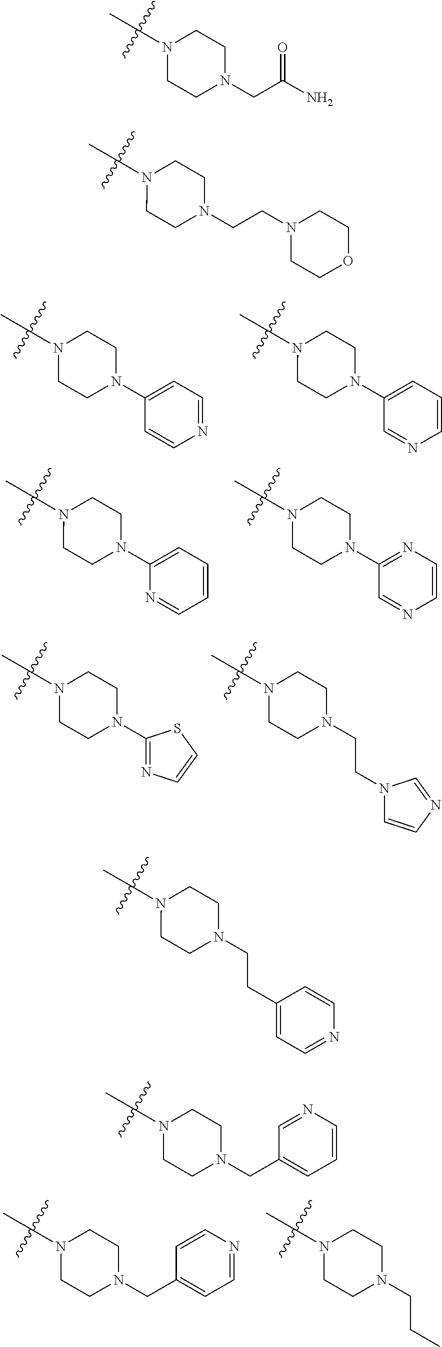

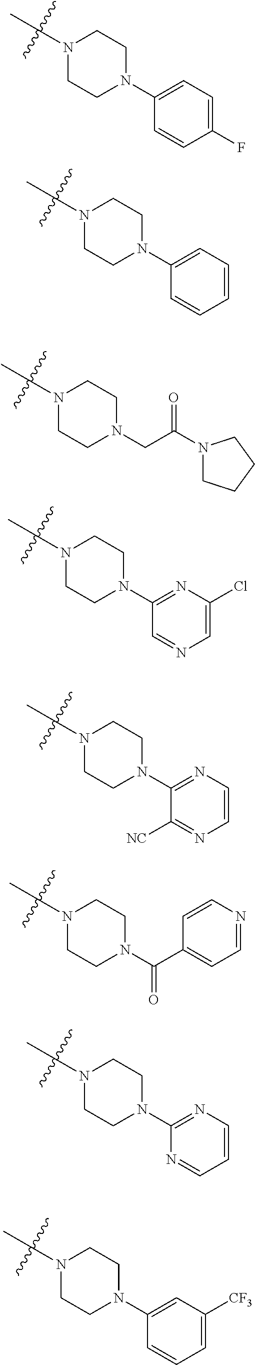

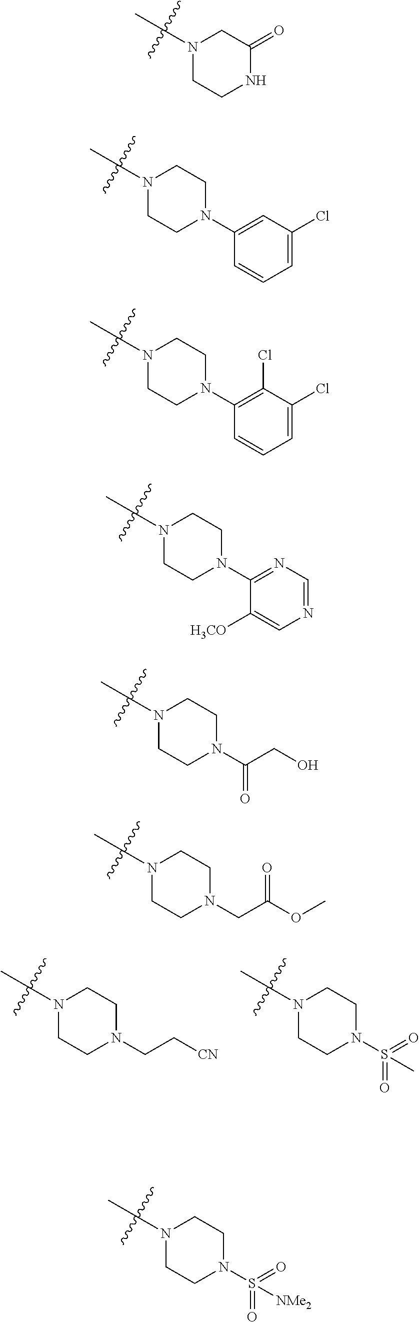

Preferred R₃ groups of formula (I) are listed below, wherein the substitute may be the specific ones as defined here or may be one or multiple substitutes as defined above:

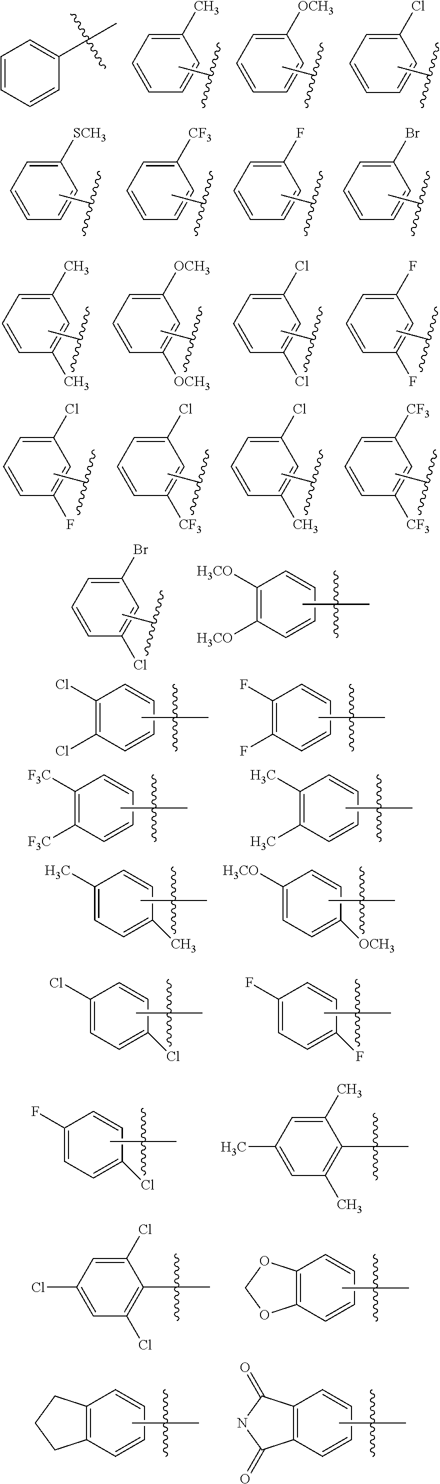

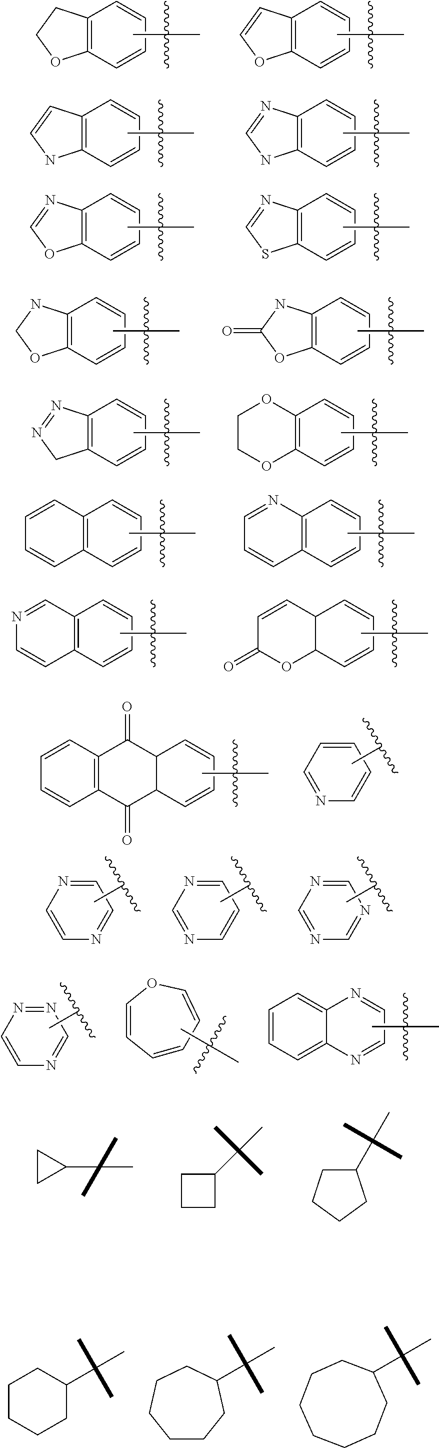

Preferred L is selected from O, S, SO, CO, SO₂, CO₂, NR₆, (CH₂)ₘ, m=0-3, CONR₄, NR₄CO, NR₄SO₂, SO₂NR₄, NR₄CO₂, NR₄SO₂NR₄, NR₄NR₄, OCONR₄, C(R₄)₂CONR₄, NR₄COC(R₄), C(R₄)₂SO, C(R₄)₂SO₂, C(R₄)₂SO₂NR₄, C(R₄)₂NR₄, C(R₄)₂NR₄CO, C(R₄)₂NR₄CO₂, C(R₄)═NNR₄, C(R₄)═N—O, C(R₄)₂NR₄NR₄, C(R₄)₂NR₄SO₂NR₄, C(R₄)₂NR₄CONR₄.

R₄ is independently selected from hydrogen or an optionally substituted C₁₄ aliphatic group.

Preferably, the compounds of the invention may be compounds of formula (I) wherein

R₁ groups of formula (I) are listed below:

—CH₃, —CH₂CH₃, —CH₂CH(CH₃)₂, Cyclopropanyl, Ph, —CH₂Ph, —CH₂PhOMe.

R₂ is selected from:

(i) amino, alkyl amino, aryl amino, heteroaryl amino;

(ii) C₁-C₆ alkyl, C₂-C₆ alkenyl, C₂-C₆ alkynyl;

(iii) heterocyclic, herteroaryl; and

(iv) groups of the formula (Ia):

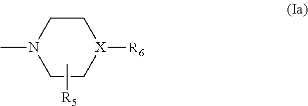

wherein:

R₅ represents hydrogen, C₁-C₄ alkyl, oxo;

X is CH, when R₆ is hydrogen; or X—R₆ is O; or X is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl) sulfonamido, and mono- and di-(C₁-C₆alkyl)aminocarbonyl, each of which is substituted with from 0 to 4 substituents independently chosen from halogen, hydroxy, cyano, amino, —COOH and oxo;

L represents O, S, SO, CO, SO₂, CO₂, NR₆, (CH₂)ₘ, m=0-3, CONR₄, NR₄CO, NR₄SO₂, SO₂NR₄, NR₄CO₂, NR₄COR₄, NR₄SO₂NR₄, NR₄NR₄, OCONR₄, C(R₄)₂CONR₄, NR₄COC(R₄), C(R₄)₂SO, C(R₄)₂SO₂, C(R₄)₂SO₂NR₄, C(R₆)₂NR₄, C(R₄)₂NR₄CO, C(R₄)₂NR₄CO₂, C(R₄)═NNR₄, C(R₄)═N—O, C(R₄)₂NR₄NR₄, C(R₄)₂NR₄SO₂NR₄, C(R₄)₂NR₄CONR₄.

R₄ is independently selected from hydrogen or an optionally substituted C₁₋₄ aliphatic group.

R₃ is selected from:

(i) C₁-C₆ alkyl, C₂-C₆ alkenyl, C₂-C₆ alkynyl;

(ii) heterocyclic,

(iii) Ar.

Ar represents heteroaryl or aryl, each of which is substituted with from 0 to 4 substituents independently chosen from:
(1) halogen, hydroxy, amino, cyano, —COOH, —SO₂NH₂, oxo, nitro and alkoxycarbonyl; and
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- and di-(C₁-C₆alkyl)amino, C₁-C₆ alkylsulfonyl, mono- and di-(C₁-C₆alkyl) sulfonamido and mono- and di-(C₁-C₆alkyl)aminocarbonyl; phenylC₀-C₄alkyl and (4- to 7-membered heterocycle)-C₀-C₄alkyl, each of which is substituted with from 0 to 4 secondary substituents independently chosen from halogen, hydroxy, cyano, oxo, imino, C₁-C₄alkyl, C₁-C₄alkoxy and C₁-C₄haloalkyl.

A, B, W independently represents S, or O, or NR₄, or CR₄;

K is selected from

i) absence;

ii) O, S, SO, SO₂;

iii) (CH₂)ₘ, m=0-3, O(CH₂)ₚ, p=1-3, (CH₂)_{q}O, q=1-3.

iv) NR_{S}

R₇ represents hydrogen, alkyl, cycloalkyl, alkenyl, alkynyl, alkylthio, aryl, arylalkyl.

More preferably, the compounds of the invention may be compounds of formula (I) wherein

R₁ represents —CH₃, —CH₂CH₃, —CH₂CH(CH₃)₂, Cyclopropanyl, Ph.

R₂ is selected from:

amino, alkyl amino, aryl amino, heteroaryl amino and groups of the formula (Ia):

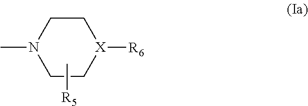

wherein:

R₅ represents hydrogen, C₁-C₄ alkyl, oxo;

X is CH, when R₆ is hydrogen; or X—R₆ is O; or X is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl) sulfonamido, and mono- and di-(C₁-C₆alkyl)aminocarbonyl, each of which is substituted with from 0 to 4 substituents independently chosen from halogen, hydroxy, cyano, amino, —COOH and oxo;

L represents O, S, CO, SO₂, CO₂, NR₄, (CH₂)ₘ, m=0-3, CONR₄, NR₄CO, NR₄SO₂, SO₂NR₄, NR₄CO₂, NR₄COR₄, NR₄SO₂NR₄, NR₄NR₄, OCONR₄, C(R₄)₂CONR₄, NR₄COC(R₄), C(R₄)₂SO, C(R₄)₂SO₂, C(R₄)₂SO₂NR₄, C(R₄)₂NR₄, C(R₄)₂NR₄CO, C(R₄)₂NR₄CO₂, C(R₄)═NNR₄.

R₄ is independently selected from hydrogen or an optionally substituted C₁₋₄ aliphatic group.

R₃ is selected from heteroaryl or aryl, each of which is substituted with from 0 to 4 substituents independently chosen from:
(1) halogen, hydroxy, amino, cyano, —COOH, —SO₂NH₂, oxo, nitro and alkoxycarbonyl; and
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- and di-(C₁-C₆alkyl)amino, C₁-C₆ alkylsulfonyl, mono- and di-(C₁-C₆alkyl) sulfonamido and mono- and di-(C₁-C₆alkyl)aminocarbonyl; phenylC₀-C₄alkyl and (4- to 7-membered heterocycle)-C₀-C₄alkyl, each of which is substituted with from 0 to 4 secondary substituents independently chosen from halogen, hydroxy, cyano, oxo, imino, C₁-C₄alkyl, C₁-C₄alkoxy and C₁-C₄haloalkyl.

A, B, W independently represents S, or O, or NR₄, or CR₄;

K is selected from

i) absence;

ii) O, S, SO, SO₂;

iii) (C₁₋₁₂)ₘ, m=0-3, O(CH₂)ₚ, p=1-3, (CH₂)_{q}O, q=1-3.

iv) NR_{S}

R₇ represents hydrogen, alkyl, cycloalkyl, alkenyl, alkynyl, alkylthio, aryl, arylalkyl.

Most preferably, R₁ represents, —CH₃, —CH₂CH₃;

R₂ is selected from:

alkyl amino, aryl amino, heteroaryl amino and groups of the formula (Ia):

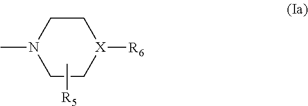

wherein:

R₅ represents hydrogen, C₁-C₄ alkyl, oxo;

X is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl) sulfonamido, and mono- and di-(C₁-C₆alkyl)aminocarbonyl, each of which is substituted with from 0 to 4 substituents independently chosen from halogen, hydroxy, cyano, amino, —COOH and oxo;

L represents O, S, NR₄, (CH₂)ₘ, m=0-3, CONR₄, NR₄CO, NR₄SO₂, SO₂NR₄, NR₄CO₂, NR₄COR₆, NR₄SO₂NR₄, C(R₄)₂CONR₄, C(R₄)₂SO₂, C(R₄)₂SO₂NR₄, C(R₄)₂NR₄, C(R₄)₂NR₄CO;

R₄ is independently selected from hydrogen or an optionally substituted C₁₋₄ aliphatic group.

R₃ is selected from heteroaryl or aryl, each of which is substituted with from 0 to 4 substituents independently chosen from:
(1) halogen, hydroxy, amino, cyano, —COOH, —SO₂NH₂, oxo, nitro and alkoxycarbonyl; and
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- and di-(C₁-C₆alkyl)amino, C₁-C₆ alkylsulfonyl, mono- and di-(C₁-C₆alkyl) sulfonamido and mono- and di-(C₁-C₆alkyl)aminocarbonyl; phenylC₀-C₄alkyl and (4- to 7-membered heterocycle)C₀-C₄alkyl, each of which is substituted with from 0 to 4 secondary substituents independently chosen from halogen, hydroxy, cyano, oxo, imino, C₁-C₄alkyl, C₁-C₄alkoxy and C₁-C₄haloalkyl.

A, B, W independently represents S, or O, or NR4, or CR4;

K is selected from

i) absence;

ii) O, S;

iii) NR7; R7 represents hydrogen, alkyl.

Preferred heterocyclic groups in compounds of formula (I) include

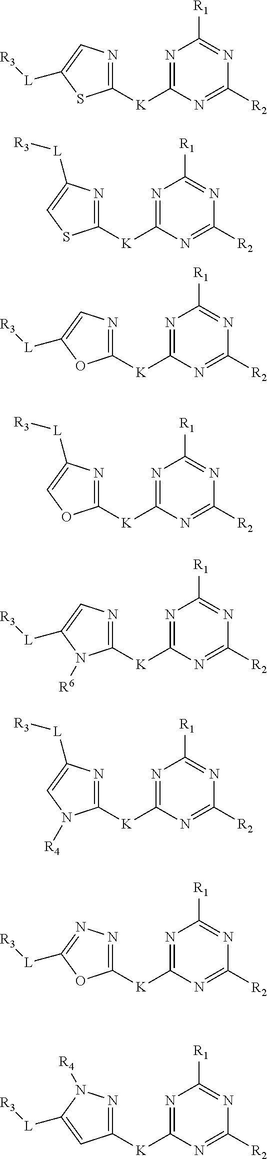

Which optionally may be substituted.

According to another embodiment, the present invention relates to a compound of formula (I) wherein R₁ is methyl.

According to another embodiment, the present invention relates to a compound of formula (I) wherein R₁ is ethyl.

According to another embodiment, the present invention relates to a compound of formula (I) wherein R₁ is isopropyl.

According to another embodiment, the present invention relates to a compound of formula (I) wherein R₁ is phenyl.

According to another embodiment, the present invention relates to a compound of formula (I) wherein R₁ is cyclopropanyl.

According to another embodiment, the present invention relates to a compound of formula (I) wherein R₂ is methyl-piperazinyl.

According to another embodiment, the present invention relates to a compound of formula (I) wherein R₂ is (2-hydroxylethyl)-piperazinyl.

According to another embodiment, the present invention relates to a compound of formula (I) wherein L is oxygen.

According to another embodiment, the present invention relates to a compound of formula (I) wherein L is CO.

According to another embodiment, the present invention relates to a compound of formula (I) wherein L is NHCO.

According to another embodiment, the present invention relates to a compound of formula (I) wherein L is CONH.

According to another embodiment, the present invention relates to a compound of formula (I) wherein L is NR₄COC(R₄).

According to another embodiment, the present invention relates to a compound of formula (I) wherein L is NH.

According to another embodiment, the present invention relates to a compound of formula (I) wherein L is S.

According to another embodiment, the present invention relates to a compound of formula (I) wherein L is SO.

According to another embodiment, the present invention relates to a compound of formula (I) wherein L is SO₂.

According to another embodiment, the present invention relates to a compound of formula (I) wherein A is N.

Examples of specific compounds of the present invention are those compounds defined in the following:

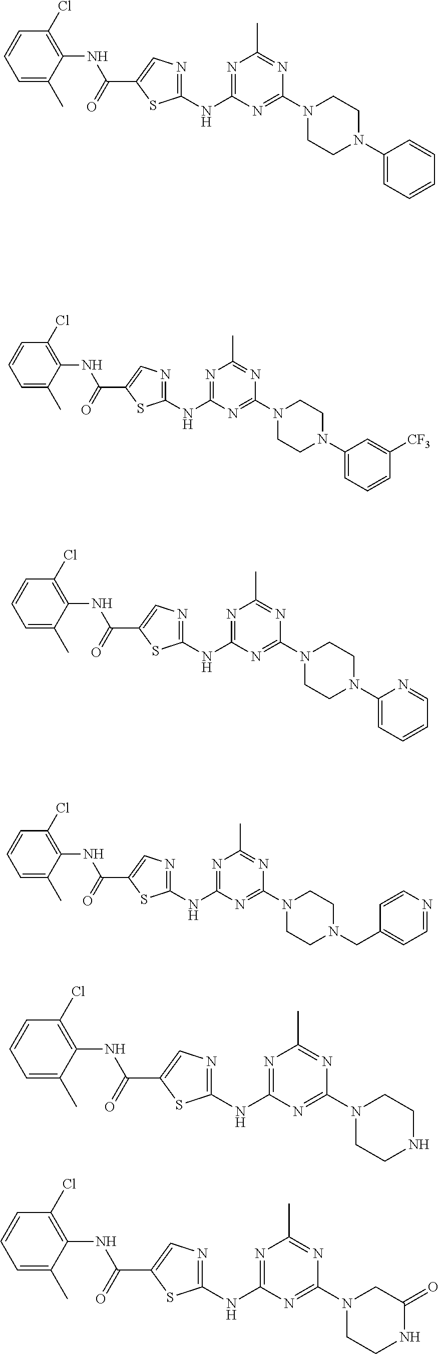

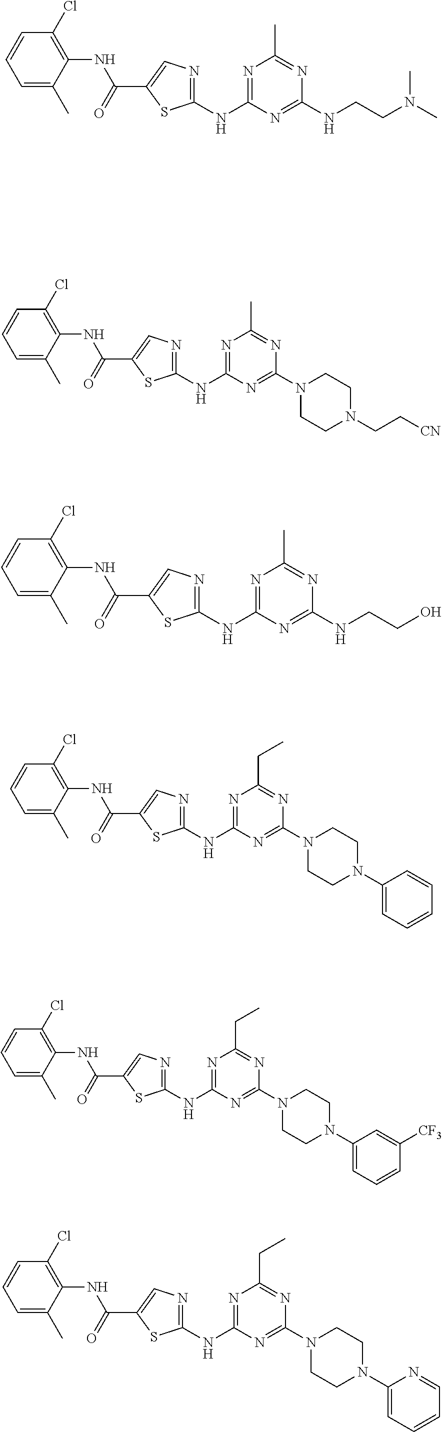

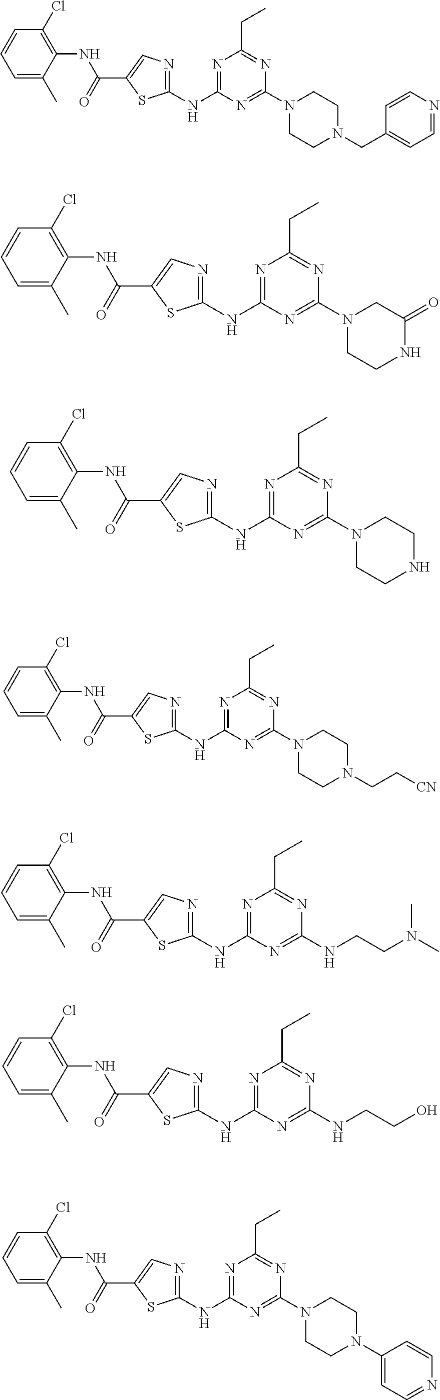

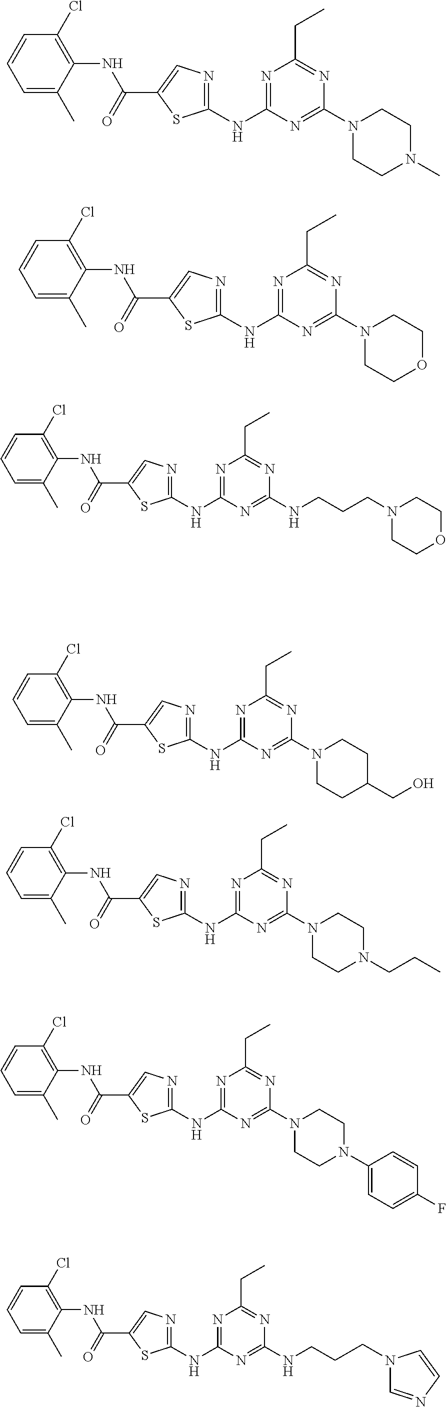

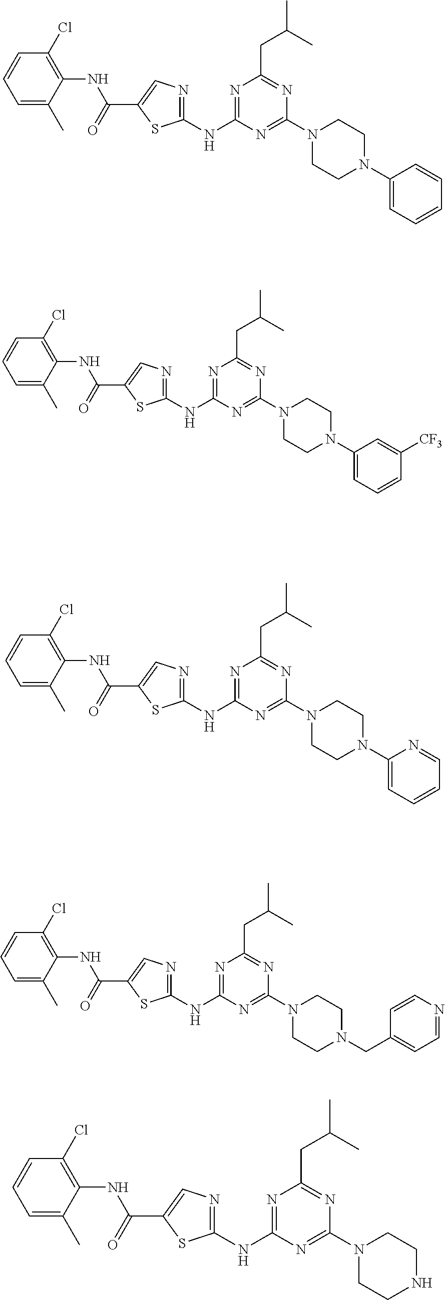

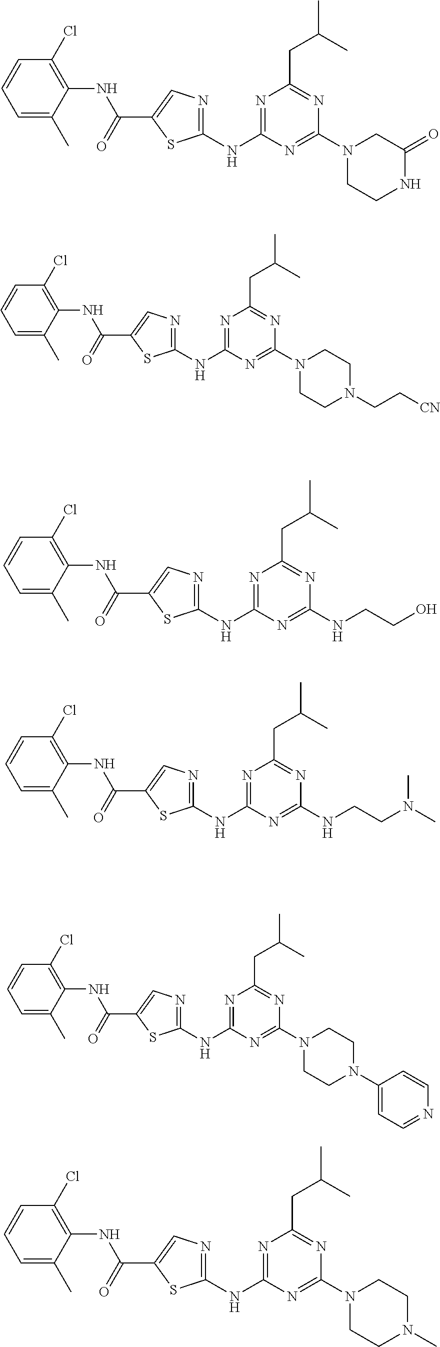

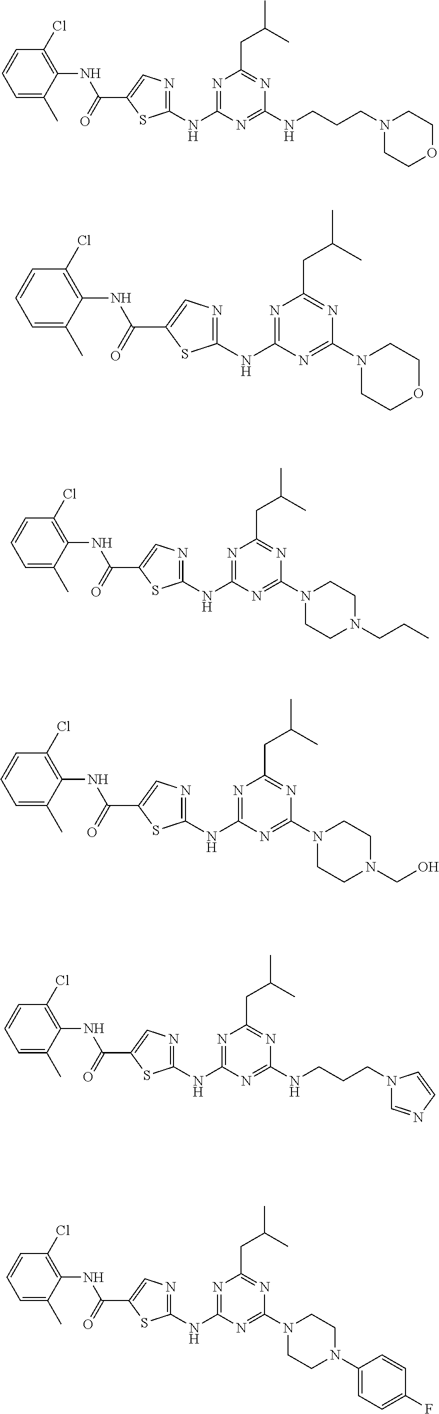

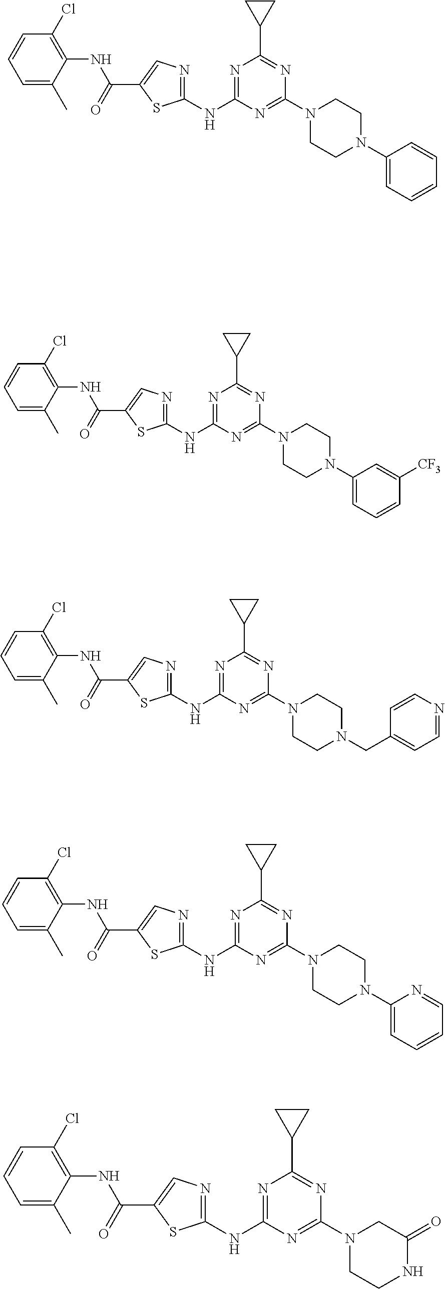

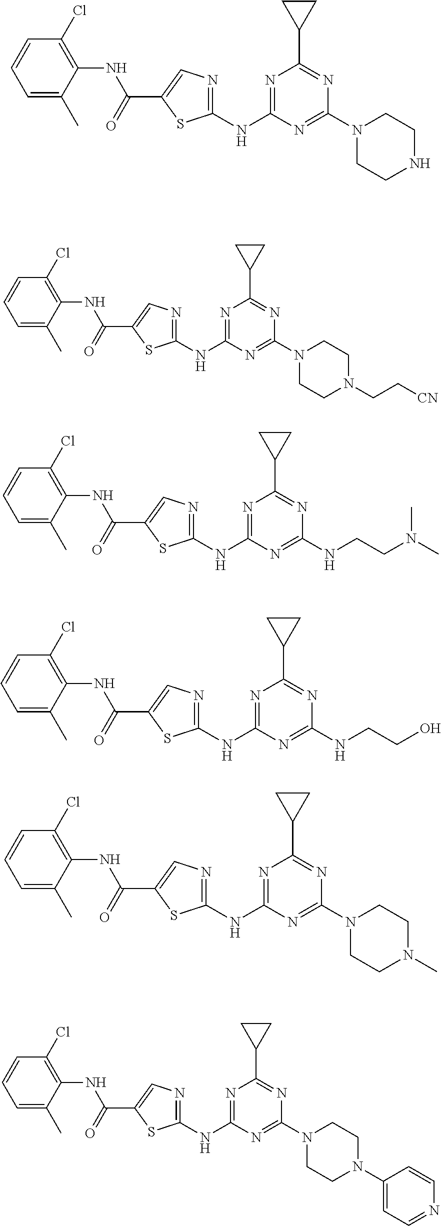

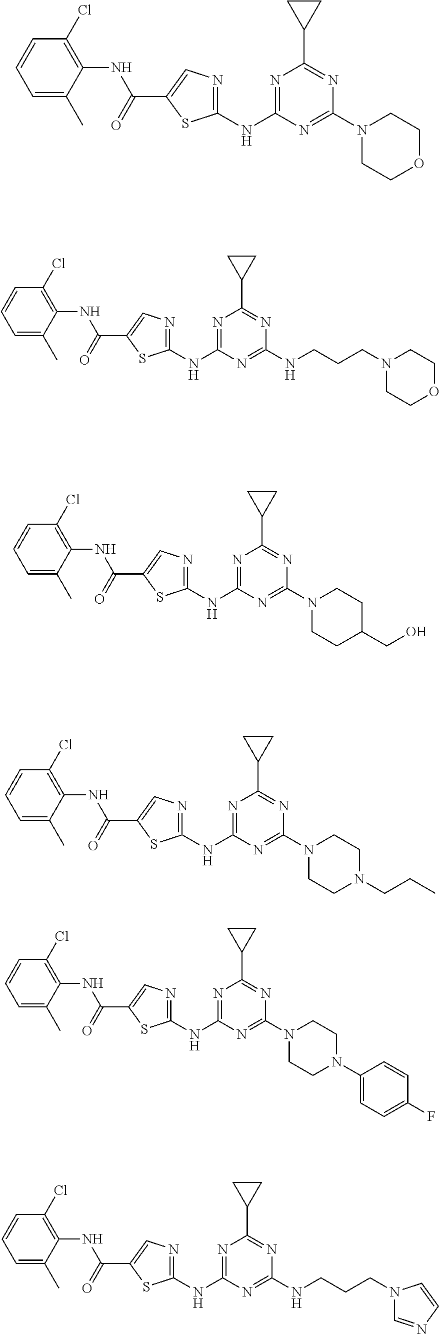

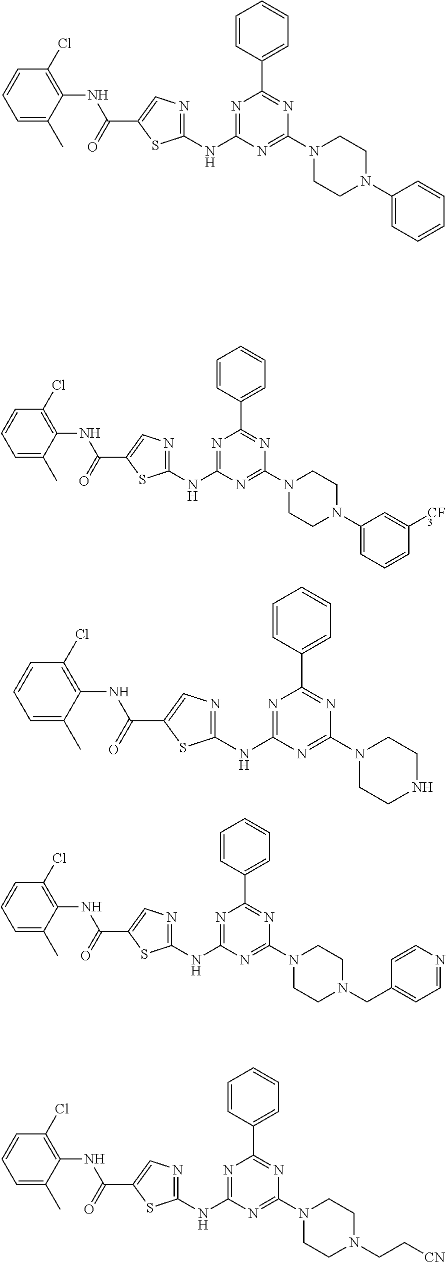

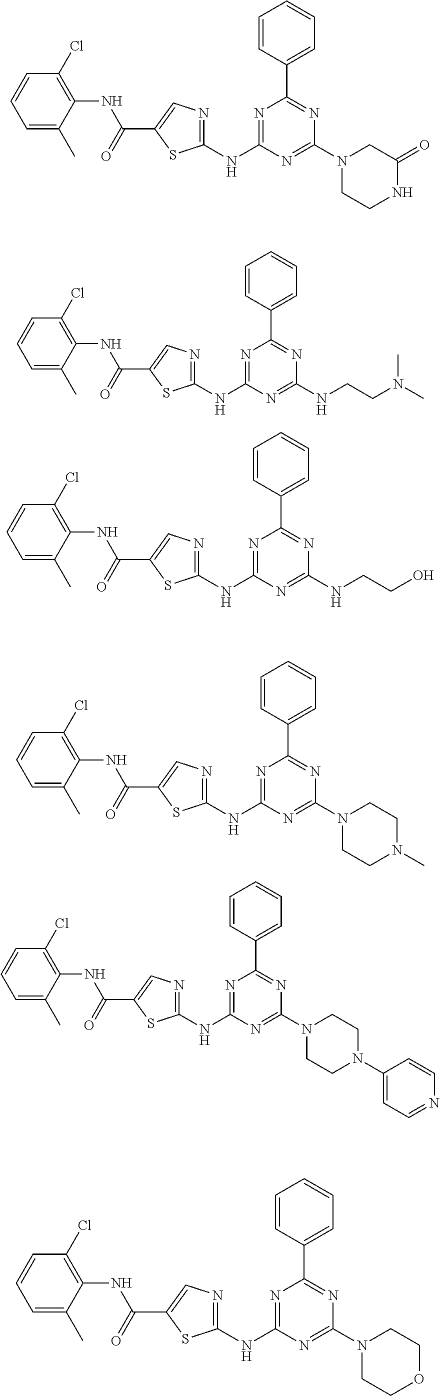

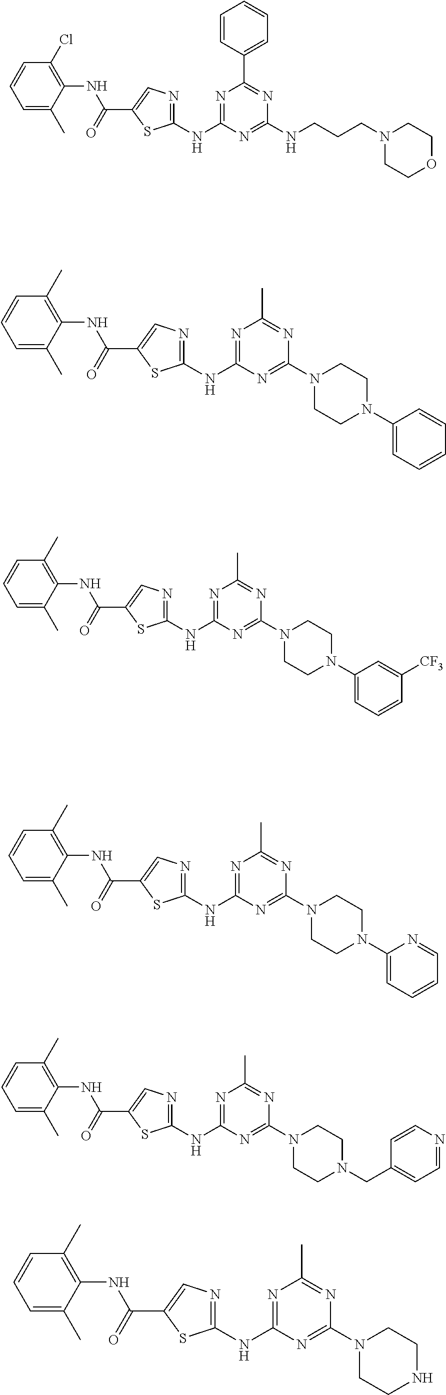

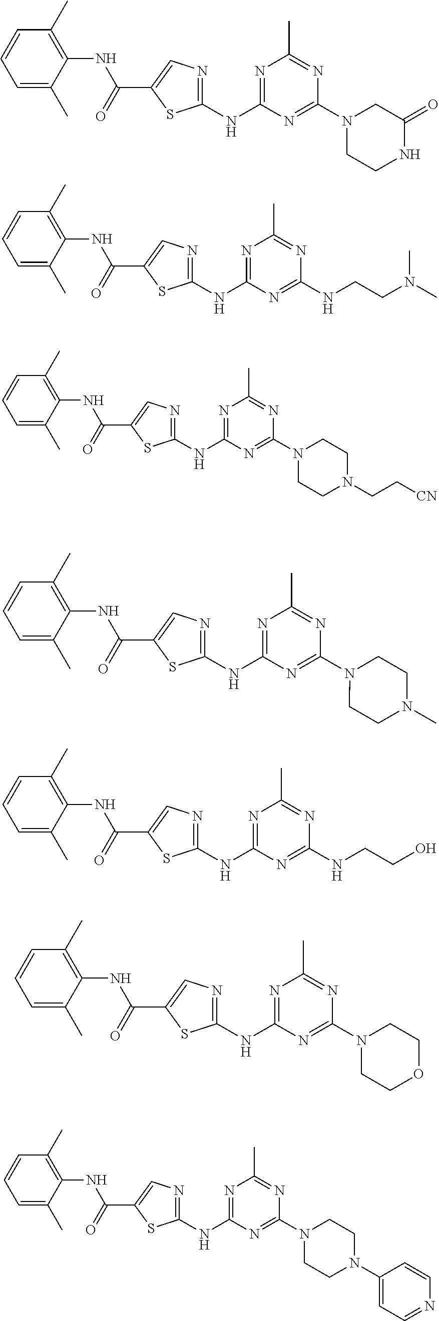

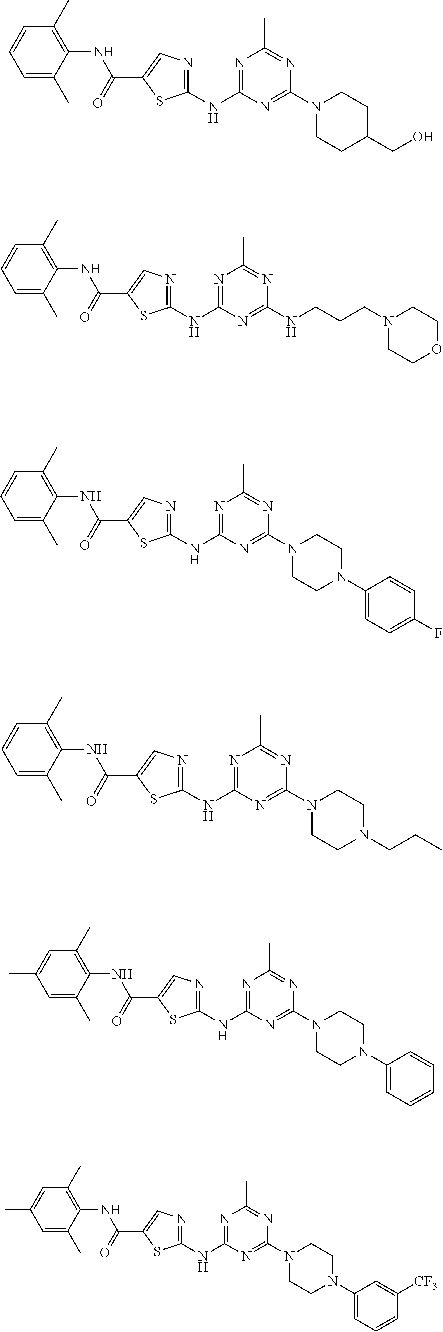

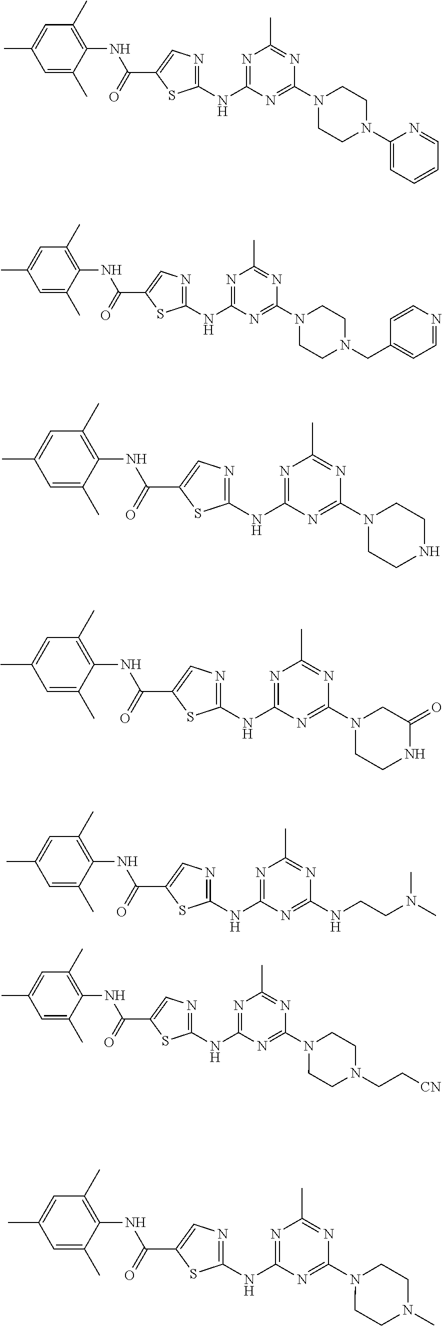

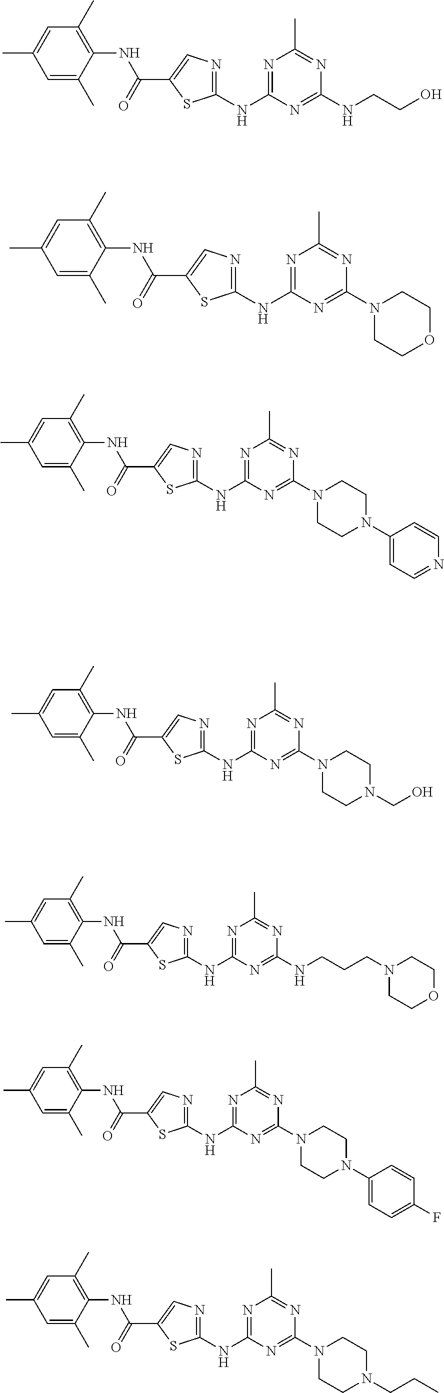

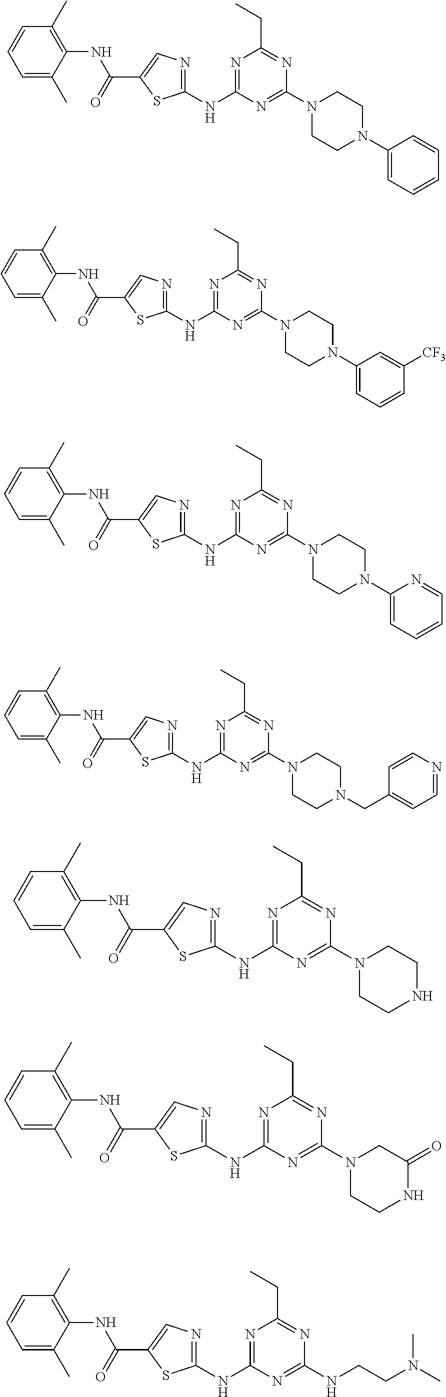

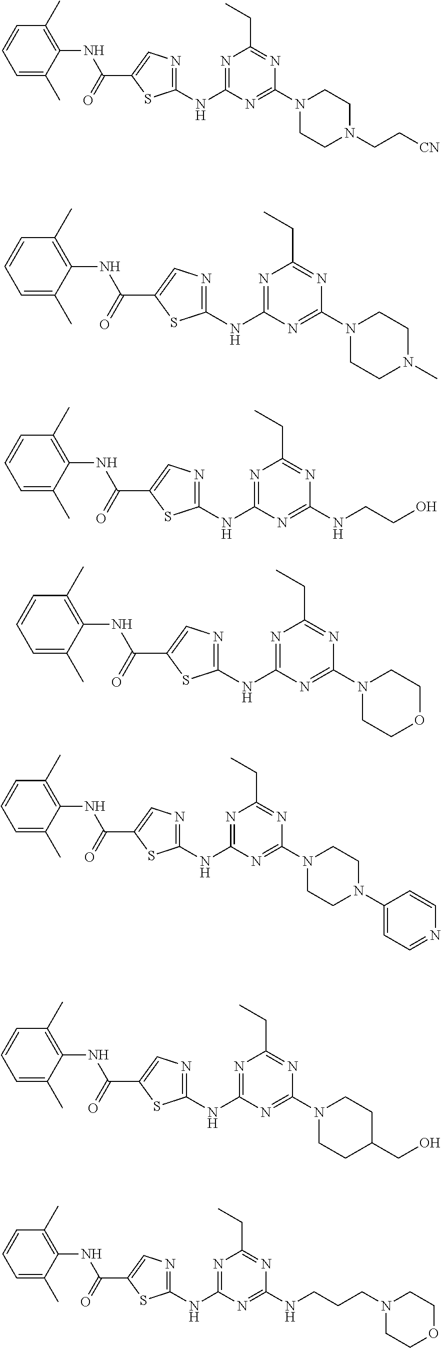

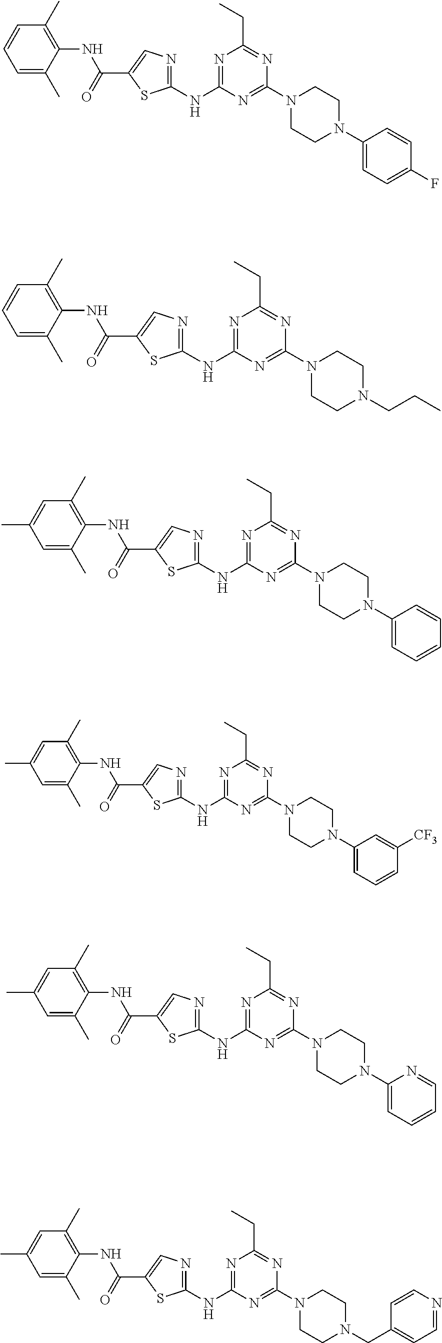

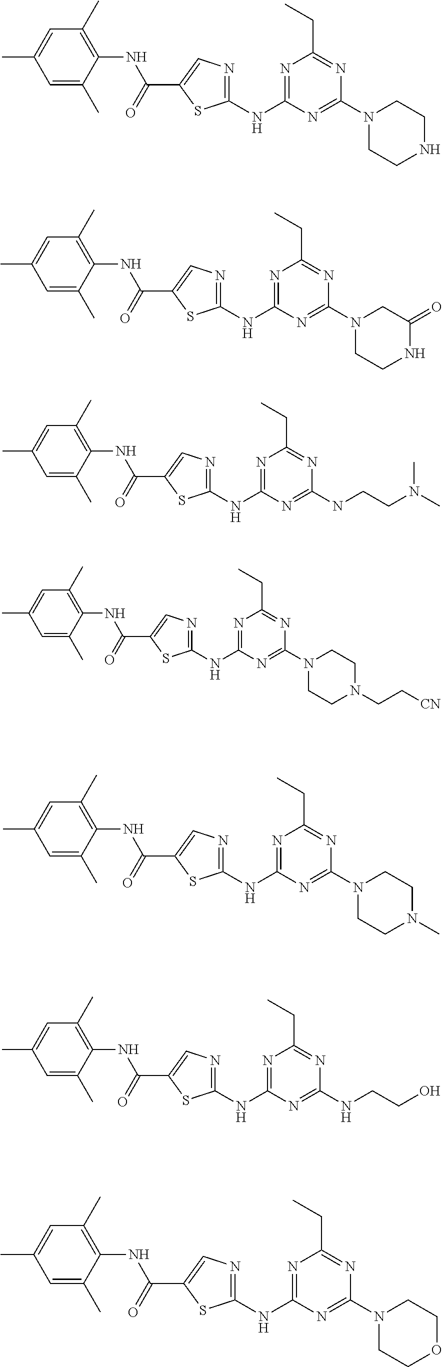

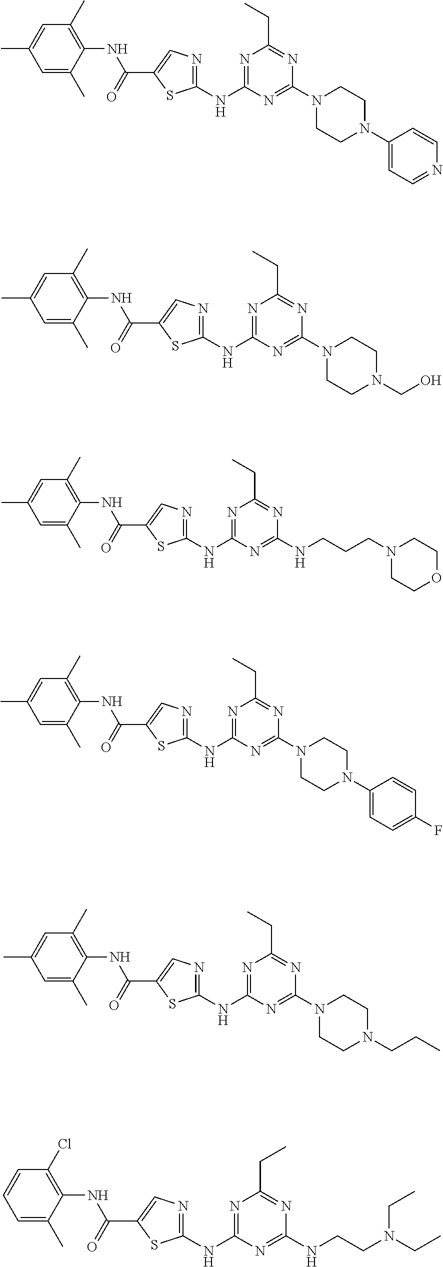

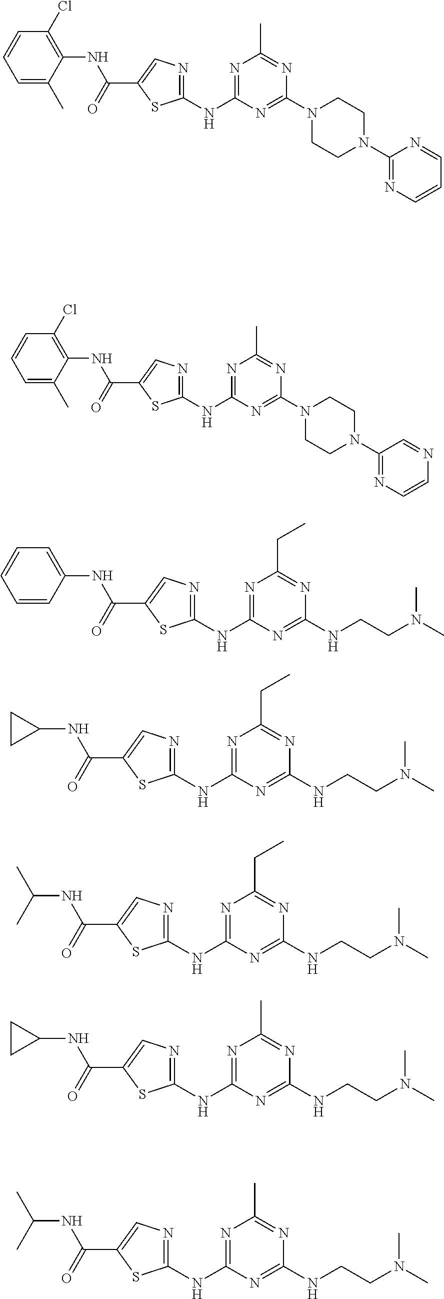

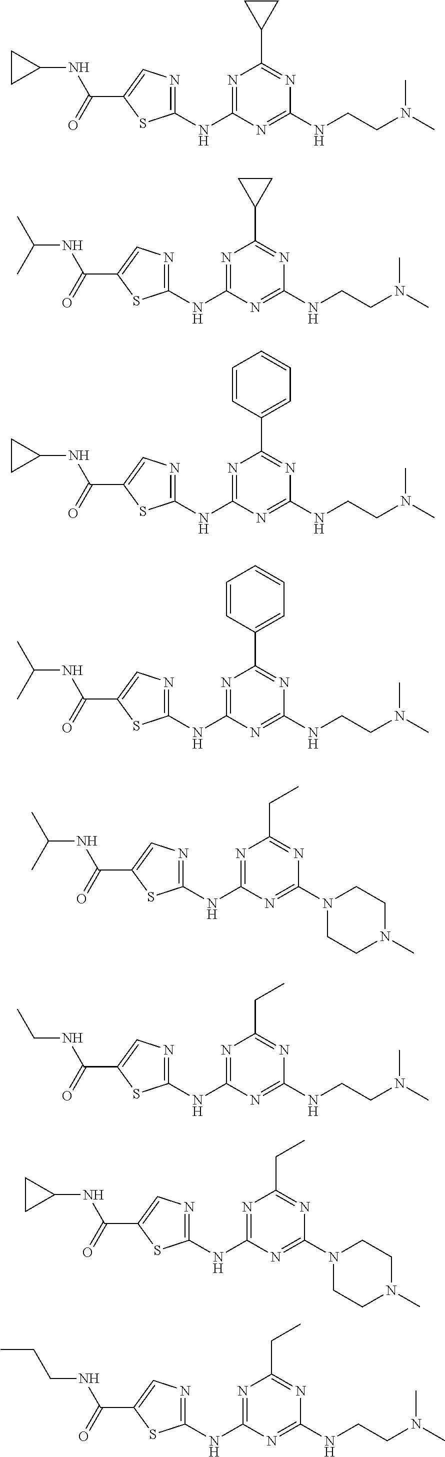

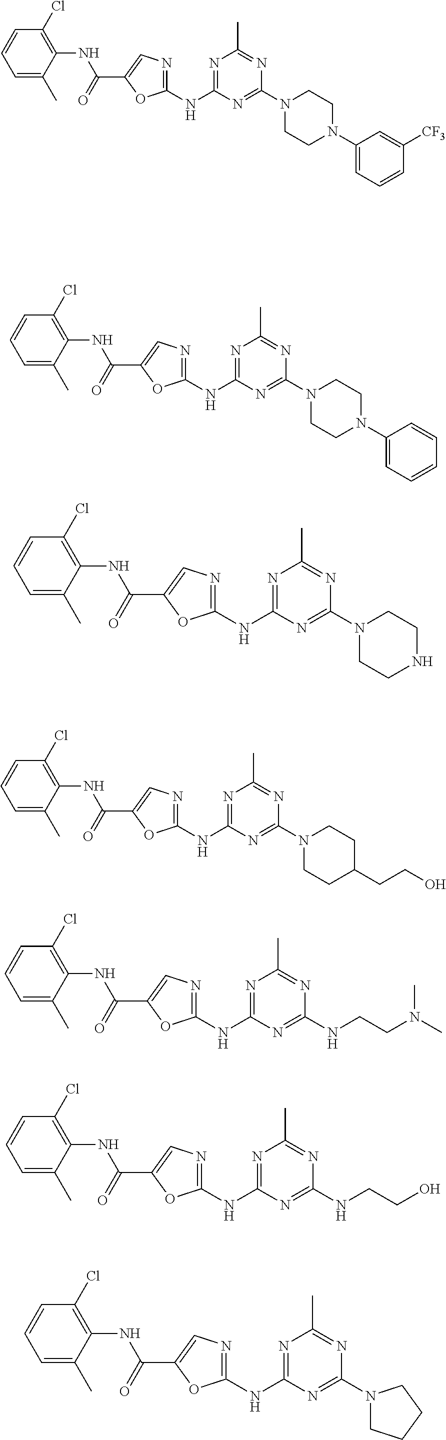

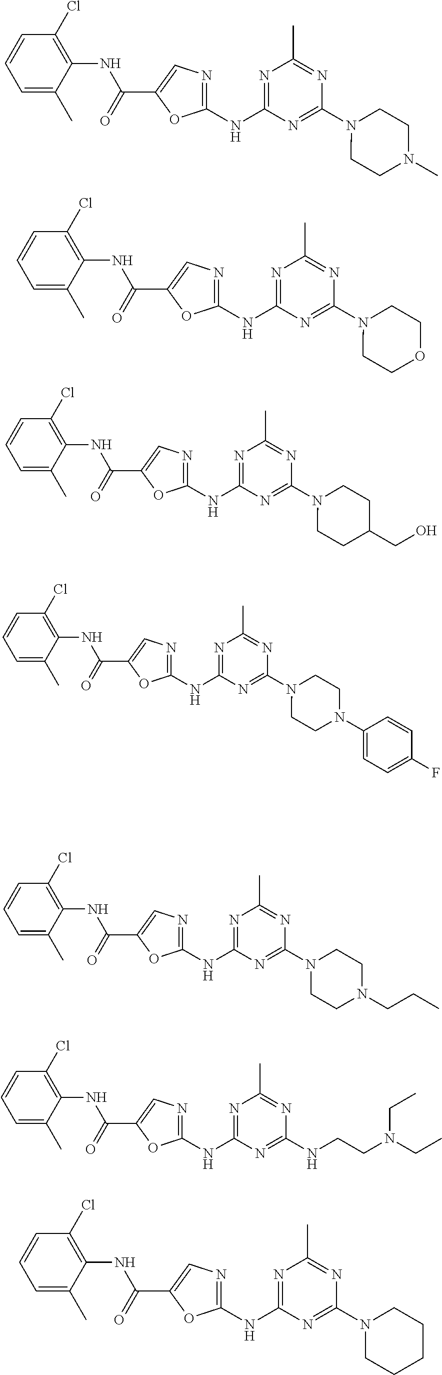

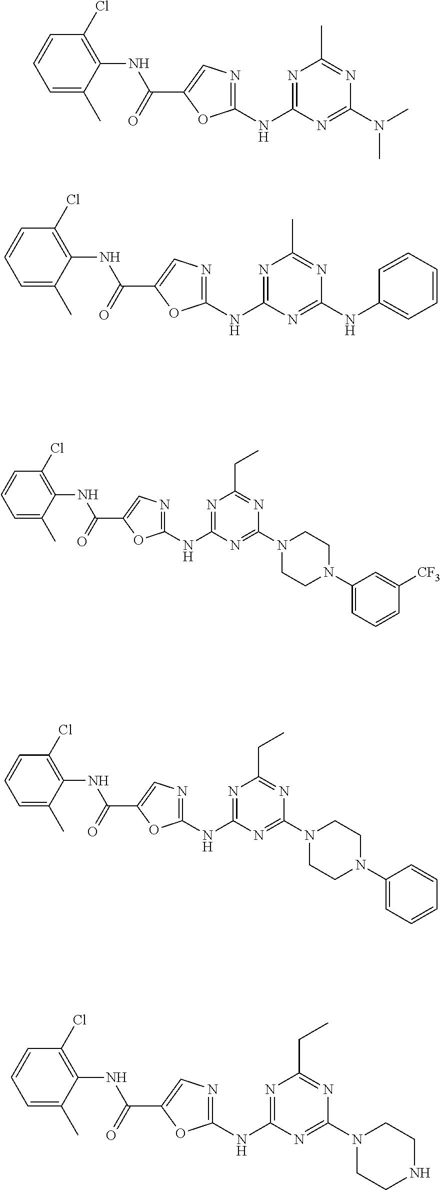

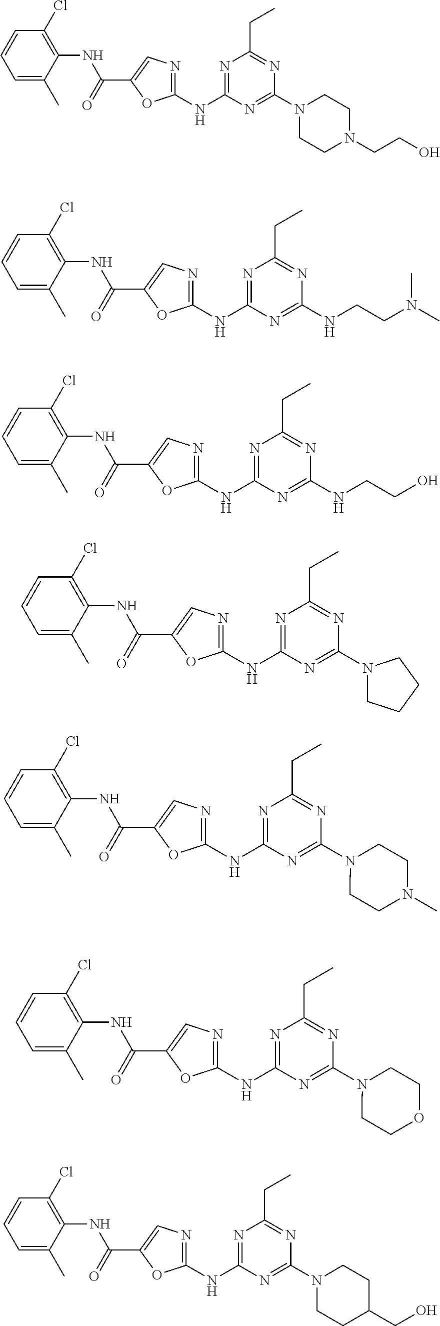

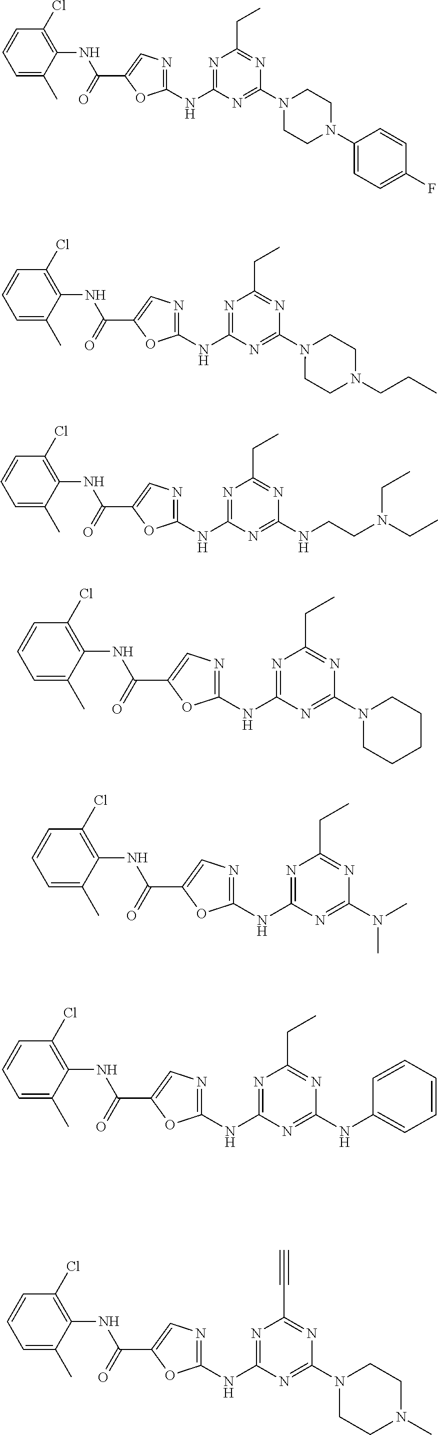

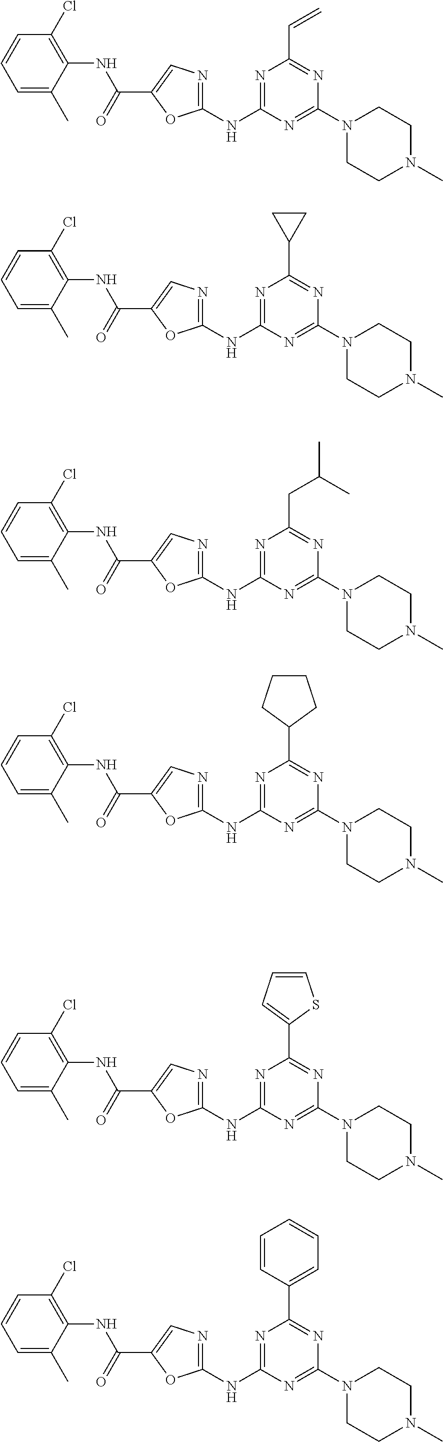

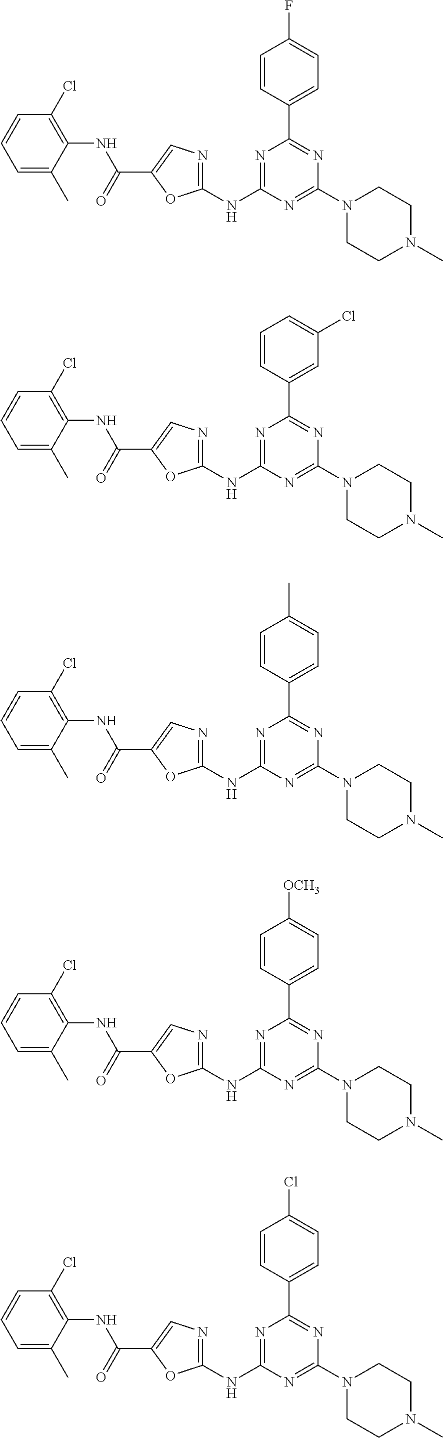

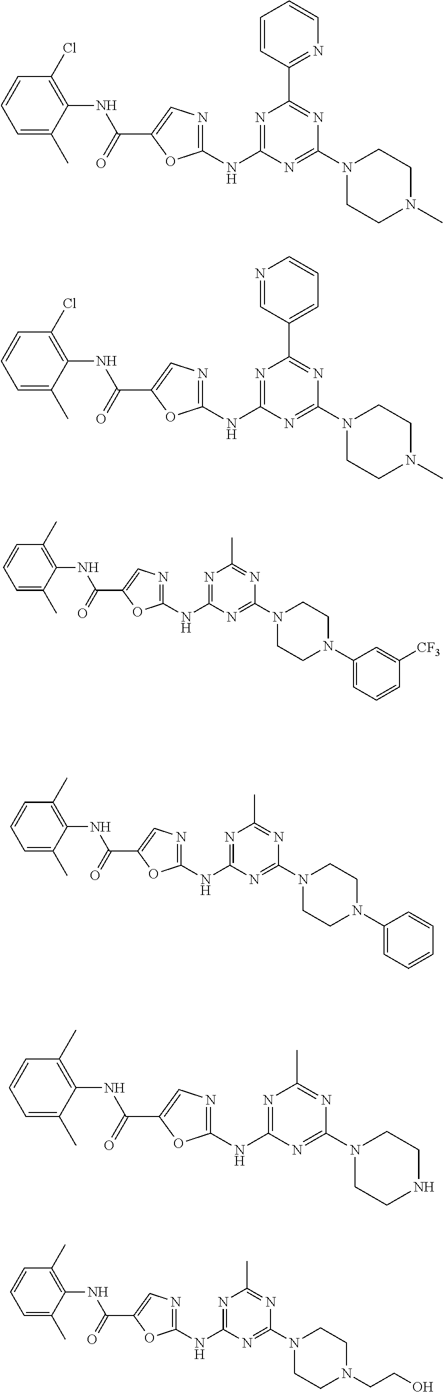

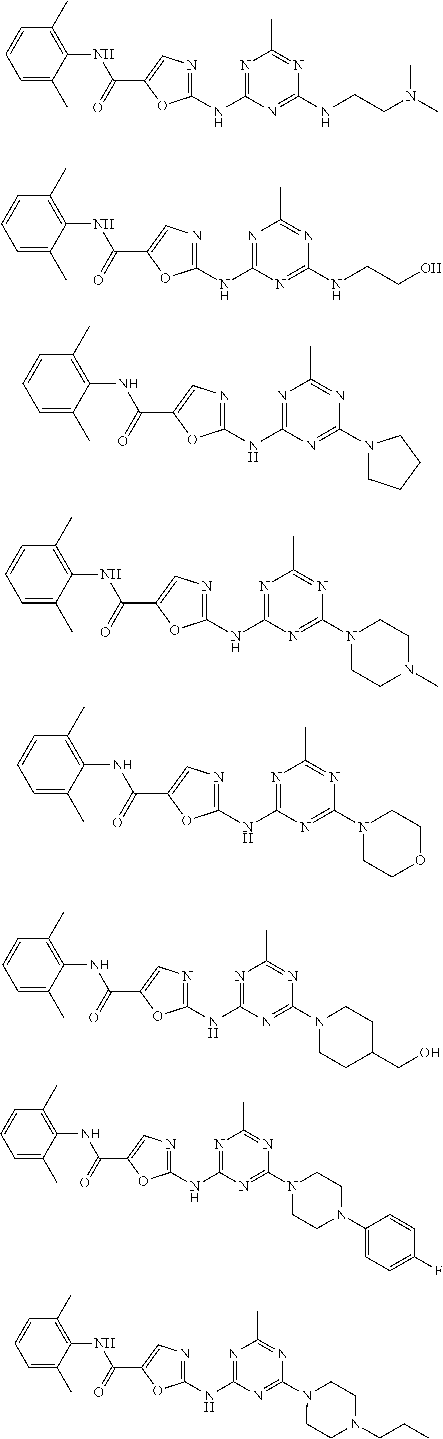

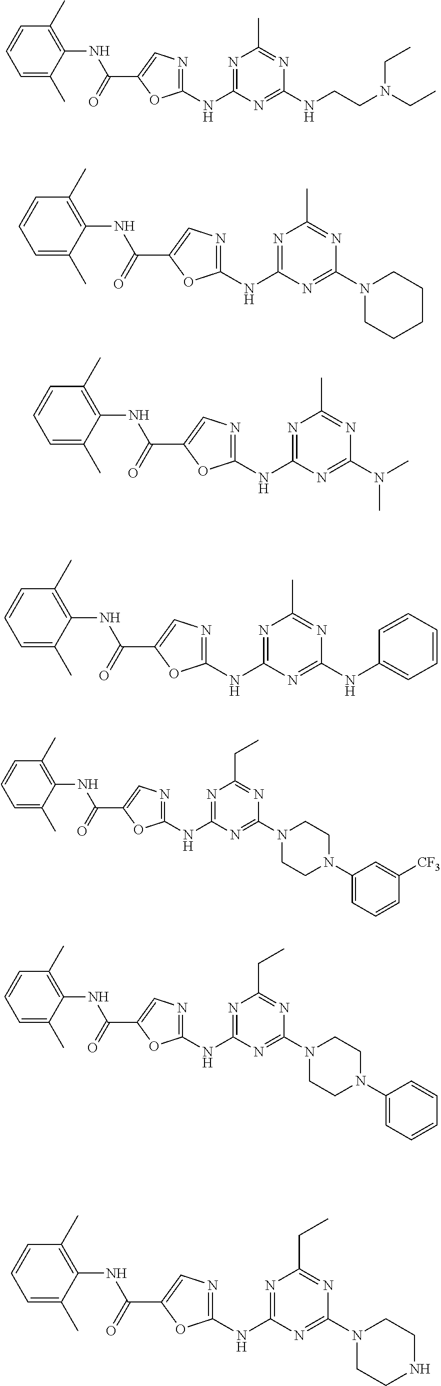

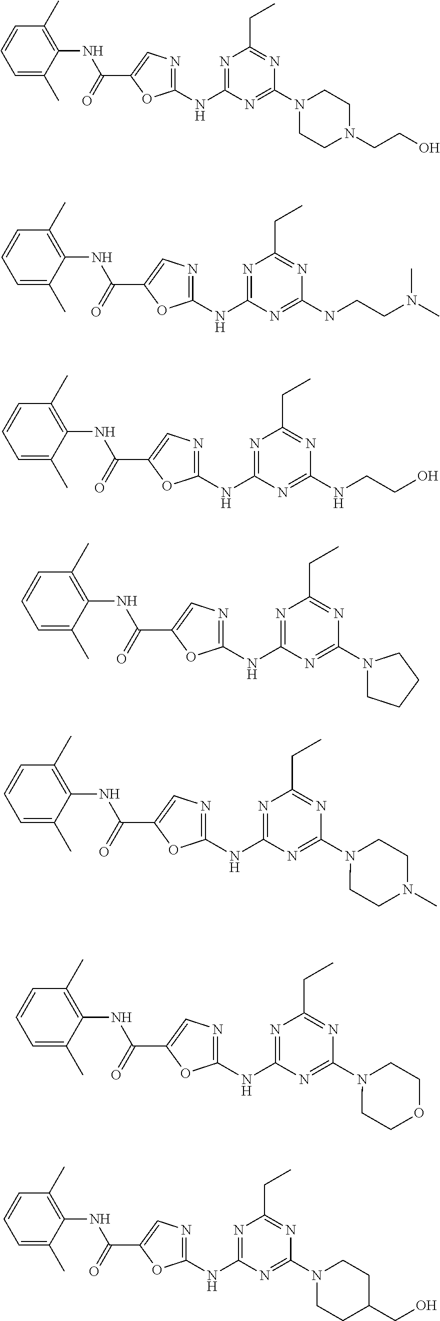

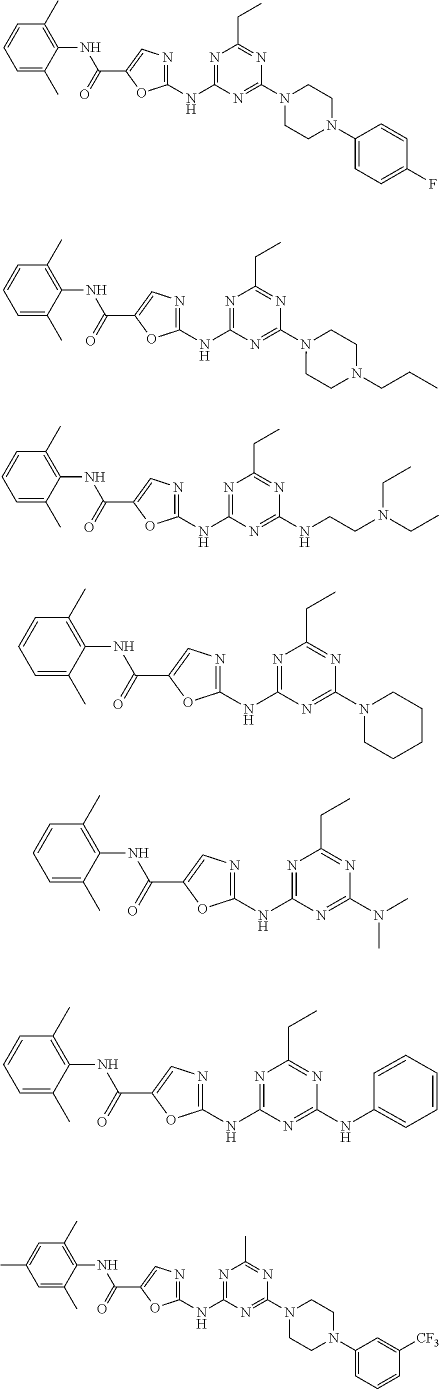

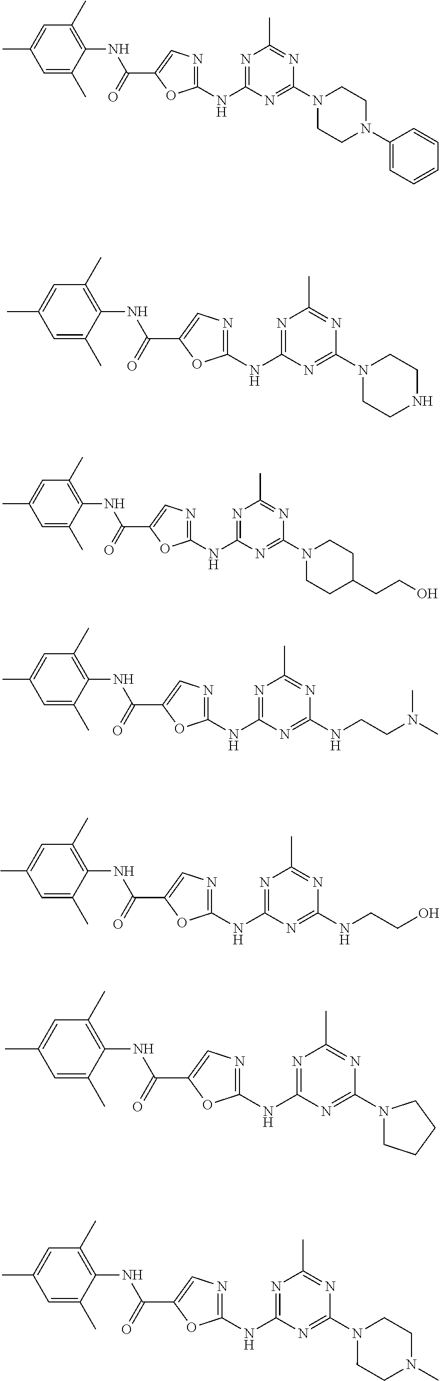

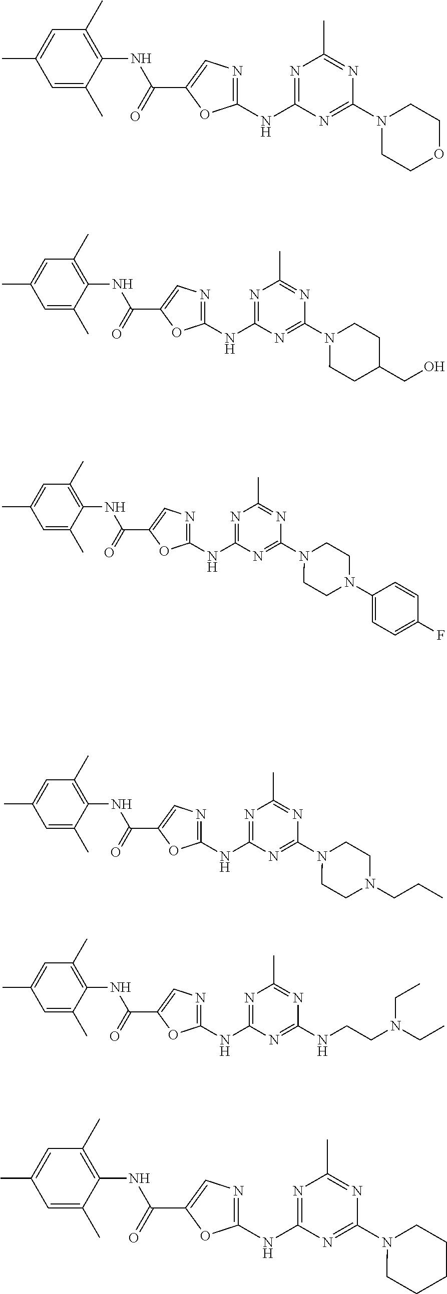

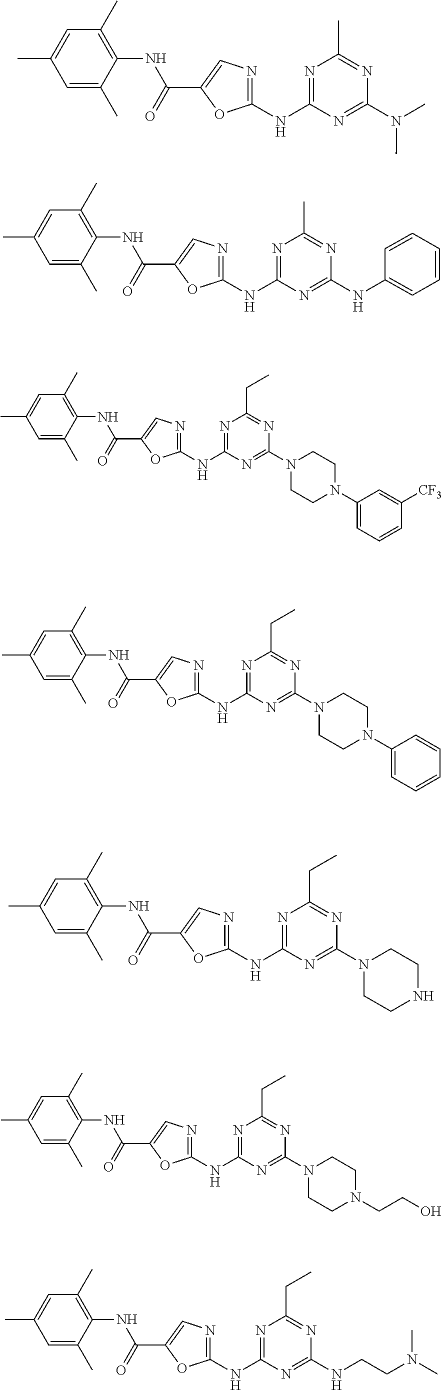

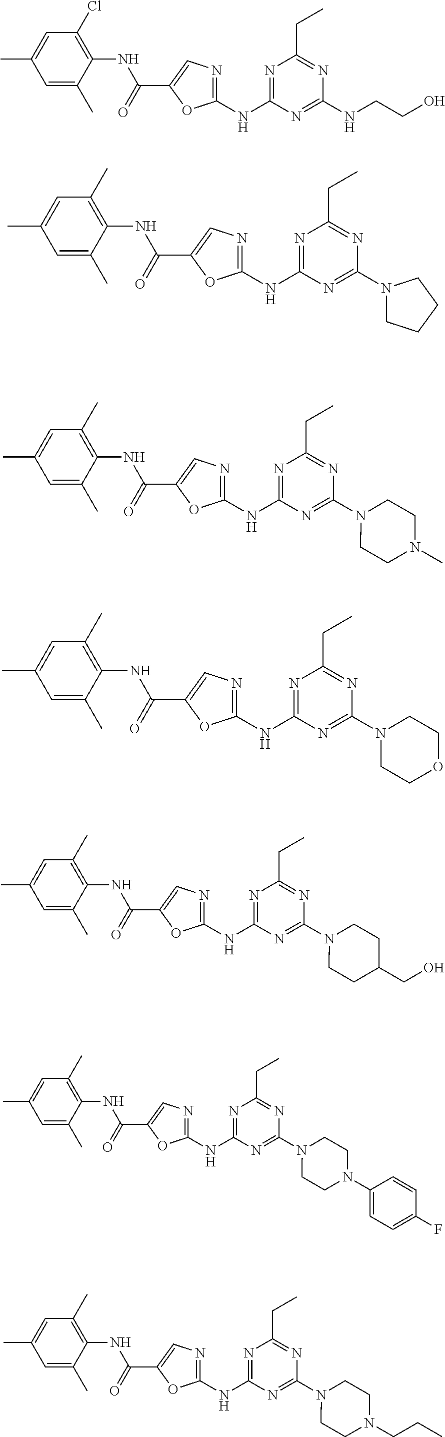

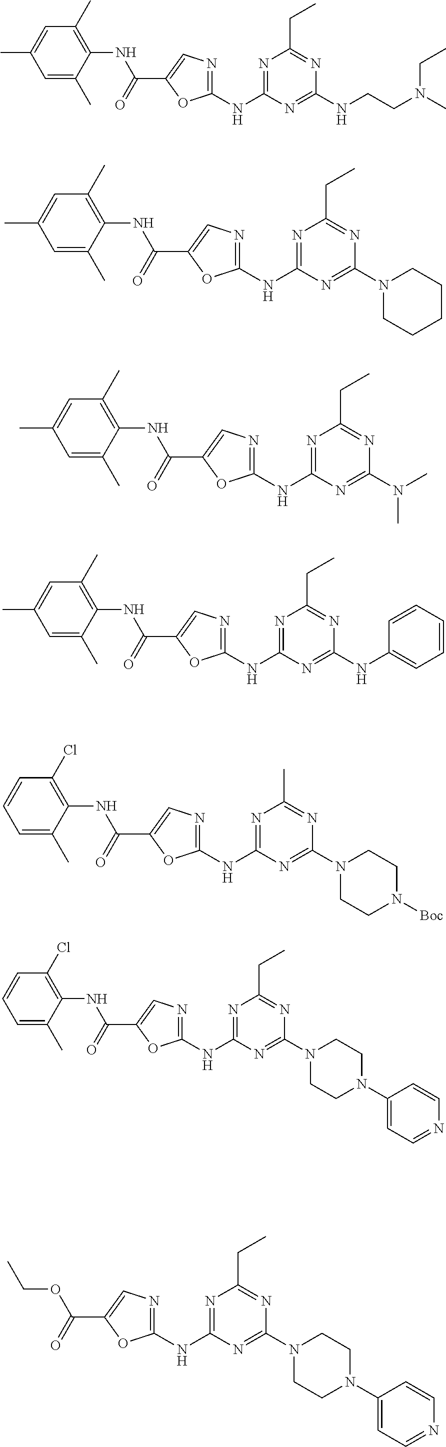

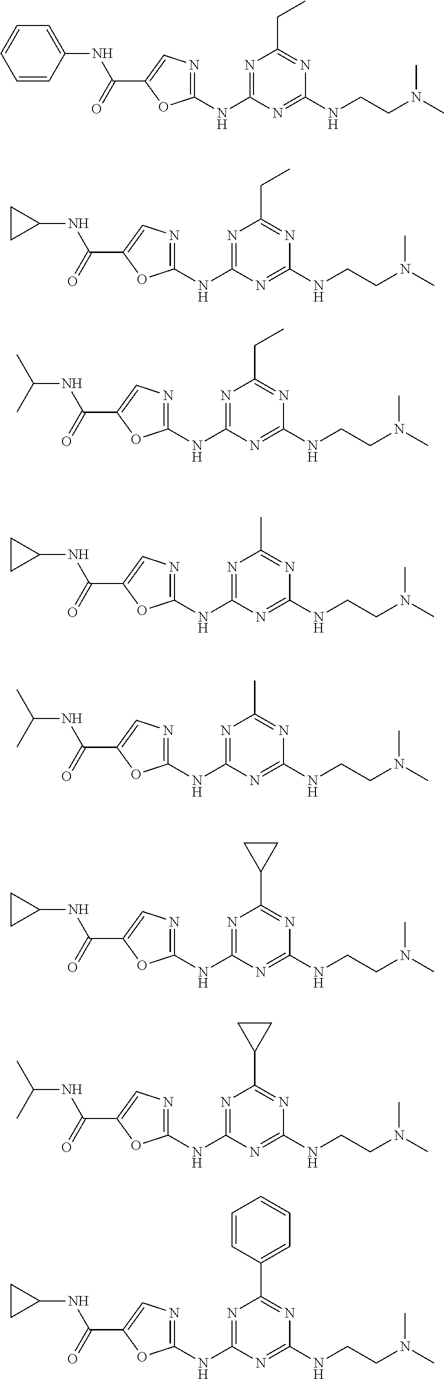

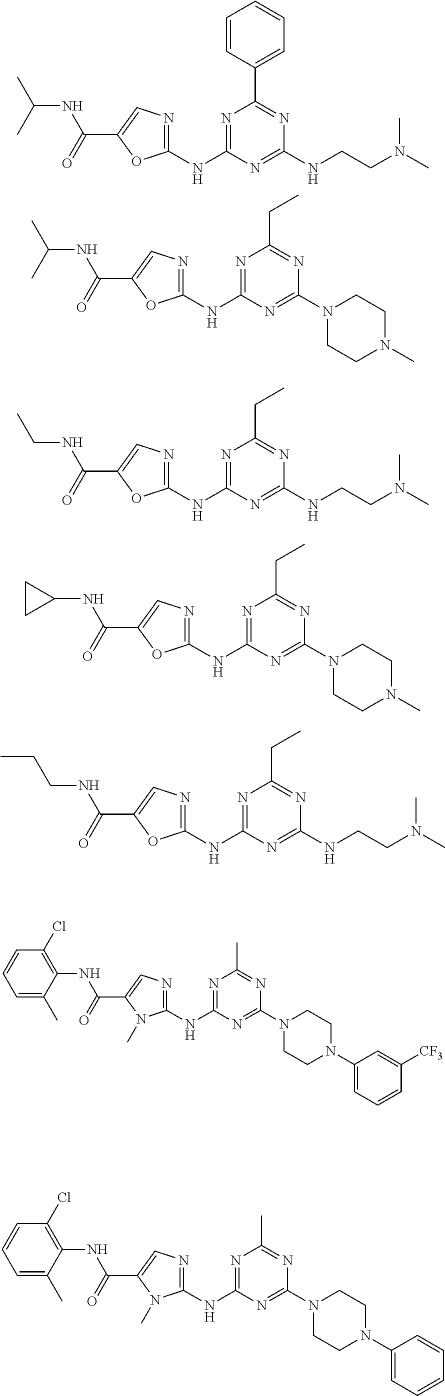

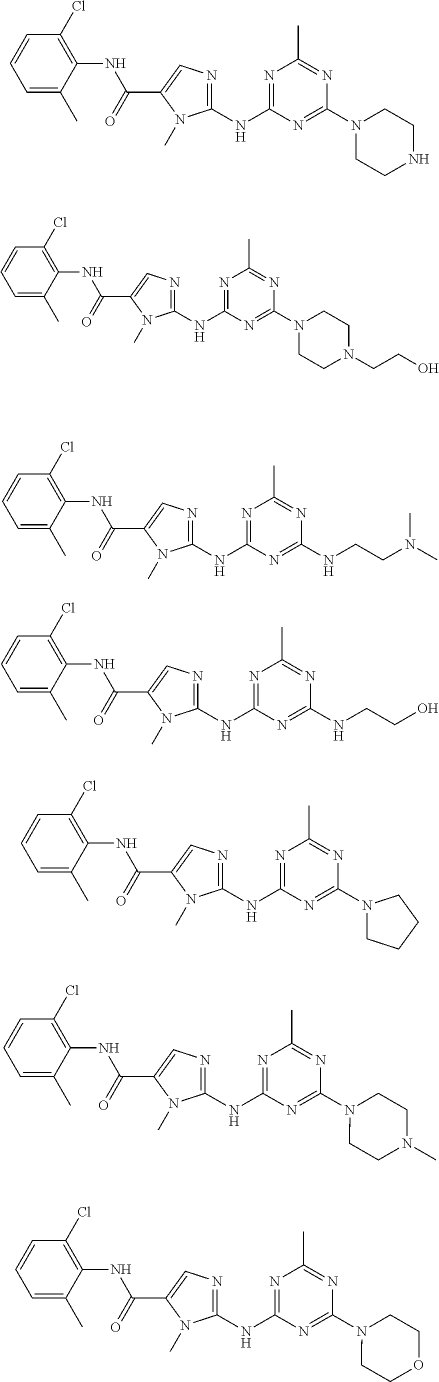

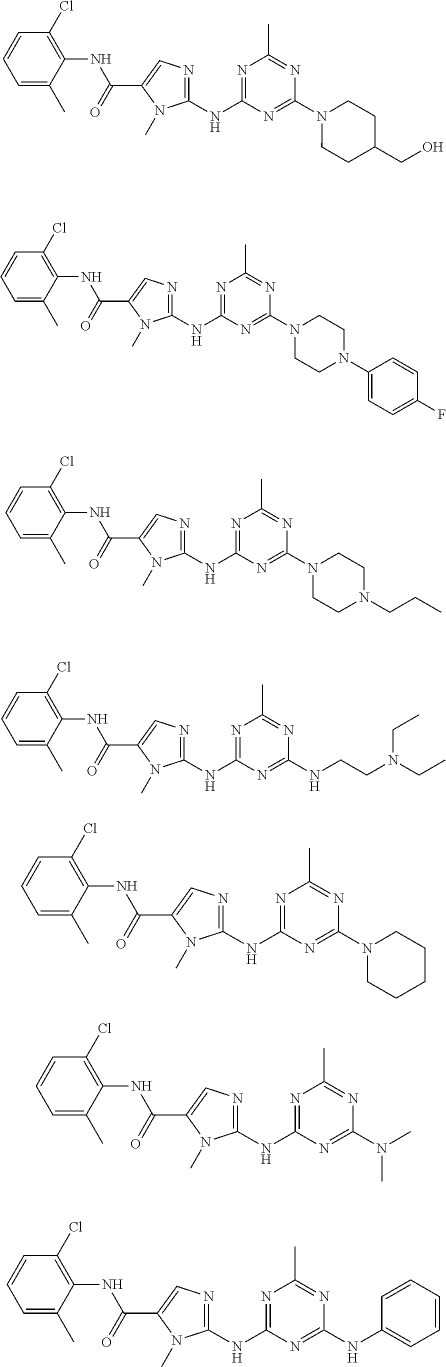

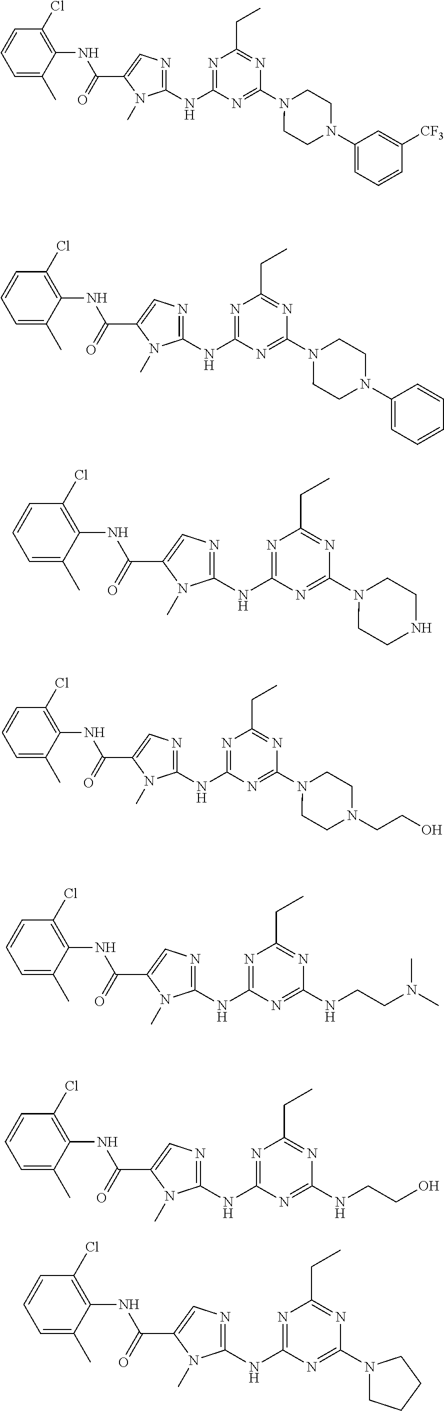

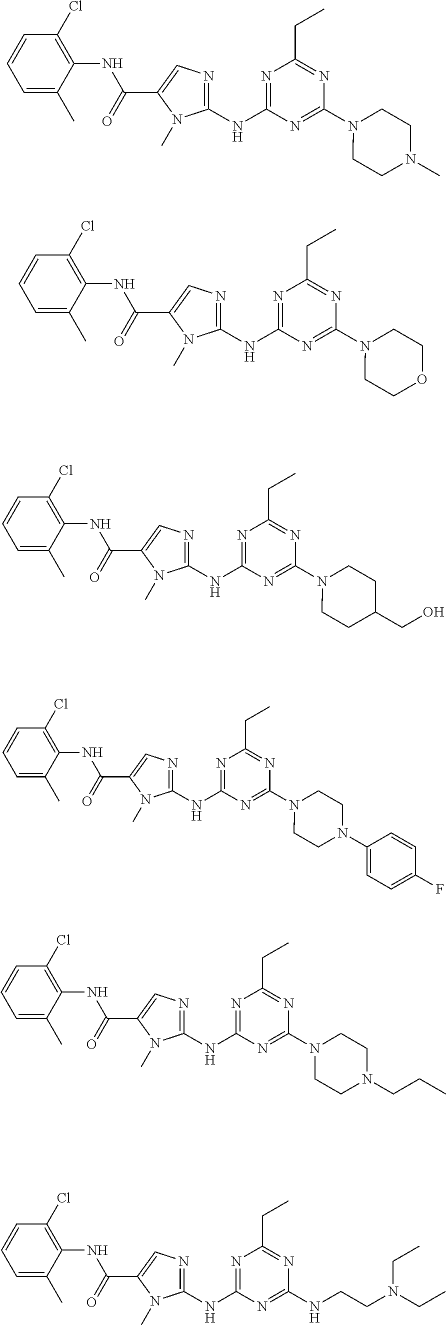

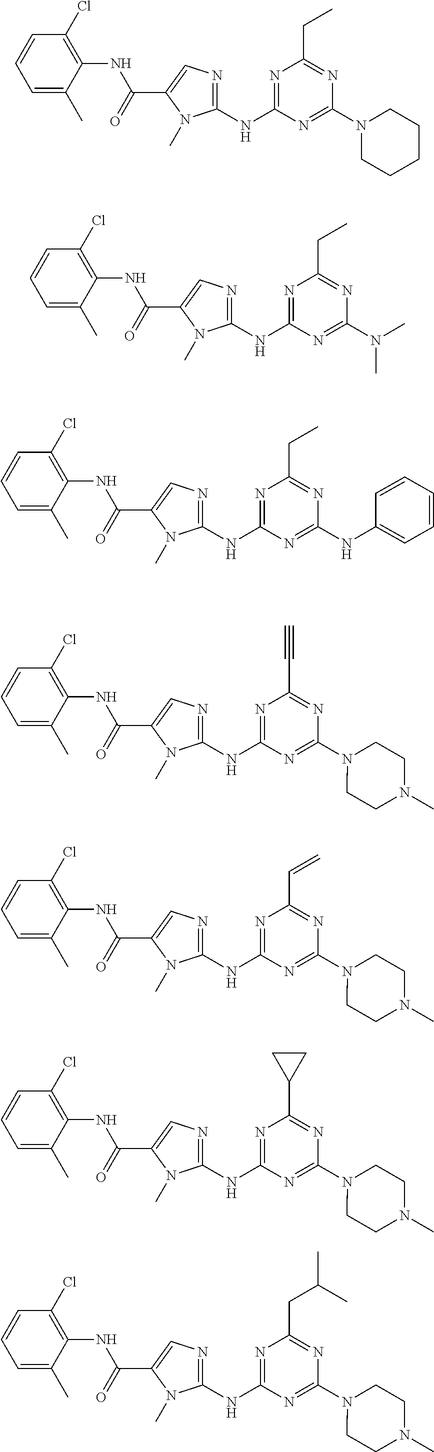

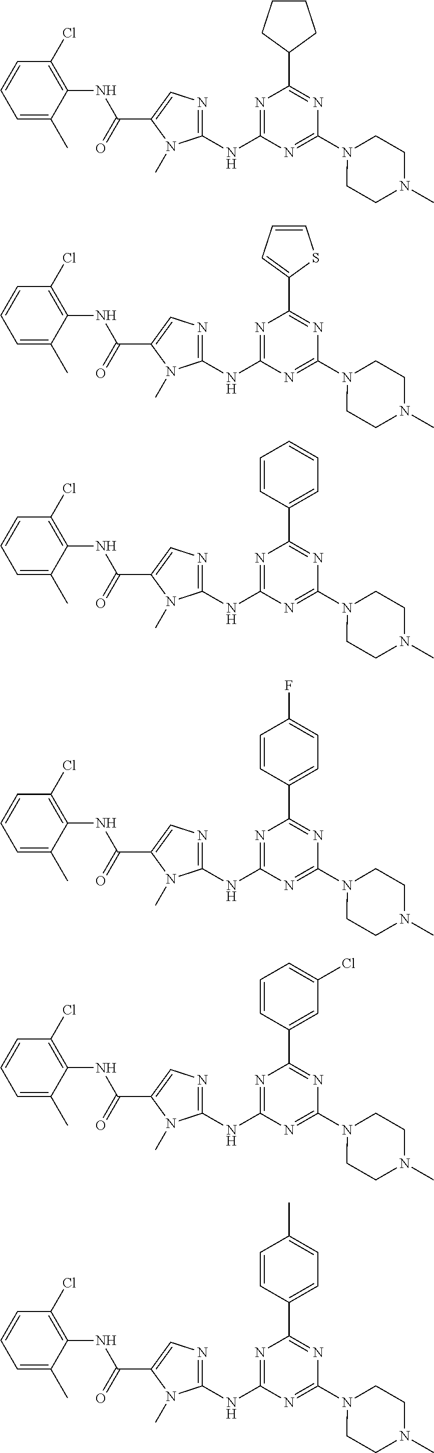

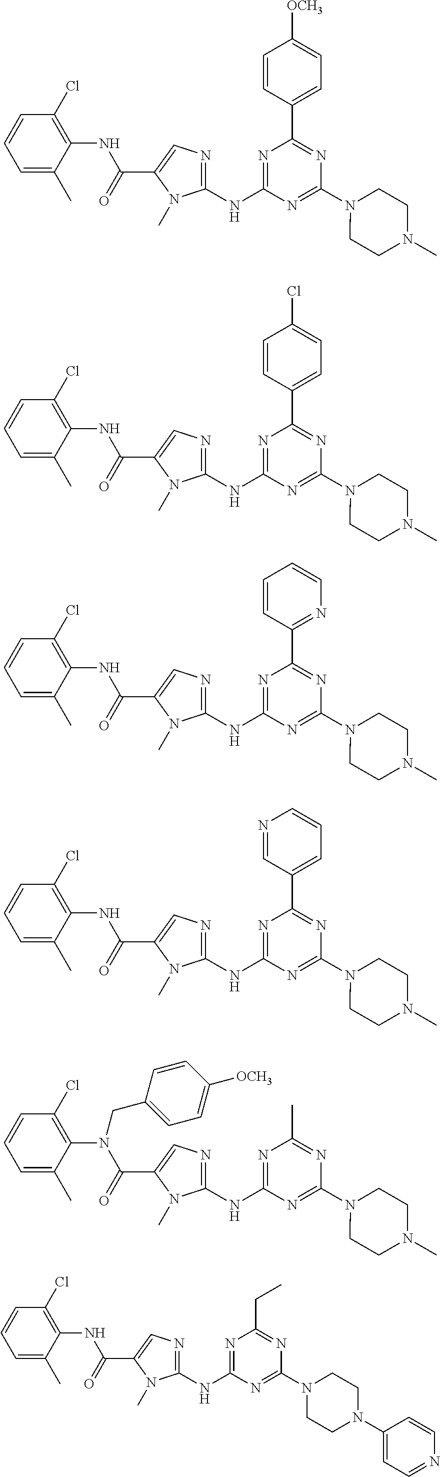

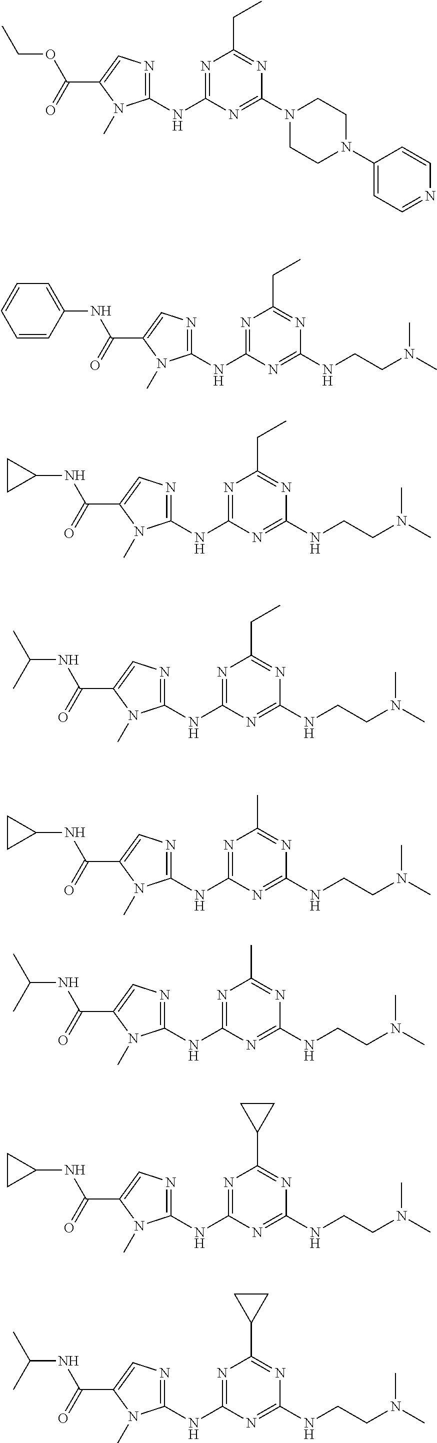

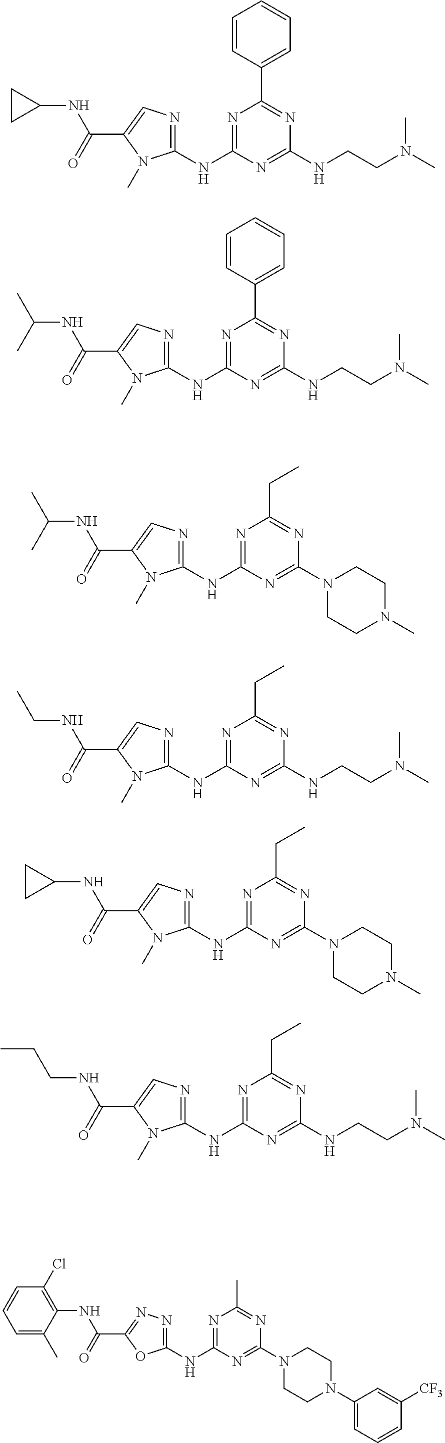

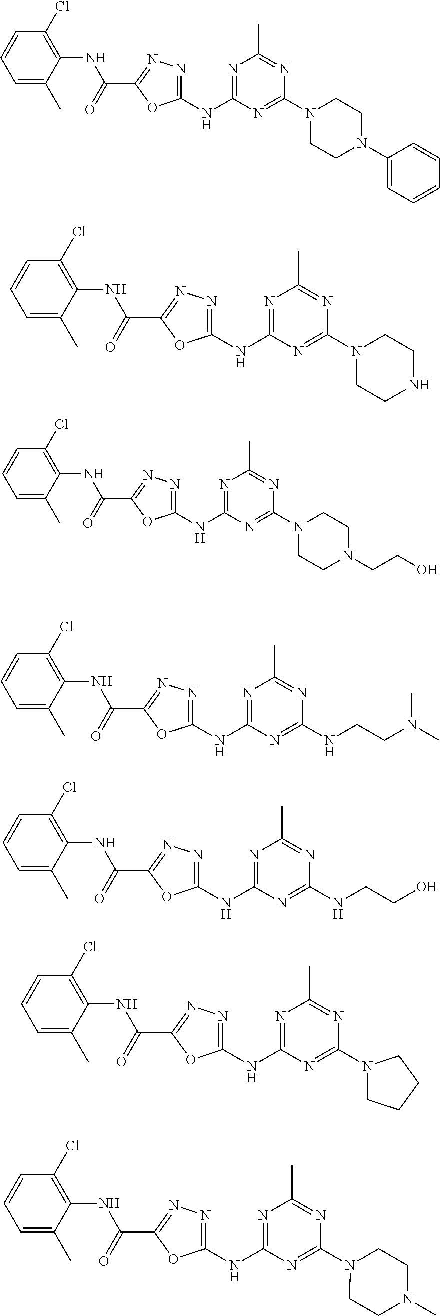

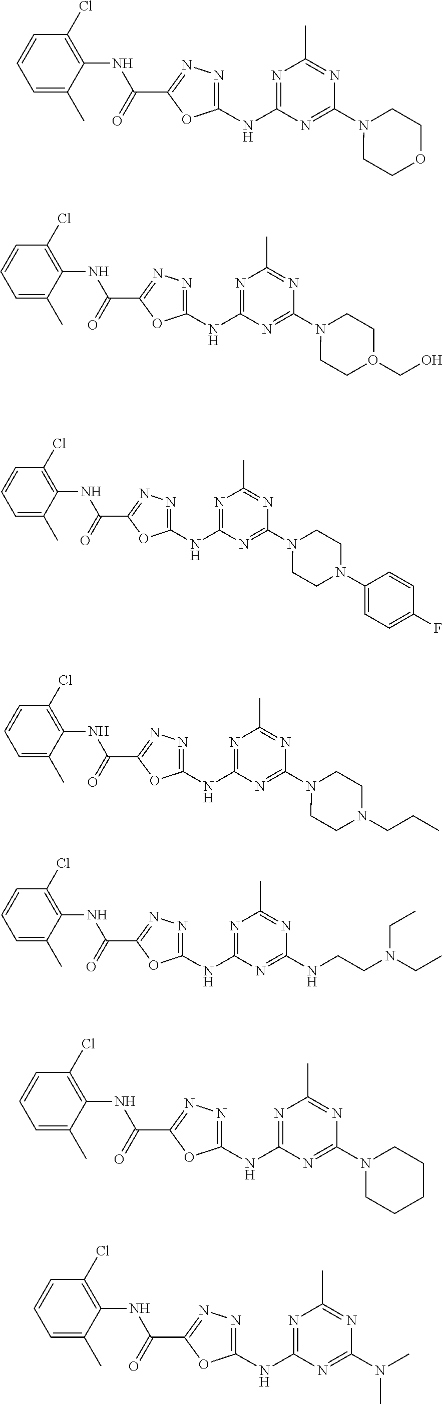

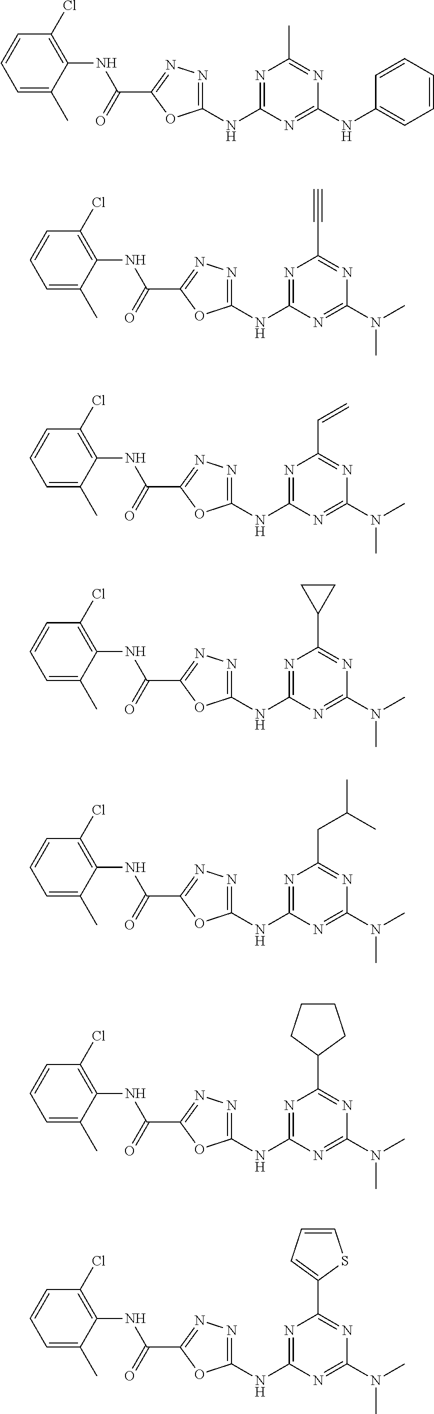

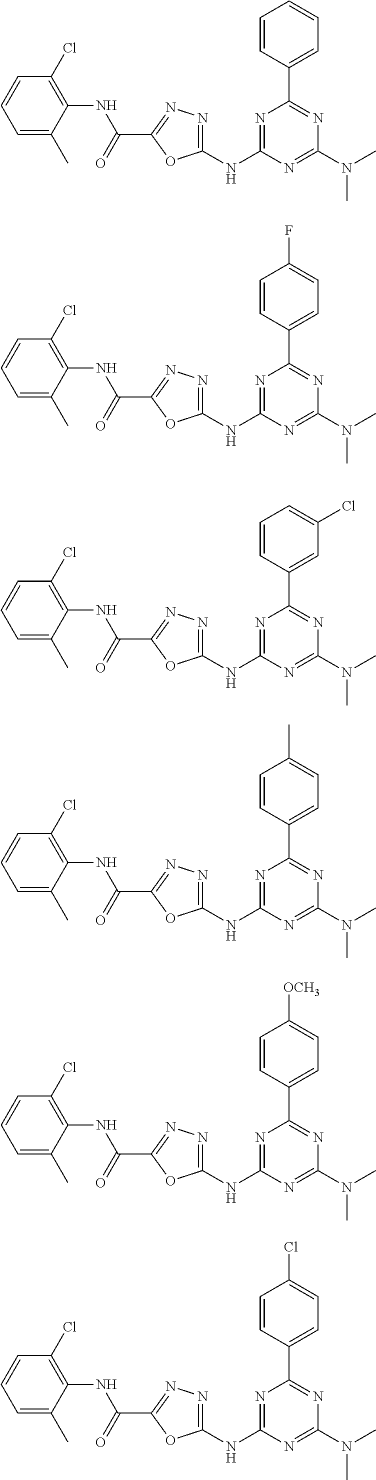

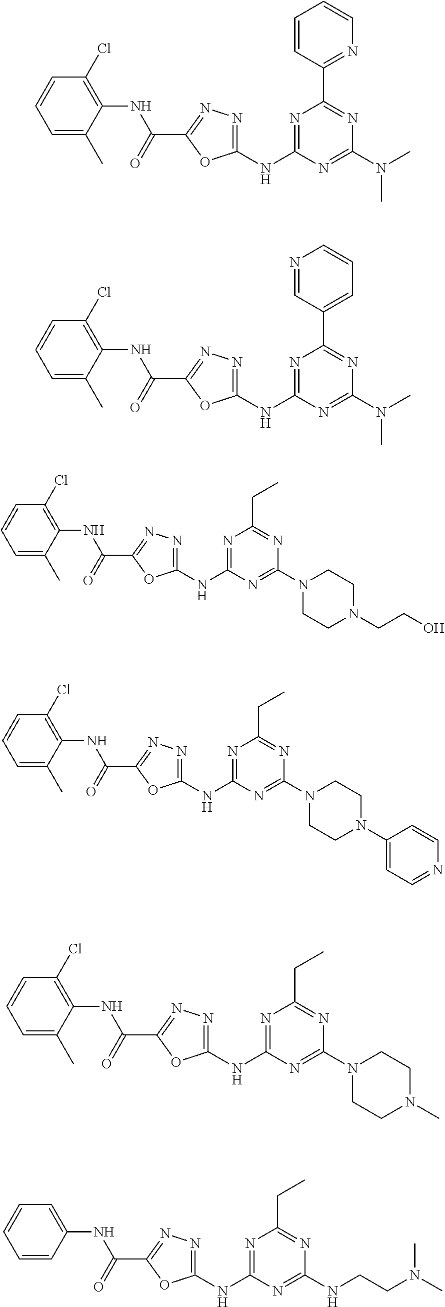

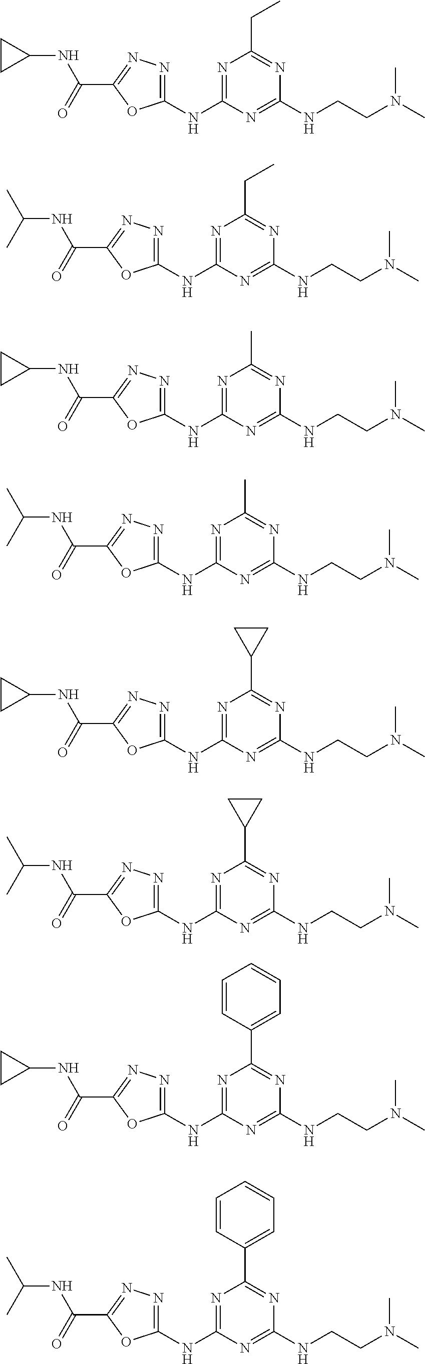

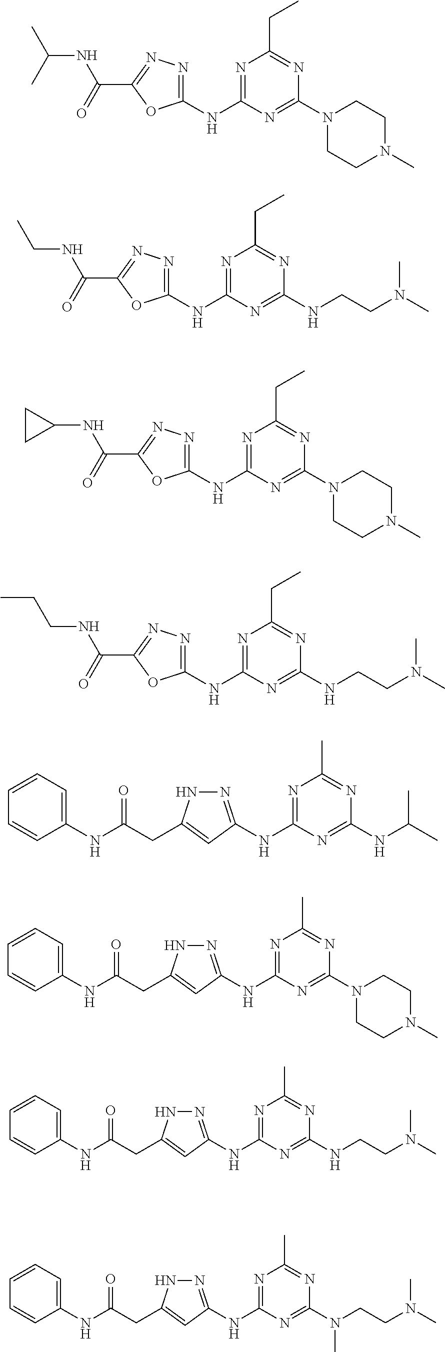

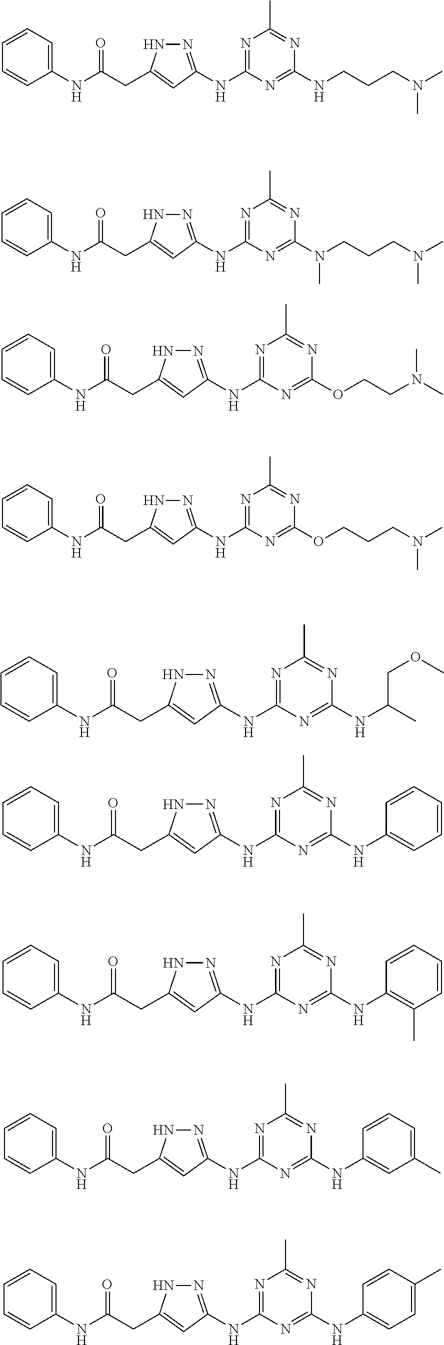

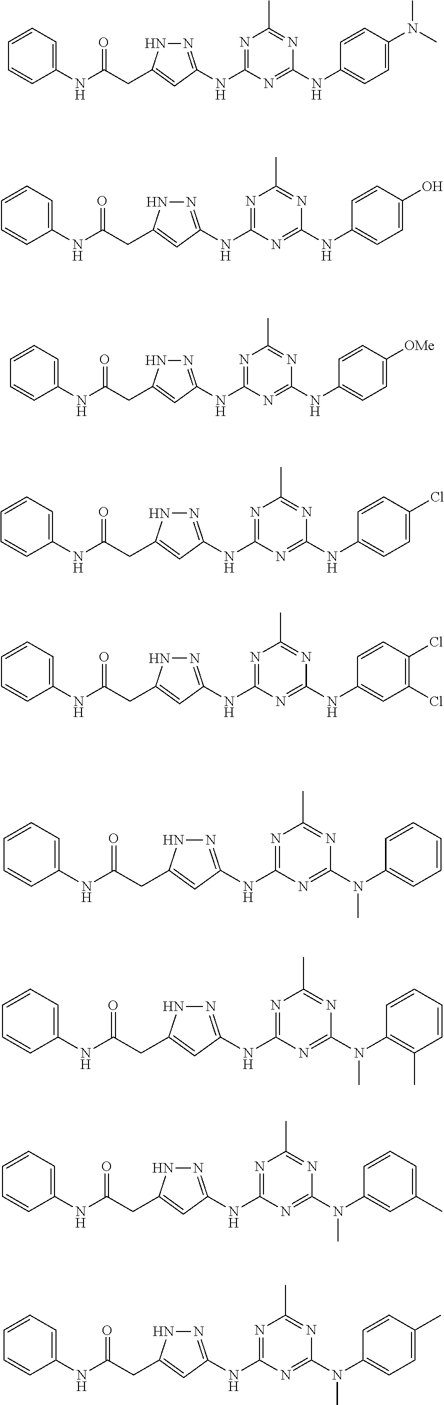

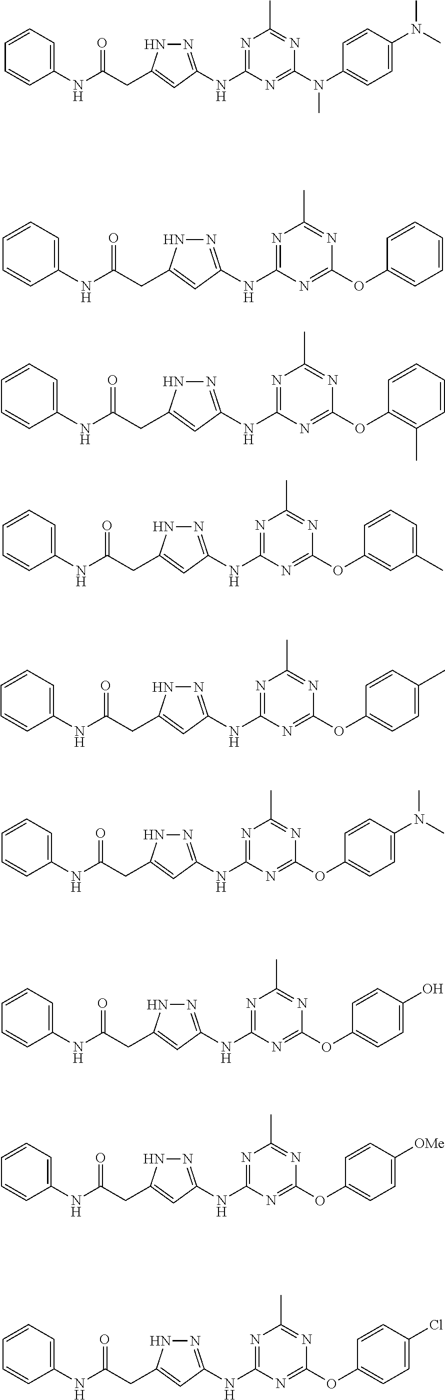

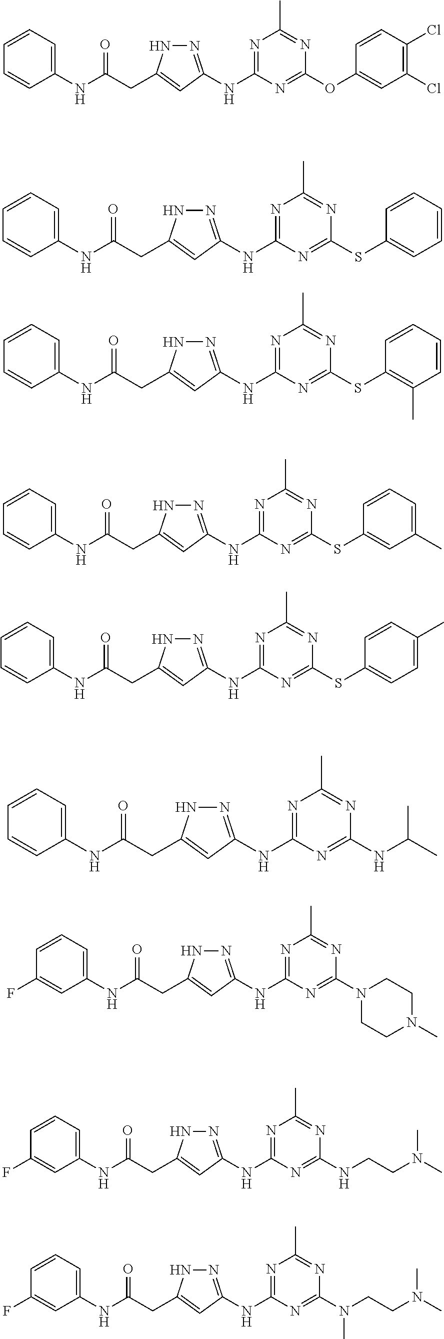

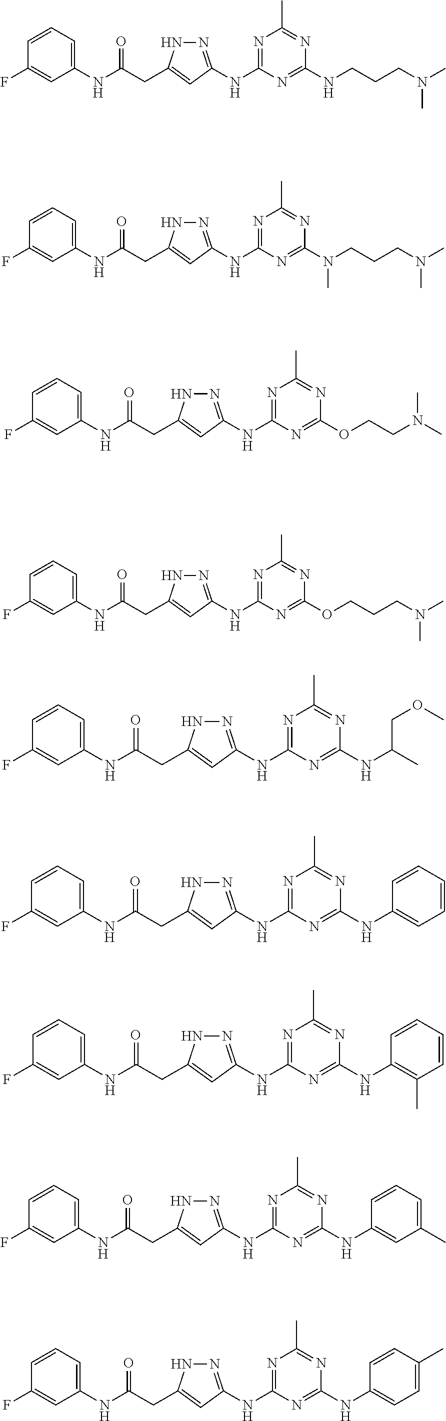

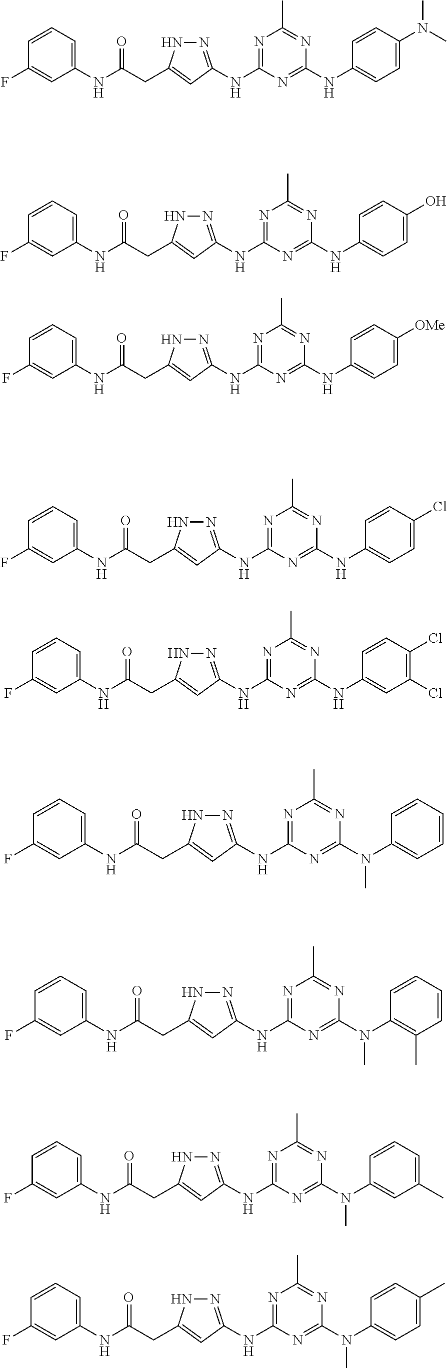

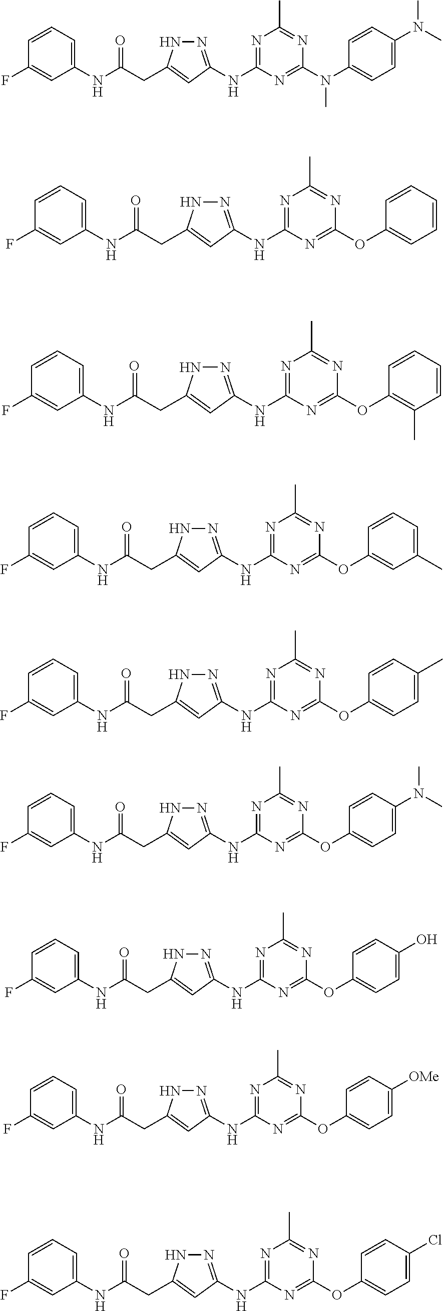

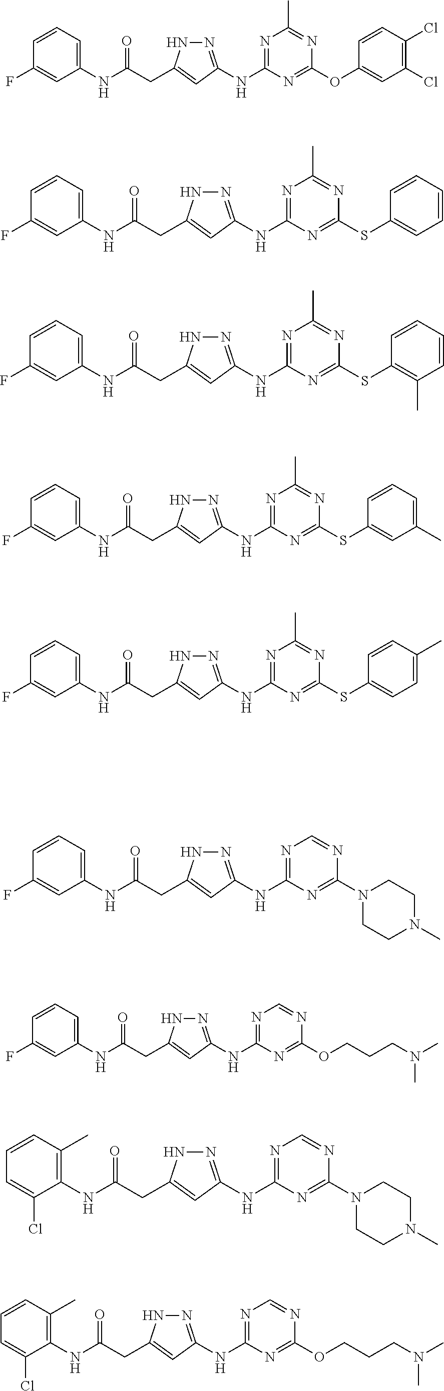

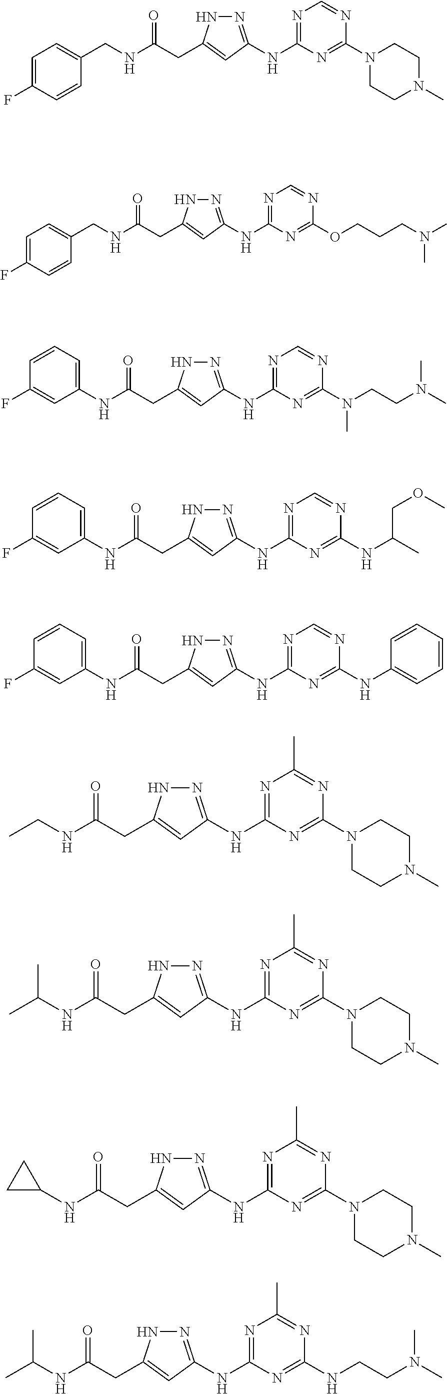

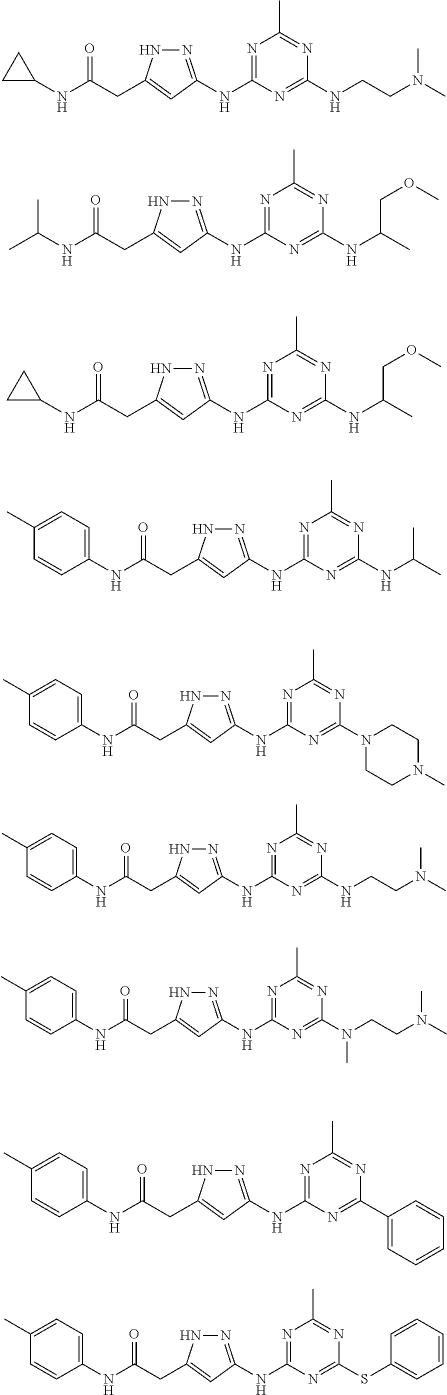

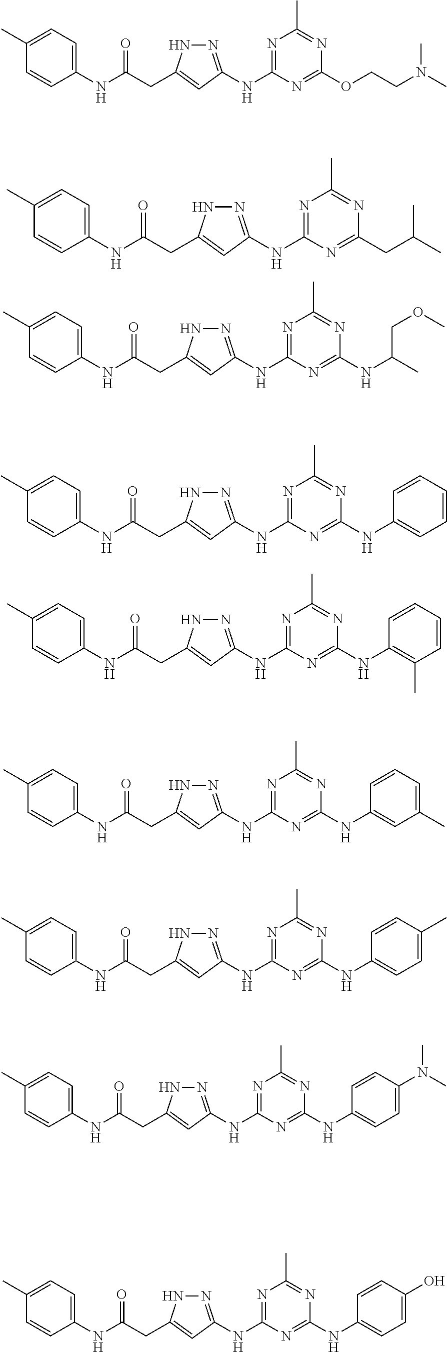

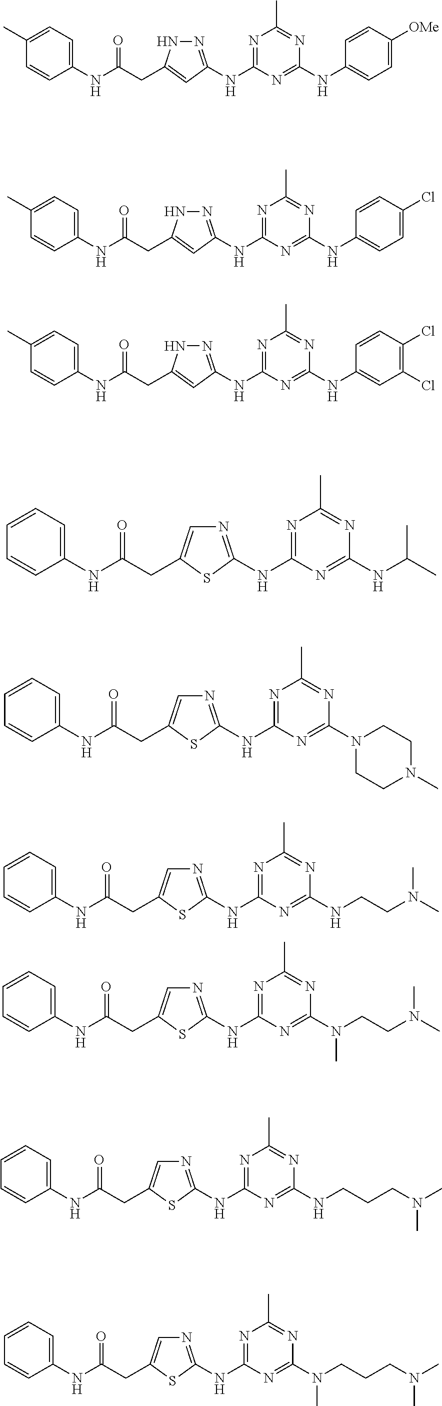

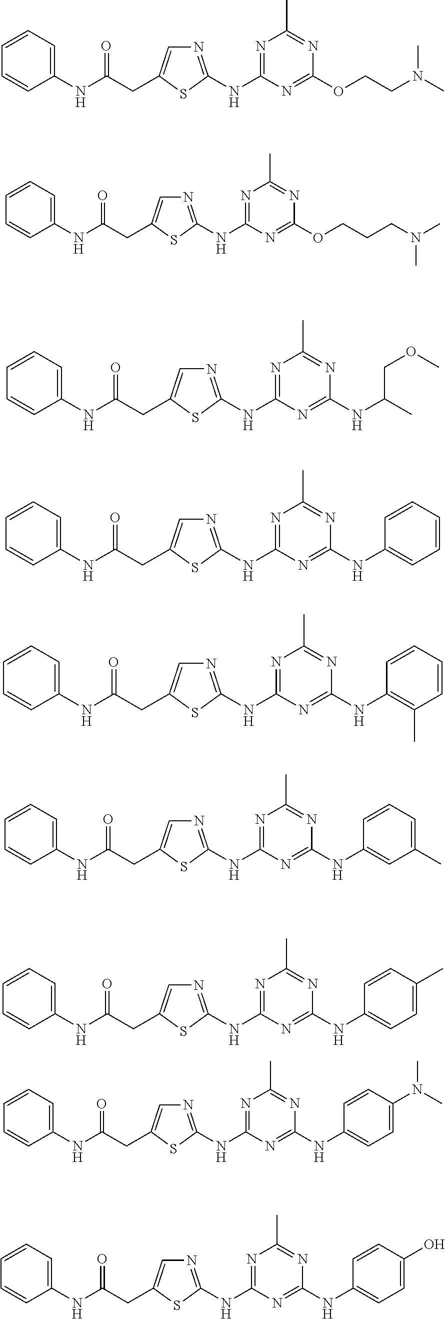

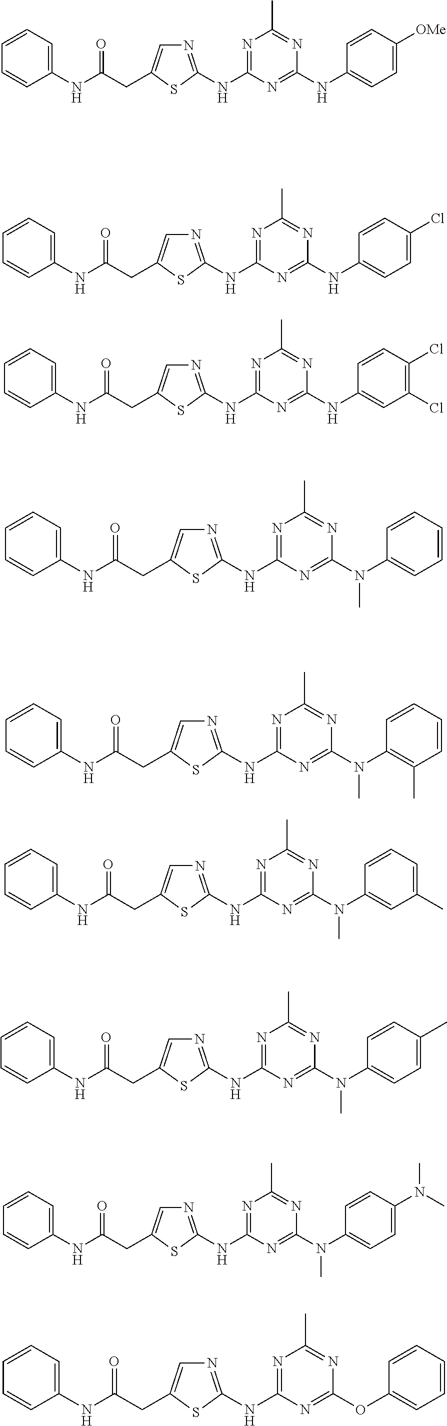

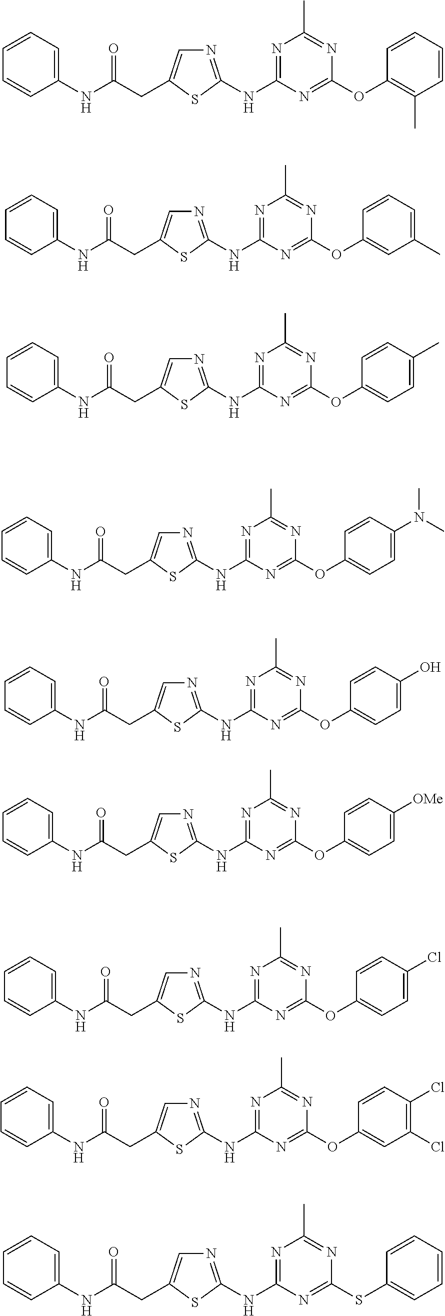

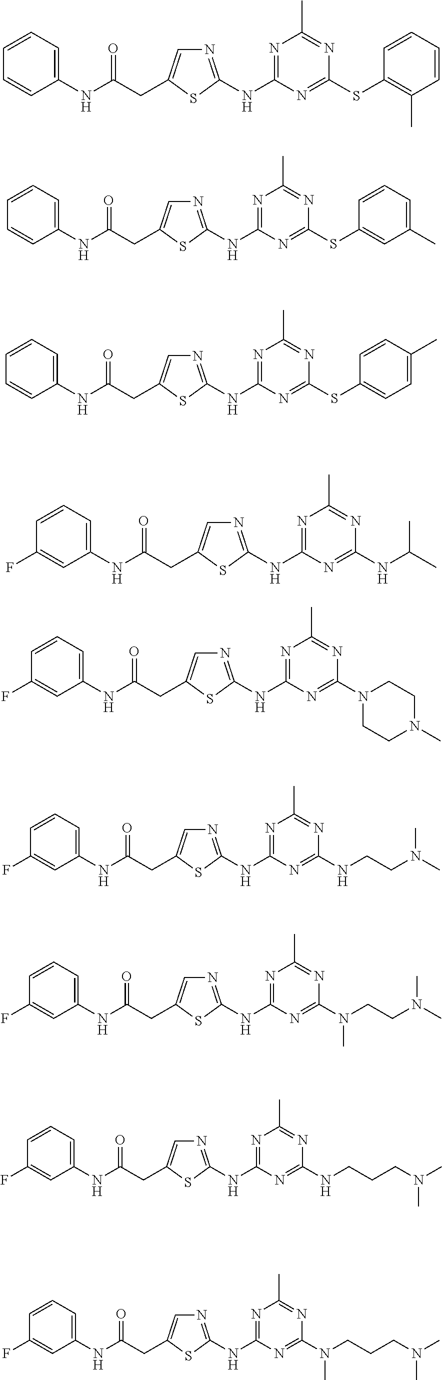

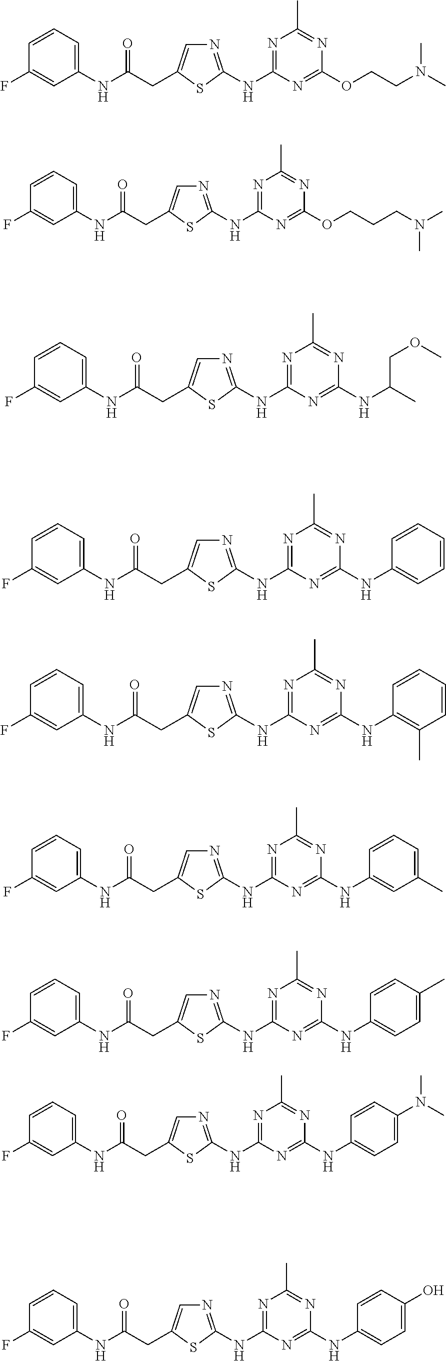

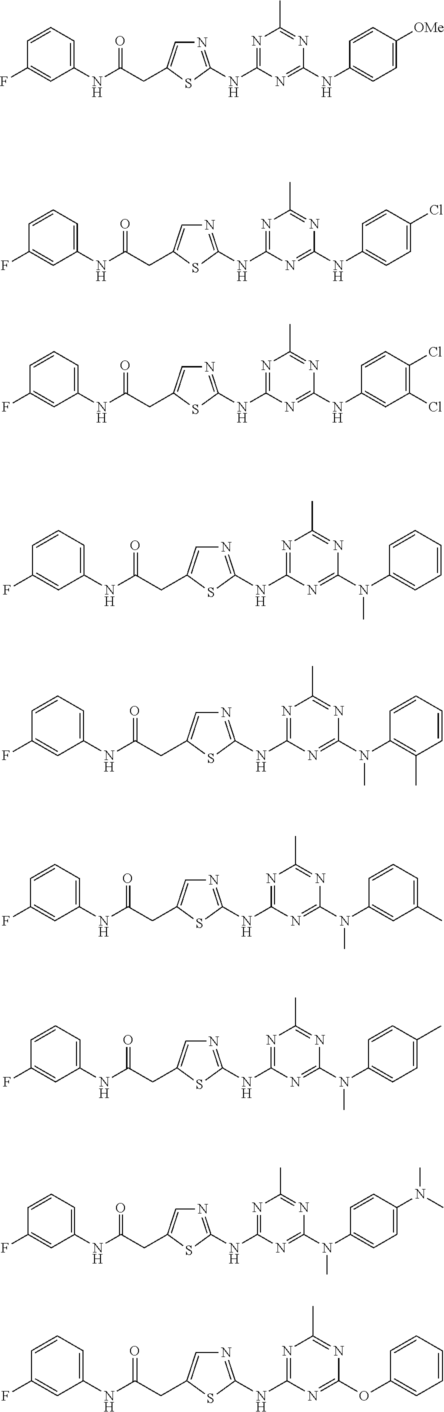

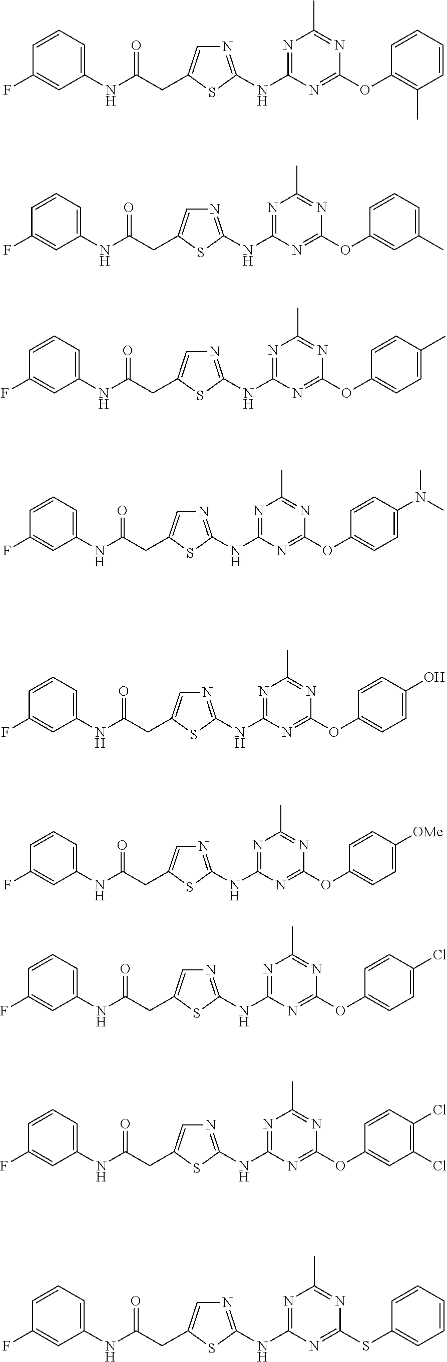

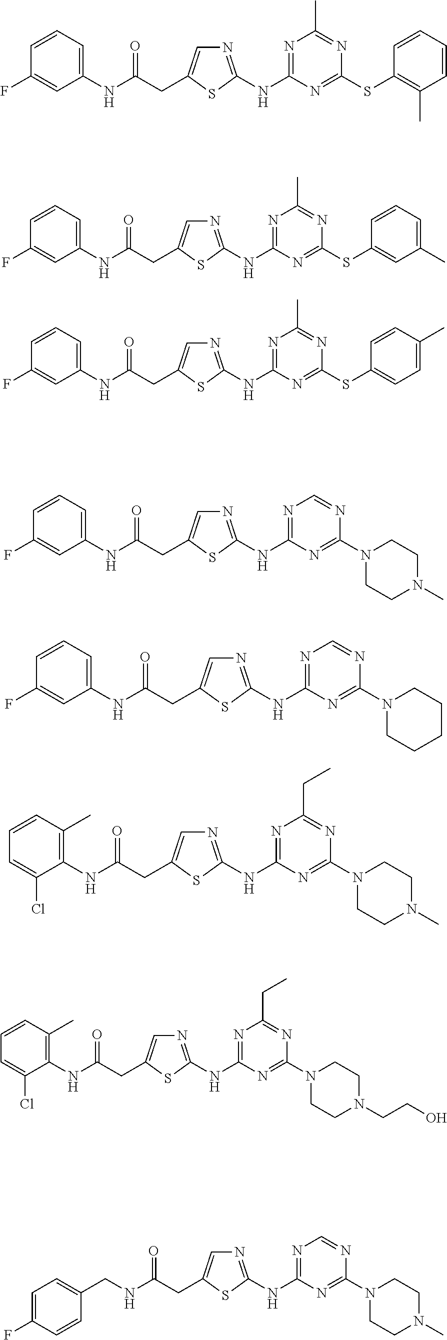

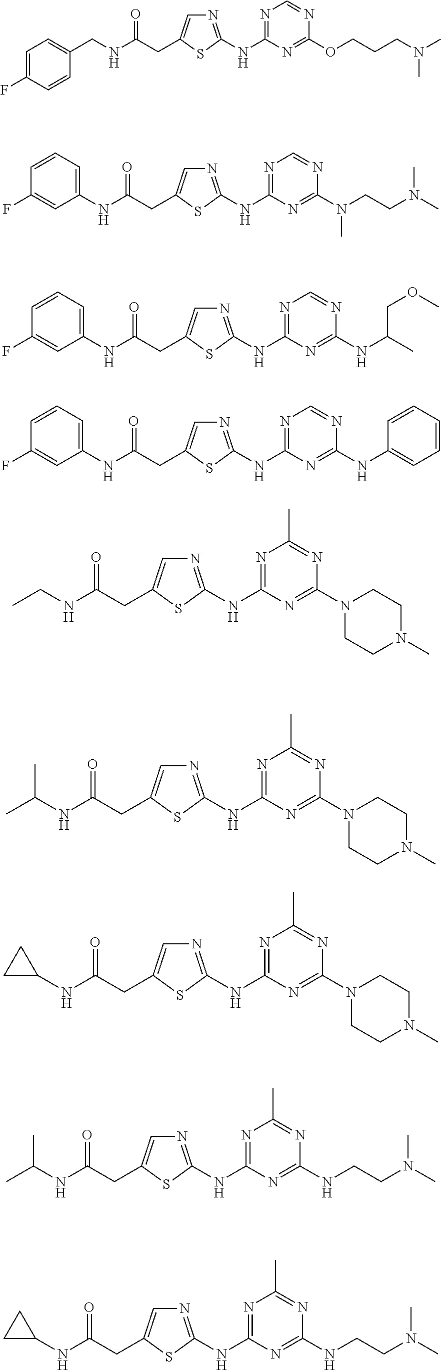

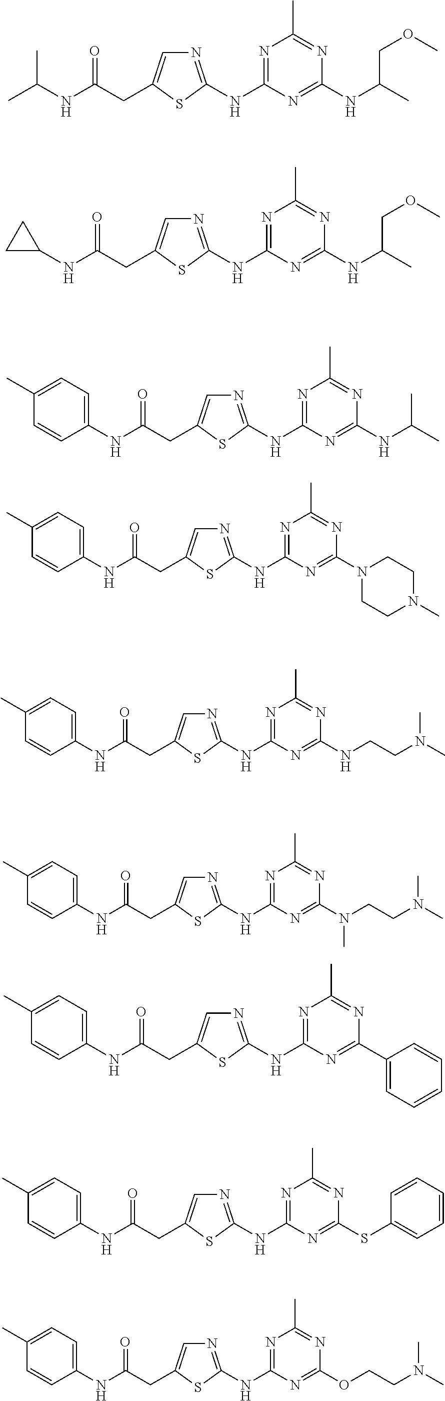

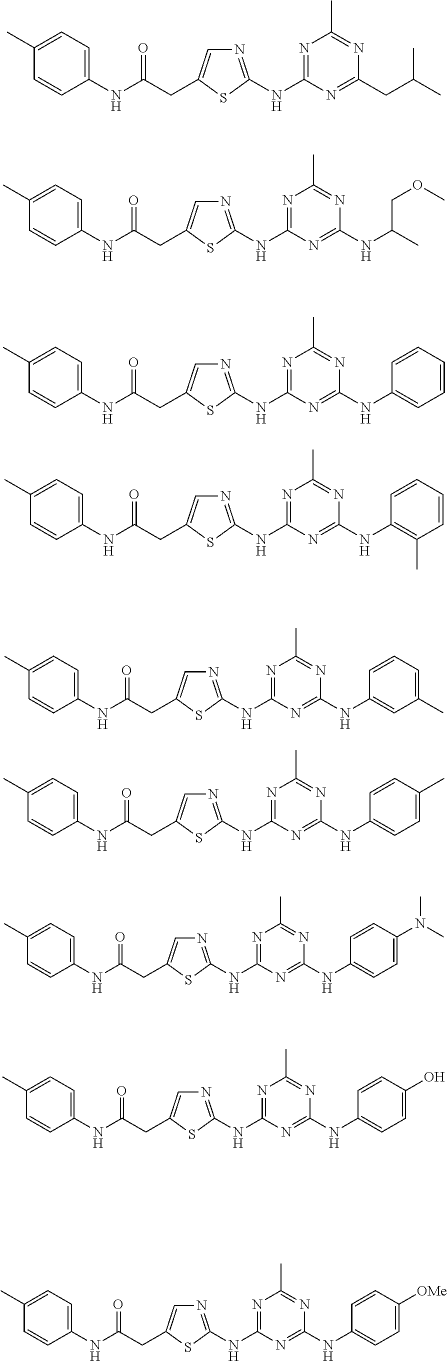

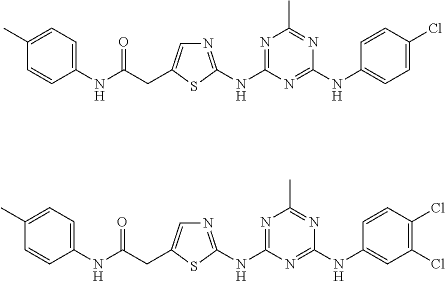

In another embodiment, a method of preparing the inventive compounds is provided. The compounds of the present invention can be generally prepared using cyanuric chloride as a starting material. Compounds of formula (I) or formula (II) may contain various stereoisomers, geometric isomers, tautomeric isomers, and the like. All of possible isomers and their mixtures are included in the present invention, and the mixing ratio is not particularly limited.

The triazine derivative compounds of formula (I) or formula (II) in this invention can be prepared by known procedure in the prior art. The examples could be found in US Patent Application Publication No. 2005/0250945A1; US Patent Application Publication No. 2005/0227983A1; PCT WO 05/007646A1; PCT WO 05/007648A2; PCT WO 05/003103A2; PCT WO 05/011703 A1; and I Med. Chem. (2004), 47(19), 4649-4652. Starting materials are commercially available from suppliers such as Sigma-Aldrich Corp. (St. Louis, Mo.), or may be synthesized from commercially available precursors using established protocols. By way of example, a synthetic route similar to that shown in any of the following Schemes may be used, together with synthetic methods known in the art of synthetic organic chemistry, or variations thereon as appreciated by those skilled in the art. Each variable in the following schemes refers to any group consistent with the description of the compounds provided herein.

In the Schemes that follow the term “reduction” refers to the process of reducing a nitro functionality to an amino functionality, or the process of transforming an ester functionality to an alcohol. The reduction of a nitro group can be carried out in a number of ways well known to those skilled in the art of organic synthesis including, but not limited to, catalytic hydrogenation, reduction with SnCl₂ and reduction with titanium bichloride. The reduction of an ester group is typically performed using metal hydride reagents including, but not limited to, diisobutyl-aluminum hydride (DIBAL), lithium aluminum hydride (LAH), and sodium borohydride. For an overview of reduction methods see: Hudlicky, M. Reductions in Organic Chemistry, ACS Monograph 188, 1996. In the Schemes that follow, the term “hydrolyze” refers to the reaction of a substrate or reactant with water. More specifically, “hydrolyze” refers to the conversion of an ester or nitrite functionality into a carboxylic acid. This process can be catalyzed by a variety of acids or bases well known to those skilled in the art of organic synthesis.

The compounds of formula (I) or formula (II) may be prepared by use of known chemical reactions and procedures. The following general preparative methods are presented to aid one of skill in the art in synthesizing the inhibitors, with more detailed examples being presented in the experimental section describing the working examples.

Heterocyclic amines are defined in formula (III). Some of heterocyclic amines are commercially available, others may be prepared by known procedure in the prior art (Katritzky, et al. Comprehensive Heterocyclic Chemistry; Permagon Press: Oxford, UK, 1984, March. Advanced Organic Chemistry, 3rd Ed.; John Wiley: New York, 1985), or by using common knowledge of organic chemistry.

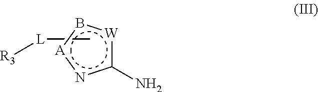

For example, heterocyclic amine with an amide link (IIIa) can be prepared from commercial compounds as illustrated in Scheme 1. By Route 1, the amine is first protected by Boc or other appropriate protecting group; after hydrolysis, the acid can be converted to corresponding amide; followed by removal of protecting group, the desired amine can be obtained. Alternatively, by Route 2, the acid, which is either commercially available, or made from its ester form, can also be converted to the desired compound (IIIa). A lot of heterocyclic amines can be prepared by this way.

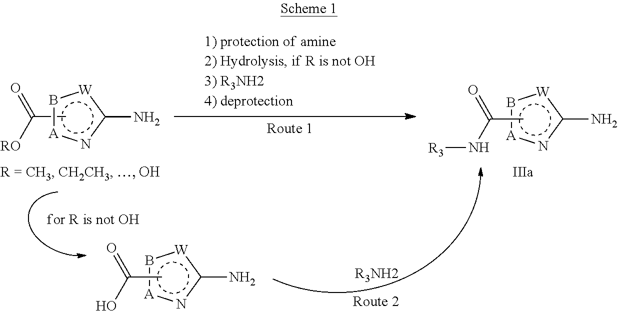

Substituted heterocyclic amines can also be generated using standard methods (March, J. Advanced Organic Chemistry, 4th Ed.; John Wiley, New York (1992); Larock, R. C. Comprehensive Organic Transformations, 2nd Ed., John Wiley, New York (1999); PCT No. WO 99/32106). As shown in Scheme 2, heterocyclic amines can be commonly synthesized by reduction of nitroheteros using a metal catalyst, such as Ni, Pd, or Pt, and H₂ or a hydride transfer agent, such as formate, cyclohexadiene, or a borohydride (Rylander. Hydrogenation Methods; Academic Press: London, UK (1985)). Nitroheteros may also be directly reduced using a strong hydride source, such as LAH, (Seyden-Penne. Reductions by the Alumino- and Borohydrides in Organic Synthesis; VCH Publishers: New York (1991)), or using a zero valent metal, such as Fe, Sn or Ca, often in acidic media. Many methods exist for the synthesis of nitroaryls (March, J. Advanced Organic Chemistry, 4th Ed.; John Wiley, New York (1992); Larock, R. C. Comprehensive Organic Transformations, 2nd Ed., John Wiley, New York (1999))).

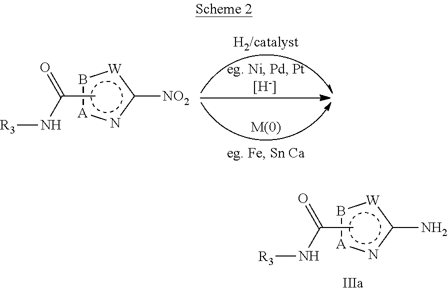

Nitroheteroaryls may be further elaborated prior to reduction. Nitroheteros substituted with potential leaving groups (eg. F, Cl, Br, etc.) may undergo substitution reactions on treatment with nucleophiles, such as thiolate (exemplified in Scheme 3) or phenoxide. Nitroaryls may also undergo Ullman-type coupling reactions (Scheme 3).

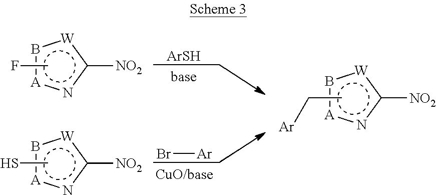

Scheme 4 illustrates one of the methods to prepare those heterocyclic amines as in Formula Mb, where L is carbonyl. These heterocyclic amines are readily available from reactions of a heterocyclic amine with a substituted aryl carbonyl chloride. Acetyl protection of the amine, which can be easily removed after the Friedel-Crafts reaction, is preferred. These carbonyl linked heterocyclic amines can be further converted to methylene (IIIc) or hydroxyl methylene (IIId) linked ones by appropriate reduction.

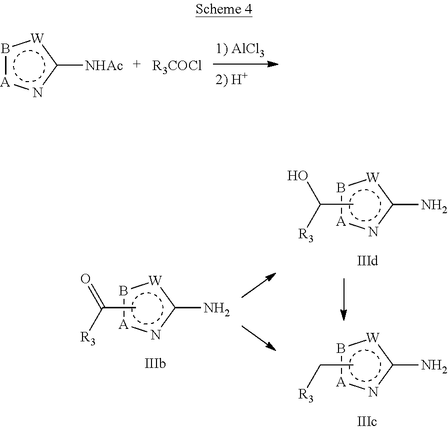

As illustrated in Scheme 5,2-amino thiazole-5-carboxamide or 2-amino-oxazole-5-carboxamide (IIId) are available by the reaction of thiourea or urea with an appropriate ethoxyacrylamide in the presence of NBS, which can be made from the reaction of 3-ethyoxyacryloyl chloride with a corresponding amino compound R′—NH₂. The 3-ethyoxyacryloyl chloride can be prepared from the corresponding acid or ester.

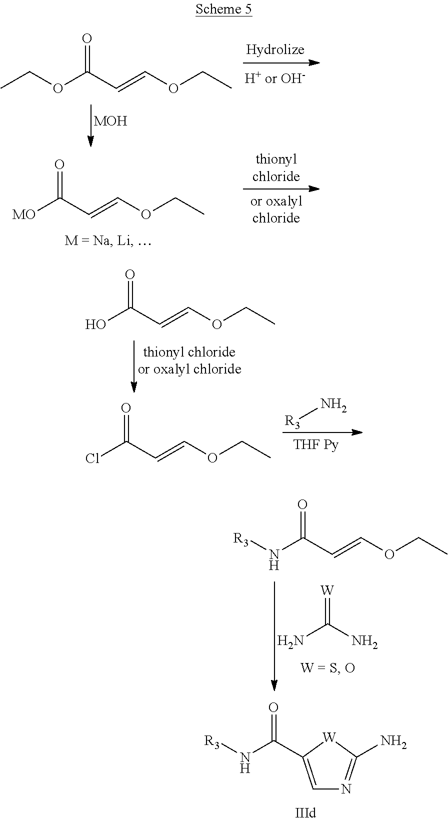

The preparation of the compound of formula (IV) in this invention can be carried out by methods known in the art (e.g., J. Med. Chem. 1996, 39, 4354-4357; J. Med. Chem. 2004, 47, 600-611; J. Med. Chem. 2004, 47, 6283-6291; J. Med. Chem. 2005, 48, 1717-1720; J. Med. Chem. 2005, 48, 5570-5579; U.S. Pat. No. 6,340,683 B1; JOC, 2004, 29, 7809-7815).

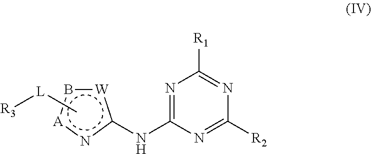

Scheme 6 illustrated the synthesis method for compounds with alkyl or aryl as R₁. The 6-alkyl or aryl substituted dichloro-triazine (b) may be synthesized by the methods known in the art (e.g., J. Med. Chem. 1999, 42, 805-818 and J. Med. Chem. 2004, 47, 600-611) from cyanuric chloride (a) and Grignard reagents. Triazine derivatives can be formed from the reaction of a 6-alkyl or aryl substituted dichloro-triazine (b) with heterocyclic amine, followed by reaction with HR₂. Alternatively, the monochloro-triazine (c) can be converted to amino triazine (d), which can react with YR₂, to give a triazine derivative (IV). Also, dichloro-triazine (b) can react with HR₂, followed by reaction with heterocyclic amine to give triazine derivative (IV). Further more, monochloro-triazine (e) can be converted to amino triazine (f), which can react with a leaving-group-attached heterocyclic compound (g), to give a triazine derivative (IV).

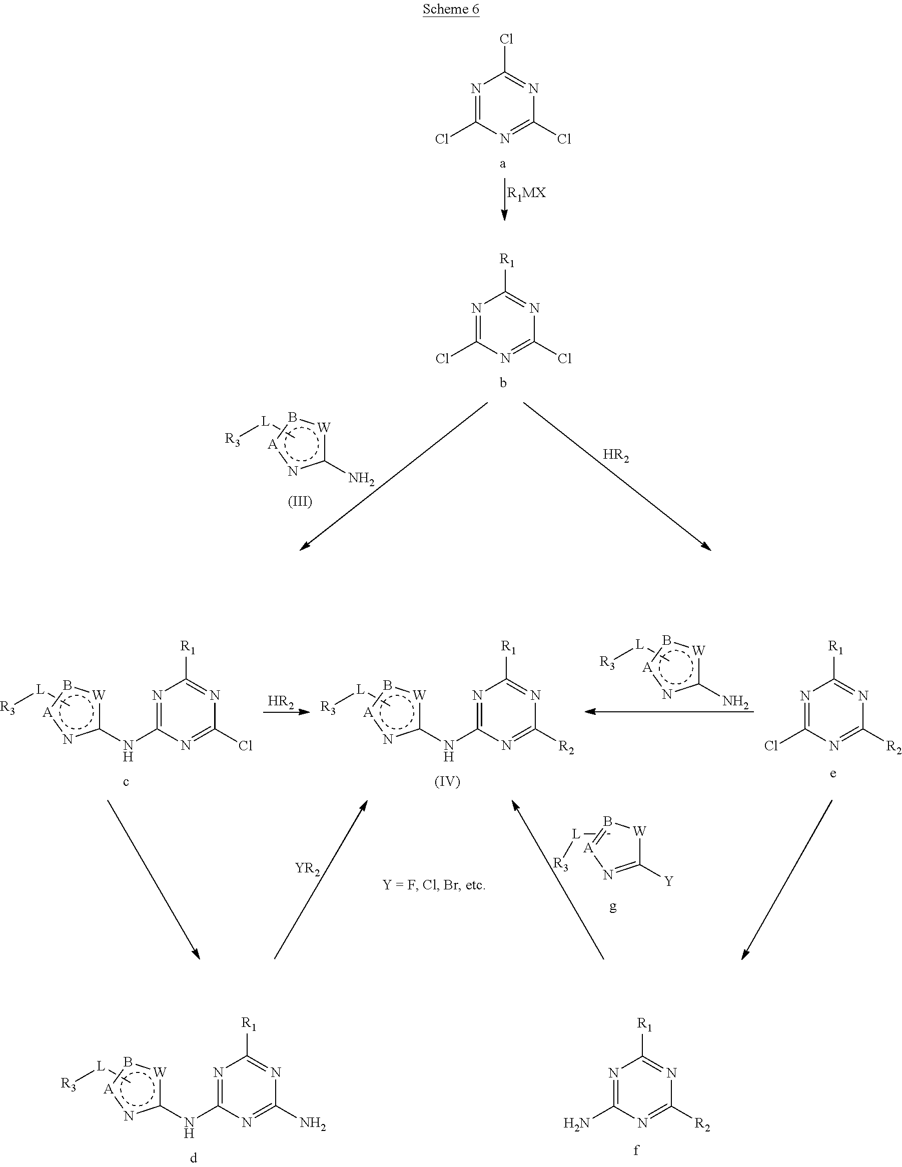

As shown in Scheme 7, the triazine derivative can also be synthesized by the reaction of cyanuric chloride with a sequence of heterocyclic amines and HR₂ to give 2,4-disubstituted-6-chloro-1,3,5-triazines. The displacement of the last chlorine by amine, hydrazine, hydroxyl or other nucleophilic group can be achieved by increasing the temperature, affording the trisubstituted-1,3,5-triazines (IV).

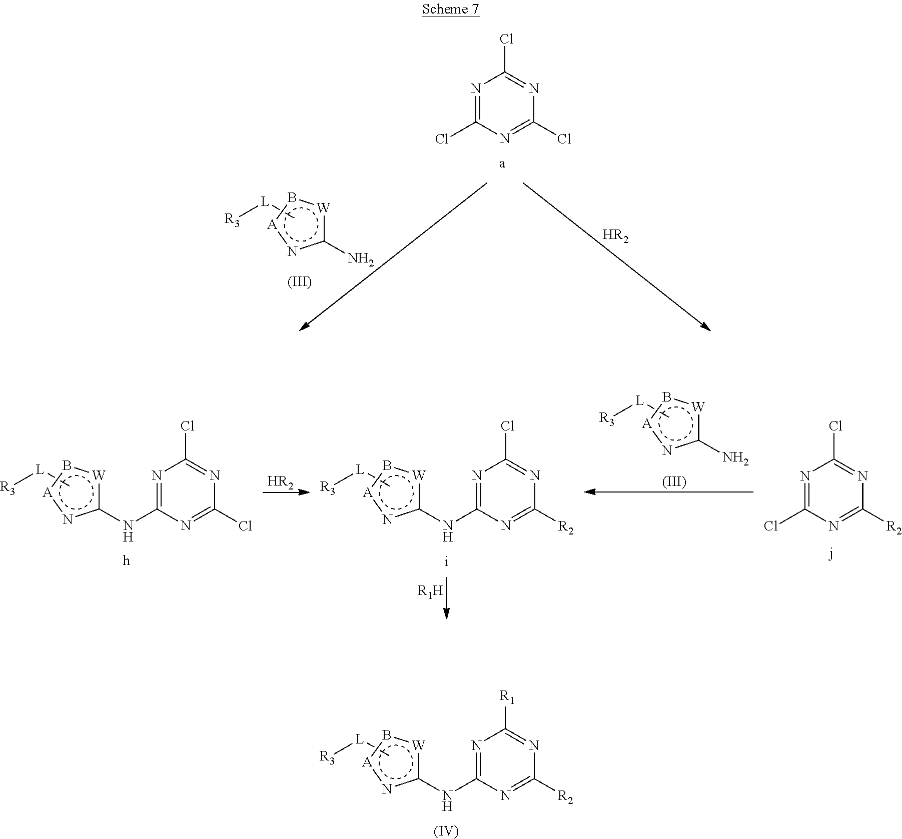

Furthermore, as shown in Scheme 7, the triazine derivative can be synthesized by the reaction of tri, di- or mono chloride triazine with a heterocyclic amines then the R₃-L can be added to the heterocyclic moiety. For example, an amide moiety can be added this way, where is triazines (IV).

Other triazine derivatives of formula (I), where K is not NH, can be prepared in a

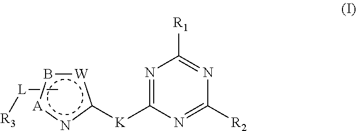

The reaction is preferably conducted in the presence of an inert solvent. There is no particular restriction on the nature of the solvent to be employed, provided that it has no adverse effect on the reaction or on the reagents involved and that it can dissolve the reagents, at least to some extent. Examples of suitable solvents include: aliphatic hydrocarbons, such as hexane, heptane, ligroin and petroleum ether; aromatic hydrocarbons, such as benzene, toluene and xylene; halogenated hydrocarbons, especially aromatic and aliphatic hydrocarbons, such as methylene chloride, chloroform, carbon tetrachloride, dichloroethane, chlorobenzene and the dichlorobenzenes; esters, such as ethyl formate, ethyl acetate, propyl acetate, butyl acetate and diethyl carbonate; ethers, such as diethyl ether, diisopropyl ether, tetrahydrofuran, dioxane. dimethoxyethane and diethylene glycol dimethyl ether; ketones, such as acetone, methyl ethyl ketone, methyl isobutyl ketone, isophorone and cyclohexanone; nitro compounds, which may be nitroalkanes or nitroaranes, such as nitroethane and nitrobenzene; nitriles, such as acetonitrile and isobutyronitrile; amides, which may be fatty acid amides, such as formamide, dimethylformamide, dimethylacetamide and hexamethylphosphoric triamide; and sulphoxides, such as dimethyl sulphoxide and sulpholane.

The reaction can take place over a wide range of temperatures, and the precise reaction temperature is not critical to the invention. In general, we find it convenient to carry out the reaction at a temperature of from −50° C. to 100° C.

The present invention provides compositions of matter that are formulations of one or more active drugs and a pharmaceutically-acceptable carrier. In this regard, the invention provides a composition for administration to a mammalian subject , which may include a compound of formula (I) or formula (II), or its pharmaceutically acceptable salts.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₁₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The pharmaceutically acceptable compositions of this invention may be orally administered in any orally acceptable dosage form including, but not limited to, capsules, tablets, troches, elixirs, suspensions, syrups, wafers, chewing gums, aqueous suspensions or solutions.

The oral compositions may contain additional ingredients such as: a binder such as microcrystalline cellulose, gum tragacanth or gelatin; an excipient such as starch or lactose, a disintegrating agent such as alginic acid, corn starch and the like; a lubricant such as magnesium stearate; a glidant such as colloidal silicon dioxide; and a sweetening agent such as sucrose or saccharin or flavoring agent such as peppermint, methyl salicylate, or orange flavoring. When the dosage unit form is a capsule, it may additionally contain a liquid carrier such as a fatty oil. Other dosage unit forms may contain other various materials which modify the physical form of the dosage unit, such as, for example, a coating. Thus, tablets or pills may be coated with sugar, shellac, or other enteric coating agents. A syrup may contain, in addition to the active ingredients, sucrose as a sweetening agent and certain preservatives, dyes and colorings and flavors. Materials used in preparing these various compositions should be pharmaceutically or veterinarally pure and non-toxic in the amounts used.

For the purposes of parenteral therapeutic administration, the active ingredient may be incorporated into a solution or suspension. The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

The pharmaceutical forms suitable for injectable use include sterile solutions, dispersions, emulsions, and sterile powders. The final form should be stable under conditions of manufacture and storage. Furthermore, the final pharmaceutical form should be protected against contamination and should, therefore, be able to inhibit the growth of microorganisms such as bacteria or fungi. A single intravenous or intraperitoneal dose can be administered. Alternatively, a slow long-term infusion or multiple short-term daily infusions may be utilized, typically lasting from 1 to 8 days. Alternate day dosing or dosing once every several days may also be utilized.

Sterile, injectable solutions may be prepared by incorporating a compound in the required amount into one or more appropriate solvents to which other ingredients, listed above or known to those skilled in the art, may be added as required. Sterile injectable solutions may be prepared by incorporating the compound in the required amount in the appropriate solvent with various other ingredients as required. Sterilizing procedures, such as filtration, may then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

Suitable pharmaceutical carriers include sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. In all cases, the final form, as noted, must be sterile and should also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

In accordance with the invention, there are provided compositions containing triazine derivatives and methods useful for the in vivo delivery of triazine derivatives in the form of nanoparticles, which are suitable for any of the aforesaid routes of administration.

U.S. Pat. Nos. 5,916,596, 6,506,405 and 6,537,579 teach the preparation of nanoparticles from the biocompatible polymers, such as albumin. Thus, in accordance with the present invention, there are provided methods for the formation of nanoparticles of the present invention by a solvent evaporation technique from an oil-in-water emulsion prepared under conditions of high shear forces (e.g., sonication, high pressure homogenization, or the like).

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

In accordance with the invention, the compounds of the invention may be used to treat diseases associated with cellular proliferation or hyperproliferation, such as cancers which include but are not limited to tumors of the nasal cavity, paranasal sinuses, nasopharynx, oral cavity, oropharynx, larynx, hypopharynx, salivary glands, and paragangliomas. The compounds of the invention may also be used to treat cancers of the liver and biliary tree (particularly hepatocellular carcinoma), intestinal cancers, particularly colorectal cancer, ovarian cancer, small cell and non-small cell lung cancer, breast cancer, sarcomas (including fibrosarcoma, malignant fibrous histiocytoma, embryonal rhabdomysocarcoma, leiomysosarcoma, neuro-fibrosarcoma, osteosarcoma, synovial sarcoma, liposarcoma, and alveolar soft part sarcoma), neoplasms of the central nervous systems (particularly brain cancer), and lymphomas (including Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma).

The compounds and methods of the present invention, either when administered alone or in combination with other agents (e.g., chemotherapeutic agents or protein therapeutic agents described below) are also useful in treating a variety of disorders, including but not limited to, for example: stroke, cardiovascular disease, myocardial infarction, congestive heart failure, cardiomyopathy, myocarditis, ischemic heart disease, coronary artery disease, cardiogenic shock, vascular shock, pulmonary hypertension, pulmonary edema (including cardiogenic pulmonary edema), pleural effusions, rheumatoid arthritis, diabetic retinopathy, retinitis pigmentosa, and retinopathies, including diabetic retinopathy and retinopathy of prematurity, inflammatory diseases, restenosis, asthma, acute or adult respiratory distress syndrome (ARDS), lupus, vascular leakage, protection from ischemic or reperfusion injury such as ischemic or reperfusion injury incurred during organ transplantation, transplantation tolerance induction; ischemic or reperfusion injury following angioplasty; arthritis (such as rheumatoid arthritis, psoriatic arthritis or osteoarthritis); multiple sclerosis; inflammatory bowel disease, including ulcerative colitis and Crohn's disease; lupus (systemic lupus crythematosis); graft vs. host diseases; T-cell mediated hypersensitivity diseases, including contact hypersensitivity, delayed-type hypersensitivity, and gluten-sensitive enteropathy (Celiac disease); Type 1 diabetes; psoriasis; contact dermatitis (including that due to poison ivy); Hashimoto's thyroiditis; Sjogren's syndrome; Autoimmune Hyperthyroidism, such as Graves' disease; Addison's disease (autoimmune disease of the adrenal glands); autoimmune polyglandular disease (also known as autoimmune polyglandular syndrome); autoimmune alopecia; pernicious anemia; vitiligo; autoimmune hypopituitarism; Guillain-Barre syndrome; other autoimmune diseases; cancers, including those where kineses such as Src-family kineses are activated or overexpressed, such as colon carcinoma and thymoma, or cancers where kinase activity facilitates tumor growth or survival; glomerulonephritis, serum sickness; uticaria; allergic diseases such as respiratory allergies (asthma, hayfever, allergic rhinitis) or skin allergies; mycosis fungoides; acute inflammatory responses (such as acute or adult respiratory distress syndrome and ischemialreperfusion injury); dermatomyositis; alopecia greata; chronic actinic dermatitis; eczema; Behcet's disease; Pustulosis palmoplanteris; Pyoderma gangrenum; Sezary's syndrome; atopic dermatitis; systemic sclerosis; morphea; peripheral limb ischemia and ischemic limb disease; bone disease such as osteoporosis, osteomalacia, hyperparathyroidism, Paget's disease, and renal osteodystrophy; vascular leak syndromes, including vascular leak syndromes induced by chemotherapies or immunomodulators such as IL-2; spinal cord and brain injury or trauma; glaucoma; retinal diseases, including macular degeneration; vitreoretinal disease; pancreatitis; vasculatides, including vasculitis, Kawasaki disease, thromboangiitis obliterans, Wegener s granulomatosis, and Behcet's disease; scleroderma; preeclampsia; thalassemia; Kaposi's sarcoma; von Hippel Lindau disease; and the like.

In accordance with the invention, the compounds of the invention may be used to treat diseases associated with undesired cellular proliferation or hyperproliferation comprising identifying the mammal afflicted with said disease or condition and administering to said afflicted mammal a composition comprising the compound of formula I, wherein the disease or condition is associated with a kinase.

In accordance with the invention, the compounds of the invention may be used to treat diseases associated with undesired cellular proliferation or hyperproliferation comprising identifying the mammal afflicted with said disease or condition and administering to said afflicted mammal a composition comprising a compound of formula (I) or formula (II), wherein the disease or condition is associated with a tyrosine kinase.

In accordance with the invention, the compounds of the invention may be used to treat diseases associated with undesired cellular proliferation or hyperproliferation comprising identifying the mammal afflicted with said disease or condition and administering to said afflicted mammal a composition comprising a compound of formula (I) or formula (II), wherein the disease or condition is associated with the kinase that is a serine kinase or a threonine kinase.

In accordance with the invention, the compounds of the invention may be used to treat diseases associated with undesired cellular proliferation or hyperproliferation comprising identifying the mammal afflicted with said disease or condition and administering to said afflicted mammal a composition comprising the compound of formula (I) or formula (II), wherein the disease or condition is associated with the kinase that is a Src family kinase.

In accordance with the invention, the compounds of the invention may be used to treat diseases associated with undesired cellular proliferation or hyperproliferation comprising identifying the mammal afflicted with said disease or condition and administering to said afflicted mammal a composition comprising the compound of formula (I) or foimula (II), wherein the disease or condition is associated with the kinase that is a Aurora family kinase.

The invention also provides methods of treating a mammal afflicted with the above diseases and conditions. The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

In one aspect, the invention compounds are administered in combination with chemotherapeutic agent, an anti-inflammatory agent, antihistamines, chemotherapeutic agent, immunomodulator, therapeutic antibody or a protein kinase inhibitor, e.g., a tyrosine kinase inhibitor, to a subject in need of such treatment.

The method includes administering one or more of the inventive compounds to the afflicted mammal. The method may further include the administration of a second active agent, such as a cytotoxic agent, including alkylating agents, tumor necrosis factors, intercalators, microtubulin inhibitors, and topoisomerase inhibitors. The second active agent may be co-administered in the same composition or in a second composition. Examples of suitable second active agents include, but are not limited to, a cytotoxic drug such as Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil 131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-□a; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogemanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

In accordance with the invention, the compounds and compositions may be used at sub-cytotoxic levels in combination with other agents in order to achieve highly selective activity in the treatment of non-neoplastic disorders, such as heart disease, stroke and neurodegenerative diseases (Whitesell et al., Curr Cancer Drug Targets (2003), 3(5), 349-58).

The exemplary therapeutical agents that may be administered in combination with invention compounds include EGFR inhibitors, such as gefitinib, erlotinib, and cetuximab. Her2 inhibitors include canertinib, EKB-569, and GW-572016. Also included are Src inhibitors, dasatinib, as well as Casodex (bicalutamide), Tamoxifen, MEK-1 kinase inhibitors, MARK kinase inhibitors, PI3 inhibitors, and PDGF inhibitors, such as imatinib, Hsp90 inhibitors, such as 17-AAG and 17-DMAG. Also included are anti-angiogenic and antivascular agents which, by interrupting blood flow to solid tumors, render cancer cells quiescent by depriving them of nutrition. Castration, which also renders androgen dependent carcinomas non-proliferative, may also be utilized. Also included are IGF1R inhibitors, inhibitors of non-receptor and receptor tyrosine kineses, and inhibitors of integrin.

The pharmaceutical composition and method of the present invention may further combine other protein therapeutic agents such as cytokines, immunomodulatory agents and antibodies. As used herein the term “cytokine” encompasses chemokines, interleukins, lymphokines, monokines, colony stimulating factors, and receptor associated proteins, and functional fragments thereof. As used herein, the term “functional fragment” refers to a polypeptide or peptide which possesses biological function or activity that is identified through a defined functional assay. The cytokines include endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

Other therapeutic agents for the combinatory therapy include cyclosporins (e.g., cyclosporin A), CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and for gpn39 (i.e., CD154), fusion proteins constructed from CD40 and gp39 (CD40Ig and CD8gp39), inhibitors, such as nuclear translocation inhibitors, of NF-kappa B function, such as deoxyspergualin (DSG), cholesterol biosynthesis inhibitors such as HM:G CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, TNF-a inhibitors such as tenidap, anti-TNF antibodies or soluble TNF receptor, and rapamycin (sirolimus or Rapamune) or derivatives thereof.

When other therapeutic agents are employed in combination with the compounds of the present invention they may be used for example in amounts as noted in the Physician Desk Reference (PDR) or as otherwise determined by one having ordinary skill in the art.

The following examples are provided to further illustrate the present invention but, of course, should not be construed as in any way limiting its scope.

All experiments were performed under anhydrous conditions (i.e. dry solvents) in an atmosphere of argon, except where stated, using oven-dried apparatus and employing standard techniques in handling air-sensitive materials. Aqueous solutions of sodium bicarbonate (NaHCO3) and sodium chloride (brine) were saturated.

Analytical thin layer chromatography (TLC) was carried out on Merck Kiesel gel 60 F254 plates with visualization by ultraviolet and/or anisaldehyde, potassium permanganate or phosphomolybdic acid dips.

NMR spectra: 1H Nuclear magnetic resonance spectra were recorded at 400 MHz. Data are presented as follows: chemical shift, multiplicity (s=singlet, d=doublet, t=triplet, q=quartet, qn=quintet, dd=doublet of doublets, m=multiplet, bs=broad singlet), coupling constant (J/Hz) and integration. Coupling constants were taken and calculated directly from the spectra and are uncorrected.

Low resolution mass spectra: Electrospray (ES+) ionization was used. The protonated parent ion (M+H) or parent sodium ion (M+Na) or fragment of highest mass is quoted. Analytical gradient consisted of 10% ACN in water ramping up to 100% ACN over 5 minutes unless otherwise stated.

### Example 1

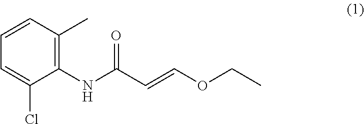

A mixture of ethyl β-ethoxyacrylate (26.50 g, 183 mmol) and 2 N sodium hydroxide (110 mL, 220 mmol) was refluxed for 2 h and cooled to 0° C. water was removed under vacc., and the yellow solids were triturated with toluene and evaporated to give the sodium β-ethoxyacrylate (25 g, 97%). The mixture of sodium β-thoxyacrylate (10.26 g, 74.29 mmol) and thionyl chloride (25 mL, 343 mmol) was refluxed for 2 h, and evaporated. to give the β-ethoxyacryloyl chloride crude product, which was used without purification. To a cold stirring solution of 3-ethoxyacryloyl chloride in THF (100 mL) was added 2-chloro-6-methylaniline (6.2 mL, 50.35 mmol) and pyridine (9 ml, 111 mmol). The mixture was then warmed and stirred overnight at room temperature. Water was added at 0-10° C., extracted with EtOAc. The organic layer was washed with CuSO₄ (3×50 mL) and the resulting solution was passed a pad of silica gel, concentrated under vacuum to give solids. The solids was diluted with toluene and kept. at 0° C. The solid was collected by vacuum filtration, washed with water and dried to give 5.2 g (43% yield) of compound 1, (E)-N-(2-chloro-6-methylphenyl)-3-ethoxyacrylamide). ¹H NMR (500 Hz, CDCl₃) δ 1.26 (t, 3H, J=7 Hz), 2.15 (s, 3H), 3.94 (q, 2H, J=7 Hz), 5.58 (d, 1H, J=12.4 Hz), 7.10-7.27 (m, 21-1, J=7.5 Hz), 7.27-7.37 (d, 1H, J=7.5 Hz), 7.45 (d, 1H, J=12.4 Hz); ESI-MS: calcd for (C₁₂H₁₄ClNO₂) 239. found 240 MH⁺).

### Example 2

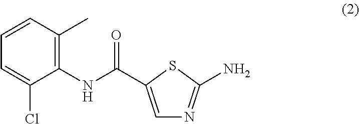

To a mixture of compound 1 (5.30 g, 22.11 mmol) in 1,4-dioxane (100 mL) and water (70 mL) was added NBS (4.40 g, 24.72 mmol) at −10 to 0° C. The slurry was warmed and stirred at 20-22° C. for 3 h. Thiourea (1.85 g, 26.16 mmol) was added and the mixture heated to 100° C. After 2 h, the resulting solution was cooled to 20-22° C. and conc. ammonium hydroxide (6 mL) was added dropwise. The resulting slurry was concentrated under vacuum to about half volume and cooled to 0-5° C. The solid was collected by vacuum filtration, washed with cold water, and dried to give 5.4 g (90% yield) of compound 2 as deep-yellow solids. ¹H NMR (500 MHz, DMSO-d₆) δ 2.19 (s, 3H), 7.09-7.29 (m, 2H, J=7.5), 7.29-7.43 (d, 1H, J=7.5), 7.61 (s, 2H), 7.85 (s, 1H), 9.63 (s, 1H); ESI-MS: calcd for (C₁₁H₁₀ClN₃OS) 267. found 268 MH⁺).

### Example 3

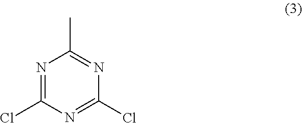

A solution of methylmagnesium bromide in ether (3M, 30 ml, 90 mmole) was added dropwise to a stirred solution of cyanuric chloride (3.91 g, 21.20 mmole) in anhydrous dichloromethane at −10° C. After the addition was complete, the reaction mixture was stirred at −5° C. for 4 h, after which time water was added dropwise at a rate such that the temperature of the reaction stayed below 10° C. After warming to room temperature, the reaction mixture was diluted with additional water and methylene chloride and passed through a pad of cilite. The organic layer was dried and evaporated to give 2,4-dichloro-6-methyl-1,3,5-triazine of 4 as yellow solids (3.02 g, 87%). ¹H NMR (CDCl₃) δ 2.70 (s, 3H).

### Example 4

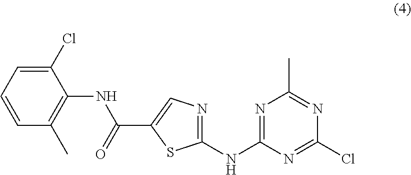

A solution of Compound 3 (560 mg, 3.41 mmole), diisopropylamine (1.00 ml, 5.74 mmole) and Compound 2 (700 mg, 2.65 mmole) in THF (40 mL) was stirred at 0° C. for 30 min, then at room temperature for 2 hours. Water was added to the reaction mixture, and the aqueous mixture was extracted twice with EtOAc. The combined extracts were washed with brine, dried, and evaporated in vacuo. Column chromatography provided Compound 4 as light yellow solids (350 mg, 33%). ¹H NMR (500 MHz, DMSO-d₆) δ 2.19 (s, 3H), 2.49 (s, 3H), 7.36-7.58 (m, 3H), 8.23 (br, 1H), 9.61 (br, 1H), 11.63 (br, 1H); ESI-MS: calcd for (C₁₅H₁₂Cl₂N₆OS) 394. found 395 (MH⁺).

### Example 5

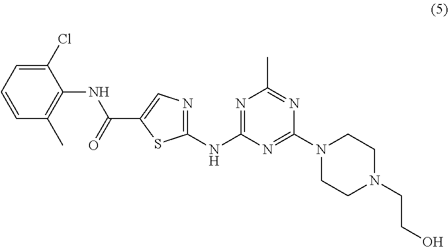

A mixture of 4 (100 mg, 0.25 mmol), diisopropylethylamine (0.08 mL, 0.50 mmol), and 1-(2-hydroxyethyl)piperazine (100 mg, 0.77 mmol) in 1,4-dioxane (15 mL) was refluxed for 12 h. The mixture was concentrated under vacuum, and water was added. The solid was collected by filtration, triturated successively with H₂O, aqueous MeOH, and Et₂O (2×) and dried in vacuoto give 5 as light yellow solids (55 g, 45%). ¹H NMR (500 MHz, DMSO-d₆) δ11.97 (br s, 1H), 10.00 (s, 1H), 8.28 (s, 1H), 7.40 (d, J=7.6 Hz, 1H), 7.29-7.24 (m, 2H), 4.45 (t, J=5.4 Hz, 1H), 3.87-3.81 (m, 4H), 3.52 (q, J=6.0 Hz, 2H), 2.46 (m, 4H), 2.42 (t, J=6.0 Hz, 2H), 2.30 (s, 3H), 2.23 (s, 3H). ESI-MS: calcd for (C₂₁H₂₅ClN₈O₂S) 488. found 489 (MH⁺).

### Example 6

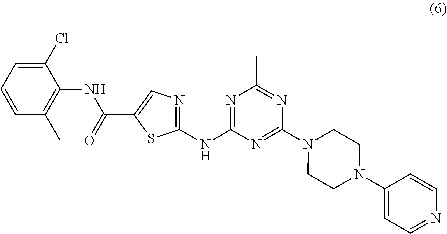

Compound 6 was prepared by the same procedure as was used in the preparation of Compound 5. Light yellow solids were obtained (42% yield). ESI-MS: calcd for (C₂₄H₂₄ClN₉OS) 521. found 522 (MH⁺).

### Example 7

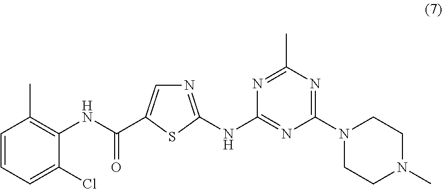

A mixture of 4 (200 mg, 0.51 mmol), diisopropylethylamine (0.35 mL, 2.03 mmol), and 1-methyl piperazine (304 mg, 3.04 mmol) in DMSO (10 mL) was heated at 70° C. for 13 h. The mixture was extracted by ethyl acetate and the combined organic layers were washed with water and brine. The crude product was recrystallized by MeOH/CHCl₃ (23 mg, 9.9%). ¹H NMR (500 MHz, DMSO-d₆) δ 12.01 (br s, 1H), 10.0 (br s, 1H), 8.29 (s, 1H), 7.42 (d, J=7.4 Hz, 1H), 7.30-7.24 (m, 2H), 3.89 (m, 4H), 2.54-2.43 (m, 4H), 2.34-2.23 (m, 9H), ESI-MS: calcd for (C₂₀H₂₃ClN₈OS) 458. found 459 (M+H⁺). HPLC: retention time: 9.8 min; purity 93%.

### Example 8

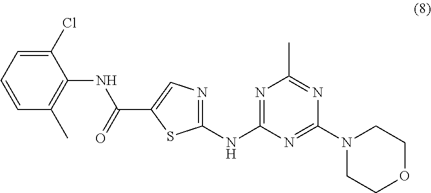

Compound 8 was prepared by the same procedure as was used in the preparation of Compound 5. Light yellow solids were obtained (94% yield). ESI-MS: calcd for (C₁₉H₂₀ClN₇O₂S) 445. found 446 (MH⁺).

### Example 9

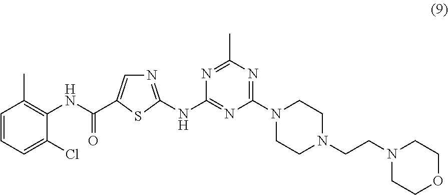

A mixture of 4 (100 mg, 0.25 mmol), diisopropylethylamine (0.07 mL, 0.391 mmol), and 4-(2-(piperazin-1-yl)ethyl) morpholine (152 mg, 0.76 mmol) in 1,4-dioxane (15 mL) was refluxed for 12 h. The mixture was concentrated under vacuum, and water was added. The mixture was extracted by ethyl acetate and the combined organic was concentrated. The crude product was passed on a pad of silica gel by using 2 to 10% MeOH—NH₃/CH₂Cl₂ (10 mg, 7%). ¹H NMR (500 MHz, DMSO-d₆) δ11.97 (br s, 1H), 9.99 (s, 1H), 8.28 (s, 1H), 7.40 (d, J=7.0 Hz, 1H), 7.30-7.24 (m, 2H), 3.87-3.80 (m, 4H), 3.54 (m, 4H), 2.58-2.41 (m, 12H), 2.34 (s, 3H), 2.23 (s, 3H), ESI-MS: calcd for (C₂₅H₃₂ClN₉O₂S) 557. found 558 (MH⁺). HPLC: retention time: 9.92 min.; purity: 97%.

### Example 10

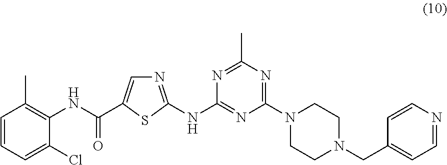

A mixture of 4 (125 mg, 0.32 mmol), diisopropylethylamine (0.085 mL, 0.48 mmol), and 1-(pyridin-4-ylmethyl)piperazine (168 mg, 0.95 mmol) in 1,4-dioxane (15 mL) was refluxed for 12 h. The mixture was concentrated under vacuum, and water was added. The mixture was extracted by ethyl acetate and the combined organic layer was concentrated. The crude product was purified by slica gel chromatography) by using 5% to 10% MeOH/EtOAc (15 mg, 9%). ¹H NMR (500 MHz, DMSO-d₆) δ11.97 (br s, 1H), 9.97 (s, 1H), 8.52-8.50 (d, J=5.0 Hz, 2H), 8.27 (s, 1H), 7.40-7.35 (m, 4H), 7.29-7.24 (m, 2H), 3.86-3.80 (m, 4H), 3.57 (s, 2H), 2.53-2.41 (m, 4H), 2.33 (s, 3H), 2.23 (s, 3H). ESI-MS: calcd for (C₂₅H₂₆ClN₉OS) 535. found 536 (MH⁺). HPLC: retention time: 11.55 min.; purity: 90%.

### Example 11

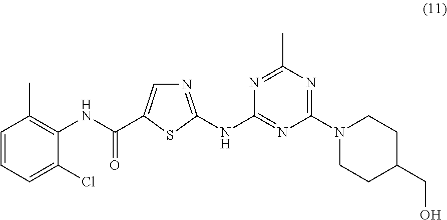

A mixture of 4 (125 mg, 0.32 mmol), diisopropylethylamine (0.085 mL, 0.48 mmol), and piperidin-4-yl-methanol (109 mg, 0.95 mmol) in 1,4-dioxane (15 mL) was refluxed for 12 h. The mixture was concentrated under vacuum, and water was added. The mixture was extracted by ethyl acetate and the combined organic layer was concentrated. The crude product was purified by slica gel chromatography) by using 10% MeOH/EtOAc (30 mg, 20%). ¹H NMR (500 MHz, DMSO-d₆) δ11.97 (br s, 1H), 9.98 (s, 1H), 8.28 (s, 1H), 7.40 (d, J=7.5 Hz, 1H), 7.30-7.24 (m, 2H), 4.79-4.72 (m, 2H), 4.51 (t, J=5.5 Hz, 1H), 3.26 (m, 2H), 3.10-2.90 (m, 2H), 2.33 (s, 3H), 2.23 (s, 3H), 1.80-1.61 (m, 2H), 1.20-1.0 (m, 2H); ESI-MS: calcd for (C₂₁H₂₄ClN₇O₂S) 473. found 474 (MH⁺). HPLC: retention time: 8.45 min.; purity: 98%.

### Example 12

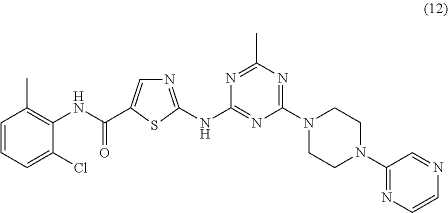

A mixture of 4 (125 mg, 0.32 mmol), diisopropylethylamine (0.085 mL, 0.48 mmol), and 2-(piperazin-1-yl)pyrazine (156 mg, 0.95 mmol) in 1,4-dioxane (15 mL) was refluxed for 12 h. The mixture was concentrated under vacuum, and water was added. The mixture was extracted by ethyl acetate and the combined organic layer was concentrated. The crude product was purified by slica gel chromatographyl by using 10% MeOH/EtOAc (30 mg, 18%). ¹H NMR (500 MHz, DMSO-d₆) δ12.02 (br s, 1H), 10.0 (s, 1H), 8.38 (d, J=1.2 Hz, 1H), 8.29 (s, 1H), 8.10 (s, 1H), 7.86 (d, J=2.5 Hz, 1H), 7.40 (d, J=7.5 Hz, 1H), 7.32-7.24 (m, 2H), 4.10-3.90 (m, 4H), 3.70-3.58 (m, 4H), 2.34 (s, 3H), 2.23 (s, 3H), ESI-MS: calcd for (C₂₃H₂₃ClN₁₀OS) 522. found 523 (MH⁺). HPLC: retention time: 24 min.; purity: 92%.

### Example 13

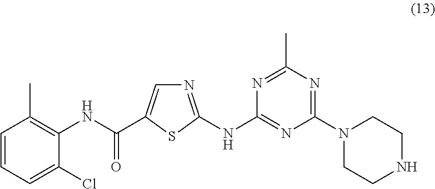

A mixture of 4 (200 mg, 0.51 mmol), diisopropylethylamine (0.35 mL, 2.03 mmol), and piperazine (436 mg, 5.07 mmol) in 1,4-dioxane (25 mL) was refluxed for 12 h. The mixture was concentrated under vacuum, and water was added. The mixture was extracted by ethyl acetate and the combined organic layer was concentrated. The crude product was purified by column chromatography by using 10% MeOH/CH₂Cl₂ and then recrystallized with MeOH/Chloroform (11 mg, 7%). ¹H NMR (500 MHz, DMSO-d₆) δ11.95 (br s, 1H), 10.0 (s, 1H), 8.29 (s, 1H), 7.40 (d, J=7.5 Hz, 1H), 7.29-7.24 (m, 2H), 3.90-3.70 (m, 4H), 2.90-2.69 (m, 4H), 2.30 (s, 3H), 2.23 (s, 3H), ESI-MS: calcd for (C₁₉H₂₁ClN₈OS) 444. found 445 (MH⁺). HPLC: retention time: 9.24 min.; purity: 93%.

### Example 14

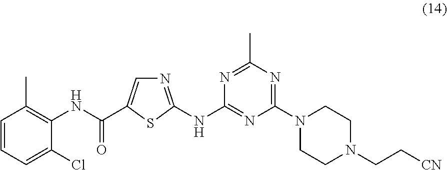

A mixture of 4 (125 mg, 0.32 mmol), diisopropylethylamine (0.19 mL, 1.1 mmol), and 3-(piperazin-1-yl)propanenitrile (220 mg, 1.59 mmol) in 1,4-dioxane (15 mL) was refluxed for 12 h. The mixture was concentrated under vacuum, and water was added. The mixture was extracted by ethyl acetate and the combined organic layer was concentrated. The crude product was recrystallized by MeOH/Chloroform (15 mg, 12%). ¹H NMR (500 MHz, DMSO-d₆) δ12.00 (br s, 1H), 9.99 (s, 1H), 8.28 (s, 1H), 7.41 (d, J=7.4 Hz, 1H), 7.29-7.24 (m, 2H), 3.92-3.79 (m, 4H), 2.71 (t, J=7 Hz, 2H), 2.61 (t, J=6.5 Hz, 2H), 2.55-2.45 (m, 4H), 2.31 (s, 3H), 2.24 (s, 3H), ESI-MS: calcd for (C₂₂H₂₄ClN₉OS) 497. found 498 (MH⁺). HPLC: retention time: 12.16 min; purity: 88%.

### Example 15

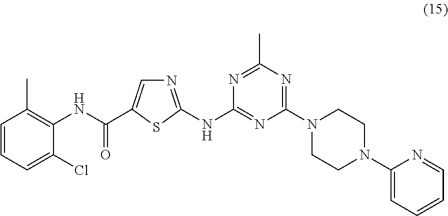

A mixture of 4 (100 mg, 0.25 mmol), diisopropylethylamine (0.07 mL, 1.5 mmol), and 1-(pyridin-2-yl)piperazine (83 mg, 0.508 mmol) in 1,4-dioxane (15 mL) was refluxed for 12 h. The mixture was concentrated under vacuum, and water was added. The mixture was extracted by ethyl acetate and the combined organic layer was concentrated. The crude product was recrystallized by MeOH/CH₂Cl₂ (8 mg, 6%). ¹H NMR (500 MHz, DMSO-d₆) δ 12.01 (br s, 1H), 10.0 (r s, 1H), 8.29 (s, 1H), 8.12 (d, J=3.5 Hz, 1H), 7.52-7.60 (m, 1H), 7.41 (d, J=7.4 Hz, 1H), 7.30-7.24 (m, 2H), 6.91 (d, J=9 Hz, 1H), 6.68-6.64 (m, 1H), 4.02-3.92 (m, 4H), 3.68-3.58 (m, 4H), 2.34 (s, 3H), 2.25 (s, 3H), ESI-MS: calcd for (C₂₄H₂₄ClN₉OS) 521. found 522 (MH⁺). HPLC: retention time: 15 min; purity: 89%.

### Example 16

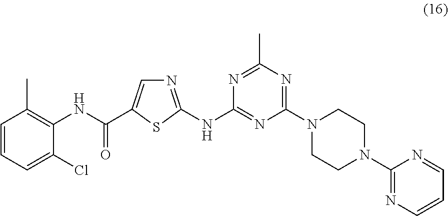

A mixture of 4 (100 mg, 0.25 mmol), diisopropylethylamine (0.07 mL, 1.5 mmol), and 2-(piperazin-1-yl)pyrimidine (83 mg, 0.508 mmol) in 1,4-dioxane (10 mL) was refluxed for 12 h. The mixture was concentrated under vacuum, and water was added. The mixture was extracted by ethyl acetate and the combined organic layer was concentrated. The crude product was recrystallized by MeOH/CHCl₃ (2 mg, 1.5%). ¹H NMR (500 MHz, DMSO-d₆) δ 12.01 (br s, 1H), 9.98 (s, 1H), 8.38 (d, J=4.5 Hz, 2H), 8.28 (s, 1H), 7.40 (d, J=7.4 Hz, 1H), 7.30-7.24 (m, 2H), 6.67 (t J=3 Hz, 1H), 4.12-3.85 (m, 8H), 2.34 (s, 3H), 2.25 (s, 3H), ESI-MS: calcd for (C₂₃H₂₃ClN₁₀OS) 522. found 523 (MH⁺). HPLC: retention time: 25 min; purity 97%.

### Example 17

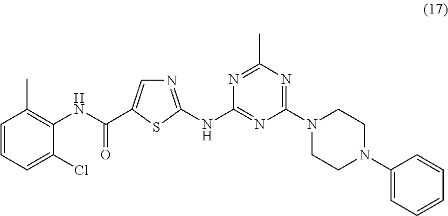

A mixture of 4 (100 mg, 0.25 mmol), diisopropylethylamine (0.07 mL, 1.5 mmol), and 1-phenylpiperazine (82 mg, 0.508 mmol) in 1,4-dioxane (10 mL) was refluxed for 12 h. The mixture was concentrated under vacuum, and water was added. The mixture was extracted by ethyl acetate and the combined organic layer was concentrated. The crude product was recrystallized by MeOH/CHCl₃ (12 mg, 9%). ¹H NMR (500 MHz, DMSO-d₆) δ 12.01 (br s, 1H), 10.0 (s, 1H), 8.30 (s, 1H), 7.41 (d, J=7.4 Hz, 1H), 7.35-7.24 (m, 4H), 6.99 (d, J=8 Hz, 2H), 6.81 (t, J=7 Hz, 1H), 4.12-3.90 (m, 4H), 3.30-3.10 (m, 4H), 2.34 (s, 3H), 2.24 (s, 3H), ESI-MS: calcd for (C₂₅H₂₅ClN₈OS) 520. found 521 (MH⁺). HPLC: retention time: 34 min; purity 89%.

### Example 18

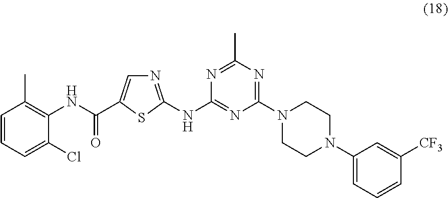

A mixture of 4 (100 mg, 0.25 mmol), diisopropylethylamine (0.07 mL, 1.5 mmol), and 1-(3-(trifluoromethyl)phenyl)piperazine (117 mg, 0.508 mmol) in 1,4-dioxane (10 mL) was refluxed for 12 h. The mixture was concentrated under vacuum, and water was added. The mixture was extracted by ethyl acetate and the combined organic layer was concentrated. The crude product was recrystallized by MeOH/CHCl₃ (11 mg, 7%). ¹H NMR (500 MHz, DMSO-d₆) δ12.01 (br s, 1H), 10.0 (s, 1H), 8.30 (s, 1H), 7.48-7.40 (d, J=7.4 Hz, 2H), 7.34-7.24 (m, 4H), 7.09 (d, J=8 Hz, 1H), 4.12-3.90 (m, 4H), 3.45-3.30 (m, 4H), 2.34 (s, 3H), 2.24 (s, 3H), ESI-MS: calcd for (C₂₆H₂₄ClF₃N₈OS) 588. found 589 (MH⁺). HPLC: retention time: 39 min; purity 93%.

### Example 19

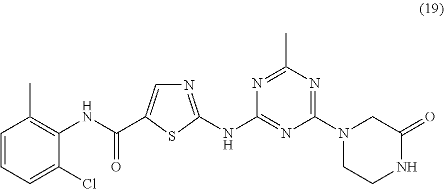

A mixture of 4 (300 mg, 0.25 mmol), diisopropylethylamine (0.66 mL, 3.8 mmol), and piperazin-2-one (761 mg, 7.61 mmol) in DMSO (10 mL) was heated at 65° C. for 13 h. The mixture was extracted by ethyl acetate and the combined organic layers were washed with water and brine. The crude product was recrystallized by MeOH/CHCl₃ (30 mg, 8.6%). ¹H NMR (500 MHz, DMSO-d₆) δ12.01 (br s, 1H), 10.0 (br s, 1H), 8.30 (s, 1H), 8.15 (br s, 1H), 7.41 (d, J=7.4 Hz, 1H), 7.30-7.24 (m, 2H), 4.45-4.32 (m, 1H), 4.10-3.92 (m, 1H), 2.62-2.49 (m, 4H), 3.45-3.30 (m, 4H), 2.34 (s, 3H), 2.24 (s, 3H), ESI-MS: calcd for (C₁₉H₁₉ClN₈O₂S) 458. found 481 (M+Na⁺). HPLC: retention time: 12.7 min; purity 90%.

### Example 20

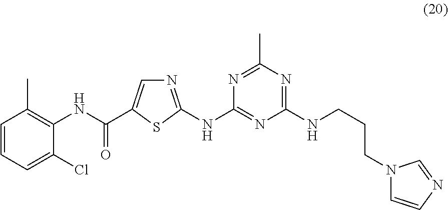

A mixture of 4 (300 mg, 0.76 mmol) and 3-(1H-imidazol-1-yl)propan-1-amine (821 mg, 4.6 mmol) in 2-propanol (10 mL) was heated at 85° C. for 5 h. The mixture was extracted by ethyl acetate and the combined organic layers were washed with sodium bicarbonate, water and brine. The crude product was recrystallized by MeOH/CHCl₃ (30 mg, 8.1%). ¹H NMR (500 MHz, DMSO-d₆) δ12.01 (br s, 1H), 10.05 (br s, 1H), 8.30 (s, 1H), 7.62 (s, 1H), 7.41 (d, J=7.4 Hz, 1H), 7.30-7.24 (m, 2H), 7.17 (s, 1H), 6.83 (s, 1H), 4.06 (t, J=7 Hz, 2H), 2.32-2.24 (m, 8H), 2.05-1.95 (m, 2H) ESI-MS: calcd for C₂₁H₂₂ClN₉OS) 483 found 484 (M+H⁺). HPLC: retention time: 7.9 min; purity 90.5%.

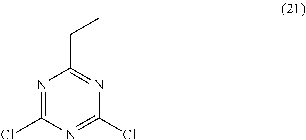

A solution of ethylmagnesium bromide in ether (3M, 15 ml, 45 mmole) was added dropwise to a stirred solution of cyanuric chloride (5.64 g, 30.58 mmole) in anhydrous dichloromethane at −10° C. After the addition was complete, the reaction mixture was stirred at −5° C. for 1 h, after which time water was added dropwise at a rate such that the temperature of the reaction stayed below 10° C. After warming to room temperature, the reaction mixture was diluted with additional water and methylene chloride and passed through a pad of cilite, washed by saturated ammonium chloride, dried and concentrated to give 2,4-dichloro-6-ethyl-1,3,5-triazine 21 as yellow liquid, which solidified after storied in the refrigerator (5.20 g, 96%). ¹H NMR (CDCl³) δ 2.95 (q, J=7.5 Hz. 2H), 1.38 (t, J=7.5 Hz. 3H).

### Example 22

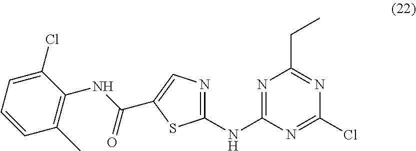

A solution of Compound 2 (1.07 g, 4.11 mmole), diisopropylamine (10.78 ml, 4.47 mmole) and Compound 21 (1.10 g, 6.18 mmole) in THF (70 mL) was stirred at 0° C. for 8 hours, then cold 5% NaHCO₃ was added to the reaction mixture, and the aqueous mixture was extracted twice with EtOAc. The combined extracts were washed with brine, dried, and concentrated in vacuo until a lot of precipitate formed. After filtration, the solids were washed by ethyl acetate dried to give 22 (800 mg, 48%), which was used without purification for the next step reactions.

### Example 23

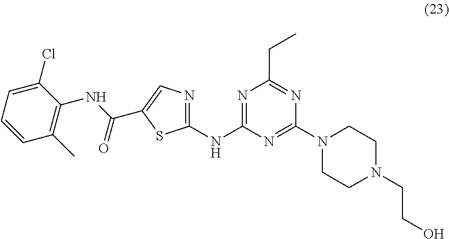

A mixture of 22 (258 mg, 0.63 mmol), diisopropylethylamine (0.32 mL, 1 . . . 83 mmol), and 1-(2-hydroxyethyl)piperazine (280 mg, 2 . . . 15 mmol) in 1,4-dioxane (50 mL) was stirred at 70° C. overnight. The mixture was concentrated under vacuum, and water was added. The mixture was extracted by ethyl acetate and the combined organic was concentrated, passed a pad of silica gel and concentrated to give light yellow solids, which was crystallized from methanol-THF to give white solids 23 (125 mg, 39%). ¹H NMR (500 MHz, DMSO-d₆) δ11.97 (br s, 1H), 10.00 (s, 1H), 8.28 (s, 1H), 7.40 (d, J=7.6 Hz, 1H), 7.29-7.24 (m, 2H), 4.46 (t, J=5.0 Hz, 1H), 3.87-3.81 (m, 4H), 3.52 (q, J=6.0 Hz, 2H), 2.58-2.41 (m, 8H), 2.23 (s, 3H), 1.20 (t, J=7.0 Hz, 3H). ESI-MS: calcd for (C₂₂H₂₇ClN₈O₂S) 502. found 503 (MH⁺). HPLC: retention time: 12.35 min.; purity: 99%.

### Example 24

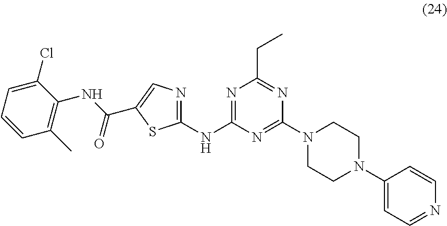

Compound 24 was prepared by the same procedure as was used in the preparation of Compound 23. White solids were obtained (29% yield). ¹H NMR (500 MHz, DMSO-d₆) δ 11.97 (br s, 1H), 10.00 (s, 1H), 8.31 (s, 1H), 8.23 (d, J=5.0 Hz, 2H), 7.40 (d, J=8.0 Hz, 1H), 7.30-7.25 (m, 2H), 7.00 (d, J=5.0 Hz, 2H), 4.00 (m, 4H), 3.70-3.65 (m, 4H), 2.61 (br, 2H), 2.24 (s, 3H), 1.25 (br, 3H). ESI-MS: calcd for (C₂₅H₂₆ClN₉OS) 535. found 536 (MH⁺). HPLC: retention time: 16.18 min.; purity: 99%.

### Example 25

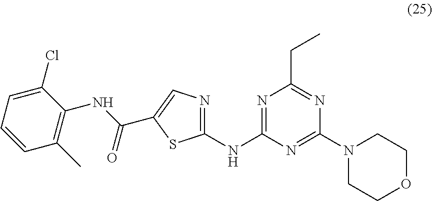

Compound 25 was prepared by the same procedure as was used in the preparation of Compound 23. White solids were obtained (50% yield). ¹H NMR (500 MHz, DMSO-d₆) δ 11.97 (br s, 1H), 10.00 (s, 1H), 8.28 (s, 1H), 7.40 (d, J=7.5 Hz, 1H), 7.30-7.25 (m, 2H), 3.84 (m, 4H), 3.70-3.65 (m, 4H), 2.61 (br, 2H), 2.23 (s, 3H), 1.25 (br, 3H). ESI-MS: calcd for (C₂₀H₂₂ClN₇O₂S) 459. found 460 (MH⁺). HPLC: retention time: 23.91 min.; purity: 99%.

### Example 26

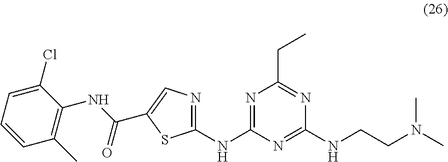

Compound 26 was prepared by the same procedure as was used in the preparation of Compound 23. White solids were obtained (22% yield). ¹H NMR (500 MHz, DMSO-d₆) δ 11.97 (br s, 1H), 10.00 (s, 1H), 8.28 (s, 1H), 7.60 (br, 1H), 7.40 (d, J=7.6 Hz, 1H), 7.29-7.24 (m, 2H), 3.42 (m, 2H), 2.52 (m, 4H), 2.45-2.17 (m, 9H), 1.20 (m, 3H). ESI-MS: calcd for (C₂₀H₂₅ClN₈OS) 460. found 461 (MH⁺). HPLC: retention time: 10.96 min.; purity: 95%.

### Example 27

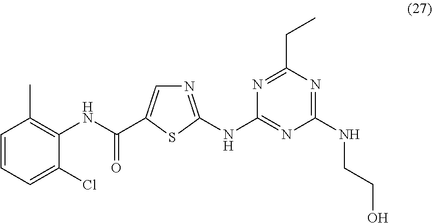

A mixture of compound 22 (250 mg, 0.61 mmol) and diisopropylethylamine (0.43 mL, 2.45 mmol), and 2-aminoethanol (373 mg, 6.13 mmol) in DMSO was heated at 70° C. for over night. The mixture was extracted by ethyl acetate and the combined organic layers were washed with water and brine. The crude product was recrystallized by MeOH/CHCl₃ to give compound 27 (23 mg, 8.6%). ¹H NMR (500 MHz, DMSO-d₆) δ11.83 (br s, 1H), 10.02 (br s, 1H), 8.31 (s, 1H), 7.80 (br s, 1H), 7.41 (d, J=7.4 Hz, 1H), 7.30-7.24 (m, 2H), 4.80-4.72 (br s, 1H), 3.62-3.38 (m, 4H), 2.58-2.50 (m, 2H), 2.24 (s, 3H), 1.20 (m, 3H); ESI-MS: calcd for C₁₈H₂₀ClN₇O₂S) 433 found 434 (M+H⁺). HPLC: retention time: 12.2 min; purity 90.6%.

### Example 28

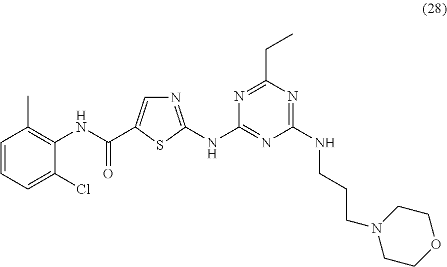

A mixture of compound 22 (250 mg, 0.61 mmol) and diisopropylethylamine (0.43 mL, 2.45 mmol), and 3-morpholinopropan-1-amine (882 mg, 6.13 mmol) was heated at 70° C. in DMSO for over night. The mixture was extracted by ethyl acetate and the combined organic layers were washed with water and brine. The crude product was recrystallized by MeOH/CHCl₃ to give compound 28 (33 mg, 10%). ¹H NMR (500 MHz, DMSO-d₆) δ11.45 (br s, 1H), 9.96 (br s, 1H), 8.28 (s, 1H), 8.10 (br s, 1H), 7.41 (d, J=7.4 Hz, 1H), 7.30-7.24 (m, 2H), 3.62-3.52 (m, 4H), 2.63-2.50 (m, 6H), 2.42-2.25 (m, 5H), 2.24 (s, 3H), 1.68-1.75 (m, 1H), 1.22 (m, 3H); ESI-MS: calcd for C₂₃H₂₉ClN₈O₂S) 516 found 517 (M+H⁺). HPLC: retention time: 12.7 min; purity 86%.

### Example 29

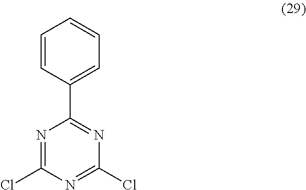

A solution of phenylmagnesium bromide in ether (3M, 16 ml, 48 mmole) was added dropwise to a stirred solution of cyanuric chloride (5.93 g, 32.16 mmole) in anhydrous dichloromethane at 5° C. After the addition was complete, the reaction mixture was stirred at 10-20° C. for 3 h. The mixture was cooled to 0° C. and added water dropwise at a rate such that the temperature of the reaction stayed below 10° C. After warming to room temperature, the reaction mixture was diluted with additional water and methylene chloride and passed through a pad of cilite, washed by saturated ammonium chloride, dried and concentrated to give 2,4-dichloro-6-phenyl-1,3,5-triazine 29 as yellow liquid, which solidified after storage in the refrigerator (1.8 g, 25%). ¹H NMR (500 MHz, CDCl₃) δ 8.50 (d, J=8.0 Hz, 2H), 7.70 (t, J=8.0 Hz, 1H), 7.55 (t, J=8.0 Hz. 2H).

### Example 30

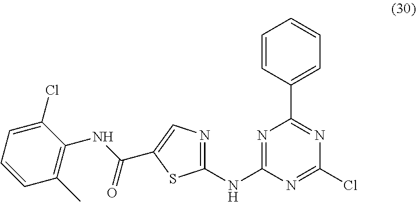

A solution of Compound 2 (500 mg, 1.87 mmole), diisopropylamine (0.33 ml, 1.87 mmole) and Compound 9 (630 mg, 2.81 mmole) in THF (30 mL) was stirred at 0° C. for 3 hours, then room temperature for additional 3 hours. Water was added to the reaction mixture, and the aqueous mixture was extracted twice with EtOAc. The combined extracts were washed with brine, dried, and concentrated in vacuo until a lot of precipitate formed. After filtration, the solids were washed by ethyl acetate dried to give compound 30 (250 mg, 29%), which was used without purification for the next step reactions.

### Example 31

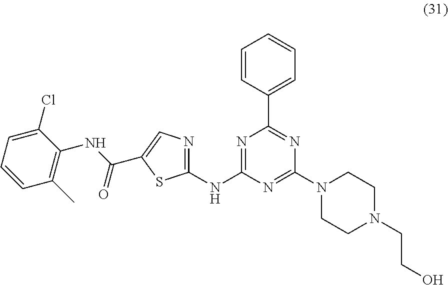

A mixture of 30 (220 mg, 0.48 mmol), diisopropylethylamine (0.30 mL, 1.72 mmol), and 1-(2-hydroxyethyl)piperazine (260 mg, 2.00 mmol) in DMSO (10 mL) was stirred at 60° C. overnight. Water was added, followed by ethyl acetate. White solids precipitated from the solution, which was filtered and washed by ethyl acetate to give the desired product 31 (180 mg, 33%). ¹H NMR (500 MHz, DMSO-d₆) δ11.97 (br s, 1H), 10.03 (s, 1H), 8.45 (br, 2H), 8.32 (s, 1H), 7.61 (t, J=7.0 Hz, 1H), 7.55 (t, J=7.5 Hz, 2H), 7.41 (d, J=8.0 Hz, 1H), 7.31-7.24 (m, 2H), 4.48 (t, J=5.0 Hz, 1H), 3.99 (m, 4H), 3.55 (q, J=6.0 Hz, 2H), 2.54 (br, 4H), 2.45 (t, J=6.0 Hz, 2H), 2.25 (s, 3H). ESI-MS: calcd for (C₂₆H₂₇ClN₈O₂S) 550. found 551 (MH⁺). HPLC: retention time: 20.02 min.; purity: 98%.

### Example 32

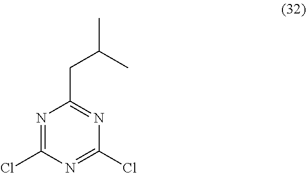

A solution of iso-butylmagnesium bromide in ether (2M, 35 ml, 70.0 mmole) was added dropwise to a stirred solution of cyanuric chloride (5.28 g, 28.63 mmole) in anhydrous dichloromethane at −5° C. After the reaction was completed as indicated by TLC, water was added water dropwise at a rate such that the temperature of the reaction stayed below 10° C. After warming to room temperature, the reaction mixture was diluted with additional water and methylene chloride and passed through a pad of cilite, washed by saturated ammonium chloride, dried and concentrated to give 2,4-dichloro-6-iso-butyl-1,3,5-triazine as yellow slurry liquid residue. The crude product was passed through a pad of silica gel eluted with 10% ethyl acetate in hexanes to give the light yellow liquid of the desired product 32 (3.0 g, 51%). ¹H NMR (500 MHz, CDCl₃) δ 2.75 (d, J=7.0 Hz, 2H), 2.29 (m, 1H), 0.97 (d, J=7.0 Hz. 6H).

### Example 33

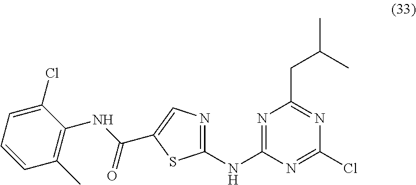

A solution of Compound 2 (1.17 g, 4.38 mmole), diisopropylamine (2.3 ml, 13.20 mmole) and Compound 32 (1.00 g, 4.85 mmole) in THF (30 mL) was stirred at 0° C. for 6 hours. 5% NaHCO₃ was added and the reaction mixture was extracted by ethyl acetate (3×). The organic layer was washed by saturated ammonium chloride, brine, dried (Na₂SO₄), and concentrated in vacuo. The residue was purified by column chromatography on silica gel (0-2% methanol in DCM) to give the desired product 33 as light-yellow solids (170 mg, 9%). ¹H NMR (500 MHz, DMSO-d₆) δ 12.98 (br s, 1H), 10.11 (s, 1H), 8.35 (s, 1H), 7.40 (d, J=7.5 Hz, 1H), 7.28 (m, 2H), 2.67 (br, 2H), 2.29 (m, 1H), 2.23 (s, 3H), 0.96 (s, 6H). ESI-MS: calcd for (C₁₈H₁₈Cl₂N₆OS) 436. found 437 (MH⁺).

### Example 34

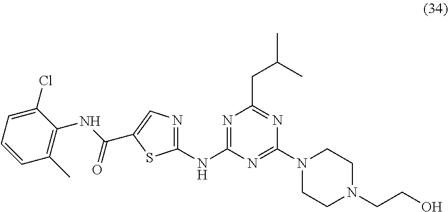

A mixture of 33 (120 mg, 0.27 mmol), diisopropylethylamine (0.17 mL, 1.00 mmol), and 1-(2-hydroxyethyl)piperazine (130 mg, 1.00 mmol) in 1,4-dioxane (12 mL) was stirred at 60° C. overnight. Water was added, followed by ethyl acetate/hexane (5/5). White solids precipitated from the solution, which was recrystallized from methanol/chloroform to give the desired product 34 as white solids (67 mg, 47%). ¹H NMR (500 MHz, DMSO-d₆) δ 11.97 (br s, 1H), 9.97 (s, 1H), 8.45 (br, 2H), 8.28 (s, 1H), 7.40 (d, J=7.6 Hz, 1H), 7.28 (m, 2H), 4.45 (t, J=5.0 Hz, 1H), 3.82 (m, 4H), 3.52 (q, J=6.0 Hz, 2H), 2.50 (br, 4H), 2.43 (t, J=6.0 Hz, 2H), 2.24 (s, 3H), 2.23 (obscured, 1H), 0.93 (s, 6H). ESI-MS: calcd for (C₂₄H₃₁ClN₈O₂S) 530. found 531 (MH⁺). HPLC: retention time: 17.16 min.; purity: 96%.

### Example 35

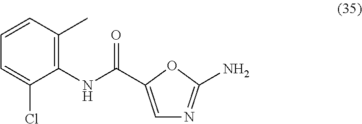

To a mixture of compound 1 (180 g, 0.75 mmol) in 1,4-dioxane (3 mL) and water (3 mL) was added NBS (160 mg, 0.90 mmol) at 0° C. The slurry was warmed and stirred at 20-22° C. for 3 h. Urea (58 mg, 0.97 mmol) was added and the mixture heated to 100° C. After 2 h, the resulting solution was cooled to 20-22° C. and conc. ammonium hydroxide (0.2 mL) was added dropwise. The resulting slurry was concentrated under vacuum. The remaining water was removed by co-evaporating with toluene. The residue was purified by column chromatography on silica gel (0-6% 2N ammonia in methanol/100-94% dichloromethane) to give compound 35 as white solids. (120 mg, 63% yield). ¹H NMR (500 MHz, DMSO-d₆) δ 9.57 (s, 1H), 7.60 (s, 1H), 7.36 (d, J=7.5 Hz, 1H,), 7.31 (s, 2H), 7.09-7.29 (m, 2H), 2.19 (s, 3H); ESI-MS: calcd for (C₁₁H₁₀ClN₃O₂) 251. found 252 (MH⁺), 250 ([M−H]⁻).

### Example 36

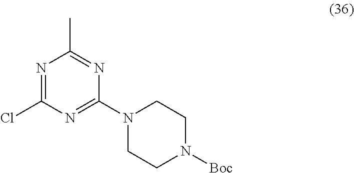

To a solution of compound 3 (0.5 g, 3.05 mmol) in DMF (5 mL) was added to a mixture of Boc-piperazine (0.57 g, 3.05 mmol), NaHCO₃ (0.51 g, 6.09 mmol) at room temperature. After the competition of the addition, the mixture was stirred at room temperature for 30 minutes. The reaction mixture was extracted with ethyl acetate and the organic layer was further washed with water (2×20 ml), brine (2×20 ml). The organic layer was dried (Na₂SO₄) and concentrated, during which white solid of 36 was formed (450 mg, 47%). This solid was used without further purification in the next step. ¹H NMR (500 MHz, DMSO-d₆) δ 3.80-3.79 (m, 2H), 3.72-3.70 (m, 2H), 3.42 (br, 4H), 2.34 (s, 3H), 1.42 (s, 9H), ESI-MS: calcd for (C₁₃H₂₀ClN₅O₂) 313 found 258 (M-56+H⁺).

### Example 37

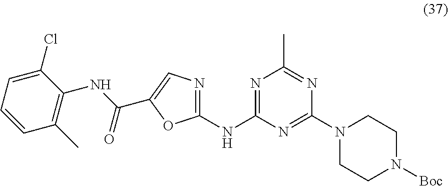

A round bottom flask was flam-dried and flushed with argon, then charged with xantphos (25 mg, 0.05 mmol) and dry 1,4-dioxane (5 mL). After degassing, Pd(OAc)₂ (5 mg, 0.02 mmol) was added, and the mixture was stirred under an inert atmosphere for 10 min. In another round-bottom flask, compound 36 (70 mg, 0.22 mmol), compound 35 (50 mg, 0.20 mmol)), and K₂CO₃ (525 mg, 3.8 mmol) were poured into dry 1,5-dioxane (7 mL). Then, the Pd(OAc)₂/xantphos solution was added with a syringe. The resulting mixture was subsequently heated to reflux under an inert atmosphere with vigorous stirring until the starting heteroaryl halide has disappeared (overnight). After cooling, the solid material was filtered off and washed with dichloromethane and methanol. The solvent was evaporated, and the resulting crude product was purified by flash column chromatography on cilica gel using EtOAc/DCM/MeOH: 80/20/2 v/v/v as eluent to provide compound 37 as white solids (33 mg, 31%). ESI-MS: calcd for (C₂₄H₂₉ClN₈O₄) 528. found 529 (MH⁺), 527 ([M−H]⁻).

### Example 38

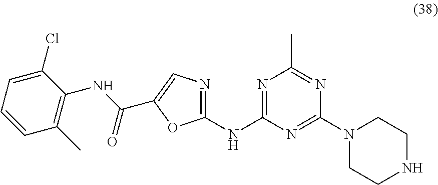

Compound 37 (30 mg, 0.06 mmol) was dissolved in dichloromethane (5 mL) and trifluoroacetic acid (1 mL) at 0° C. and the mixture was stirred at 0° C. for 3 hours. TLC was checked and the starting material was consumed. After concentration, the residue was neutralized by saturated sodium bicarbonate in water and the mixture was extracted by dichloromethane, dried over sodium sulfate and concentrated. The residue was purified by column chromatography on a silica gel (EtOAc/DCM/6% 2M NH₃:: 50/50/6) to give compound 38 as white solids (18 mg, 70%). ¹H NMR (500 MHz, DMSO-d₆) δ 9.93 (s, 1H), 7.86 (s, 1H), 7.39 (d, J=7.5 Hz, 1H), 7.30-7.25 (m, 2H), 3.71 (br, 4H), 2.68 (br, 4H), 2.26 (s, 3H), 2.21 (s, 3H); ESI-MS: calcd for (C₉H₂₁ClN₈O₂) 428. found 429 (MH⁺), 427 ([M−H]₋). HPLC: retention time: 6.09 min. purity: 96%.

### Example 39

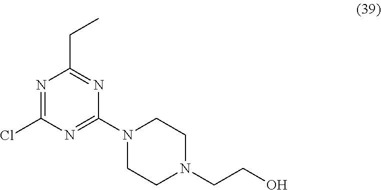

To a solution of compound 21 (1.2 g, 6.74 mmol) in THF (35 mL) was added to a mixture of 1-hydroxyethyl piperazine (600 mg, 4.60 mmol), DIPEA (0.80 mL, 4.59 mmol) and THF (35 mL) dropwise at −10° C. After the competition of the addition, the mixture was stirred at −10° C. for 30 minutes. Ammonium chloride solution was added and the mixture was extracted by ethyl acetate. The organic layer was dried (Na₂SO₄) and concentrated, during which yellow precipitate formed. The solids were collected by filtration, washed by ethyl acetate to give compound 39 as yellow solids (350 mg, 28%). ¹H NMR (500 MHz, DMSO-d₆) δ 5.36 (br, 1H), 4.73-4.53 (m, 2H), 3.77 (br, 2H), 3.55 (br, 4H), 3.15 (br, 4H), 2.63 (q, J=7.5 Hz, 2H), 1.18 (t, J=7.5 Hz, 3H); ESI-MS: calcd for (C₁₁H₁₈ClN₅O) 271. found 272 (MH⁺).

### Example 40

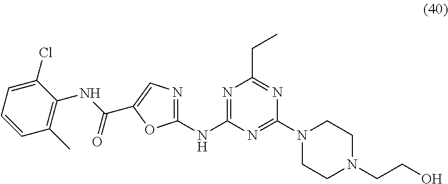

A round bottom flask was flame-dried and flushed with argon, then charged with xantphos (25 mg, 0.05 mmol) and dry 1,4-dioxane (5 mL). After degassing, Pd(OAc)₂ (5 mg, 0.02 mmol) was added, and the mixture was stirred under an inert atmosphere for 10 min. In another round-bottom flask, compound 39 (58 mg, 0.22 mmol), compound 35 (47 mg, 0.18 mmol)), and K₂CO₃ (525 mg, 3.8 mmol) were poured into dry 1,5-dioxane (15 mL). Then, the Pd(OAc)₂/xantphos solution was added with a syringe. The resulting mixture was subsequently heated to reflux under an inert atmosphere with vigorous stirring until the starting heteroaryl halide has disappeared (overnight). After cooling, the solid material was filtered off and washed with dichloromethane and methanol. The solvent was evaporated, and the resulting crude product was purified by flash column chromatography on silica gel using EtOAc/DCM/MeOH (2N NH₃): 50/50/5 v/v/v as eluent to provide compound 40 as white solids (45 mg, 51%). ¹H NMR (500 MHz, DMSO-d₆) δ 10.25 (br, 1H), 9.94 (s, 1H), 7.87 (s, 1H), 7.39 (d, J=7.5 Hz, 1H), 7.30-7.25 (m, 2H), 4.44 (t, J=5.5 Hz, 1H), 3.77 (br, 4H), 3.50 (q, J=6.0 Hz, 2H), 2.51 (m, 2H), 2.42-2.40 (m, 6H), 2.21 (s, 3H), 1.18 (t, J=8.0 Hz, 3H); ESI-MS: calcd for (C₂₂H₂₇ClN₈O₃) 486. found 487 (MH⁺), 485 ([M−H]⁻). HPLC: retention time: 8.16 min. purity: 97%.

### Example 41

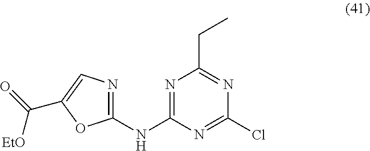

To a stirred solution of ethyl 2-aminooxazole-5-carboxylate (0.10 g, 0.64 mmol) in THF (6 mL) was added DIPEA (0.12 mL, 0.70 mmol) at 0° C. After stirring for 10 min. at the same temperature, Compound 21 (0.23 mg, 1.28 mmol) was added. The mixture was stirred at 70° C. for 8 h, cooled to room temperature, and diluted with EtOAc and washed with 5% NaHCO₃. The organic phase was concentrated and the residue was purified by chromatography on a silica gel column eluted with 1% MeOH in CH₂Cl₂ to afford Compound 41 (35 mg, 20%) as a yellow solid. ¹H NMR (500 MHz, DMSO-d₆) δ 12.43 (s, 1H, NH), 7.95 (s, 1H, Ar—H), 4.31 (dd, 2H, J=14.3 Hz, CH₂), 2.77 (dd, 2H, J=14.2 Hz, CH₂), 1.30 (t, 3H, J=6.7 Hz, CH₃), 1.23 (t, 3H, J=7.5 Hz, CH₃). MS (ESI) m/z 296 [M−H]⁻.

### Example 42

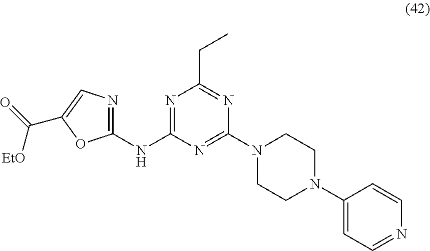

To a stirred solution of Compound 41 (0.10 g, 0.34 mmol) in Dioxane (10 mL) were added DIPEA (0.18 mL, 1.02 mmol) and 4-(pyridyl)piperazine (0.06 g, 0.37 mmol) at room temperature. The mixture was heated to 55° C. and stirred for 1 hour and cooled to room temperature. The reaction mixture was concentrated. The residue was washed with water, filtered, and dried in vacuo to obtain compound 42 (85 mg, 60%) as a yellow solid. ¹H NMR (500 MHz, DMSO-d₆) δ 11.51 (s, 1H, NH), 8.18 (d, 2H, J=5.8 Hz, Ar—H), 7.87 (s, 1H, Ar—H), 6.87 (d, 2H, J=6.5 Hz, Ar—H), 4.31 (dd, 1H, J=14.2 Hz, CH₂), 3.92 (bs, 4H), 3.44 (m, 4H) 2.56 (dd, 2H, J=15.1 Hz, CH₂), 1.29 (t, 3H, J=7.0 Hz, CH₃). 1.22 (t, 3H, J=7.5 Hz, CH₃). MS (ESI) m/z 425 [M+H]⁺.

### Example 43

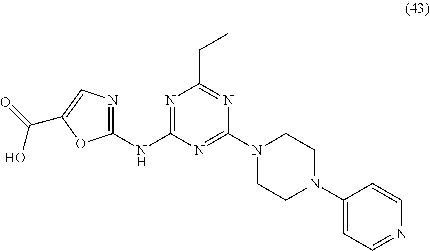

A solution of compound 42 (80 mg, 0.18 mmol) in THF-EtOH (1.3 mL, 2:3) and 6 M KOH solution (1.3 mL) was heated to 55° C. overnight under inert atmosphere. The solution was cooled to 0° C. and acidified with concd HCl to pH 1. The solution was concentrated in vacuo, and the residue was washed with water, ether, and dried in vacuo to obtain Compound 43 as a yellow solid (40 mg, 55%). ¹H NMR (500 MHz, DMSO-d₆) δ 13.59 (s, 1H, NH), 8.29 (d, 2H, J=6.6 Hz, Ar—H), 7.81 (s, 1H, Ar—H), 7.21 (d, 2H, J=7.3 Hz, Ar—H), 3.98 (bs, 4H, Ar—CH₂), 3.84-3.82 (m, 4H, Ar—CH₂), 2.59 (dd, 2H, J=15.1 Hz, CH₂), 1.22 (t, 3H, J=7.5 Hz, CH₃). MS (ESI) m/z 397 [M+H]⁺.

### Example 44

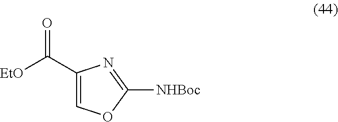

A mixture of 2-amino oxazole-4-carboxylic acid ethyl ester (0.5 g, 3.2 mmol), Boc anhydride (1.1 g, 4.8 mmol), DIPEA (0.61 mL, 3.5 mmol), DMAP (0.1 g, 0.8 mmol) and DMF (4 mL) was stirred at 40° C. overnight. The DMF was removed in vacuo and the residue taken up in ethyl acetate (40 mL). The reaction was washed with brine (2×25 mL), saturated sodium bicarbonate (25 mL), 0.01M HCl (25 mL), and dried over sodium sulfate. The organic phase was concentrated and the residue was purified by chromatography on a silica gel column eluted with Hexane/EtOAc (2:1) to afford Compound 44 (490 mg, 60%) as a light yellow solid. ¹H NMR (400 MHz, DMSO-d₆) δ 10.90 (s, 1H, NH), 8.51 (s, 1H, Ar—H), 4.25 (dd, J=7.2 Hz, 2H, CH₂), 1.44 (s, 9H, C(CH₃)₃), 1.27 (t, J=7.2 Hz, 3H, CH₃). MS (ESI) m/z 255 [M−H]⁻.

### Example 45

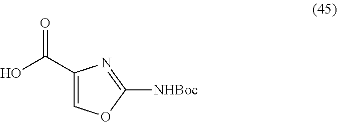

A mixture of Compound 44 (0.25 g, 0.97 mmol), 1N NaOH (2 mL) and methanol (1 mL) was stirred at 35° C. The reaction was determined to be complete by TLC after 3 h. The methanol was removed in vacuo and the aqueous layer carefully adjusted to pH 2 with 1N HCl at which time a crystalline white solid precipitated out of solution. Filtration of the white solid gave compound 45 (0.2 g, 95%). ¹H NMR (400 MHz, DMSO-d₆) δ 10.82 (s, 1H), 8.41 (s, 1H), 1.44 (s, 9H).

### Example 46

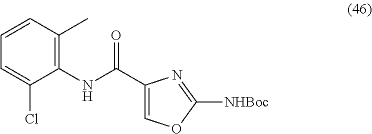

A 2 M solution of oxalyl chloride (0.44 mL, 14.5 mmol) was added dropwise to a stirred suspension of Compound 45 (0.1 g, 0.44 mmol) and DMF (2 drops) in CH₂Cl₂ (4 mL) at 0° C. The solution was warmed to room temperature, stirred at for 4 h and concentrated. The residue was coevaporated with toluene and dried in vacuo to obtain the crude acid chloride. 2-chloro-6-methylaniline (0.15 mL, 1.2 mmol) was added dropwise to a stirred solution of crude acid chloride in CH₂Cl₂ (2 mL) at 0° C. After 15 min. at the same temperature, pyridine (0.07 mL, 0.9 mmol) was added slowly. The solution was warmed to room temperature and stirred for overnight. Reaction mixture was diluted with EtOAc and washed with H₂O and brine. The EtOAc extract was separated, dried (NaSO₄), filtered, and concentrated. The residue was chromatographed on a silica gel column eluted with Hexane/EtOAc (1:1) afforded Compound 46 (30 mg, 19%) as a white solid. ¹H NMR (400 MHz, CDCl₃-d₆) δ 8.37 (s, 1H), 8.01 (s, 1H), 7.45 (s, 1H), 7.31-7.29 (m, 1H), 7.19-7.13 (m, 2H), 2.32 (s, 3H), 1.55 (s, 9H). MS (ESI) m/z 374 [M+Na]⁺.

### Example 47

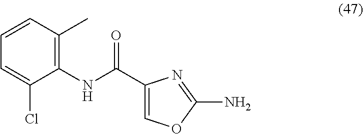

A solution of Compound 46 (30 mg, 0.85 mmol) in trifluoroacetic acid (0.2 mL) and CH₂Cl₂ (1.2 mL) mL) was stirred at 0° C. for 5 h and concentrated. The residue was chromatographed on a silica gel column eluted with CH₂Cl₂/MeOH (40:1) to give compound 47 as white solid. ¹H NMR (400 MHz, CDCl₃-d₆) δ 9.32 (s, 1H), 7.98 (s, 1H), 7.45 (s, 1H), 7.37-7.36 (m, 1H), 7.35-7.22 (m, 1H), 7.08 (s, 2H), 2.19 (s, 3H). MS (ESI) m/z 252 [M+H]⁺.

### Example 48

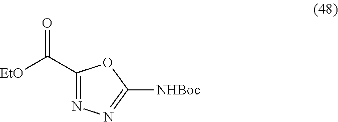

A mixture of 5-amino-1,3,4-oxadiazole-2-carboxylic acid ethyl ester (2.0 g, 12.7 mmol), Boc anhydride (4.17 g, 19.1 mmol), DIPEA (1.8 mL, 14.0 mmol), DMAP (413 mg, 3.2 mmol) and DMF (15 mL) was stirred at 40° C. overnight. The DMF was removed in vacuo and the residue taken up in ethyl acetate (40 mL). The reaction was washed with brine (2×100 mL), saturated sodium bicarbonate (100 mL), 0.01M HCl (100 mL), and dried over sodium sulfate. The organic phase was concentrated and the residue was purified by chromatography on a silica gel column eluted with Hexane/EtOAc (3:1) to afford Compound 48 (2.2 g, 67%) as a white solid. ¹H NMR (400 MHz, CDCl₃-d₆) δ 8.91 (bs, 1H), 4.51 (dd, J=14.3 Hz, 2H), 1.56 (s, 9H), 1.44 (t, J=14.3 Hz, 3H). MS (ESI) m/z 280 [M+Na]⁺.

### Example 49

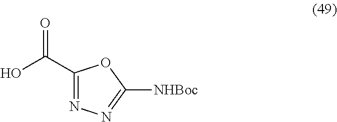

A mixture of Compound 48 (1.0 g, 3.9 mmol), 1N NaOH (10 mL) and methanol (3 mL) was stirred at 35° C. The reaction was determined to be complete by TLC after 3 h. The methanol was removed in vacuo and the aqueous layer carefully adjusted to pH 2 with 1N HCl. Solvent was removed and residue was dried in vacuo to give 49 as a white solid. ¹H NMR (400 MHz, DMSO-d₆) δ 10.95 (bs, 1H,), 1.44 (s, 9H). MS (ESI) m/z 230 [M+H]⁺.

### Example 50

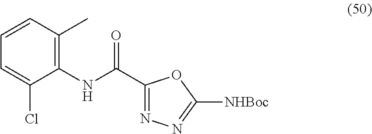

A 2 M solution of oxalyl chloride (4.4 mL, 8.74 mmol) was added dropwise to a stirred suspension of Compound 49 (1.0 g, 4.37 mmol) and DMF (30 mL) in CH₂Cl₂ (25 mL) at 0° C. The solution was warmed to room temperature, stirred at for 4 h and concentrated. The residue was coevaporated with toluene and dried in vacuo to obtain the crude acid chloride. 2-chloro-6-methylaniline (1.6 mL, 13.11 mmol) was added dropwise to a stirred solution of crude acid chloride in CH₂Cl₂ (20 mL) at 0° C. After 15 min. at the same temperature, pyridine (0.45 mL, 5.24 mmol) was added slowly. The solution was warmed to room temperature and stirred for overnight. Reaction mixture was concentrated and the residue was chromatographed on a silica gel column eluted with CH₂Cl₂/MeOH (40:1) afforded Compound 50 as a white solid. ¹H NMR (400 MHz, DMSO-d₆) δ 11.61 (s, 1H), 10.83 (s, 1H), 7.41-7.99 (m, 1H), 7.29-7.28 (m, 2H), 1.24 (s, 3H), 1.49 (s, 9H). MS (ESI) m/z 375 [M+Na]⁺.

### Example 51

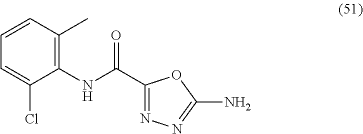

A solution of Compound 50 (0.3 g, 0.85 mmol) dissolved in a mixture of CH₂Cl₂ and TFA (7 mL 6:1). The mixture was stirred at 0° C. to room temperature for 3 h. Reaction mixture was concentrated and the residue was chromatographed on a silica gel column eluted with CH₂Cl₂/MeOH (40:1) afforded Compound 51 as a white solid. ¹H NMR (400 MHz, DMSO-d₆) δ 10.51 (s, 1H), 7.66 (s, 2H),), 7.40-7.38 (m, 1H), 7.28-7.27 (m, 2H), 3.03 (s, 3H). MS (ESI) m/z 253 [M+Na]⁺

### Example 52

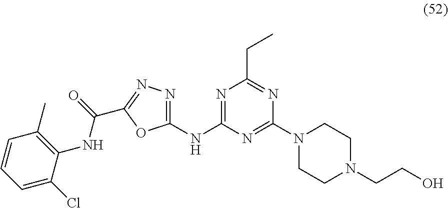

Compound 51 (0.05 g, 0.2 mmol), Compound 39 (0.07 g, 0.24 mmol), Pd(OAc)₂ (6 mg, 0.02 mmol), Xantphos (36 mg, 0.04 mmol) and K₂CO₃ (0.55 g, 4.0 mmol) were added in 2-5 mL a screw capped microwave vial. Dioxane:DMF (2.5 mL, 1.5:1) was added and vial was sealed with a cap. The mixture was allowed to stir at 180° C. for 10 min. under microwave (Biotage, Initiator 2.0) condition. Reaction mixture was filtered and the solid was washed with CH₂Cl₂ and MeOH, concentrated. The residue was chromatographed on a silica gel column eluted with 4% MeOH in CH₂Cl₂ afforded Compound 52 as a white solid (21 mg, 22%). ¹H NMR (500 MHz, DMSO-d₆) δ 11.95 (s, 1H), 10.70 (s, 1H), 7.42-7.29 (m, 3H), 4.42 (t, 1H, J=10.7 Hz), 3-82-3.81 (m, 4H), 3.51 (dd, J=9.3 Hz, 2H) 2.56 (dd, J=15.1 Hz, 2H), 2.44-2.40 (m, 2H), 2.24 (s, 3H), 1.20 (t, 3H, J=15.1 Hz). MS (ESI) m/z 488 [M+H]⁺.

### Example 53

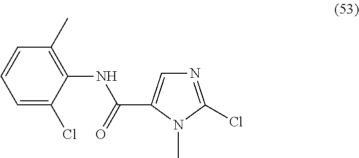

A 1.6 M solution of n-butyllithium in hexanes (2.27 ml, 3.6 mmol) was added dropwise to a solution of 2-chloro-1-methyl-1H-imidazole* (0.4 g, 3.45 mmol) in anhydrous THF (10 ml) at −78° C. The reaction mixture was maintained below −78° C. during the addition. After 15 min, a solution of 2-chloro-6-methylphenyl isocyanate (0.64 g, 3.79 mmol) in THF (5 ml) was added and the solution was stirred at −78° C. for further 2 h. The solution was quenched by the addition of aq NH₄Cl and partitioned between EtOAc and water. The organic layer was separated and washed with brine, dried and concentrated. The crude product was purified on silica gel chromatography by using 30% EtOAc/Hexane, to give compound 53 as white solid (353 mg, 36% yield). ¹H NMR (500 Hz, DMSO-d₆) δ 9.96 (s, 1H), 7.82 (s, 1H), 7.40 (dd, J=1.7, 5.74 Hz, 1H), 7.27 (m, 2H), 3.83 (s, 3H,), 2.23 (s, 3H); ESI-MS: calcd for (C12H11Cl2N3O) 282. found 282 (M−H⁺). HPLC: retention time: 17.83 min.; purity: 96%.

### Example 54

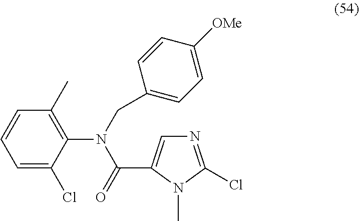

Sodium hydride (95% dispersion, 0.023 g, 0.85 mmol) was added to a solution of Compound 53 (0.2 g, 0.71 mmol) in DMF (10 mL). After 30 min, the mixture was treated with 4-methoxybenzyl chloride (115 uL, 0.85 mmol) and tetrabutylammonium iodide (0.043 g, 0.12 mmol) and then stirred at room temperature for 13 h. The reaction mixture was partitioned between EtOAc and water. The organic layer was separated and washed with brine, dried and concentrated. The crude product was purified on silica gel chromatography by using 15% EtOAc/Hexane to give compound 54 as white solid (254 mg, 89% yield). ¹H NMR (500 Hz, DMSO-d₆) δ 7.37 (d, J=7.80 Hz, 1H), 7.29 (t, J=7.78 Hz, 1H), 7.22 (d, J=7.21 Hz, 1H), 7.14 (d, J=5.25 Hz, 2H), 6.82 (d, J=6.72 Hz, 2H), 5.03 (d, J=14 Hz, 1H), 4.59 (d, J=14 Hz, 1H), 3.76 (s, 3H,), 3.71 (s, 3H), 1.84 (s, 3H); ESI-MS: calcd for (C20H19Cl2N3O2) 403. found 404 (M+H⁺).

### Example 55

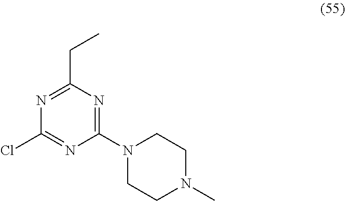

A mixture of Compound 21 (1.0 g, 5.65 mmol), 5% aq NaHCO₃ (10 ml), and 1-methyl piperazine (0.51 g, 5.15 mmol) in THF:acetone:water (52:13:13) was stirred at room temperature for 12 h. The reaction mixture was partitioned between EtOAc and water. The organic layer was separated and washed with brine, dried and concentrated. The crude product 55 was used in the next reaction without further purification.

### Example 56

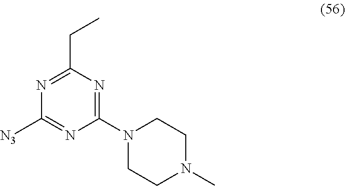

A mixture of Compound 55 (1.0 g, 4.14 mmol) and NaN₃ (0.81 g, 12.45 mmol) in 20 ml of DMF was stirred at room temperature for 13 h. The reaction mixture was partitioned between EtOAc and water. The organic layer was separated and washed with brine, water and concentrated on the rotavapor. The crude product was passed on the pad of silica gel by using 5% MeOH/DCM to give compound 56 as white solid (0.6 g, 59%). ESI-MS: calcd for (C10H16N8) is 248 and found 249 (M+H⁺).

### Example 57

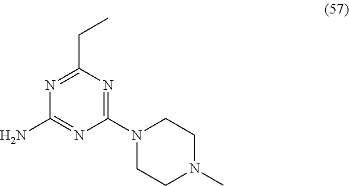

A mixture of Compound 56 (1.2 g, 4.83 mmol) and 52 mg of 10% palladium-on-carbon in 30 ml of absolute ethanol was stirred at 25° C. under 1 atm of hydrogen for 2 h. The catalyst was filtered through the celite and washed ethanol. The filterate was concentrated on the rotavapor to give compound 57 as light yellow solid (1.05 g, 98%). ¹H NMR (500 Hz, DMSO-d₆) δ 6.70 (br s, 2H), 3.65 (m, 4H,), 2.35 (m, 2H), 2.25 (m, 4H), 2.15 (s, 3H), 1.1 (t, J=7.5 Hz, 3H); ESI-MS: calcd for (C₁₀H₁₈N₆) 222 found 223 (M+H⁺).

### Example 58

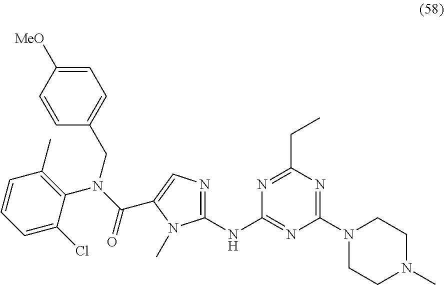

A mixture of Compound 54 (100 mg, 0.25 mmol), Compound 57 (55.1 mg, 0.25 mmol), Pd(OAc)₂ (5.5 mg, 0.025 mmol), Xantphos (29 mg, 0.05 mmol) and NaO^{t}Bu (26 mg, 0.27 mmol) and 1,4-dioxane (3 ml) were added in 2-5 mL of a screw capped microwave vial. The mixture was subjected to microwave heating using a Biotage, Initiator 2.0 at 180° C. for a period of 7 min. Reaction mixture was filtered and the solid was washed with CH₂Cl₂ and MeOH, concentrated. The residue was chromatographed on a silica gel column eluted with 5% NH₃/MeOH in CH₂Cl₂ to give compound 58. ESI-MS: calcd for (C₃₀H₃₆ClN₉O₂) 589. found 590 (M+H⁺). This crude was used in the next reaction without further purification.

### Example 59

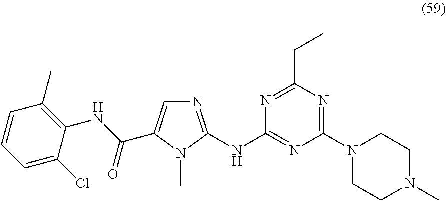

A solution of Compound 58 (55 mg, 0.09 mmol) dissolved in a mixture of CH₂Cl₂ and TFA (2 ml, 1:1) was treated with triflic acid (0.03 ml, 0.33 mmol) and stirred at room temperature for 3 h. The reaction mixture was adjusted to the pH 9 by using saturated NaHCO₃ and extracted with dichloromethane. The organic layer was evaporated and purified by column chromatography using 5% NH₃/MeOH in dichlromethane to afford yellow solids of compound 59. ESI-MS: calcd for (C₂₂H₂₈ClN₉O) 469. found 470 (M+H⁺). HPLC: retention time: 6.53 min.; purity: 93%.

### Example 60

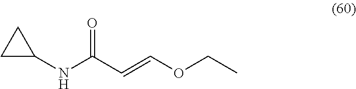

A mixture of ethyl β-ethoxyacrylate (26.50 g, 183 mmol) and 2 N sodium hydroxide (110 mL, 220 mmol) was refluxed for 2 h and cooled to 0° C. water was removed under vacc., and the yellow solids were triturated with toluene and evaporated to give the sodium β-ethoxyacrylate (25 g, 97%). The mixture of sodium β-thoxyacrylate (10.26 g, 74.29 mmol) and thionyl chloride (25 mL, 343 mmol) was refluxed for 2 h, and evaporated to give the β-ethoxyacryloyl chloride crude product, which was used without purification. To a cold stirring solution of 3-ethoxyacryloyl chloride (7.3 g, 54.2 mmol) in THF (70 mL) was added cyclopropyl amine (8.3 mL, 119.2 mmol) and pyridine (8.8 ml, 108 mmol). The mixture was then warmed and stirred overnight at room temperature. Water was added and extracted with EtOAc. The organic layer was evaporated and the resulting residue was purified by silica gel using 3:1 (hexane-EtoAc), concentrated under vacuum to give 2.7 g (34% yields) of compound 60. ¹H NMR (400 MHz, CDCl₃) δ 7.50 (d, J=12 Hz, 1H), 5.60 (br s, 1H), 5.24 (d, J=11.5 Hz, 1H), 3.94 (q, J=6.0 Hz, 2H), 2.70 (m, 1H), 1.35 (t, J=6.8 Hz, 3H), 0.75 (q, J=6.0 Hz, 2H), 0.49 (br, 2H); ESI-MS: calcd for (C₈H₁₃NO₂) 155. found 156 (MH⁺).

### Example 61

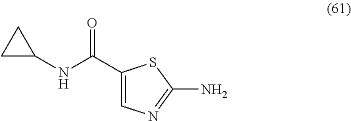

To a mixture of compound 60 (0.5 g, 3.23 mmol) in 1,4-dioxane (25 mL) and water (17 mL) was added NBS (0.6 g, 3.55 mmol) at room temperature. The slurry was warmed and stirred at 20-22° C. for 3 h. Thiourea (0.26 g, 3.42 mmol) was added and the mixture was refluxed to 100° C. After 2 h, the resulting solution was cooled to 20-22° C. and conc. ammonium hydroxide (6 mL) was added drop-wise. The resulting slurry was concentrated under vacuum to about half volume and cooled to 0-5° C. The solid was collected by vacuum filtration, washed with cold water, and dried to give 0.5 g (85% yields) of compound 61 as deep-yellow solids. ¹H NMR (500 MHz, CDCl₃) δ 10.61 (br s, 1H), 7.62 (d, 3.6 Hz, 1H), 7.12 (s, 1H), 6.96 (br s, 2H), 2.13 (m, 4H), 2.07 (m, 1H); ESI-MS: calcd for (C₇H₉N₃OS) 183. found 184 (MH⁺).

### Example 62

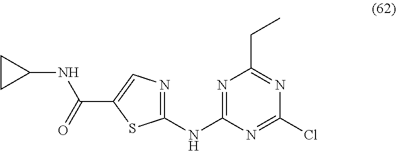

A solution of Compound 61 (0.5 g, 2.73 mmol), diisopropylamine (0.5 ml, 3.0 mmol) and Compound 21 (1.10 g, 6.18 mmole) in THF (15 mL) was stirred at 0° C. for 8 hours, then cold 5% NaHCO₃ was added to the reaction mixture, and the aqueous mixture was extracted twice with EtOAc. The combined extracts were washed with brine, dried, and concentrated in vacuo until a lot of precipitate formed. After filtration, the solids were washed by ethyl acetate dried to give 62 (140 mg, 16%), which was used in the next step without purification for the next step reactions.

### Example 63

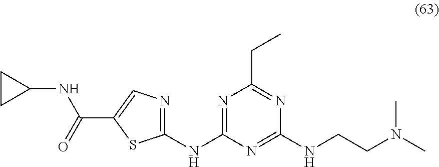

A mixture of compound 62 (0.25 g, 0.78 mmol) and diisopropylethylamine (0.53 mL, 3.07 mmol), and N,N-dimethylethane-1,2-diamine (0.27 g, 3.07 mmol) in DMSO was heated at 70° C. for over night. The mixture was extracted by ethyl acetate and the combined organic layers were washed with water and brine. The crude product was recrystallized by MeOH/CHCl₃ (25 mg, 6%). ¹H NMR (400 MHz, DMSO-d₆) δ11.75 (br s, 1H), 8.32 (d, J=3.6 Hz, 1H), 7.90 (br s, 1H), 7.5 (br s, 1H), 3.42 (m, 2H), 2.52 (m, 2H), 2.45 (m, 2H), 2.23 (s, 3H), 2.17 (m, 3H), 1.20 (m, 4H), 0.6 (m, 2H), 0.45 (m, 2H); ESI-MS: calcd for (C₁₆H₂₄N₈OS) 376. found 377 (M+H⁺). HPLC: retention time: 12.2 min; purity 90.6%.

### Example 64

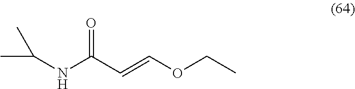

A mixture of ethyl β-ethoxyacrylate (26.50 g, 183 mmol) and 2 N sodium hydroxide (110 mL, 220 mmol) was refluxed for 2 h and cooled to 0° C. water was removed under vac., and the yellow solids were triturated with toluene and evaporated to give the sodium β-ethoxyacrylate (25 g, 97%). The mixture of sodium β-methoxyacrylate (10.26 g, 74.29 mmol) and thionyl chloride (25 mL, 343 mmol) was refluxed for 2 h, and evaporated to give the β-ethoxyacryloyl chloride crude product, which was used without further purification. To a cold stirring solution of 3-ethoxyacryloyl chloride (7.3 g, 54.2 mmol) in THF (70 mL) was added isopropylamine (13.8 mL, 162.2 mmol) and pyridine (8.8 ml, 108 mmol). The mixture was then warmed and stirred overnight at room temperature. Water was added and extracted with EtOAc. The organic layer was evaporated and the resulting residue was purified by silica gel using 1:1 (hexane-EtoAc), concentrated under vacuum to give 2.3 g of compound 64. ¹H NMR (400 MHz, CDCl₃) δ 7.50 (d, J=12.4 Hz, 1H), 5.20 (m, 2H), 4.18 (m, 1H), 3.94 (q, J=6.0 Hz, 2H), 1.31 (t, J=5.2 Hz, 3H), 1.16 (s, 3H), 1.14 (s, 3H); ESI-MS: calcd for (C₈H₁₅NO₂) 157. found 158 (MH⁺).

### Example 65

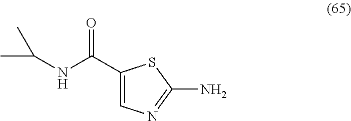

To a mixture of compound 64 (1.0 g, 6.37 mmol) in 1,4-dioxane (50 mL) and water (34 mL) was added NBS (1.19 g, 7.00 mmol) at room temperature. The slurry was warmed and stirred at 20-22° C. for 3 h. Thiourea (0.51 g, 6.75 mmol) was added and the mixture was refluxed to 100° C. After 2 h, the resulting solution was cooled to 20-22° C. and conc. ammonium hydroxide (6 mL) was added drop-wise. The resulting slurry was concentrated under vacuum to about half volume and cooled to 0-5° C. The solid was collected by vacuum filtration, washed with cold water, and dried to give 0.8 g (85% yields) of compound 65 as deep-yellow solids. ¹H NMR (400 MHz, CDCl₃) δ 10.61 (br s, 1H), 7.62 (d, J=3.6 Hz, 1H), 7.12 (s, 1H), 6.96 (br s, 2H), 2.13 (m, 4H), 2.07 (m, 1H); ESI-MS: calcd for (C₇H₉N₃OS) 183. found 184 (MO.

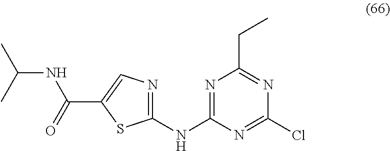

### Example 66

A solution of Compound 65 (0.5 g, 2.7 mmol), diisopropylamine (0.52 ml, 2.97 mmol) and Compound 21 (1.10 g, 6.18 mmol) in THF (15 mL) was stirred at 0° C. for 8 hours, then cold 5% NaHCO₃ was added to the reaction mixture, and the aqueous mixture was extracted twice with EtOAc. The combined extracts were washed with brine, dried, and concentrated in vacuo until a lot of precipitate formed. After filtration, the solids were washed by ethyl acetate dried to give 66 (371 mg, 42%), which was used in the next step without purification for the next step reactions.

### Example 67

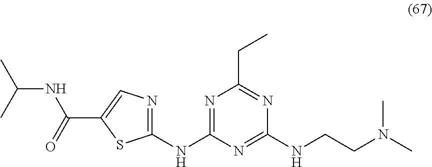

A mixture of compound 66 (0.25 g, 0.78 mmol) and diisopropylethylamine (0.53 mL, 3.07 mmol), and N,N-dimethylethane-1,2-diamine (0.27 g, 3.07 mmol) in DMSO was heated at 70° C. for over night. The mixture was extracted by ethyl acetate and the combined organic layers were washed with water and brine. The crude product was recrystallized by MeOH/CHCl₃ (30 mg, 10%). ¹H NMR (400 MHz, DMSO-d₆) δ 11.75 (br s, 1H), 8.32 (d, J=3.6 Hz, 1H), 7.90 (br s, 1H), 7.5 (br s, 1H), 3.42 (m, 2H), 2.52 (m, 2H), 2.45 (m, 2H), 2.23 (s, 3H), 2.17 (m, 3H), 2.15 (m, 1H), 1.12 (s, 3H), 1.1 (s, 3H), 1.0 (s, 3H); ESI-MS: calcd for (C₁₆H₂₆N₈OS) 378 found 379 (M+H⁺). HPLC: retention time: 6.2 min; purity 84.5%.

### Example 68

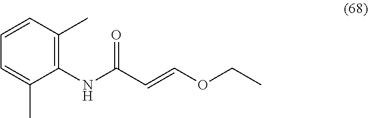

A mixture of ethyl β-ethoxyacrylate (26.50 g, 183 mmol) and 2 N sodium hydroxide (110 mL, 220 mmol) was refluxed for 2 h and cooled to 0° C. water was removed under vac., and the yellow solids were triturated with toluene and evaporated to give the sodium β-ethoxyacrylate (25 g, 97%). The mixture of sodium β-methoxyacrylate (10.26 g, 74.29 mmol) and thionyl chloride (25 mL, 343 mmol) was refluxed for 2 h, and evaporated to give the β-ethoxyacryloyl chloride crude product, which was used in the next step without further purification. To a cold stirring solution of 3-ethoxyacryloyl chloride (5.0 g, 36.23 mmol) in THF (40 mL) was added 2,6-dimethylaniline (4.3 g, 35.5 mmol) and pyridine (4.4 ml, 54.34 mmol). The mixture was then warmed and stirred overnight at room temperature. Water was added and extracted with EtOAc. The organic layer was evaporated and the resulting residue was purified by silica gel using 1:1 (hexane-EtOAc), concentrated under vacuum to give 2.3 g of compound 68. ¹H NMR (400 MHz, DMSO-d₆) δ 8.95 (s, 1H), 7.39 (d, J=12.4 Hz, 1H), 7.01 (s, 3H), 5.53 (d, J=12.4 Hz, 1H), 3.92 (q, J=5.6 Hz, 2H), 2.08 (s, 6H), 1.24 (t, J=5.2 Hz, 3H), ESI-MS: calcd for (C₁₃H₁₇NO₂) 219. found 220 (MH⁺).

### Example 69

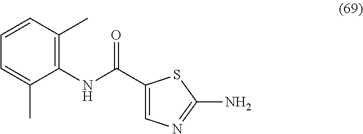

To a mixture of compound 68 (2.5 g, 11.41 mmol) in 1,4-dioxane (100 mL) and water (70 mL) was added NBS (2.13 g, 12.55 mmol) at room temperature. The slurry was warmed and stirred at 20-22° C. for 3 h. Thiourea (0.92 g, 12.09 mmol) was added and the mixture was refluxed to 100° C. After 2 h, the resulting solution was cooled to 20-22° C. and conc. ammonium hydroxide (10 mL) was added drop-wise. The resulting slurry was concentrated under vacuum to about half volume and cooled to 0-5° C. The solid was collected by vacuum filtration, washed with cold water, and dried to give 1.4 g (36% yields) of compound 69 as deep-brown solids. ¹H NMR (400 MHz, CDCl₃) δ 9.32 (s, 1H), 7.80 (s, 1H), 7.50 (s, 2H), 7.06 (br s, 3H), 2.12 (s, 6H), calcd for (C₁₂H₁₃N₃OS) 247. found 248 (MH⁺).

### Example 70

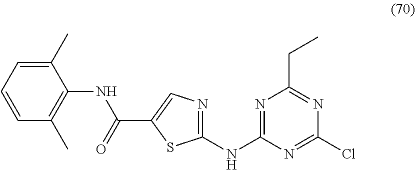

A solution of Compound 69 (0.5 g, 2.02 mmol), diisopropylethylamine (0.39 ml, 2.22 mmol) and Compound 21 (0.54 g, 3.02 mmol) in THF (15 mL) was stirred at 0° C. for 8 hours, then cold 5% NaHCO₃ was added to the reaction mixture, and the aqueous mixture was extracted twice with EtOAc. The combined extracts were washed with brine, dried, and concentrated in vacuo until a lot of precipitate formed. After filtration, the solids were washed by ethyl acetate dried to give 70 (309 mg, 64%), which was used in the next step without purification for the next step reactions.

### Example 71

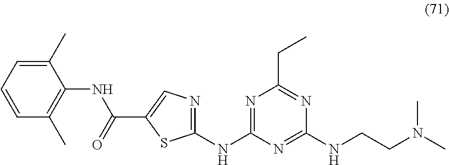

A mixture of compound 70 (0.4 g, 1.03 mmol) and diisopropylethylamine (0.72 mL, 4.12 mmol), and N,N-dimethylethane-1,2-diamine (0.36 g, 4.12 mmol) in DMSO was heated at 70° C. for over night. The mixture was extracted by ethyl acetate and the combined organic layers were washed with water and brine. The crude product was recrystallized by MeOH/CHCl₃ (309 mg, 64%). ¹H NMR (400 MHz, DMSO-d₆) δ11.75 (br s, 1H), 9.63 (d, J=5.2 Hz, 1H), 8.22 (s, 1H), 7.70 (br s, 1H), 7.08 (m, 3H), 3.42 (m, 2H), 2.52 (m, 2H), 2.45 (m, 2H), 2.23 (m, 12H), 1.1 (m, 3H); ESI-MS: calcd for C₂₁H₂₈N₈₀S) 440 found 441 (M+H⁺). HPLC: retention time: 16.2 min; purity 98.4%

### Example 72

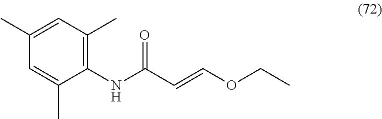

A mixture of ethyl β-ethoxyacrylate (26.50 g, 183 mmol) and 2 N sodium hydroxide (110 mL, 220 mmol) was refluxed for 2 h and cooled to 0° C. water was removed under vac., and the yellow solids were triturated with toluene and evaporated to give the sodium β-ethoxyacrylate (25 g, 97%). The mixture of sodium β-methoxyacrylate (10.26 g, 74.29 mmol) and thionyl chloride (25 mL, 343 mmol) was refluxed for 2 h, and evaporated to give the β-ethoxyacryloyl chloride crude product, which was used in the next step without further purification. To a cold stirring solution of 3-ethoxyacryloyl chloride (5.0 g, 36.23 mmol) in THF (40 mL) was added 2,4,6-trimethylaniline (4.8 g, 35.5 mmol) and DIPEA (9.47 ml, 54.34 mmol). The mixture was then warmed and stirred overnight at room temperature. Water was added and extracted with EtOAc. The organic layer was evaporated and the resulting solids were triturated with EtOAc and filtered the solids to give 1.5 g (18%) of compound 72. ¹H NMR (400 MHz, DMSO-d₆) δ 8.88 (s, 1H), 7.39 (d, 12.4 Hz, 1H), 6.89 (s, 1H), 6.82 (s, 1H), 5.53 (d, J=12.4 Hz, 1H), 3.92 (q, J=5.6 Hz, 2H), 2.28 (s, 3H), 2.18 (s, 3H), 2.03 (s, 3H), 1.24 (t, J=5.2 Hz, 3H).

### Example 73

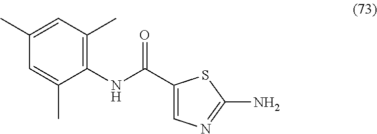

To a mixture of compound 72 (1.9 g, 8.15 mmol) in 1,4-dioxane (50 mL) and water (35 mL) was added NBS (1.52 g, 8.97 mmol) at room temperature. The slurry was warmed and stirred at 20-22° C. for 3 h. Thiourea (0.64 g, 8.64 mmol) was added and the mixture was refluxed to 100° C. After 2 h, the resulting solution was cooled to 20-22° C. and conc. ammonium hydroxide (10 mL) was added drop-wise. The resulting slurry was concentrated under vacuum to about half volume and cooled to 0-5° C. The solid was collected by vacuum filtration, washed with cold water, and dried to give 0.88 g (44% yields) of compound 35 as deep-brown solids. ¹H NMR (400 MHz, CDCl₃) δ 9.24 (s, 1H), 7.78 (s, 1H), 7.48 (s, 1H), 6.86 (s, 2H), 6.57 (s, 1H), 2.07 (m, 6H), 1.99 (t, J=5.2 Hz, 3H), calcd for (C₁₃H₁₅N₃OS) 261. found 262 (MH⁺).

### Example 74

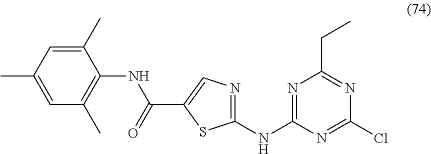

A solution of Compound 73 (0.5 g, 1.91 mmol), diisopropylethylamine (0.37 ml, 2.1 mmol) and Compound 21 (0.51 g, 2.87 mmol) in THF (15 mL) was stirred at 0° C. for 8 hours, then cold 5% NaHCO₃ was added to the reaction mixture, and the aqueous mixture was extracted twice with EtOAc. The combined extracts were washed with brine, dried, and concentrated in vacuum. The crude product was triturated with EtOAc and resulting solids were filtered off to give 74 (400 mg, 65%), which was used in the next step without further purification.

### Example 75

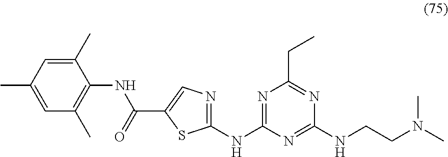

A mixture of compound 74 (0.4 g, 0.99 mmol) and diisopropylethylamine (0.69 mL, 3.98 mmol), and N,N-dimethylethane-1,2-diamine (0.35 g, 3.98 mmol) in DMSO was heated at 60° C. for over night. The mixture was extracted by ethyl acetate and the combined organic layers were washed with water and brine. The crude product was recrystallized by i-PrOH/CHCl₃ to give 37 as white solid, 39 mg, 9% yield. ¹H NMR (400 MHz, DMSO-d₆) δ 11.75 (br s, 1H), 9.52 (d, J=5.2 Hz, 1H), 8.19 (s, 1H), 7.70 (br s, 1H), 6.88 (s, 2H), 3.48 (m, 2H), 2.52 (m, 2H), 2.45 (m, 2H), 2.10 (m, 15H), 1.19 (m, 3H); ESI-MS: calcd for C₂₂H₃₀N₈OS is 454. found 455 (M+H⁺). HPLC: retention time: 17.1 min; purity 98.6%

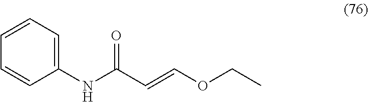

A mixture of ethyl β-ethoxyacrylate (26.50 g, 183 mmol) and 2 N sodium hydroxide (110 mL, 220 mmol) was refluxed for 2 h and cooled to 0° C. water was removed under vac., and the yellow solids were triturated with toluene and evaporated to give the sodium β-ethoxyacrylate (25 g, 97%). The mixture of sodium β-methoxyacrylate (10.26 g, 74.29 mmol) and thionyl chloride (25 mL, 343 mmol) was refluxed for 2 h, and evaporated to give the β-ethoxyacryloyl chloride crude product, which was used in the next step without further purification. To a cold stirring solution of 3-ethoxyacryloyl chloride (5.0 g, 36.23 mmol) in THF (40 mL) was added aniline (3.23 ml, 35.5 mmol) and pyridine (4.4 ml, 54.34 mmol). The mixture was then warmed and stirred overnight at room temperature. Water was added and extracted with EtOAc. The organic layer was evaporated and the resulting residue was purified by silica gel using 1:1 (hexane-EtoAc), concentrated under vacuum to give 2.71 g of compound 76. ¹H NMR (400 MHz, DMSO-d₆)

### Example 77

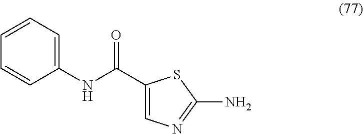

To a mixture of compound 77 (2.7 g, 14.14 mmol) in 1,4-dioxane (100 mL) and water (70 mL) was added NBS (1.14 g, 15.55 mmol) at room temperature. The slurry was warmed and stirred at 20-22° C. for 3 h. Thiourea (1.14 g, 14.98 mmol) was added and the mixture was refluxed to 100° C. After 2 h, the resulting solution was cooled to 20-22° C. and conc. ammonium hydroxide (10 mL) was added drop-wise. The resulting slurry was concentrated under vacuum to about half volume and cooled to 0-5° C. The solid was collected by vacuum filtration, washed with cold water, and dried to give 1.2 g (39% yields) of compound 77 as deep-brown solids. ¹H NMR (400 MHz, DMSO-d₆) δ 9.8 (s, 1H), 7.9 (s, Hz, 1H), 7.6 (m, 4H), 7.3 (m, 2H), 7.1 (m, 1H),; ESI-MS: calcd for (C₁₀H₉N₃OS): 219 found 220 (M+H⁺).

### Example 78

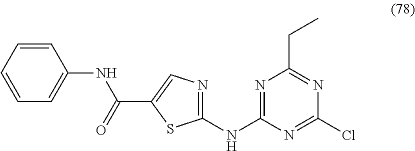

A solution of Compound 77 (0.5 g, 2.28 mmol), diisopropylethylamine (0.44 ml, 2.5 mmol) and Compound 21 (0.61 g, 3.42 mmol) in THF (30 mL) was stirred at 0° C. for 8 hours, then cold 5% NaHCO₃ was added to the reaction mixture, and the aqueous mixture was extracted twice with EtOAc. The combined extracts were washed with brine, dried, and concentrated in vacuo. The crude solids were triturated with EtOAc and after filtration, the solids were washed by ethyl acetate dried to give 78 (400 mg, 49%), which was used in the next step without further purification.

### Example 79

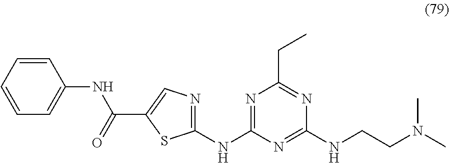

A mixture of compound 78 (0.4 g, 1.11 mmol) and diisopropylethylamine (0.77 mL, 4.4.44 mmol), and N,N-dimethylethane-1,2-diamine (0.39 g, 4.44 mmol) in DMSO was heated at 60° C. for over night. The mixture was extracted by ethyl acetate and the combined organic layers were washed with water and brine. The crude product was recrystallized by MeOH/CHCl₃ to give 79 (21 mg, 5%) ¹H NMR (400 MHz, DMSO-d₆) δ11.80 (br s, 1H), 10.05 (d, J=5.2 Hz, 1H), 8.32 (d, 7.2 Hz, 1H), 7.70 (br s, 1H), 7.66 (m, 2H), 7.1 (m, 2H), 7.05, 1H), 3.42 (m, 2H), 2.52 (m, 2H), 2.45 (m, 2H), 2.23 (m, 6H), 1.1 (m, 3H); ESI-MS: calcd for C₁₉H₂₄N₈OS) 412 found 413 (M+H⁺). HPLC: retention time: 10.2 min; purity 99%

### Example 80

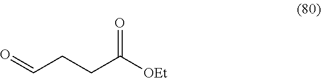

2.9 grams of 10% Pd/C was flushed with H₂, anhydrous THF was added, and the solution was reflushed with H₂. 2,6-Lutidine (21.2 g, 198 mmol) and ethyl 4-chloro-4-oxobutanoate (29.8 g, 181 mmol) were added, and the solution was stirred at room temperature under H₂ for 24 h. The reaction mixture was filtered off through celite and evaporated. The crude residue was dissolved again in CH₂Cl₂ (500 ml) and washed with Water (200 ml), 1N HCl (200 ml) and again with water. The organic layers were dried and evaporated to give 80 (20.2 g, 85%). ¹H NMR (400 MHz, DMSO-d₆) δ 9.80 (s, 1H), 4.14 (q, J=6.8 Hz, 2H), 2.78 (t, J=6.0 Hz, 2H), 2.60 (t, J=6.4 Hz, 2H), 1.24 (t, J=0.8 Hz, 3H).

### Example 81

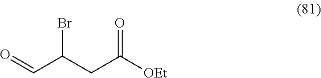

Bromine (18.0 g, 112 mmol) was added over 0.5 hour to a solution of 80 (12.36 g, 107 mmol) in diethyl ether (100 ml) and 1,4-dioxane (91 ml) and the reaction mixture stirred for one hour at room temperature. The reaction mixture was poured into CH₂Cl₂ (300 ml) and sodium hydrogen carbonate (20.09 g, 237 mmol) was added and stirred for 16 h. The solids were filtered off and the filtrate was concentrated to give 81 (22.9 g). The crude brown oil was used in the next step without further purification.

### Example 82

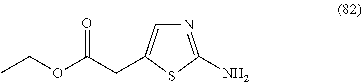

Ethyl 4-bromo-4-oxobutanoate 81 (22.9 g, 109.6 mmol) was added to a suspension of thiourea (7.5 g, 98.71 mmol) in Ethanol. The reaction mixture was refluxed for 8 h. After cooling, the resulting solids were filtered off and washed with cold EtOH to give 82 (11 g, 58.2%).

### Example 83

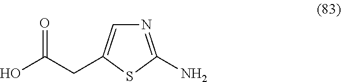

Sodium hydroxide (8 ml of a 1.0 N solution in water) was added to a solution of 82 (2.1 g) in THF (12 ml), methanol (4 ml), H₂O (4 ml). The solution was stirred overnight at ambient temperature. The organic solvents were removed under reduced pressure. The residue was acidified using 1N HCl to pH 3-4. The solvents were removed and used in the next step without further purification.

### Example 84

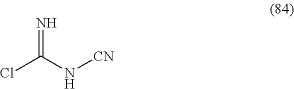

Sodiumdicyanoamide (25 g, 281 mMol) in 25 mL of H₂O was rapidly added to 125 mL of conc. HCl at −18° C. Reaction mixture was stirred at −18° C. for 15 minutes and at 35° C. for another 15 minutes. Cooling at 0° C. resulted in formation of white solid that was filtered and washed with ice-cold water to give 12.5 g (43%) of intermediate 84 which was used crude for next step.

### Example 85

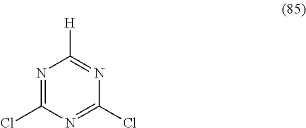

To 150 mL of CH₂Cl₂ at room temperature was added DMF (11.4 mL) followed by POCl₃ (11.4 mMol). After 5 minutes of stirring the intermediate 84 (12.5 g, 120.8 mMol) was added in portions. Reaction mixture was stirred overnight at room temperature. Next day, reaction was washed with water (3×), brine (1×), dried over Na₂SO₄, filtered and solvent was evaporated to give 7.1 g (17%) of off-white solid. ¹H NMR (400 MHz, CDCl₃) δ 8.91 (s, 1H).

### Example 86

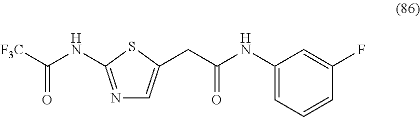

To the 83 (800 mg, 5.06 mMol) in 10 mL of DMF at 0° C. was added pyridine (0.91 mL, 11.32 mMol) followed by careful, dropwise addition of pentafluorophenyl trifluoroacetate (1.72 mL, 10.13 mMol). Reaction mixture was stirred at 0° C. for 10 minutes and 90 minutes at room temperature. 3-fluoroaniline (0.97 mL, 10.13 mMol) was added and reaction mixture was stirred overnight at room temperature. Reaction was poured into 50 mL of 1N HCl, organic layer was separated. Aqueous layer was extracted with EtOAc. Organic fractions were combined, washed with brine, dried over Na₂SO₄, filtered and solvent was evaporated to give 86 (0.4 g, 23%) and this was used in the next step without purification.

### Example 87

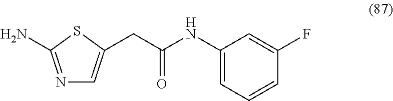

Crude amide 86 from previous step (0.4 g) was heated for 3 hours in 10 mL of methanol and 10 mL of 2N HCl. Reaction mixture was neutralized with Saturated NaHCO₃ and methanol was evaporated. Aqueous solution was extracted with EtOAc. Organic fractions were combined, washed with brine, dried over Na₂SO₄, filtered and solvent was evaporated. Flash column chromatography (silica, CH₂Cl₂/MeOH 5% to 10%) yielded desired product 87 as yellow oil (360 mg, 28% over 2 steps). ¹H NMR (400 MHz, DMSO) δ 10.26 (br s, 1H), 7.58 (m, 3H), 7.30 (m, 2H), 6.86 (m, 2H), 3.65 (s, 2H). ESI-MS: calcd for C₁₁H₁₀FN₃OS) 251. found 252 (M+H⁺).

### Example 88

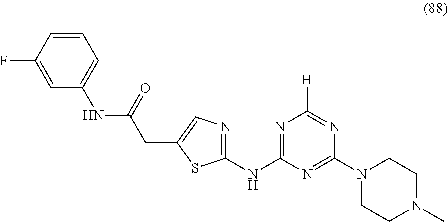

A solution of Compound 87 (0.1 g, 0.39 mmol), diisopropylamine (0.075 ml, 0.43 mmol) and Compound 85 (0.092 g, 0.62 mmol) in THF (10 mL) was stirred at 0° C. for 8 hours. DIPEA (128 μL, 95 mg, and 0.73 mMol) was added followed by 1-methylpiparezine (80 mg, 0.80 mMol) and reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 5% to 10%) yielded 88 as off-white solid (25 mg, 15%). ¹H NMR (400 MHz, DMSO) δ 11.65 (br s, 1H), 10.42 (s, 1H), 8.30 (s, 1H), 7.61 (m, 1H), 7.30 (m, 3H), 6.85 (m, 1H), 3.67 (m, 6H), 2.35 (m, 4H), 2.16 (s, 3H). ESI-MS: calcd for (C₁₉H₂₁FN₈OS) 428. found 429 (M+H⁺).

### Example 89

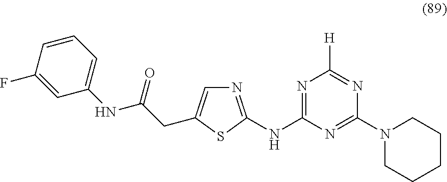

A solution of Compound 87 (0.075 g, 0.3 mmol), diisopropylamine (0.075 ml, 0.32 mmol) and Compound 85 (0.067 g, 0.45 mmol) in THF (10 mL) was stirred at 0° C. for 8 hours. DIPEA (56 μL, 0.32 mmol) was added followed by piperidine (102 mg, 1.20 mmol) and the reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 1% to 5) yielded 89 as off-white solid (10 mg, 8% over two steps). ¹H NMR (400 MHz, DMSO) δ 11.55 (br s, 1H), 10.42 (s, 1H), 8.30 (s, 1H), 7.61 (m, 1H), 7.30 (m, 3H), 6.85 (m, 1H), 3.80 (m, 6H), 1.65 (m, 2H), 1.52 (m, 4H); ESI-MS: calcd for (C₁₉H₂₀FN₇OS) 413. found 414 (M+H⁺).

### Example 90

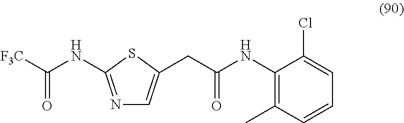

To the 83 (800 mg, 5.06 mmol) in 10 mL of DMF at 0° C. was added pyridine (0.91 mL, 11.32 mmol) followed by careful, dropwise addition of pentafluorophenyl trifluoroacetate (1.72 mL, 10.13 mmol). Reaction mixture was stirred at 0° C. for 10 minutes and 90 minutes at room temperature. 2-chloro-6-methylaniline (1.24 mL, 10.13 mmol) was added and reaction mixture was stirred overnight at room temperature. Reaction was poured into 50 mL of 1N HCl, organic layer was separated. Aqueous layer was extracted with EtOAc. Organic fractions were combined, washed with brine, dried over Na₂SO₄, filtered and solvent was evaporated to give 90 (0.25 g, 42%) and this was used in the next step without further purification.

### Example 91

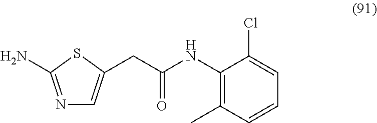

Crude amide 90 from previous step (0.25 g) was heated for 8 hours in 10 mL of methanol and 10 mL of 2N HCl. Reaction mixture was neutralized with Saturated NaHCO₃ and methanol was evaporated. Aqueous solution was extracted with EtOAc. Organic fractions were combined, washed with brine, dried over Na₂SO₄, filtered and solvent was evaporated. Flash column chromatography (silica, CH₂Cl₂/MeOH 5% to 10%) yielded desired product 91 (174 mg, 12% over 2 steps). ¹H NMR (400 MHz, DMSO) δ 9.68 (s, 1H), 7.31 (m, 1H), 7.20 (m, 2H), 6.75 (m, 3H), 3.63 (s, 2H), 2.12 (s, 3H); ESI-MS: calcd for C₁₂H₁₂ClN₃OS) 281 found 282 (M+H⁺).

### Example 92

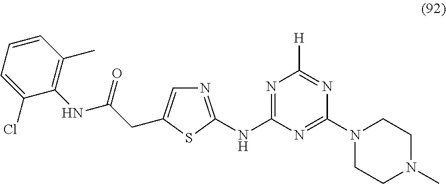

A solution of Compound 91 (0.07 g, 0.25 mmol), diisopropylamine (47 ul, 0.27 mmol) and Compound 85 (0.056 g, 0.37 mmol) in THF (10 mL) was stirred at 0° C. for 8 hours. DIPEA (0.17 mL, 1.0 mmol) was added followed by 1-methylpiparezine (0.11 ml, 1.0 mmol) and reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 5% to 10%) yielded 92 as off-white solid (27 mg, 24%). ¹H NMR (400 MHz, DMSO) δ 11.48 (br s, 1H), 9.85 (s, 1H), 8.30 (s, 1H), 7.35 (m, 1H), 7.27 (s, 1H), 7.21 (m, 2H), 3.85 (m, 6H), 2.35 (m, 4H), 2.19 (s, 3H), 2.13 (s, 3H); ESI-MS: calcd for (C₂₀H₂₃ClN₈OS) 458. found 459 (M+H⁺).

### Example 93

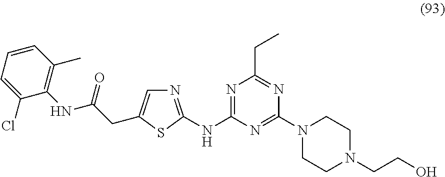

Compound 91 (111 mg, 0.38 mmol), 2-(4-(4-chloro-6-ethyl-1,3,5-triazin-2-yl)piperazin-1-yl)ethanol (39) (122 mg, 0.45 mmol), Pd(OAc)₂ (10 mg, 0.04 mmol), Xantphos (48 mg, 0.08 mmol) and K₂CO₃ (1.0 g, 7.5 mmol) were added in screw capped microwave vial. THF: DMF (2.5 mL, 1.5:1) was added and vial was sealed with a cap. The mixture was heated at 150° C. for 10 min. under microwave (Biotage, Initiator 2.0) conditions. Reaction mixture was filtered and the solid was washed with CH₂Cl₂ and MeOH, concentrated. (silica, CH₂Cl₂/MeOH 5% to 10%) yielded 93 as off-white solid (25 mg, 14%). ¹H NMR (400 MHz, DMSO) δ 11.45 (br s, 1H), 9.55 (br s, 1H), 7.33 (m, 2H), 7.14 (m, 2H), 4.40 (t, 1H, J=5.4 Hz), 3.73 (bs, 2H), 3.64 (bs, 2H), 3.51-3.47 (m, 4H), 2.49-2.35 (m, 8H), 2.11 (s, 3H), 1.15 (t, 3H, J=7.6 Hz).; ESI-MS: calcd for (C₂₃H₂₉ClN₈O₂S) 517. found 518 (M+H⁺).

### Example 94

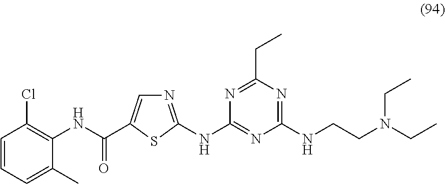

To the compound 22 (100 mg, 0.244 mMol) in 5 mL oh iPrOH was added DIPEA (170 μL, 126 mg, 0.976 mMol) and N,N-diethylehylenedialine (52 μL, 43 mg, 0.366 mMol). Reaction mixture was microwaved at 120° C. for 40 minutes. Disappearance of starting material was confirmed by TLC. Solvent was removed under reduced pressure and flash column chromatography yielded 85 mg (44%) of desired product 94. ¹H NMR (400 MHz, DMSO) δ 11.78 (bs, 1H), 9.92 (s, 1H), 8.27 (s, 1H), 7.62 (bs, 1H), 7.40 (dd, J=7.2, 1.6 Hz, 1H), 7.27 (m, 2H), 3.60-3.50 (m, 2H), 2.65−2.45 (m, 8H), 2.23 (s, 3H), 1.23 (m, 3H), 0.92 (t, J=7.2 Hz, 6H). ESI-MS: calcd for (C₂₂H₂₉ClN₈OS) 488, MS (ESI) m/z 489 [M+H]⁺.

### Example 95

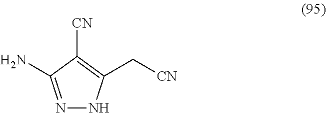

To 2-amino-1-propene-1,1,3-tricarbonitrile (15.5 g, 117.3 mMol) in 150 mL of H₂O at room temperature was added 50-60% hydrazine hydrate (7.4 mL, 7.6 g, 129.03 mMol). After solid dissolved reaction mixture was heated at 90° C. for 30 minutes and then ice cooled. Formed solid was filtered and dried overnight on high vacuum to give 13 g (75%) of product 95 that was used crude for next step.

### Example 96

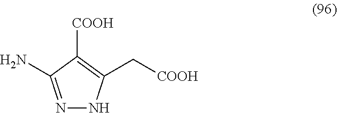

Compound 95 (13 g, 88.4 mMo) was added to 120 mL of 10N NaOH (aq) and heated at 100° C. overnight. Reaction mixture was cooled in ice bath and pH was adjusted to 3 using conc. HCl. After cooling on ice bath for 1 hr formed solid was collected, washed with H₂O, dried on air for 1 hr then washed with EtOAc and dried on high vacuum to give 13.2 g (81%) of desired product 96 that was used crude for next step.

### Example 97

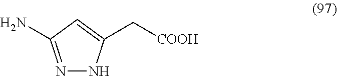

Compound 96 (13.2 g, 71.3 mMol) in 250 mL of H₂O was refluxed at 125° C. for 5 hrs. Reaction mixture was cooled at room temperature and green residue was filtered off. Filtrate was evaporated to give crude product that was dried on high vacuum to yield 10 g (99%) of desired product 97. ¹H NMR (400 MHz, DMSO) δ 6.88 (s, 2H), 5.59 (bs, 2H), 5.25 (s, 1H), 3.62 (s, 2H). ¹H NMR (400 MHz, DMSO+D₂O) δ 5.28 (s, 1H), 3.62 (s, 2H).

### Example 98

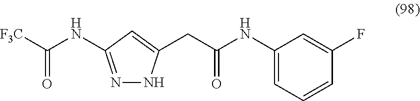

To compound 97 (700 mg, 4.96 mMol) in 7 mL of DMF at 0° C. was added pyridine (883 μL, 863 mg, 10.91 mMol) followed by careful, dropwise addition of pentafluorophenyl trifluoroacetate (1.68 mL, 2.78 g, 9.92 mMol). Reaction mixture was stirred at 0° C. for 10 minutes and 90 minutes at room temperature. 3-fluoroaniline (954 μL, 1.1 g, 9.92 mMol) was added and reaction mixture was stirred overnight at room temperature. Reaction was poured into 50 mL of 1N HCl, organic layer was separated. Aqueous layer was extracted with EtOAc. Organic fractions were combined, washed with brine, dried over Na₂SO₄, filtered and solvent was evaporated to give 1.6 g (quant.) of crude product 98 that was used in the next step without purification.

### Example 99

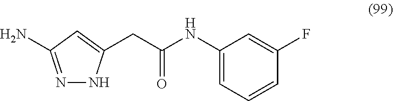

Crude amide 98 from previous step (1.6 g, 4.96 mMol) was heated for 3 hours in 10 mL of methanol and 10 mL of 2N HCl. Reaction mixture was neutralized with conc. NaHCO₃ and methanol was evaporated. Aqueous solution was extracted with EtOAc. Organic fractions were combined, washed with brine, dried over Na₂SO₄, filtered and solvent was evaporated. Flash column chromatography (silica, CH₂Cl₂/MeOH 5% to 15%) yielded desired product 99 as yellow oil (640 mg, 55% over 2 steps). ¹H NMR (400 MHz, DMSO) δ 10.26 (s, 1H), 7.58 (m, 1H), 7.30 (m, 2H), 7.12 (bs, 1H), 6.86 (m, 1H), 5.30 (s, 1H), 3.47 (s, 2H). calcd for (C₁₁FN₄O) 234, MS (ESI) m/z 235 [M+H]⁺.

### Example 100

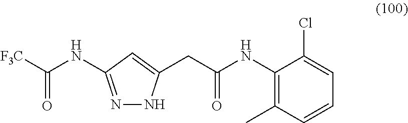

To compound 97 (700 mg, 4.96 mMol) in 7 mL of DMF at 0° C. was added pyridine (883 μL, 863 mg, 10.91 mMol) followed by careful, dropwise addition of pentafluorophenyl trifluoroacetate (1.68 mL, 2.78 g, 9.92 mMol). Reaction mixture was stirred at 0° C. for 10 minutes and 90 minutes at room temperature. 2-chloro-6-methylaniline (1.22 mL, 1.4 g, 9.92 mMol) was added and reaction mixture was stirred overnight at room temperature. Reaction was poured into 50 mL of 1N HCl, organic layer was separated. Aqueous layer was extracted with EtOAc. Organic fractions were combined, washed with brine, dried over Na₂SO₄, filtered and solvent was evaporated to give 1.79 g (quant.) of crude product 100 that was used in the next step without purification.

### Example 101

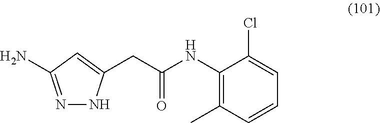

Crude amide 100 from previous step (1.79 g, 4.96 mMol) was heated for 3 hours in 10 mL of methanol and 10 mL of 2N HCl. Reaction mixture was neutralized with conc. NaHCO₃ and methanol was evaporated. Aqueous solution was extracted with EtOAc. Organic fractions were combined, washed with brine, dried over Na₂SO₄, filtered and solvent was evaporated. Flash column chromatography (silica, CH₂Cl₂/MeOH 5% to 15%) yielded desired product 101 as yellow oil (250 mg, 19% over 2 steps). ¹H NMR (400 MHz, DMSO) δ 11.20 (bs, 1H), 9.62 (s, 1H), 7.32 (m, 1H), 7.21 (m, 2H), 5.35 (s, 1H), 4.55 (bs, 2H), 3.50 (s, 2H), 2.15 (s, 3H). calcd for (C₁₂H₁₃ClN₄O) 264, MS (ESI) m/z 265 [M+H]⁺.

### Example 102

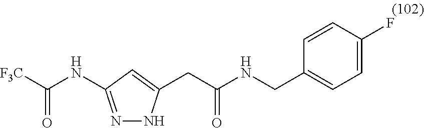

To the 97 (700 mg, 4.96 mMol) in 7 mL of DMF at 0° C. was added pyridine (883 μL, 863 mg, 10.91 mMol) followed by careful, dropwise addition of pentafluorophenyl trifluoroacetate (1.68 mL, 2.78 g, 9.92 mMol). Reaction mixture was stirred at 0° C. for 10 minutes and 90 minutes at room temperature. 4-fluorobenzylamine (1.13 mL, 1.12 g, 9.92 mMol) was added and reaction mixture was stirred overnight at room temperature. Reaction was poured into 50 mL of 1N HCl, organic layer was separated. Aqueous layer was extracted with EtOAc. Organic fractions were combined, washed with brine, dried over Na₂SO₄, filtered and solvent was evaporated to give 1.71 g (quant.) of crude product 102 that was used in the next step without purification.

### Example 103

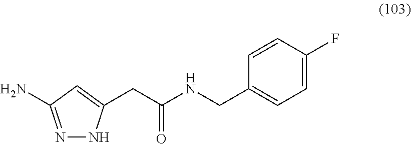

Crude amide 102 from previous step (1.71 g, 4.96 mMol) was heated for 3 hours in 10 mL of methanol and 10 mL of 2N HCl. Reaction mixture was neutralized with conc. NaHCO₃ and methanol was evaporated. Aqueous solution was extracted with EtOAc. Organic fractions were combined, washed with brine, dried over Na₂SO₄, filtered and solvent was evaporated. Flash column chromatography (silica, CH₂Cl₂/MeOH 5% to 15%) yielded desired product 103 as yellow oil (520 mg, 42% over 2 steps). ¹H NMR (400 MHz, DMSO) 11.50 (bs, 1H), 8.37 (t, J=6.0 Hz, 1H), 7.27 (m, 2H), 7.13 (m, 2H), 5.75 (bs, 2H), 5.25 (s, 1H), 4.23 (d, J=6.0 Hz, 2H), 3.17 (s, 2H). calcd for (C₁₂H₁₃FN₄O) 248, MS (ESI) m/z 249 [M+H]⁺.

### Example 104

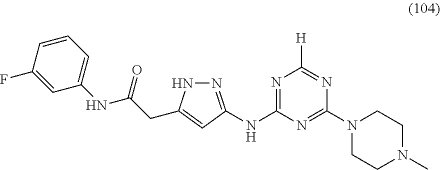

To compound 85 (100 mg, 0.67 mMol) in 5 mL of THF at room temperature was added 99 (157 mg, 0.67 mMol) and DIPEA (128 μL, 95 mg, 0.73 mMol) in 5 mL of THF. Reaction mixture was stirred for 4 hours at room temperature. DIPEA (128 μL, 95 mg, 0.73 mMol) was added followed by 1-methylpiparezine (75 μL, 67 mg, 0.67 mMol) and reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 5% to 10%) yielded 104 as off-white solid (24 mg, 9%). ¹H NMR (400 MHz, DMSO) δ 12.17 (s, 1H), 10.43 (s, 1H), 9.75 (s, 1H), 8.16 (s, 1H), 7.61 (m, 1H), 7.32 (m, 2H), 6.89 (m, 1H), 6.48 (s, 1H), 3.67 (bs, 6H), 2.29 (bs, 4H), 2.16 (s, 3H). calcd for (C₁₉H₂₂FN₉O) 411, m/z 412 [M+H]⁺.

### Example 105

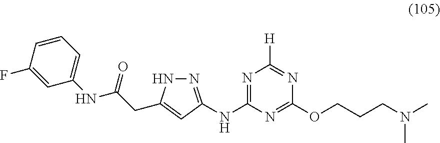

To compound 85 (100 mg, 0.67 mMol) in 5 mL of THF at room temperature was added 99 (157 mg, 0.67 mMol) and DIPEA (128 μL, 95 mg, 0.73 mMol) in 5 mL of THF. Reaction mixture was stirred for 4 hours at room temperature. DIPEA (128 μL, 95 mg, 0.73 mMol) was added followed by 3-dimethylamino-1-propanol (78 μL, 69 mg, 0.67 mMol) and reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 5% to 20%) yielded 105 as off-white solid (32 mg, 12%). ¹H NMR (400 MHz, DMSO) δ 12.29 (s, 1H), 10.42 (bs, 2H), 8.41 (s, 1H), 7.60 (m, 1H), 7.32 (m, 2H), 6.88 (m, 1H), 6.54 (s, 1H), 4.31 (t, J=6.0 Hz, 2H), 3.72 (s, 2H), 2.39 (bs, 2H), 2.19 (s, 6H), 1.83 (bs, 2H). calcd for (C₁₉H₂₃FN₈O₂) 414, m/z 415 [M+H]⁺.

### Example 106

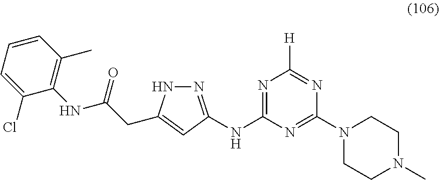

To compound 85 (70 mg, 0.47 mMol) in 5 mL of THF at room temperature was added 101 (124 mg, 0.47 mMol) and DIPEA (87 μL, 64 mg, 0.50 mMol) in 5 mL of THF. Reaction mixture was stirred for 4 hours at room temperature. DIPEA (87 μL, 64 mg, 0.50 mMol) was added followed by 1-methylpiparezine (52 μL, 47 mg, 0.47 mMol) and reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 5% to 10%) yielded 106 as off-white solid (70 mg, 34%). ¹H NMR (400 MHz, DMSO) δ 12.16 (s, 1H), 9.77 (bs, 2H), 8.17 (s, 1H), 7.34 (m, 1H), 7.22 (m, 2H), 6.54 (s, 1H), 3.72 (bs, 6H), 2.30 (bs, 4H), 2.18 (s, 3H), 2.16 (s, 3H). calcd for (C₂₀H₂₄ClN₉O) 441, m/z 442 [M+H]⁺.

### Example 107

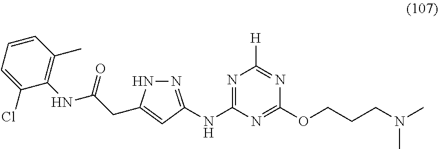

To compound 85 (70 mg, 0.47 mMol) in 5 mL of THF at room temperature was added 101 (124 mg, 0.47 mMol) and DIPEA (87 μL, 64 mg, 0.50 mMol) in 5 mL of THF. Reaction mixture was stirred for 4 hours at room temperature. DIPEA (87 μL, 64 mg, 0.50 mMol) was added followed by 3-dimethylamino-1-propanol (55 μL, 48 mg, 0.47 mMol) and reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 5% to 15%) yielded 107 as off-white solid (35 mg, 17%). ¹H NMR (400 MHz, DMSO) δ 12.29 (s, 1H), 10.42 (bs, 1H), 9.78 (s, 1H), 8.42 (s, 1H), 7.34 (m, 1H), 7.22 (m, 2H), 6.61 (s, 1H), 4.33 (t, J=6.4 Hz, 2H), 3.72 (s, 2H), 2.36 (bs, 2H), 2.17 (m, 9H), 1.83 (bs, 2H). calcd for (C₂₀H₂₅ClN₈O₂) 444, m/z 445 [M+H]⁺.

### Example 108

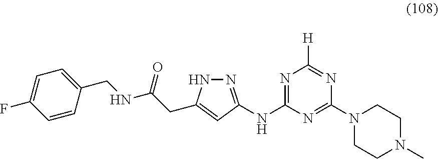

To compound 85 (100 mg, 0.67 mMol) in 5 mL of THF at room temperature was added 103 (166 mg, 0.67 mMol) and DIPEA (128 μL, 95 mg, 0.73 mMol) in 5 mL of THF. Reaction mixture was stirred for 4 hours at room temperature. DIPEA (128 μL, 95 mg, 0.73 mMol) was added followed by 1-methylpiparezine (75 μL, 67 mg, 0.67 mMol) and reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 5% to 10%) yielded 108 as off-white solid (68 mg, 24%). ¹H NMR (400 MHz, DMSO) δ 12.08 (s, 1H), 9.69 (s, 1H), 8.53 (s, 1H), 8.17 (s, 1H), 7.29 (m, 2H), 7.13 (m, 2H), 6.43 (s, 1H), 4.27 (d, J=6.0 Hz, 2H), 3.72 (bs, 4H), 3.51 (s, 2H), 2.31 (bs, 4H), 2.20 (s, 3H). calcd for (C₂₀H₂₄FN₉O) 425, m/z 426 [M+H]⁺.

### Example 109

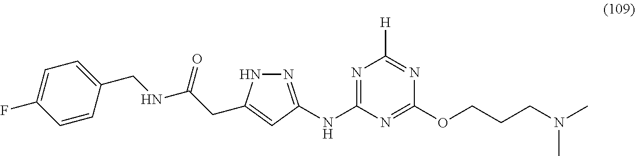

To compound 85 (100 mg, 0.67 mMol) in 5 mL of THF at room temperature was added 103 (166 mg, 0.67 mMol) and DIPEA (128 μL, 95 mg, 0.73 mMol) in 5 mL of THF. Reaction mixture was stirred for 4 hours at room temperature. DIPEA (128 μL, 95 mg, 0.73 mMol) was added followed by 3-dimethylamino-1-propanol (78 μL, 69 mg, 0.67 mMol) and reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 5% to 20%) yielded 109 as off-white solid (30 mg, 10%). ¹H NMR (400 MHz, DMSO) δ 12.20 (s, 1H), 10.38 (bs, 1H), 8.54 (s, 1H), 8.42 (s, 1H), 7.29 (m, 2H), 7.13 (m, 2H), 6.49 (s, 1H), 4.32 (t, J=6.4 Hz, 2H), 4.26 (d, J=6.0 Hz, 2H), 3.52 (s, 2H), 2.32 (t, J=6.8 Hz, 2H), 2.14 (s, 6H), 1.83 (m, 2H). calcd for (C₂₀H₂₅FN₈O₂) 428, m/z 451 [M+Na]⁺.

### Example 110

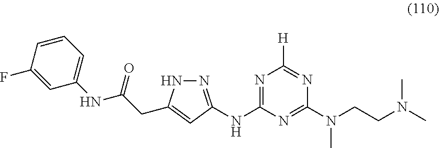

To compound 85 (100 mg, 0.67 mMol) in 5 mL of THF at room temperature was added 99 (157 mg, 0.67 mMol) and DIPEA (128 μL, 95 mg, 0.73 mMol) in 5 mL of THF. Reaction mixture was stirred for 4 hours at room temperature. DIPEA (128 μL, 95 mg, 0.73 mMol) was added followed by N,N,N-trimethylethylenediamine (87 μL, 68 mg, 0.67 mMol) and reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 5% to 20%) yielded 110 as off-white solid (37 mg, 13%). ¹H NMR (400 MHz, DMSO @ 80° C.) δ 11.95 (bs, 1H), 10.13 (s, 1H), 9.20 (bs, 1H), 8.17 (s, 1H), 7.56 (m, 1H), 7.33 (m, 2H), 6.85 (m, 1H), 6.49 (s, 1H), 3.67 (t, J=6.0 Hz, 2H), 3.08 (s, 3H), 2.48 (m, 2H), 2.21 (s, 6H). calcd for (C₁₉H₂₄FN₉O) 413, m/z 414 [M+H]⁺.

### Example 111

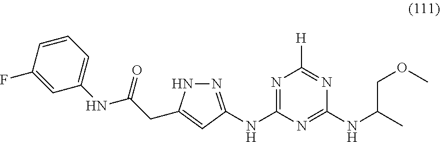

To compound 85 (100 mg, 0.67 mMol) in 5 mL of THF at room temperature was added 99 (157 mg, 0.67 mMol) and DIPEA (128 μL, 95 mg, 0.73 mMol) in 5 mL of THF. Reaction mixture was stirred for 4 hours at room temperature. DIPEA (128 μL, 95 mg, 0.73 mMol) was added followed by 1-methoxy-2-propylamine (71 μL, 60 mg, 0.67 mMol) and reaction mixture was stirred overnight at room temperature. Solvent was evaporated and crude material was re-dissolved in EtOAc (30 mL), washed with sat. NaHCO₃, brine, dried over Na₂SO₄, filtered and solvent was evaporated. Flash column chromatography (silica, CH₂Cl₂/MeOH 5% to 10%) yielded 111 as off-white solid (40 mg, 15%). ¹H NMR (400 MHz, DMSO @ 80° C.) δ 11.93 (bs, 1H), 10.09 (s, 1H), 9.10 (bs, 1H), 8.12 (s, 1H), 7.56 (m, 1H), 7.32 (m, 2H), 6.85 (m, 1H), 6.52 (s, 1H), 4.18 (m, 1H), 3.66 (bs, 2H), 3.39 (m, 1H), 3.27 (s, 3H), 1.13 (s, 3H). calcd for (C₁₈H₂₁FN₈O₂) 400, m/z 401 [M+H]⁺.

### Example 112

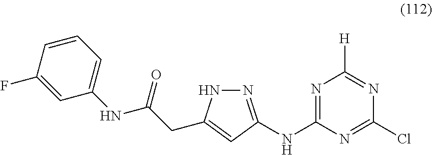

To compound 85 (342 mg, 2.28 mMol) in 20 mL of THF at room temperature was added 99 (535 mg, 2.28 mMol) and DIPEA (436 μL, 323 mg, 2.50 mMol) in 15 mL of THF. Reaction mixture was stirred for 4 hours at room temperature. Added 50 mL of H₂O, extracted with EtOAc. Organic fractions were combined, washed with sat. NaHCO₃, brine, dried over Na₂SO₄, filtered and solvent was evaporated. Flash column chromatography (silica, CH₂Cl₂/MeOH 20%) yielded 112 as yellow solid (400 mg, 51%). ¹H NMR (400 MHz, DMSO @ 80° C.) δ 12.25 (bs, 1H), 10.68 (s, 1H), 10.21 (s, 1H), 8.55 (s, 1H), 7.56 (m, 1H), 7.33 (m, 2H), 6.85 (m, 1H), 6.46 (s, 1H), 3.74 (s, 2H). calcd for (C₁₄H₁₁ClFN₇O) 347, m/z 348 [M+H]⁺.

### Example 113

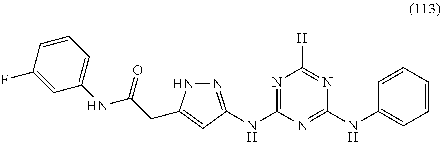

To compound 112 (100 mg, 0.29 mMol) in 5 mL of THF at room temperature was added DIPEA (55 μL, 41 mg, 0.32 mMol) followed by aniline (26 μL, 27 mg, 0.29 mMol). Reaction mixture was stirred overnight at 60° C. Added 30 mL of EtOAc, washed with sat. NaHCO₃, brine, dried over Na₂SO₄, filtered and solvent was evaporated. Flash column chromatography (silica, CH₂Cl₂/MeOH 5% to 20%) yielded 113 as light yellow solid (15 mg, 13%). ¹H NMR (400 MHz, DMSO @ 80° C.) δ 12.03 (bs, 1H), 10.10 (s, 1H), 9.43 (bs, 2H), 8.31 (s, 1H), 7.72 (m, 2H), 7.57 (m, 1H), 7.34 (m, 4H), 6.97 (bs, 1H), 6.86 (m, 1H), 6.49 (bs, 1H), 3.70 (s, 2H). calcd for (C₂₀H₁₇FN₈O) 404, m/z 405 [M+H]⁺.

### Example 114

This example illustrated Src Kinase Assays. Briefly, in a final reaction volume of 25 μL, c-SRC (h) (5-10 mU) is incubated with 8 mM MOPS pH 7.0, 0.2 mM EDTA, 250 μM KVEKIGEGTYGVVYK (Cdc2 peptide), 10 mM MgAcetate and [g-33P-ATP] (specific activity approx. 500 cpm/pmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

Table 1 shows representative data for the inhibition of Src kinase by the compounds of this invention.

TABLE 1

| Example No. | % Inhibition of c-Src @10 μM |
|---|---|
| 5 | >90 |
| 7 | >90 |
| 10 | >90 |
| 11 | >90 |
| 13 | >90 |
| 14 | >90 |
| 17 | >90 |
| 18 | >90 |
| 19 | >90 |
| 20 | >90 |
| 23 | >90 |
| 24 | >90 |
| 25 | >90 |
| 26 | >90 |
| 27 | >90 |
| 28 | >90 |
| 31 | >90 |
| 34 | >90 |
| 38 | >90 |
| 40 | >90 |
| 52 | <50 |
| 59 | >90 |
| 63 | <50 |
| 67 | <50 |
| 71 | >90 |
| 75 | >90 |
| 79 | 50-90 |
| 88 | <50 |
| 89 | <50 |
| 92 | <50 |
| 93 | <50 |
| 94 | >90 |
| 104 | <50 |
| 105 | <50 |
| 106 | <50 |
| 107 | <50 |
| 108 | <50 |
| 109 | <50 |
| 110 | <50 |
| 111 | <50 |
| 113 | <50 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A compound of the formula 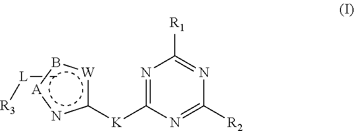
or a pharmaceutically acceptable salt thereof, wherein:
A, B, W is selected from S, O, NR₄, CR₄ or L-R₃;
R₄ is independently selected from hydrogen or an optionally substituted C₁₋₄ aliphatic group.
R₁ represents hydrogen, halogen, hydroxy, amino, cyano, alkyl, cycloalkyl, alkenyl, alkynyl, alkylthio, aryl, arylalkyl, heterocyclic, heteroaryl, heterocycloalkyl, alkylsulfonyl, alkoxycarbonyl and alkylcarbonyl.
R₂ is selected from:
(i) amino, alkyl amino, aryl amino, heteroaryl amino;
(ii) C₁-C₆ alkyl, C₂-C₆ alkenyl, C₂-C₆ alkynyl;
(iii) heterocyclic, heteroaryl; and
(iv) groups of the formula (Ia): 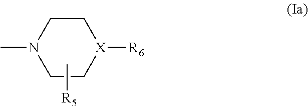
wherein:
R₅ represents hydrogen, C₁-C₄ alkyl, oxo;
X is CH, when R₆ is hydrogen; or X—R₆ is O; or X is N, R₆ represents groups of hydrogen, C₁-C₆ alkyl, C₂-C₆ alkenyl, C₂-C₆ alkynyl, C₃-C₁₀ aryl or heteroaryl, (C₃-C₇cycloalkyl)C₁-C₄alkyl, C₁-C₆ haloalkyl, C₁-C₆ alkoxy, C₁-C₆ alkylthio, C₂-C₆ alkanoyl, C₁-C₆ alkoxycarbonyl, C₂-C₆ alkanoyloxy, mono- and di-(C₃-C₈ cycloalkyl)aminoC₀-C₄alkyl, (4- to 7-membered heterocycle)C₀-C₄alkyl, C₁-C₆ alkylsulfonyl, mono- and di-(C₁-C₆ alkyl) sulfonamido, and mono- and di-(C₁-C₆alkyl)aminocarbonyl, each of which is substituted with from 0 to 4 substituents independently chosen from halogen, hydroxy, cyano, amino, —COOH and oxo;
L represents O, S, SO, CO, SO₂, CO₂, NR₄, (CH₂)ₘ, m=0-3, CONR₄, NR₄CO, NR₄SO₂, SO₂NR₄, NR₄CO₂, NR₄COR₄, NR₄SO₂NR₄, NR₄NR₄, OCONR₄, C(R₄)₂CONR₄, NR₄COC(R₄), C(R₄)₂SO, C(R₄)₂SO₂, C(R₄)₂SO₂NR₄, C(R₄)₂NR₄, C(R₄)₂NR₄CO, C(R₄)₂NR₄CO₂, C(R₄)═NNR₄, C(R₄)═N—O, C(R₄)₂NR₄NR₄, C(R₄)₂NR₄SO₂NR₄, C(R₄)₂NR₄CONR₄, O(CH₂)ₚ, S(CH₂)ₚ, p=1-3, or (CH₂)_{q}0, or (CH₂)_{q}S, q=1-3;
R₃ is selected from:
(i) C₁-C₆ alkyl, C₂-C₆ alkenyl, C₂-C₆ alkynyl;
(ii) heterocyclic,
(iii) Ar,
Ar represents heteroaryl or aryl, each of which is substituted with from 0 to 4 substituents independently chosen from:
(1) halogen, hydroxy, amino, cyano, —COOH, —SO₂NH₂, oxo, nitro and alkoxycarbonyl; and
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- and di-(C₁-C₆alkyl)amino, C₁-C₆ alkylsulfonyl, mono- and di-(C₁-C₆alkyl) sulfonamido and mono- and di-(C₁-C₆alkyl)aminocarbonyl; phenylC₀-C₄alkyl and (4- to 7-membered heterocycle)-C₀-C₄alkyl, each of which is substituted with from 0 to 4 secondary substituents independently chosen from halogen, hydroxy, cyano, oxo, imino, C₁-C₄alkyl, C₁-C₄alkoxy and C₁-C₄haloalkyl;
K is selected from:
i) absence;
ii) O, S, SO, SO₂;
iii) (CH2)ₘ, m=0-3, O(CH₂)ₚ, p=1-3, (CH₂)_{q}O, q=1-3,
iv) NR₇; and
R₇ represents hydrogen, alkyl, cycloalkyl, alkenyl, alkynyl, alkylthio, aryl, arylalkyl.

2. A process for making compound of claim 1 or its pharmaceutically acceptable salts, hydrates, solvates, crystal forms salts and individual diastereomers thereof.

3. A pharmaceutical composition comprising at least one compound of claim 1 or its pharmaceutically acceptable salts, hydrates, solvates, crystal forms salts and individual diastereomers thereof, and a pharmaceutically acceptable carrier.

4. A compound selected from the group consisting of:

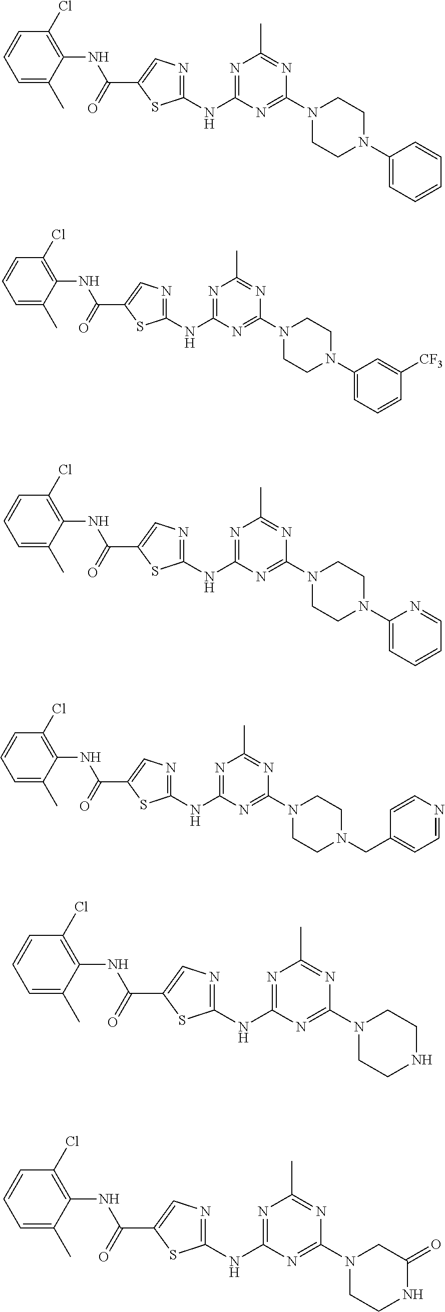

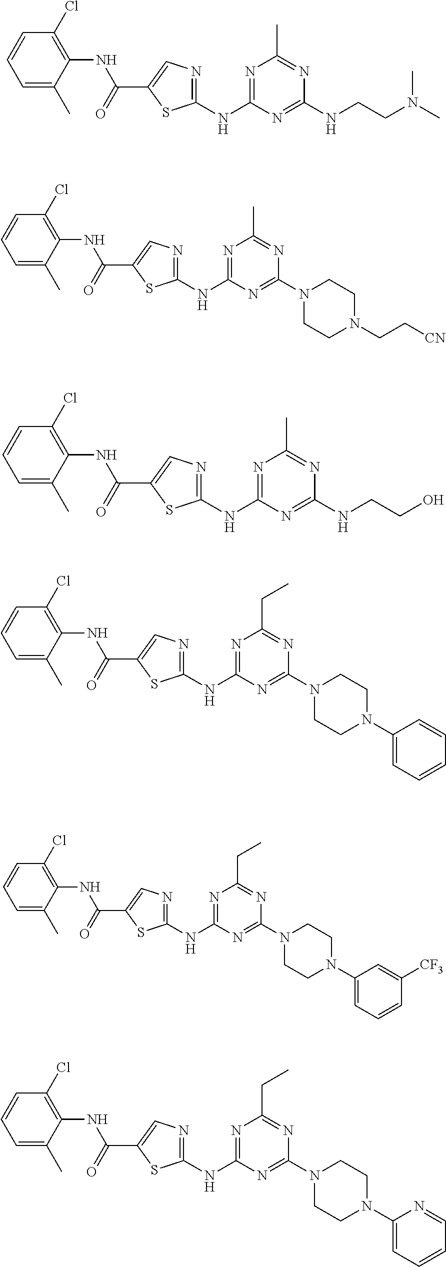

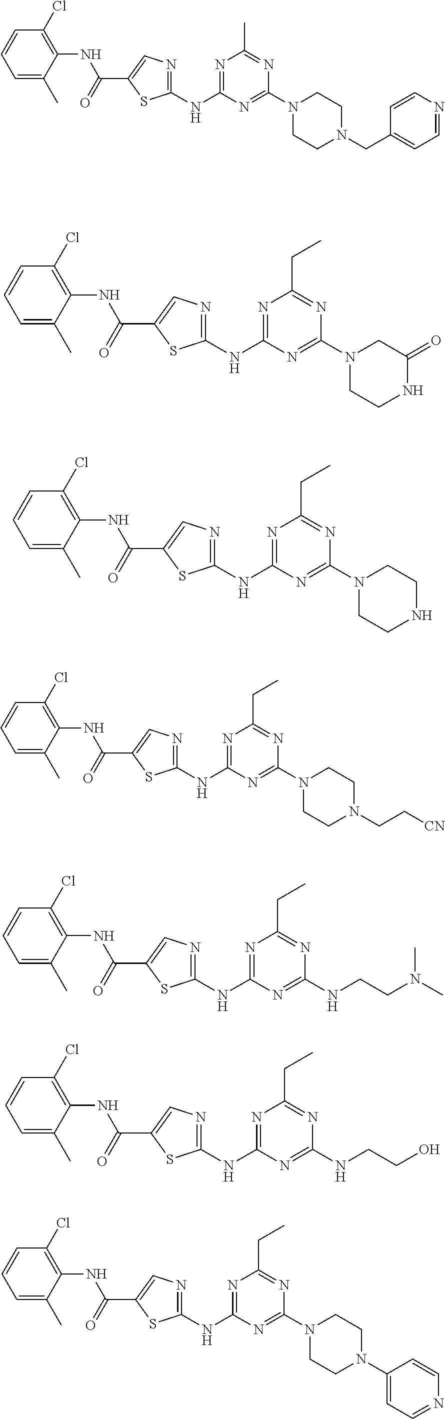

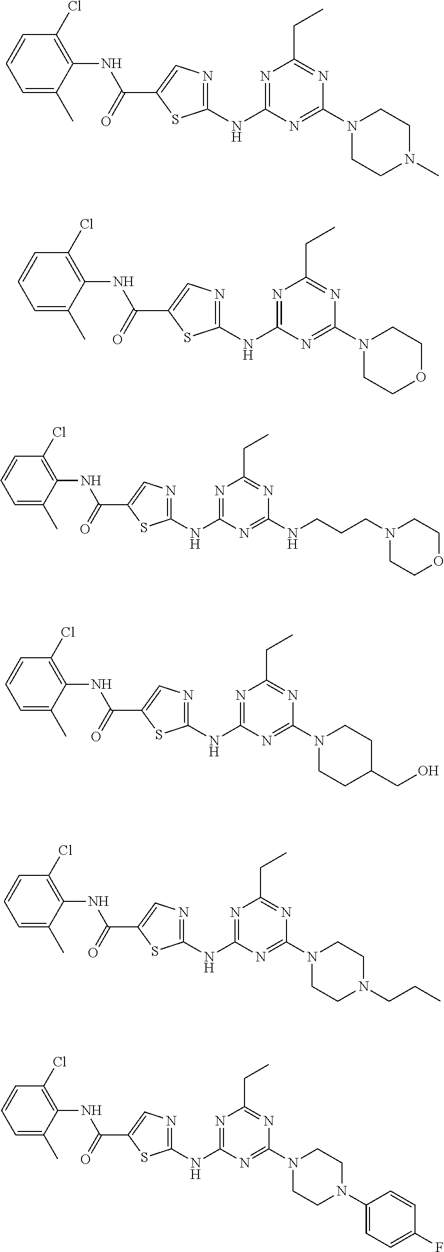

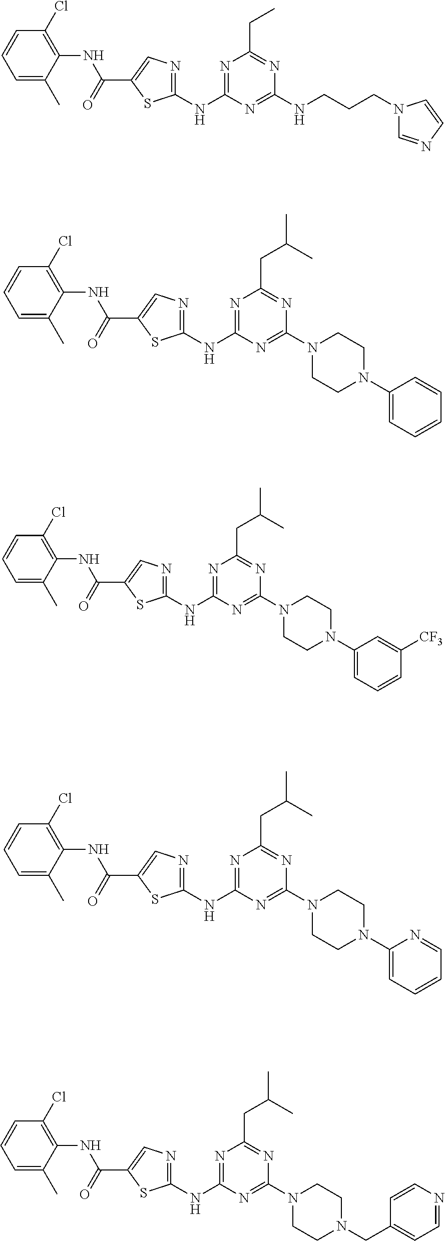

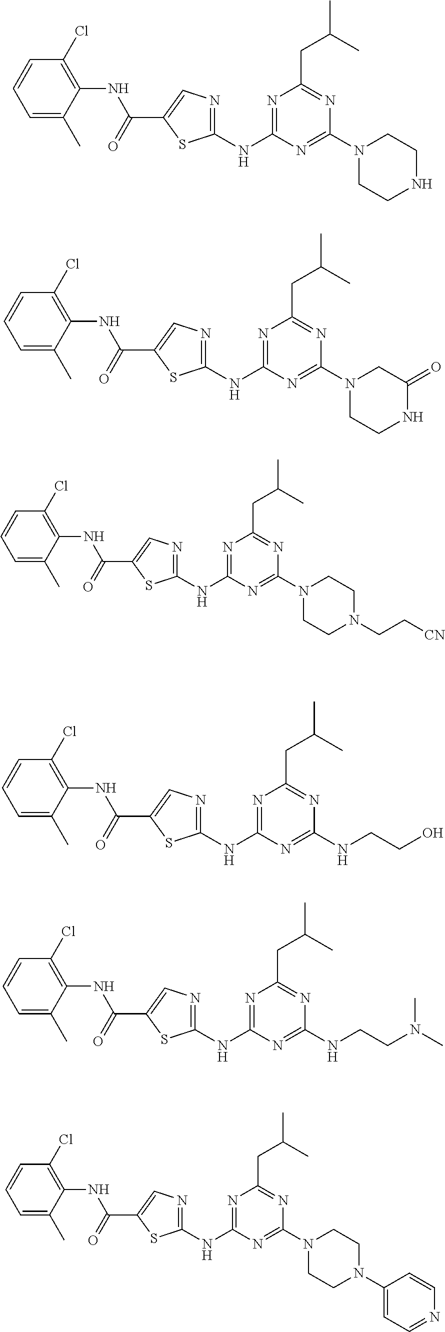

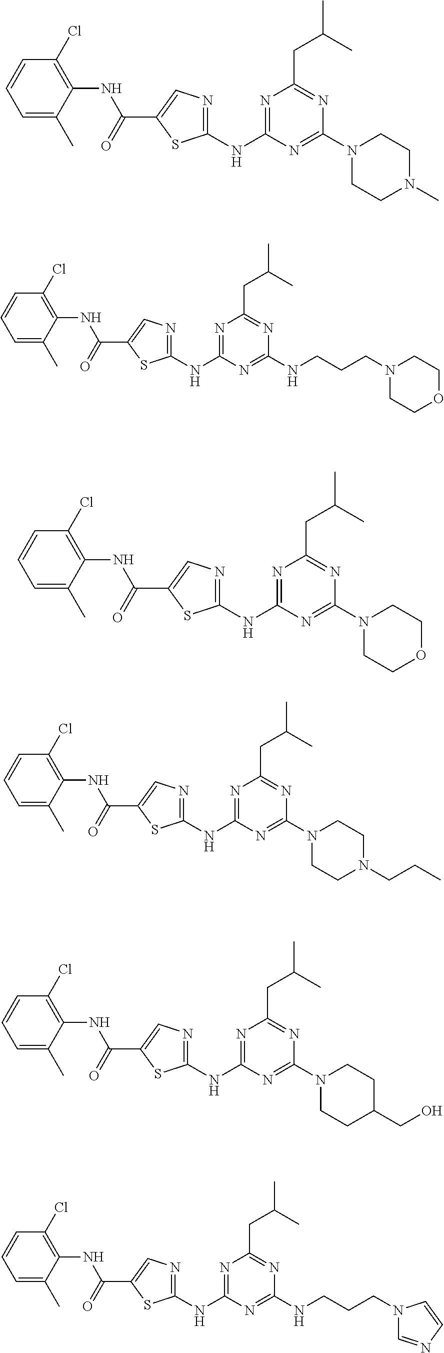

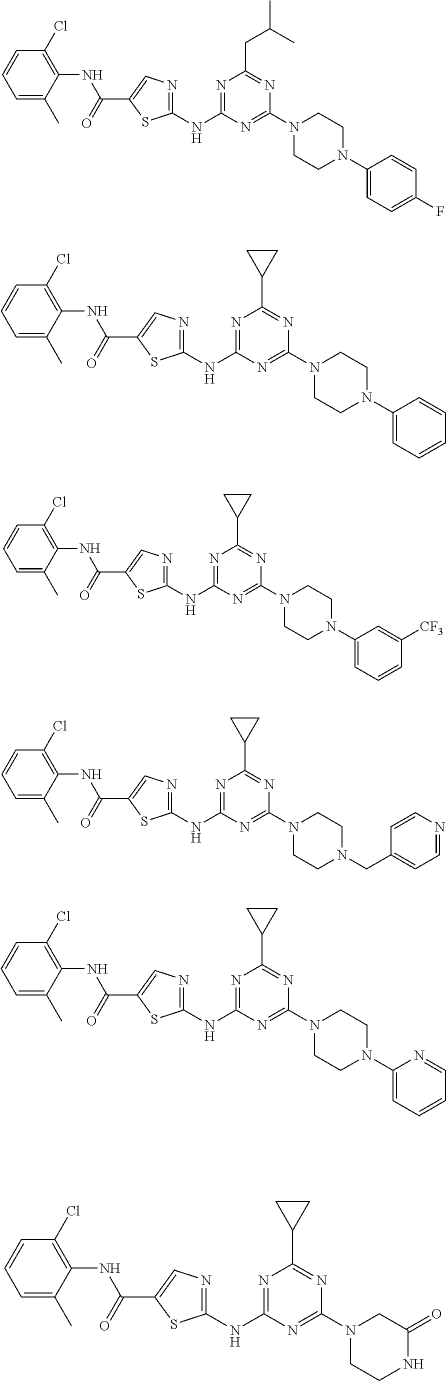

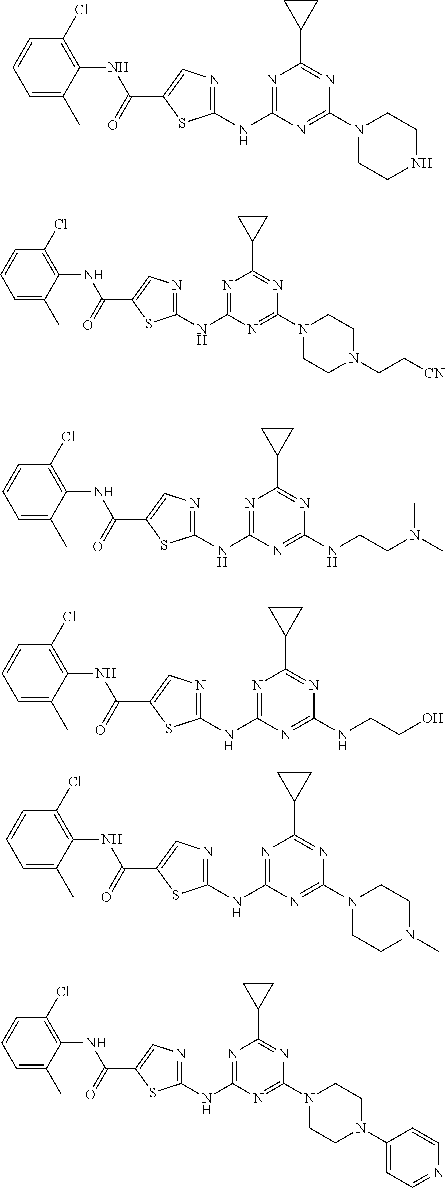

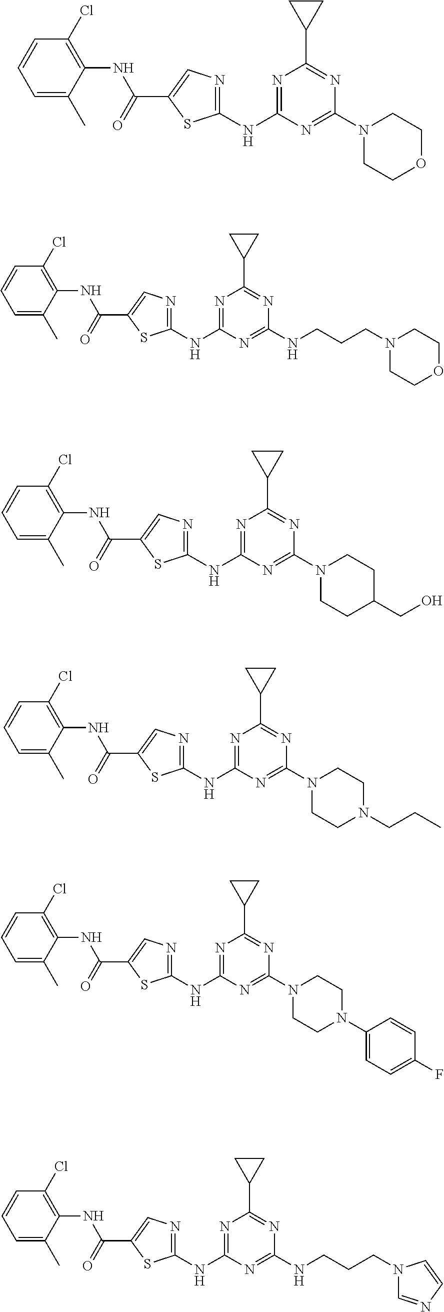

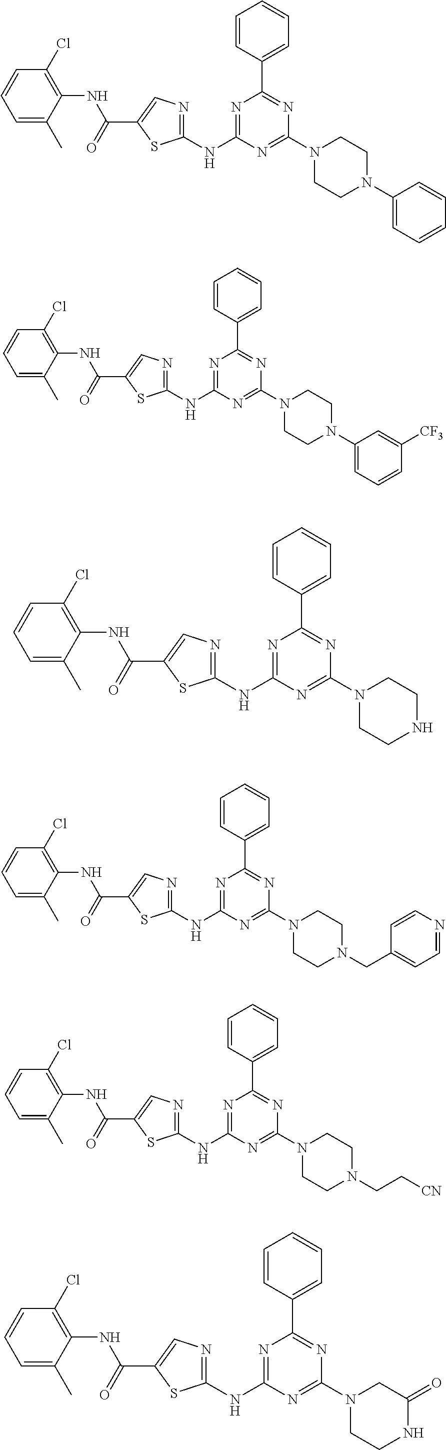

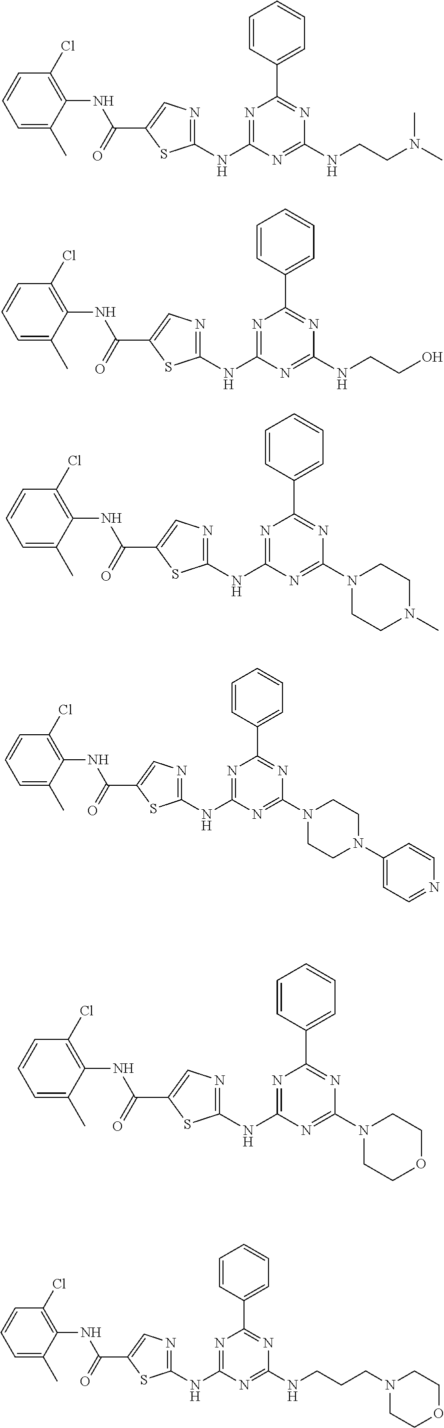

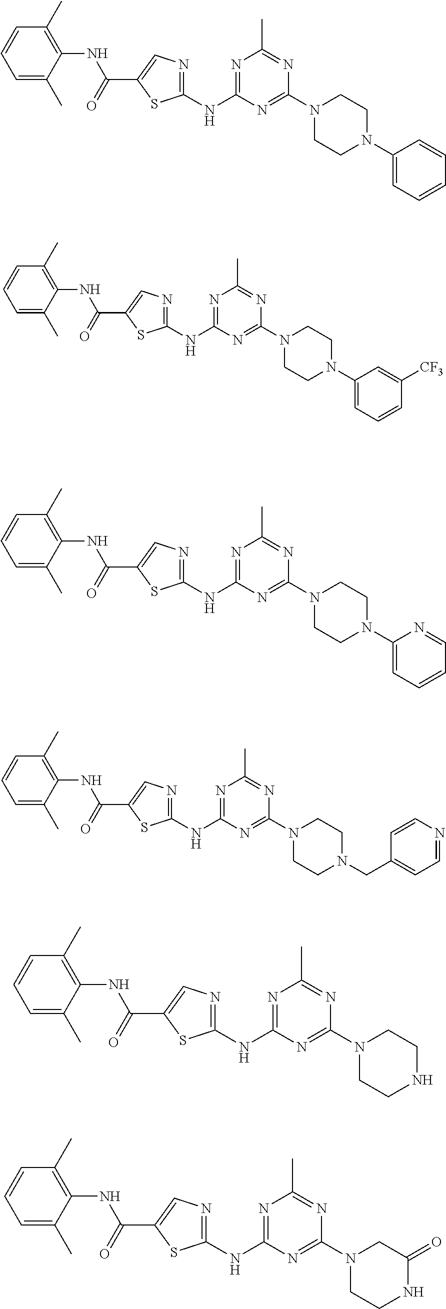

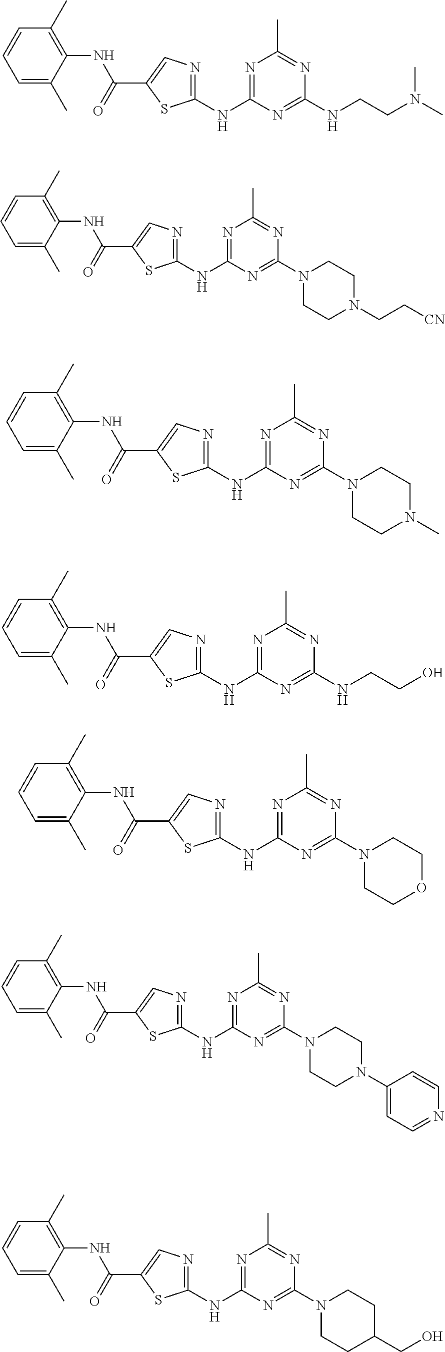

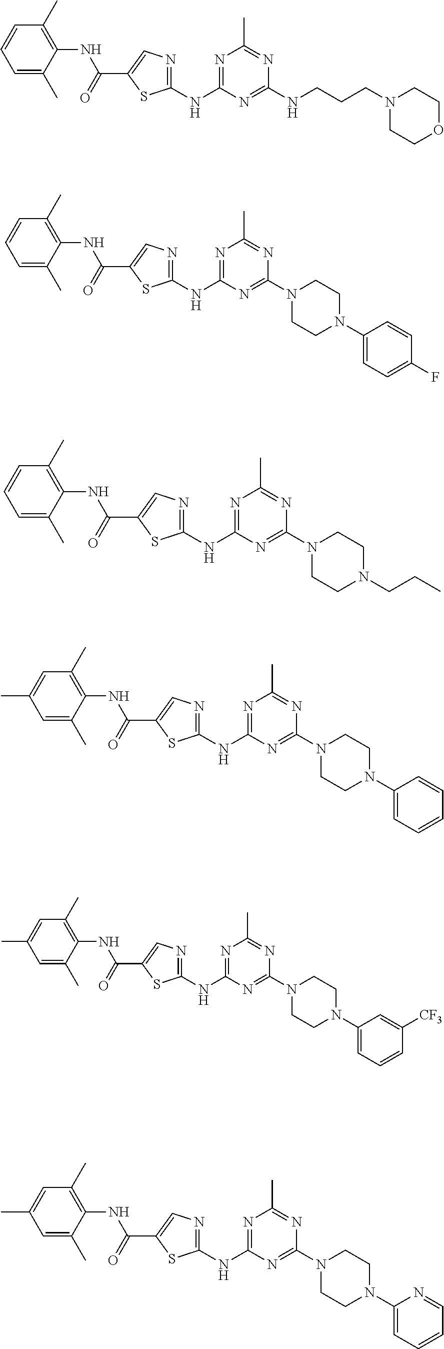

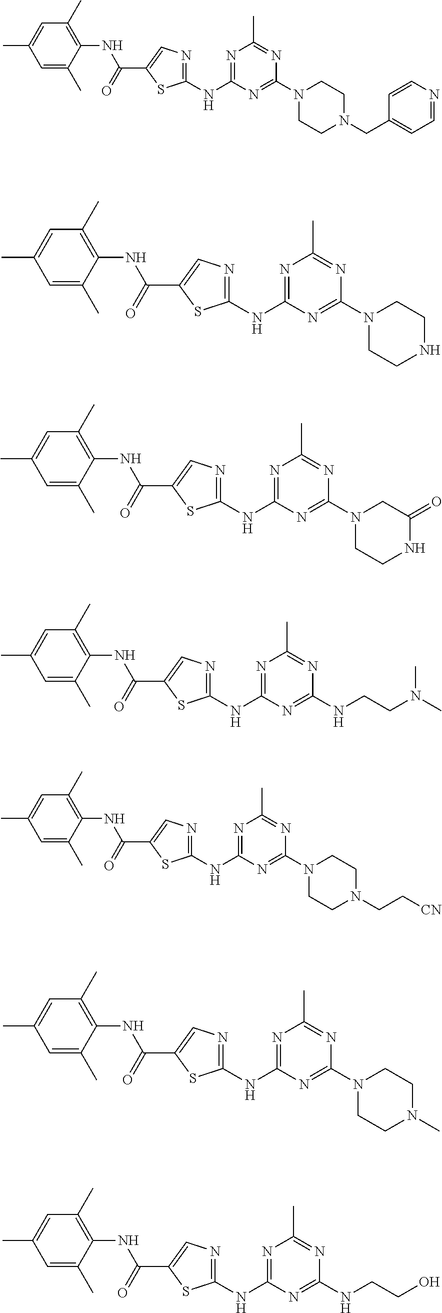

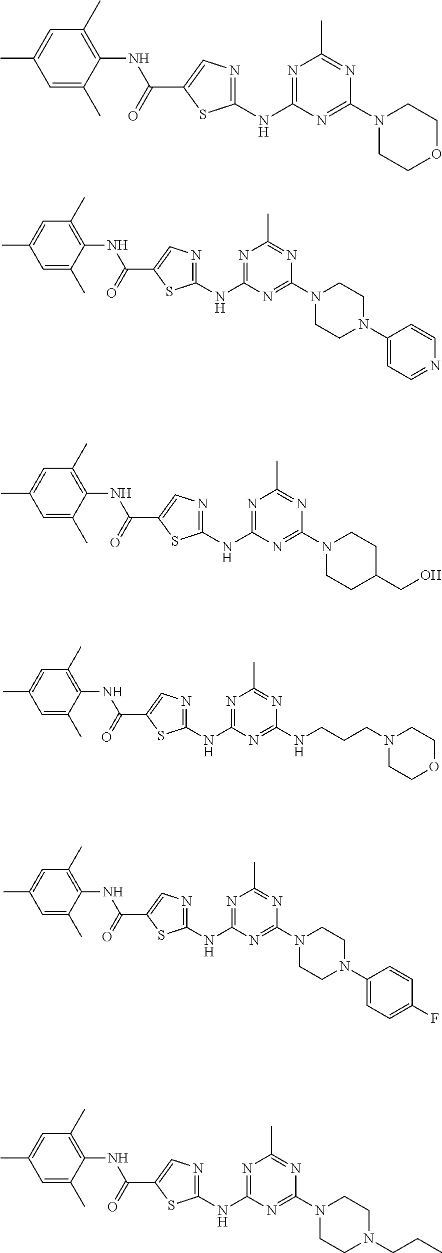

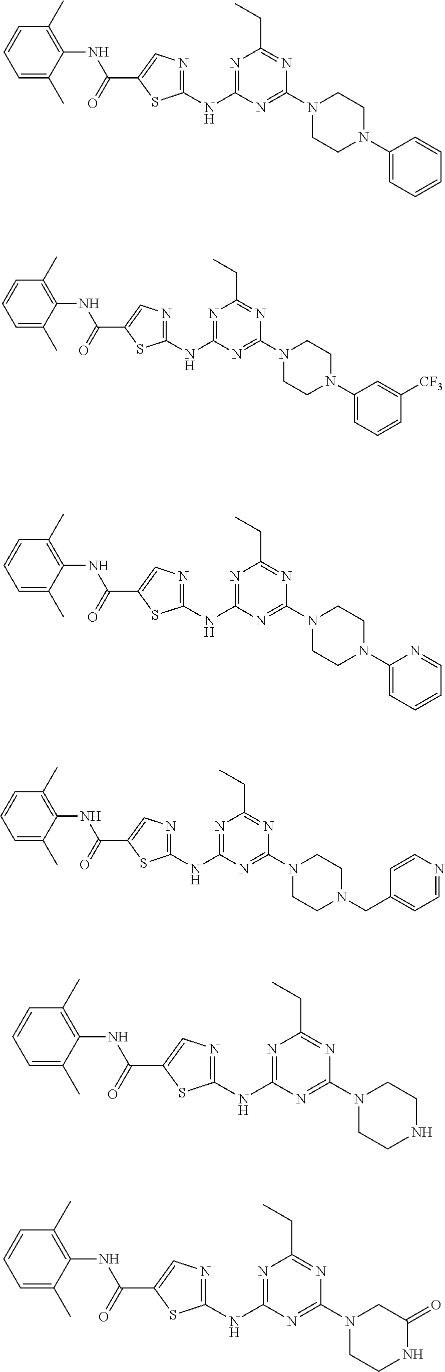

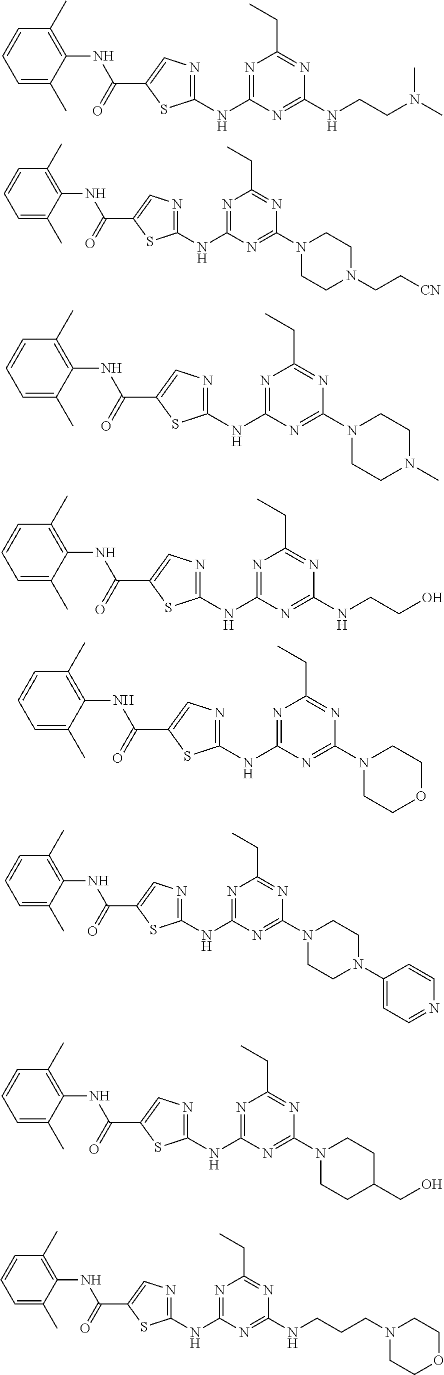

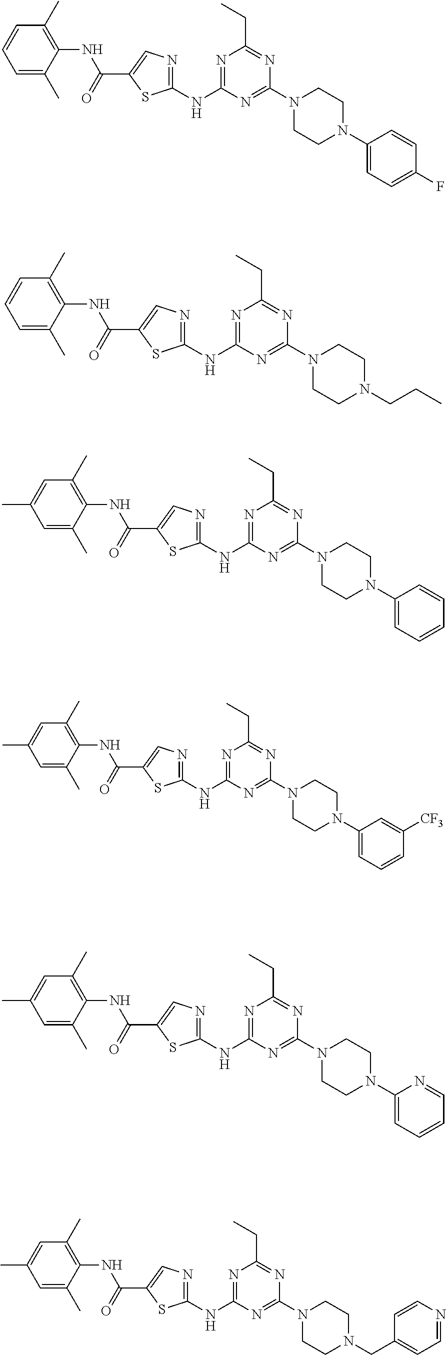

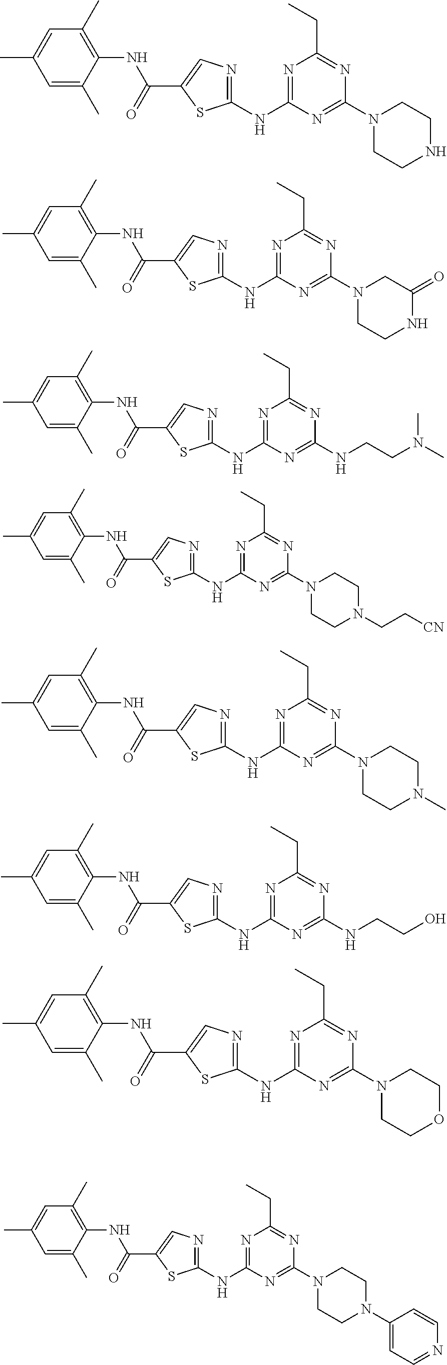

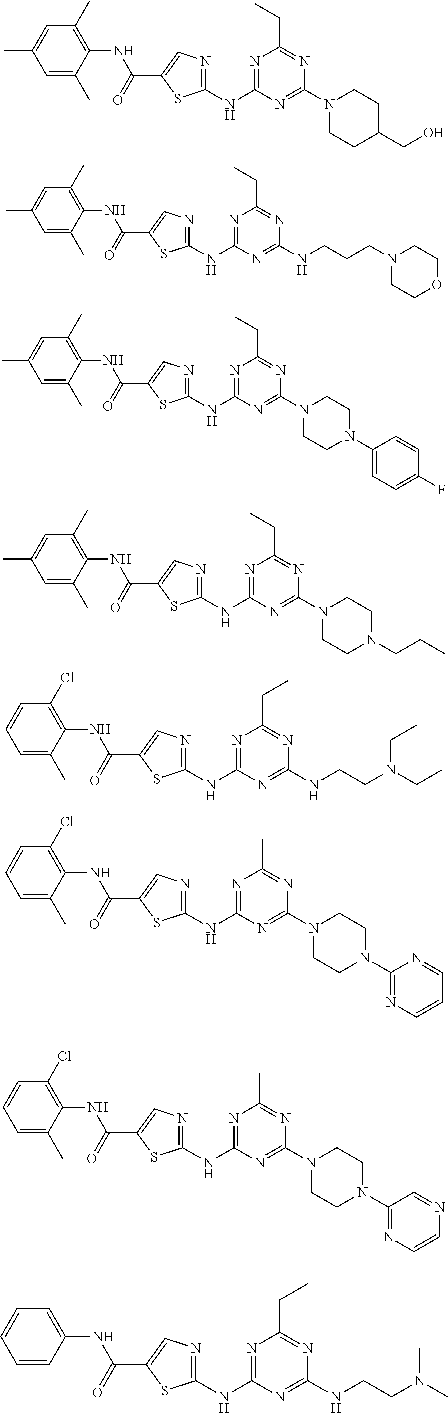

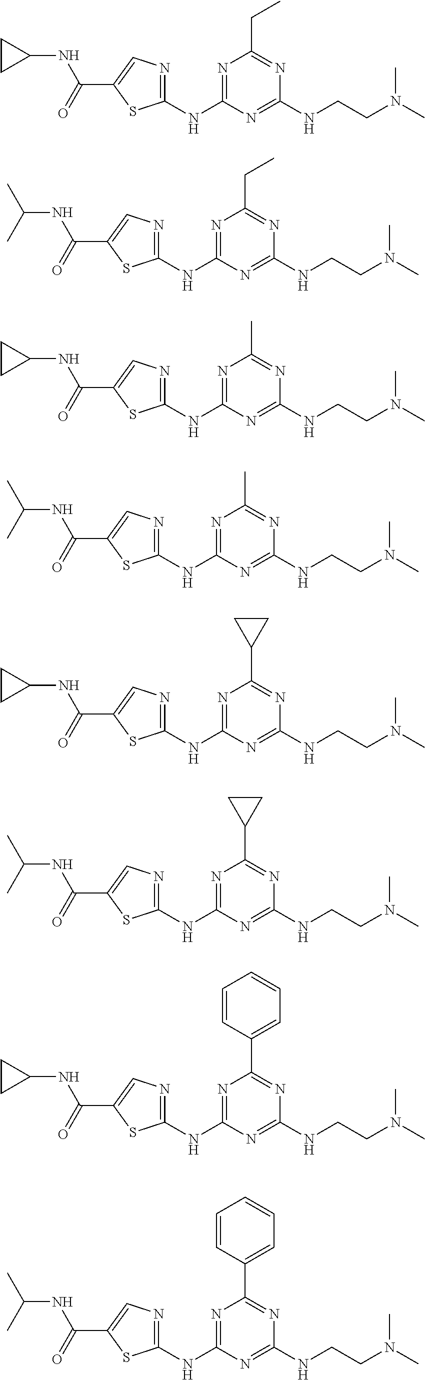

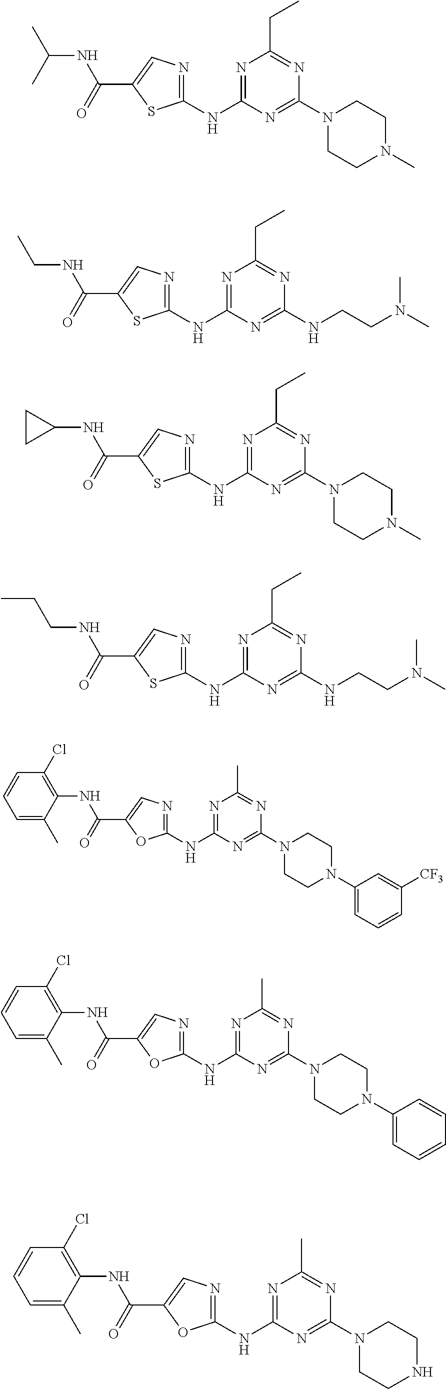

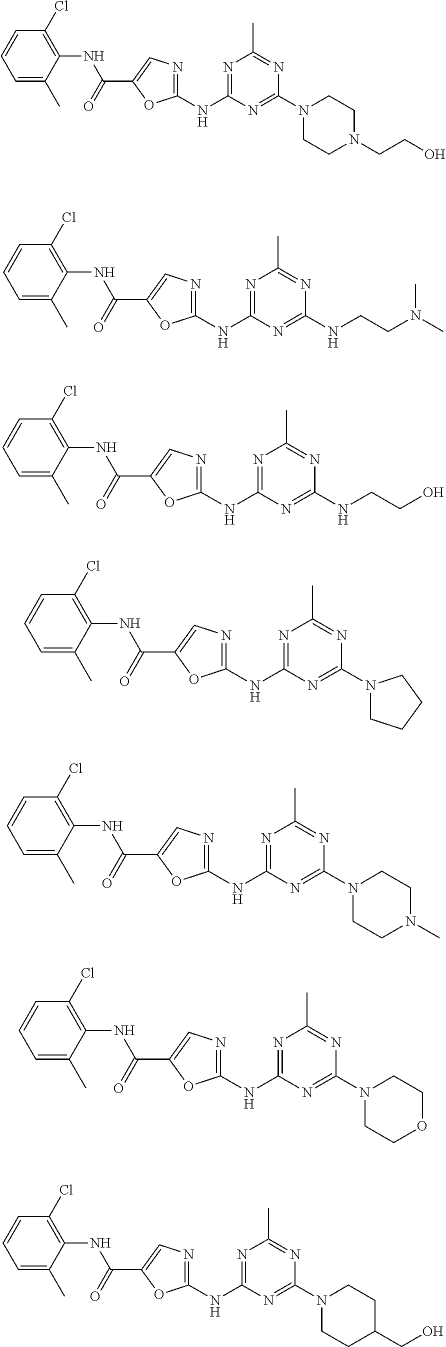

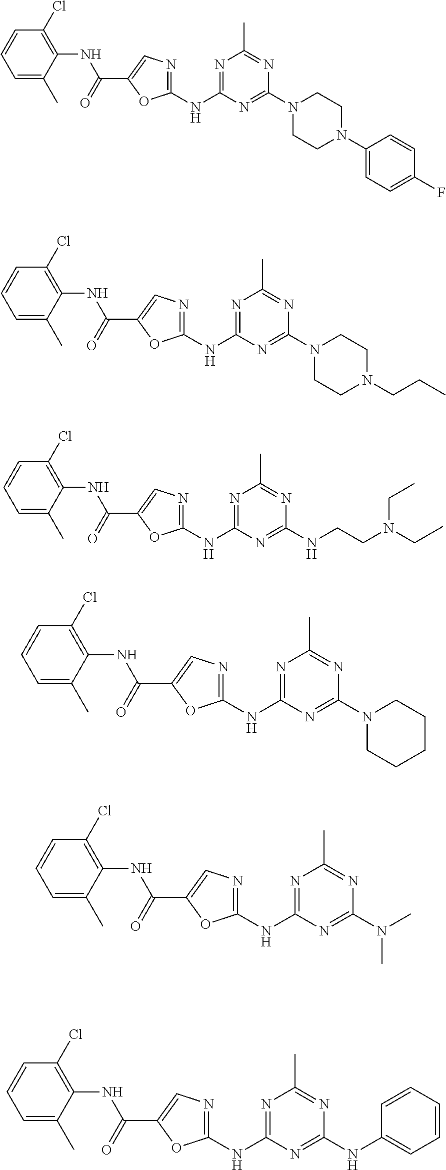

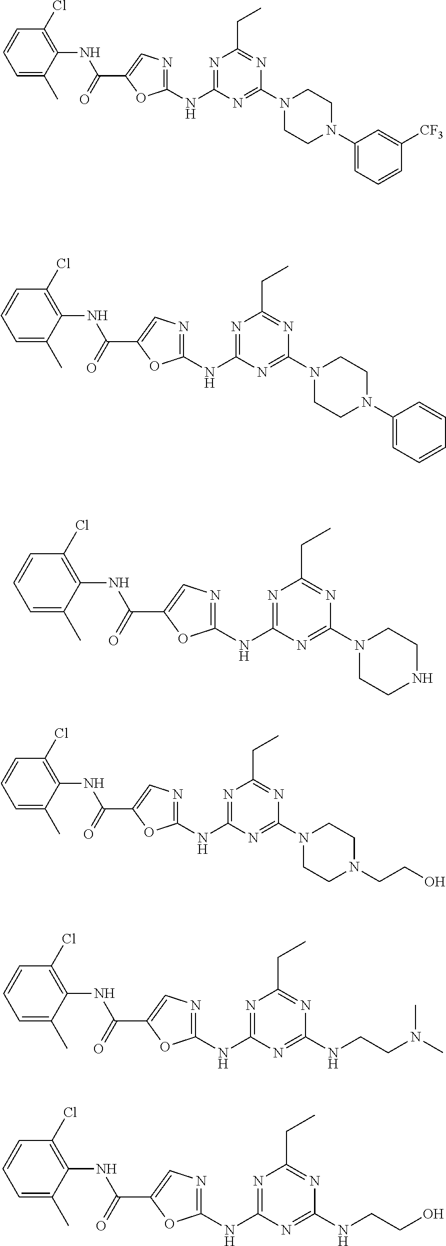

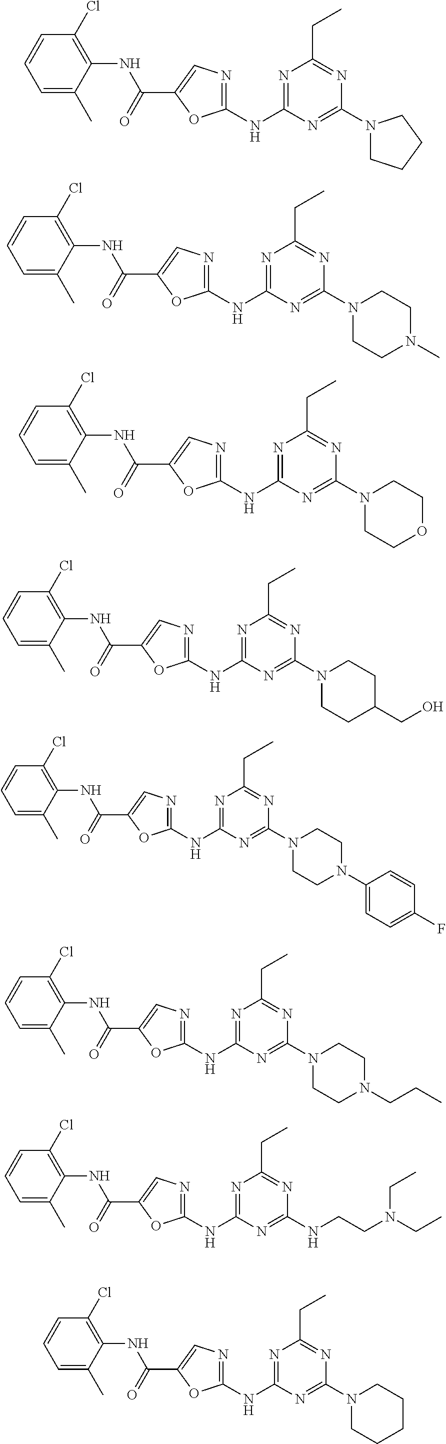

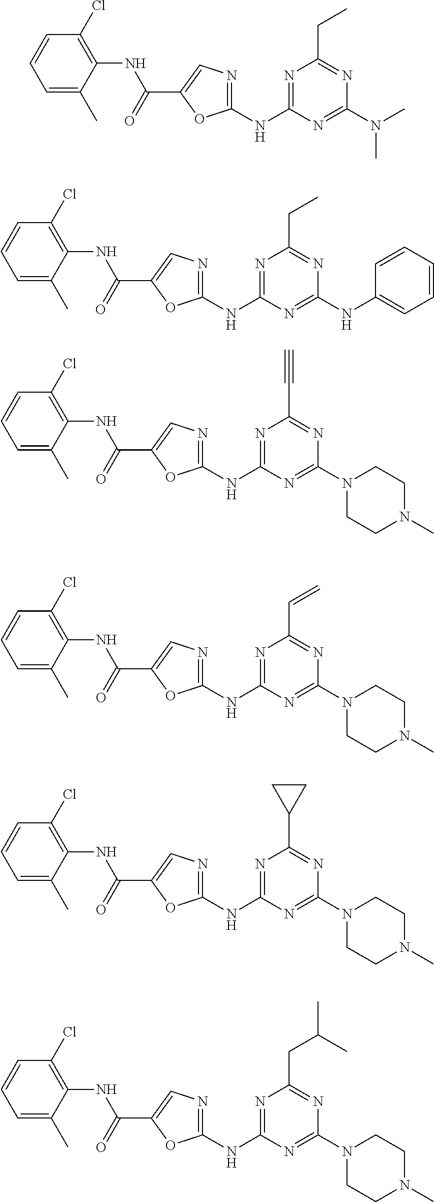

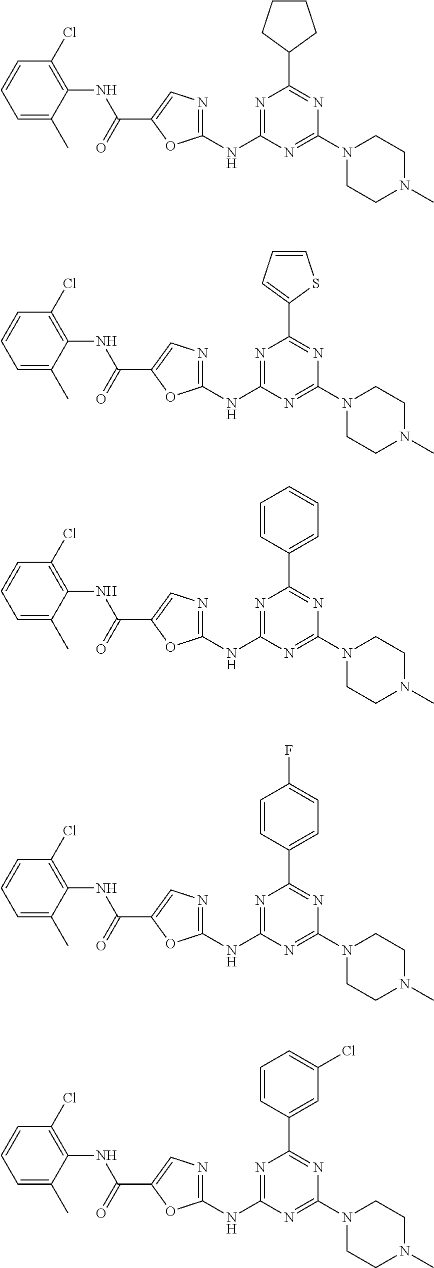

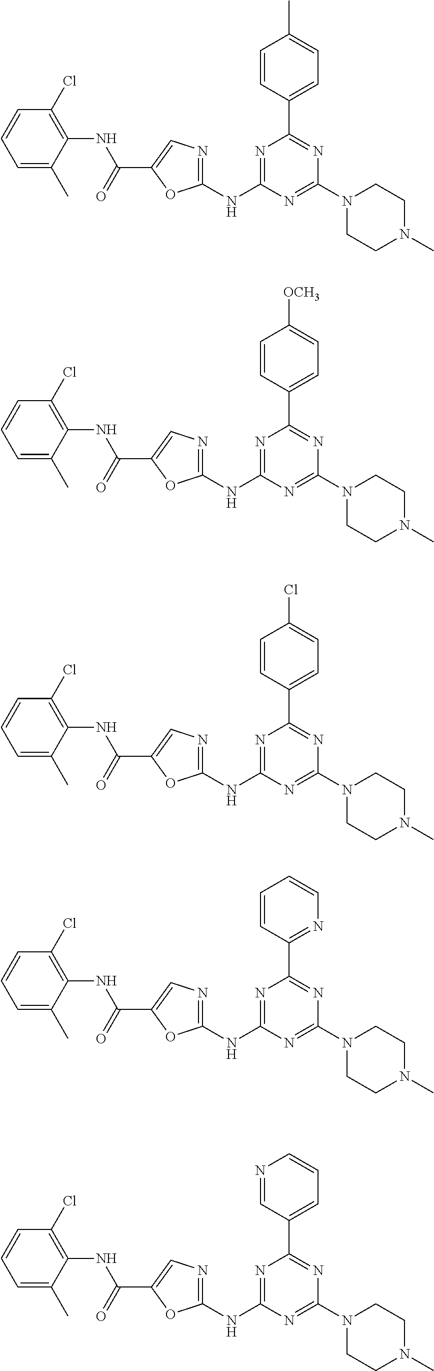

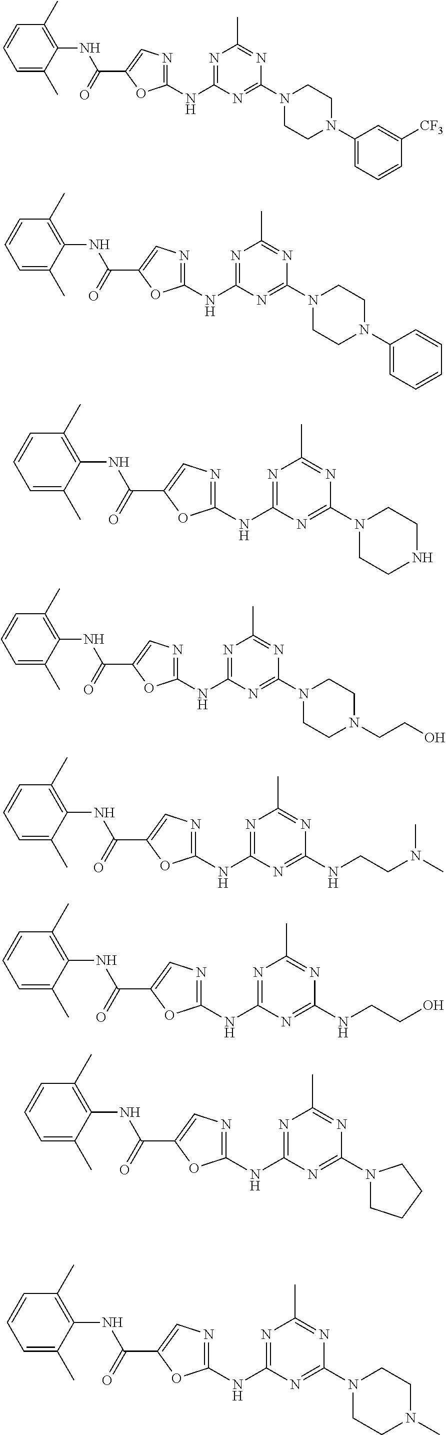

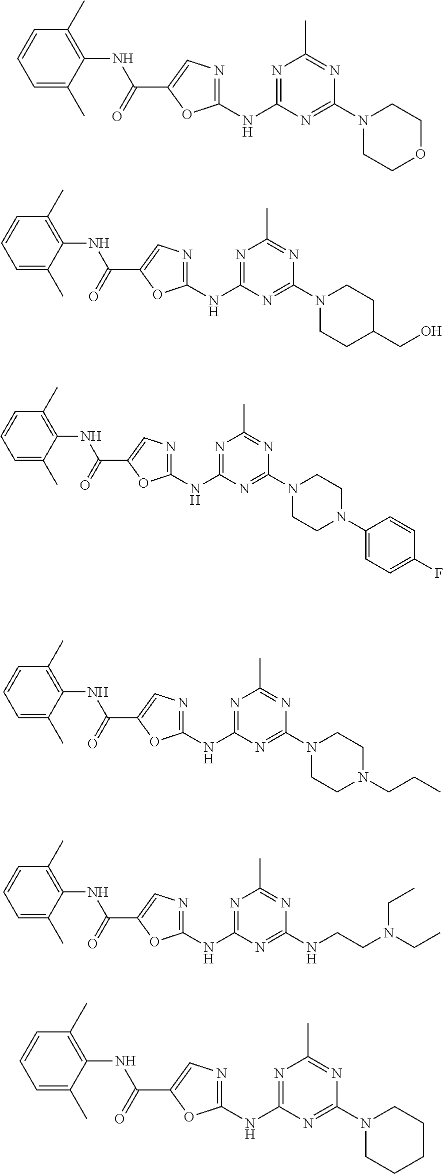

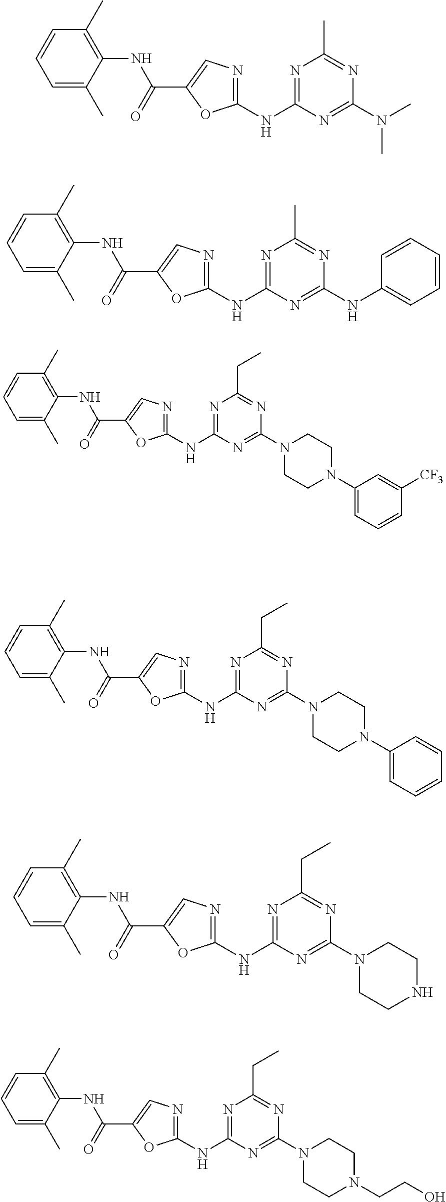

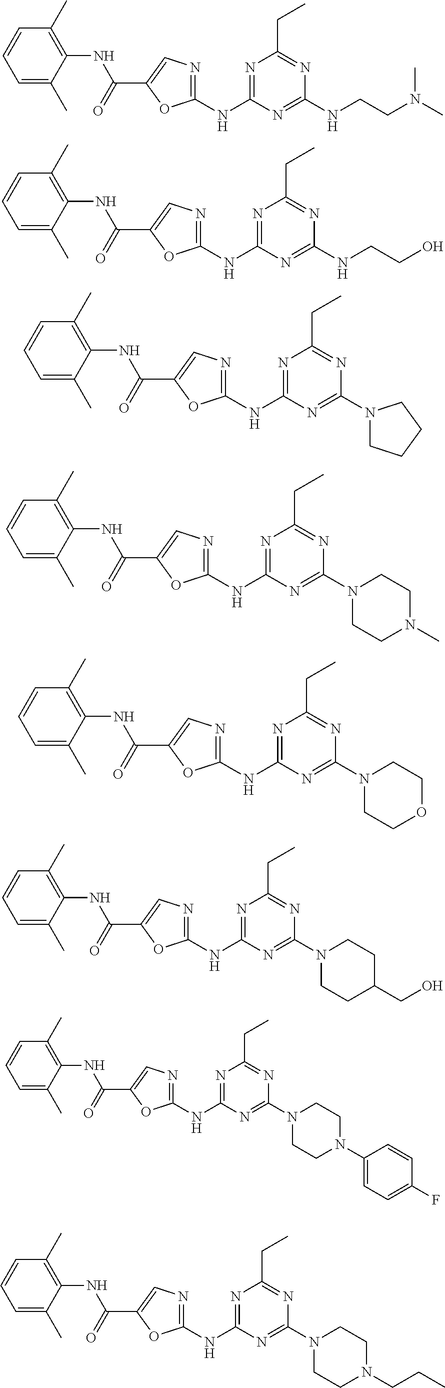

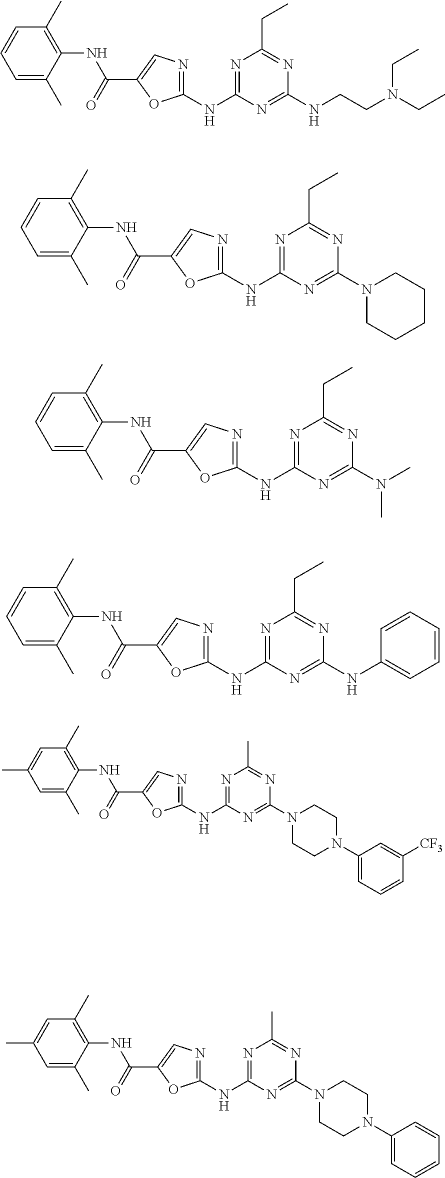

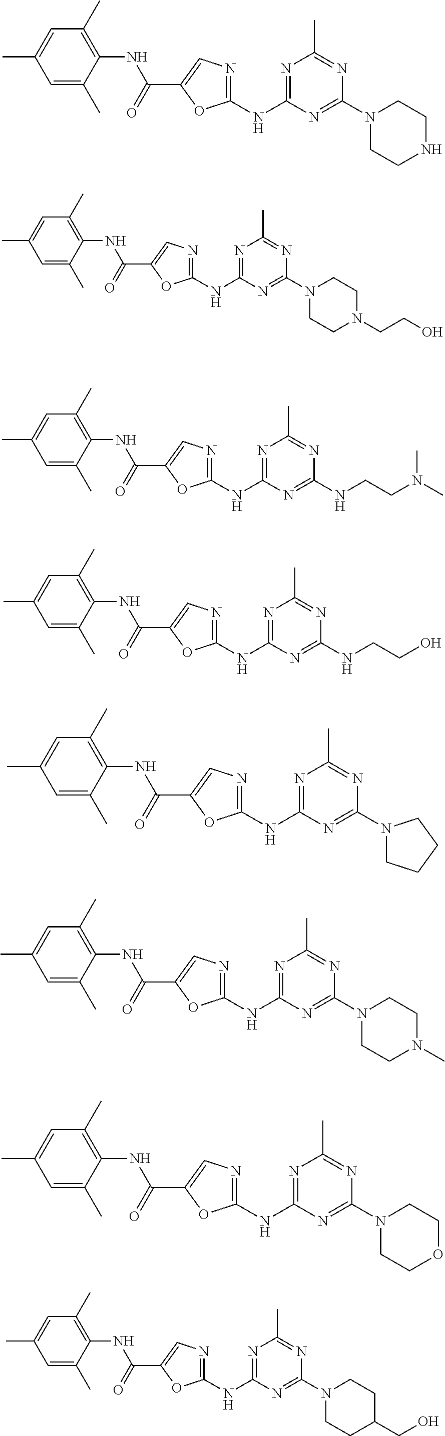

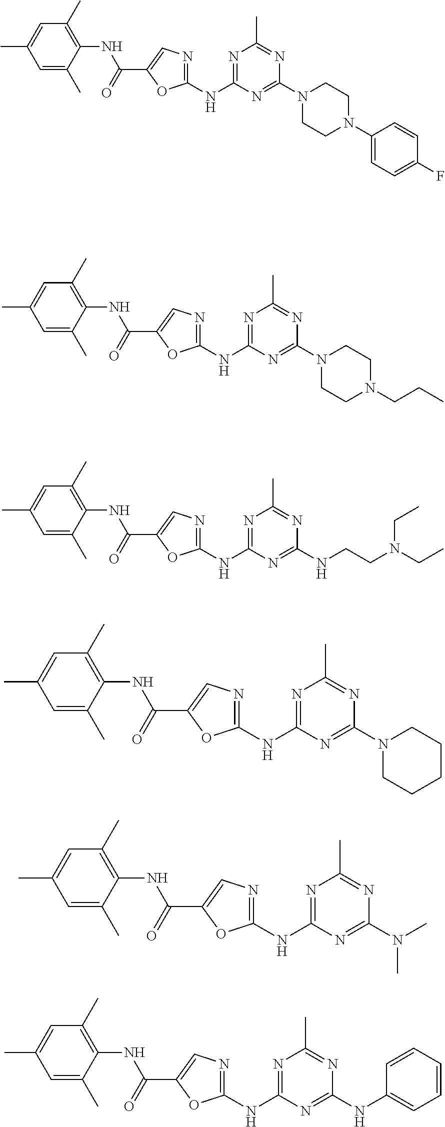

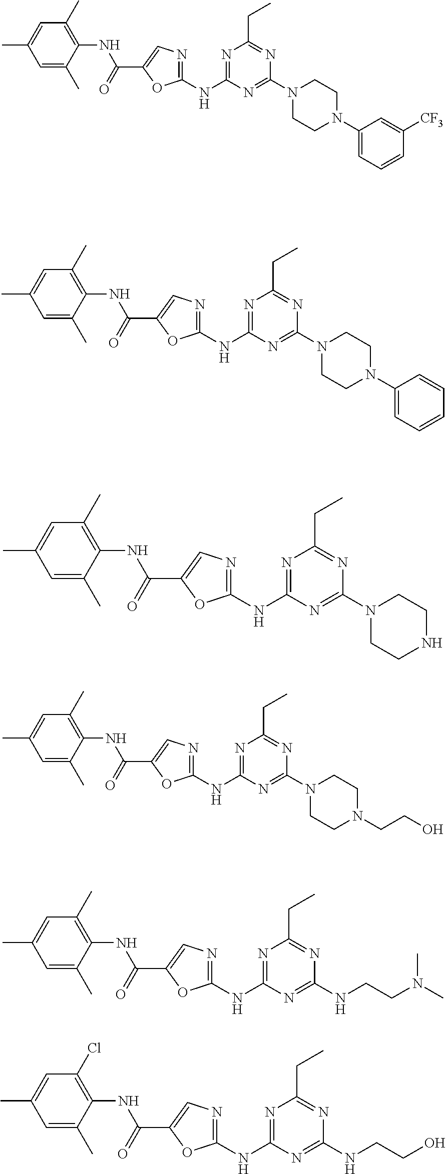

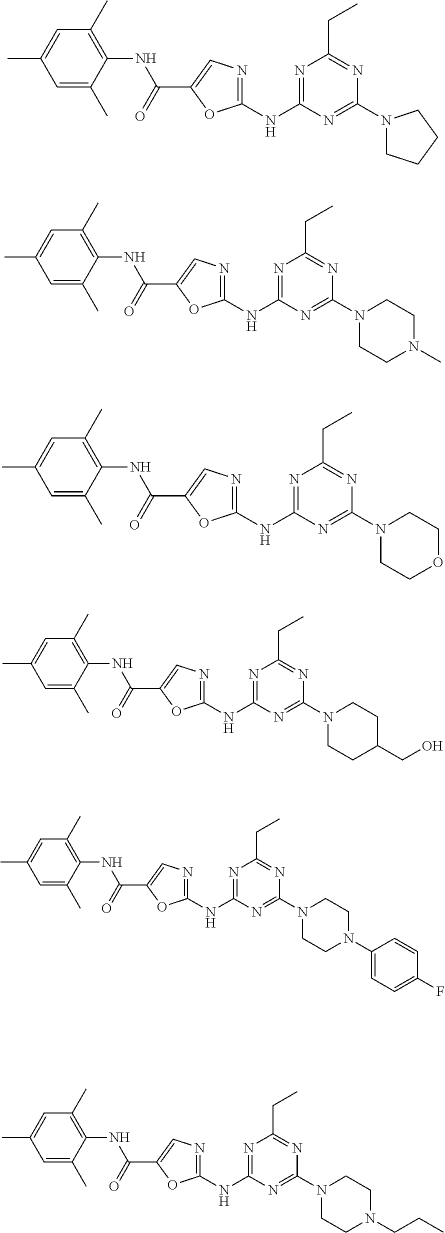

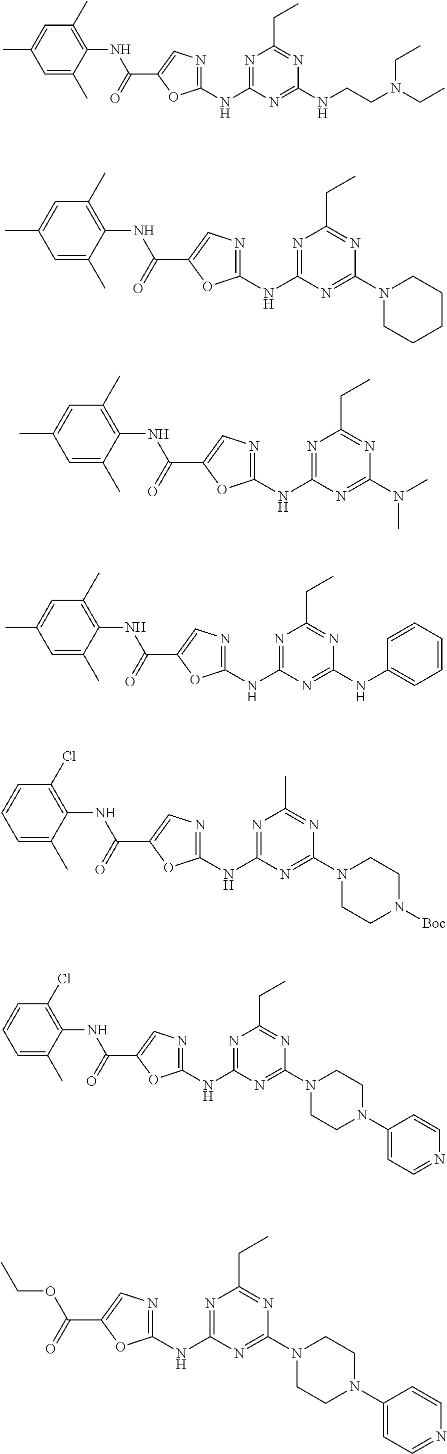

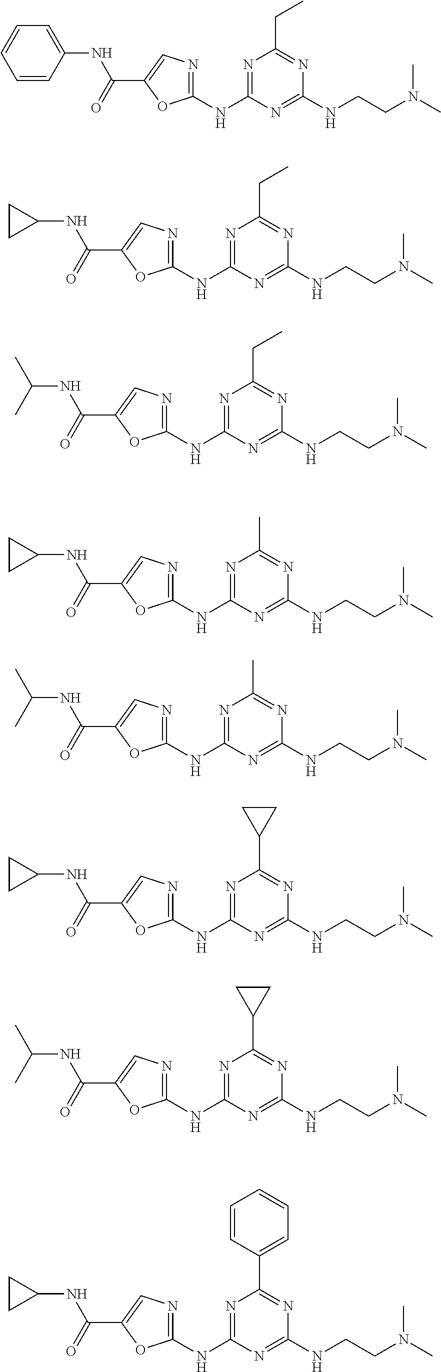

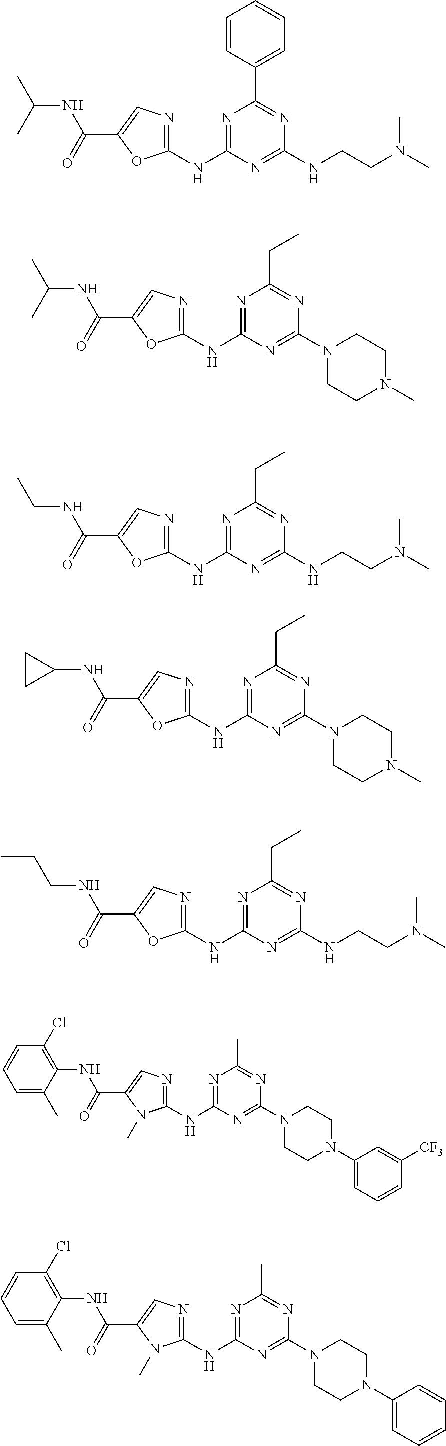

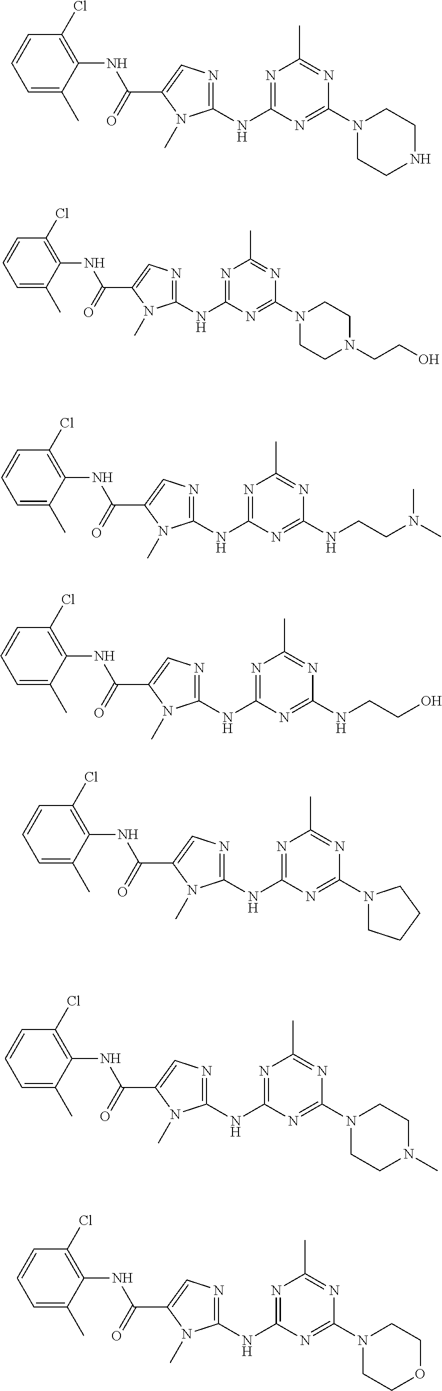

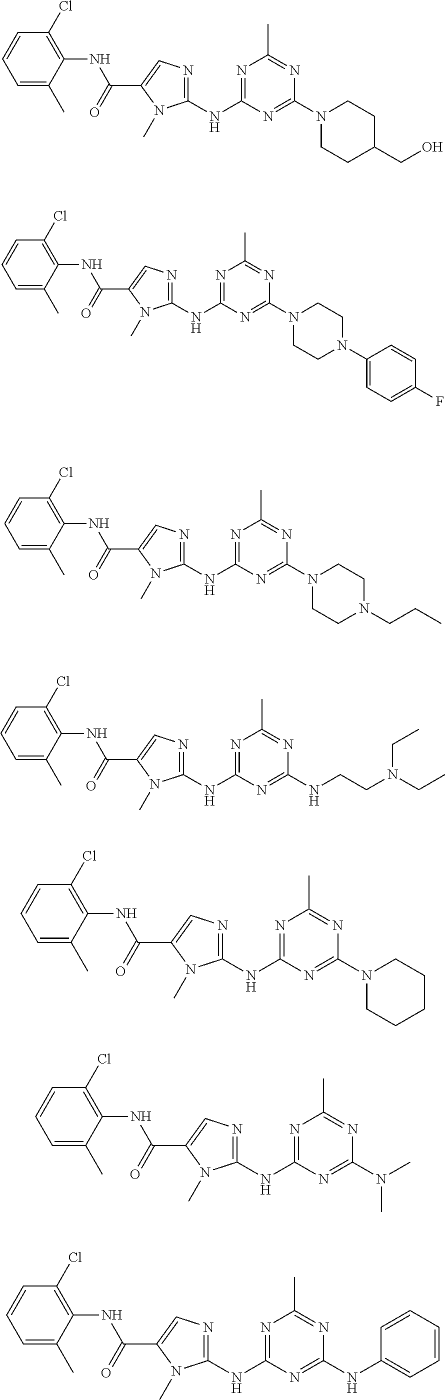

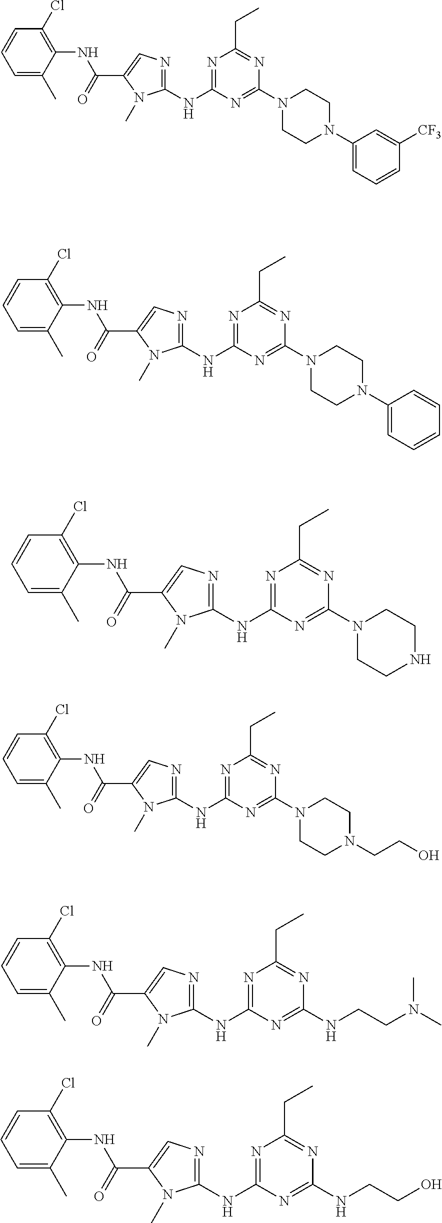

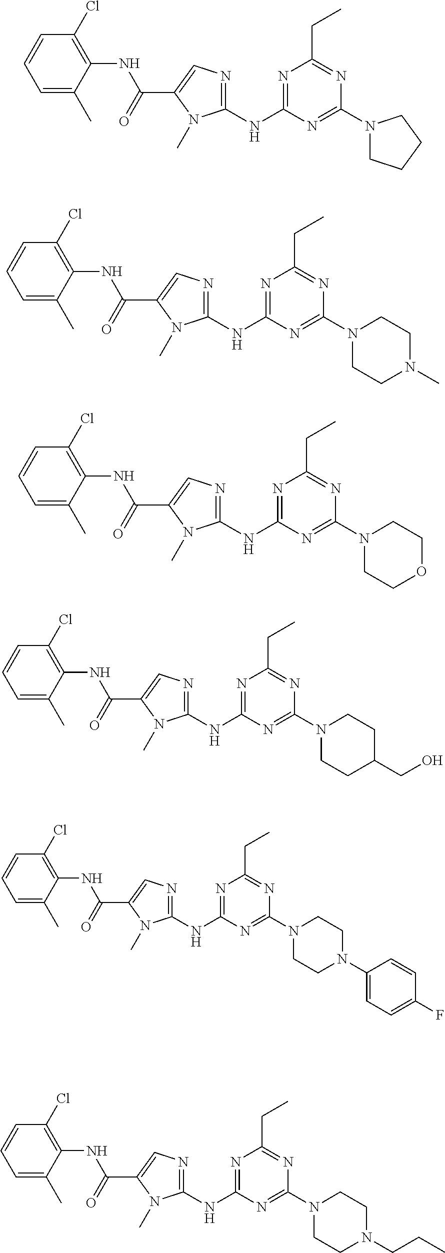

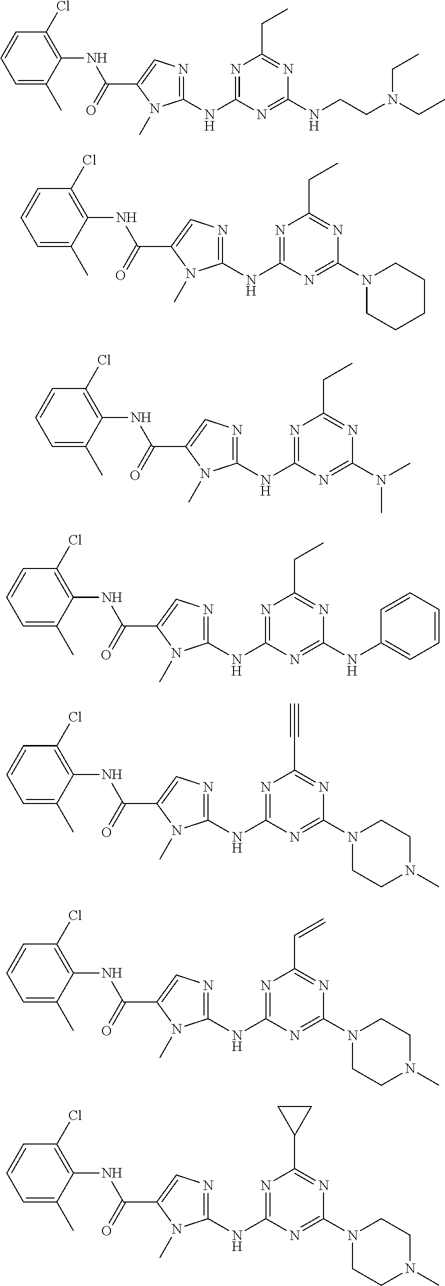

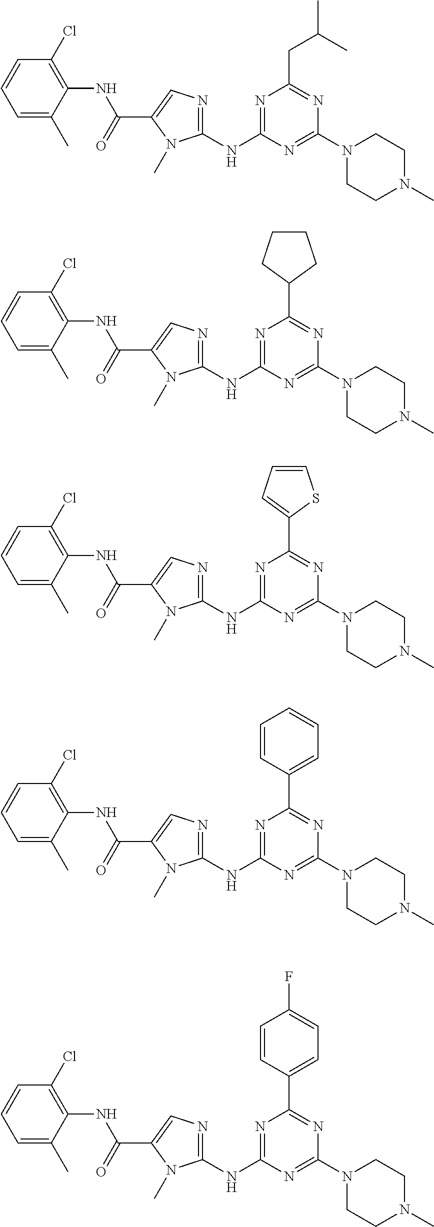

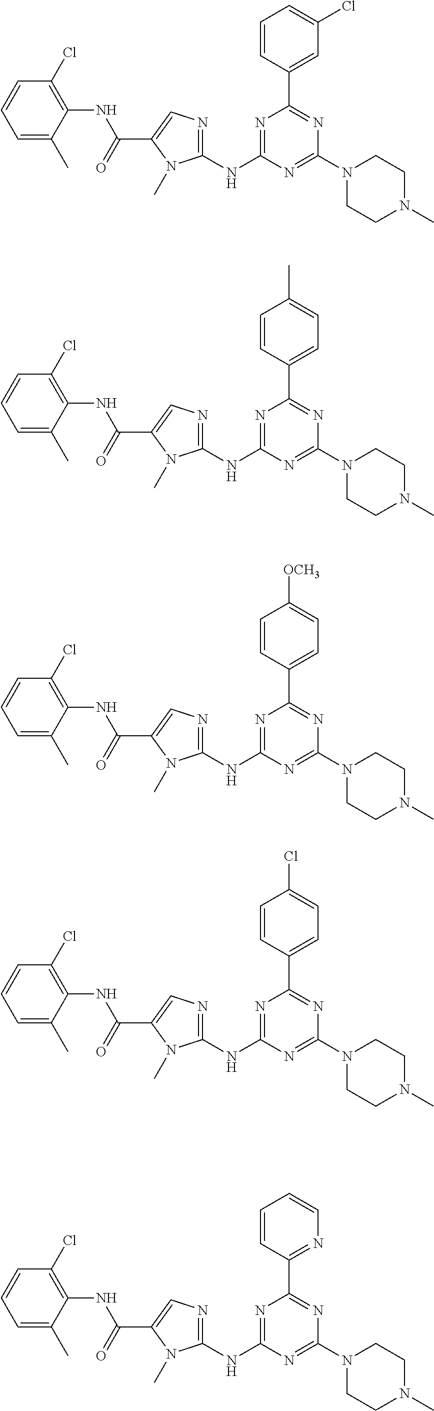

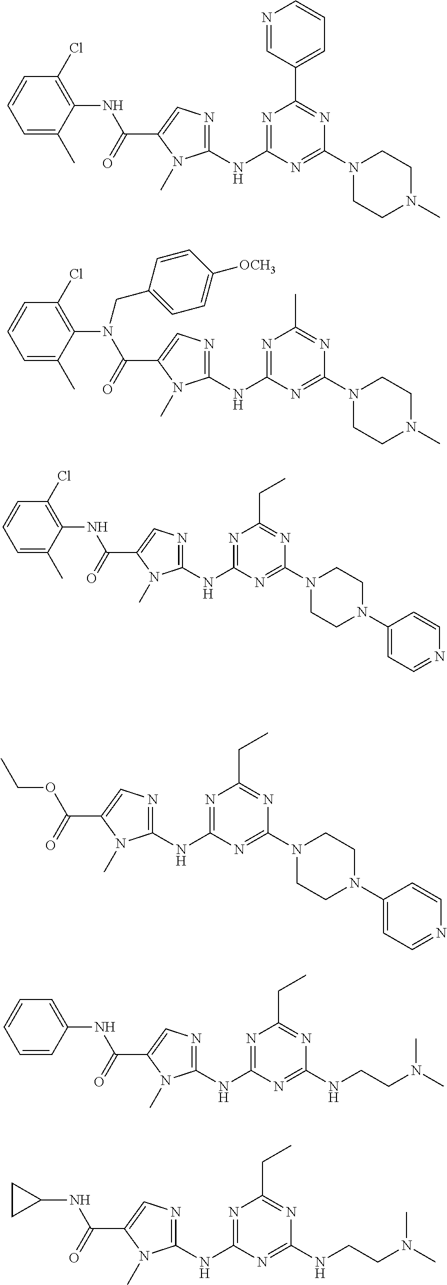

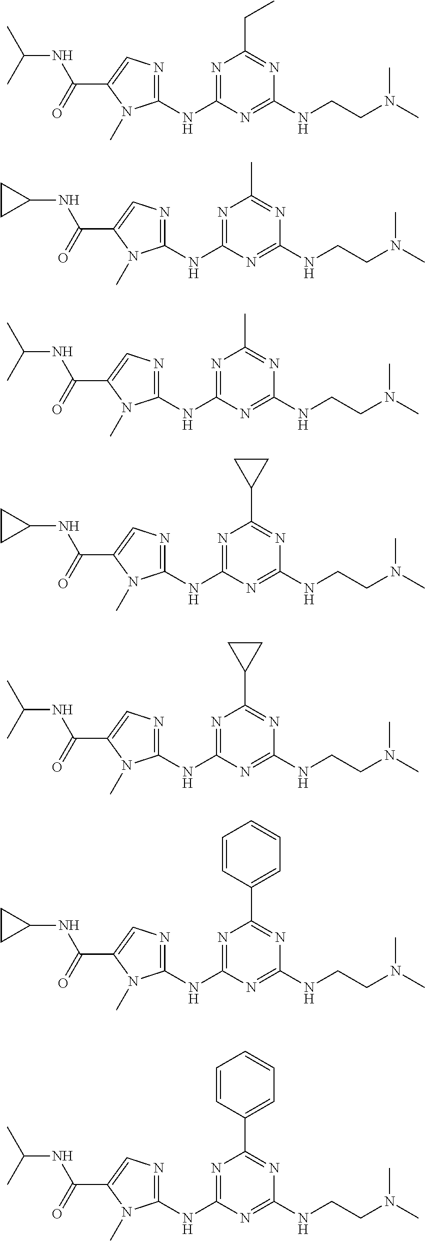

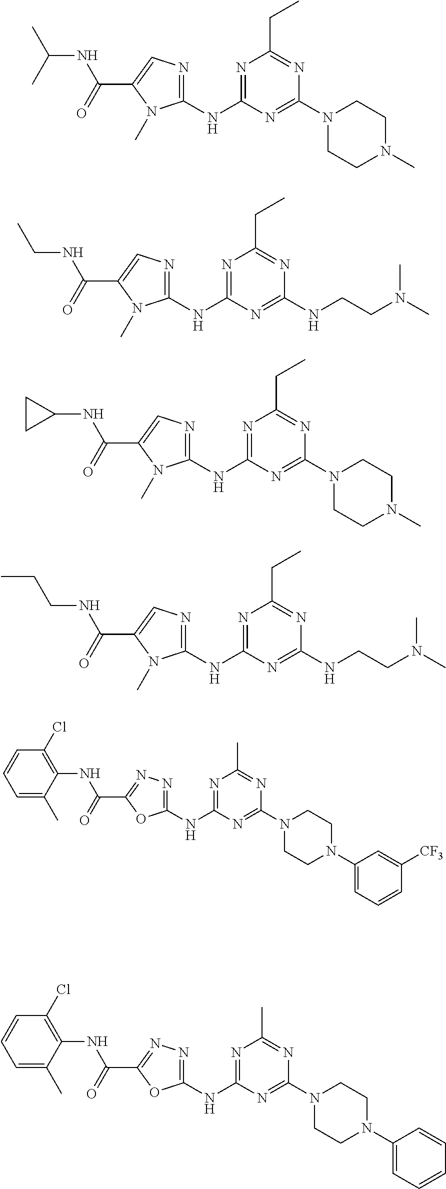

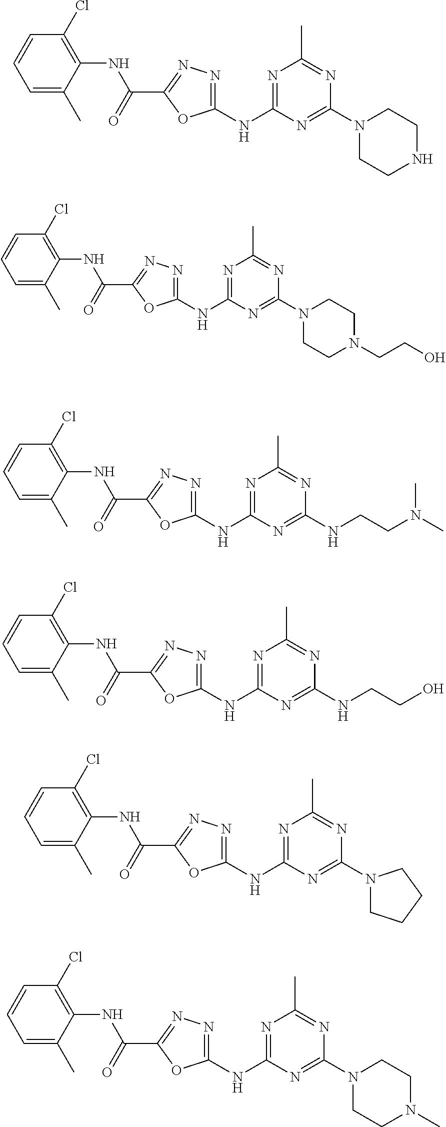

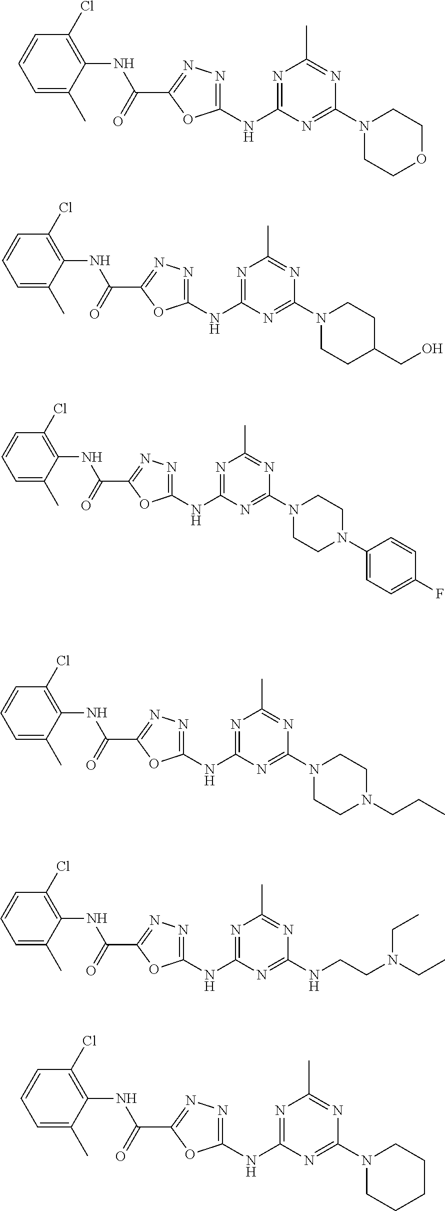

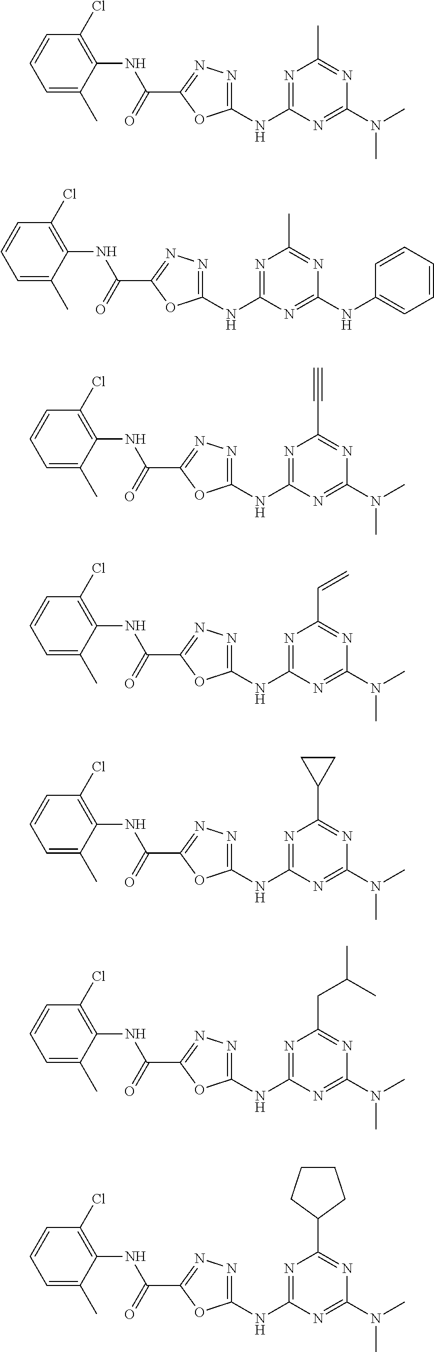

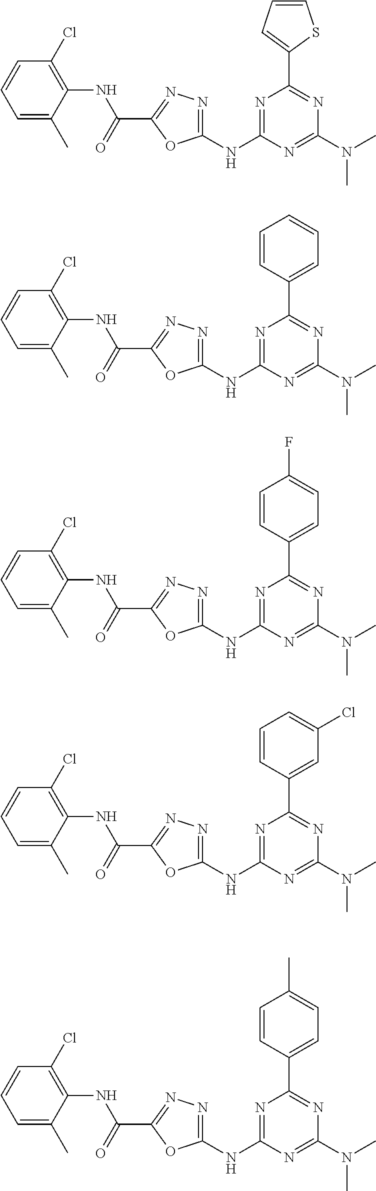

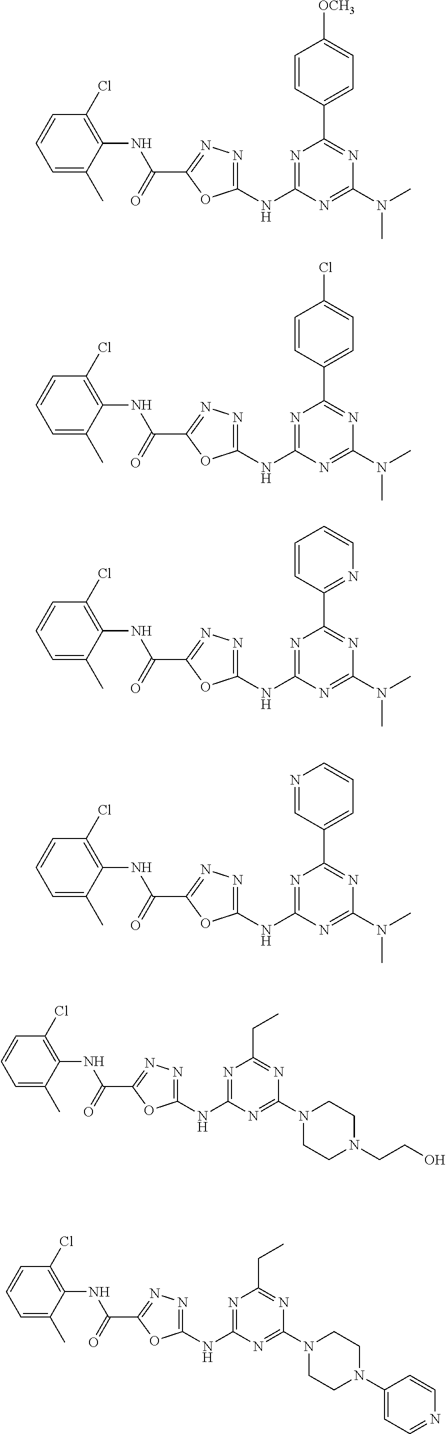

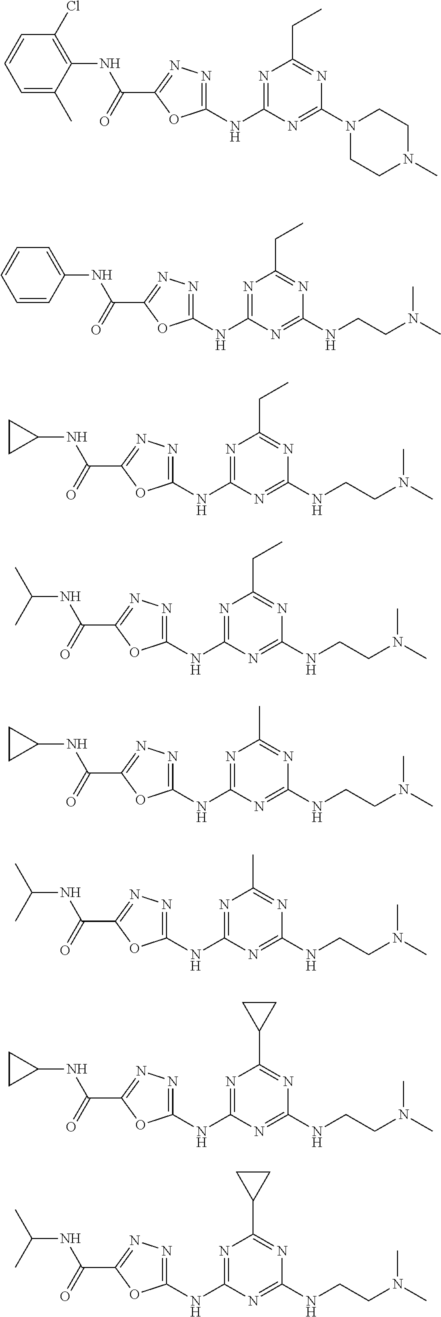

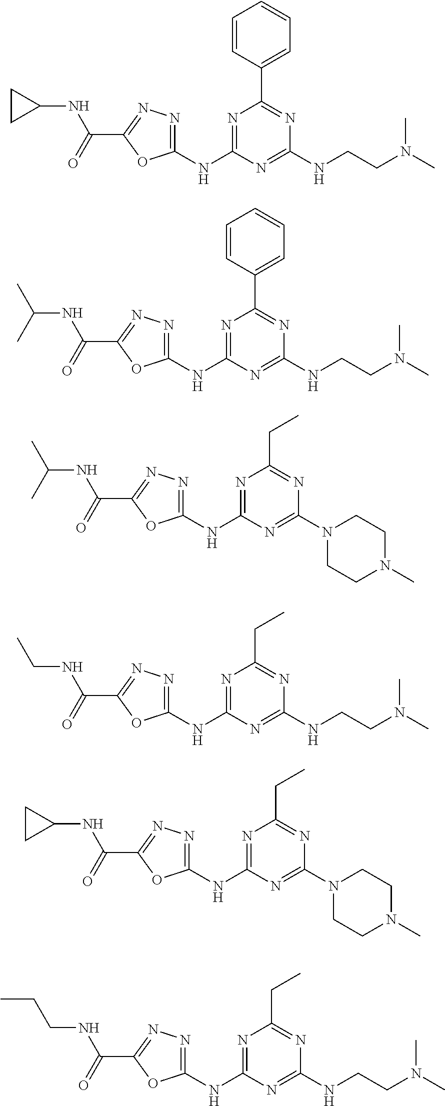

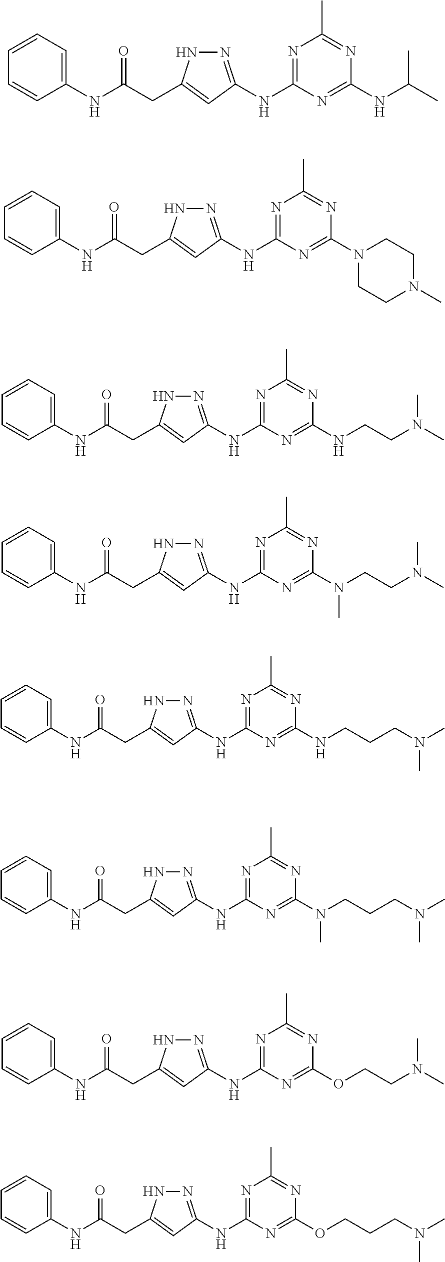

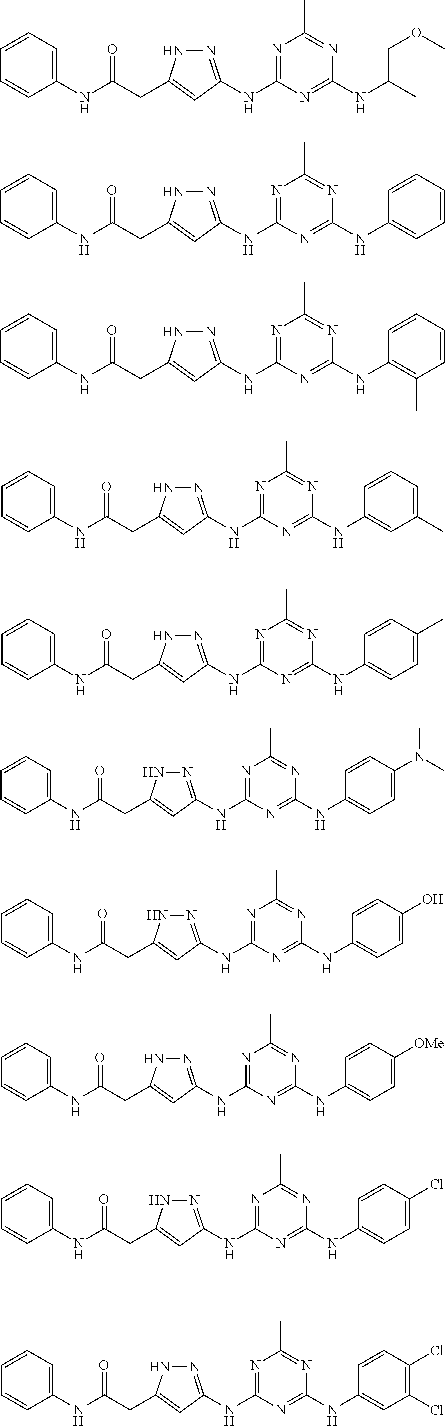

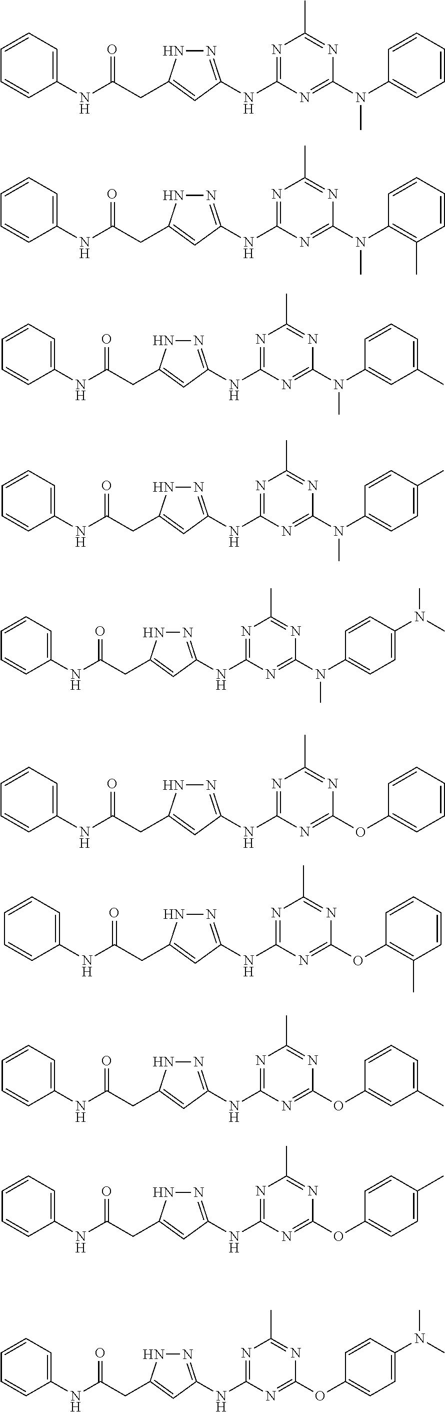

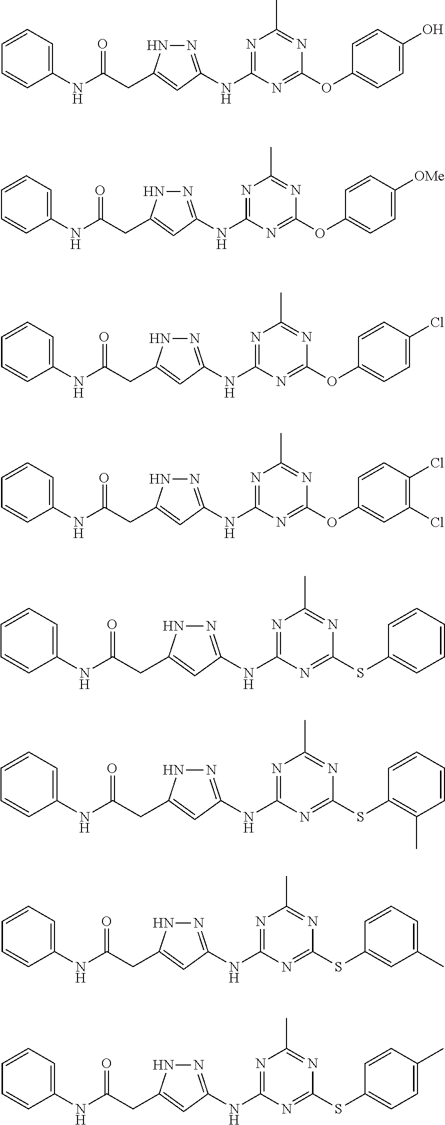

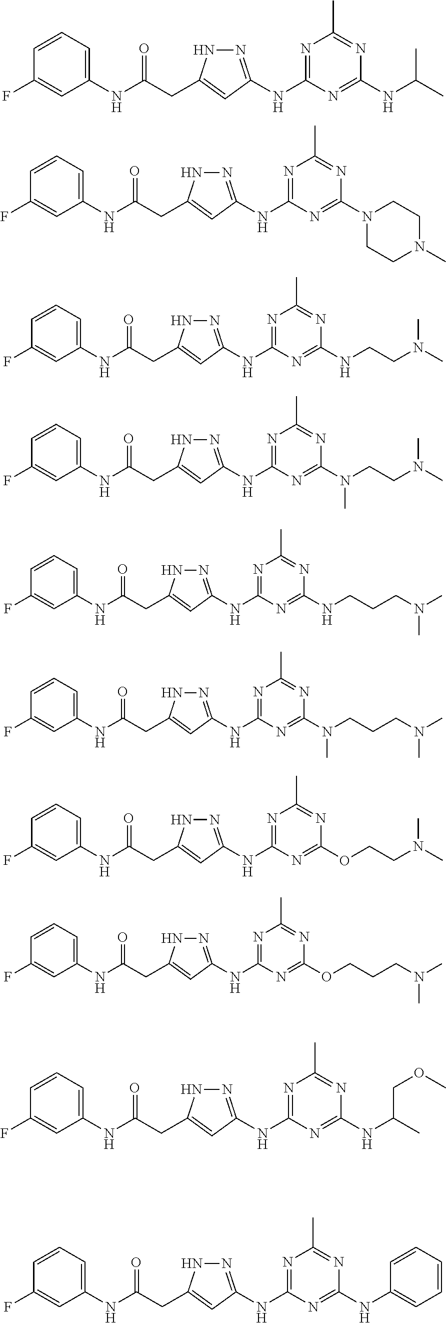

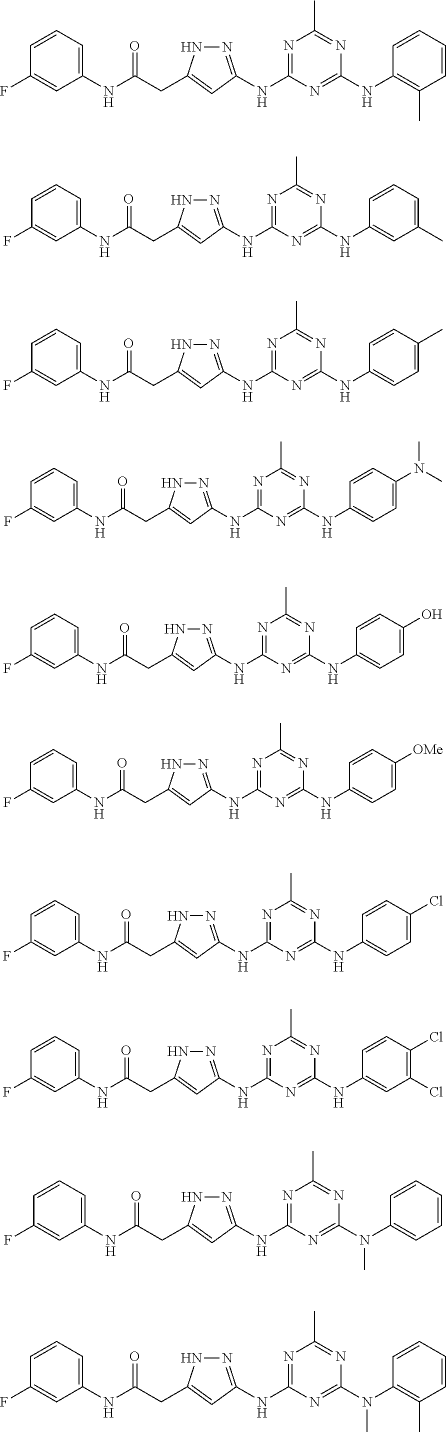

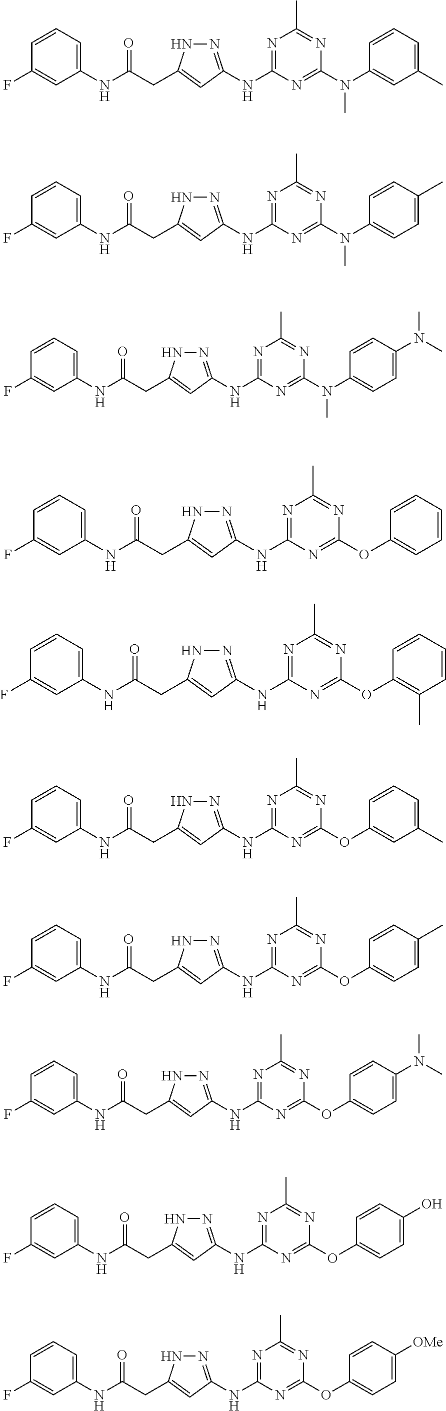

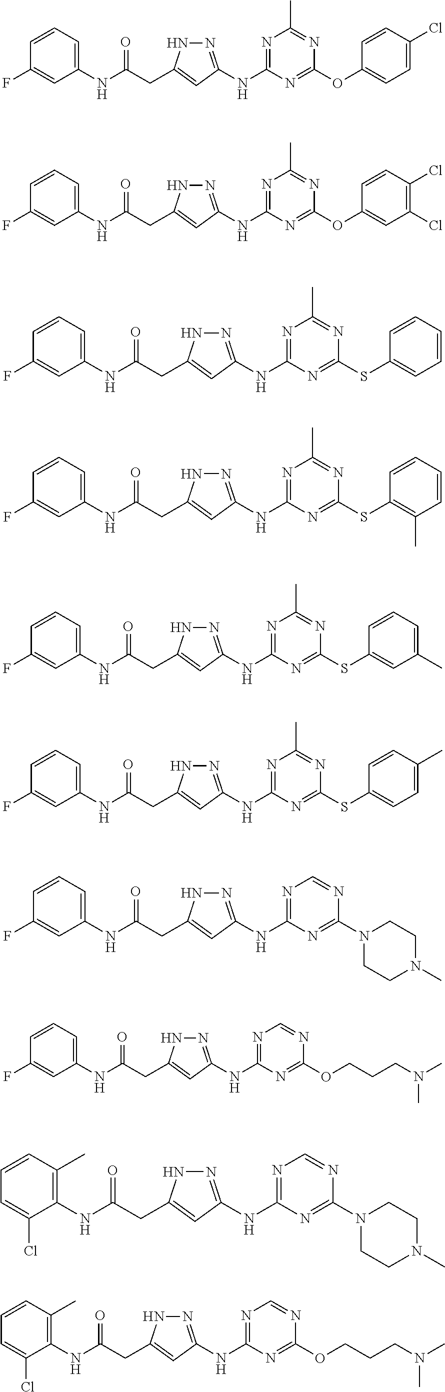

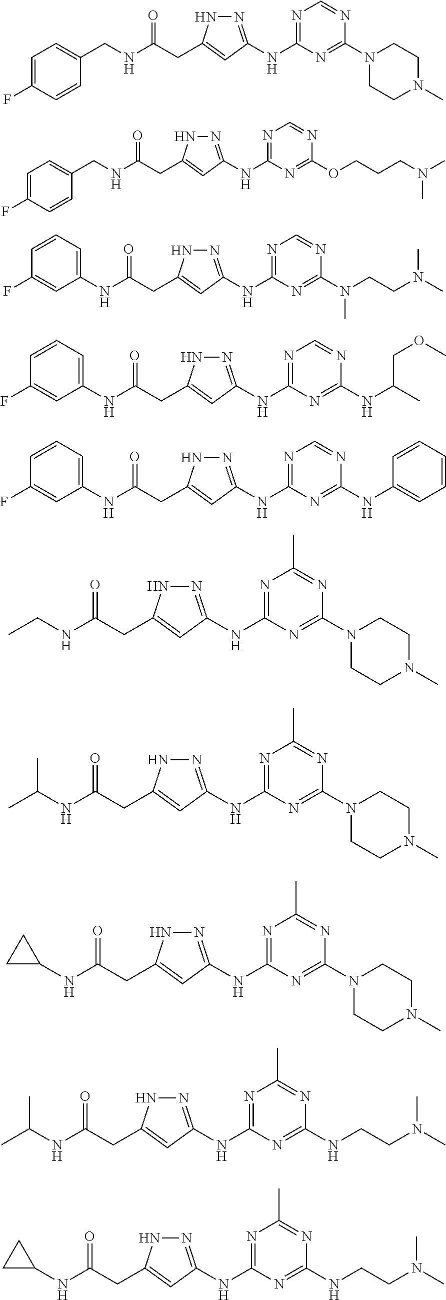

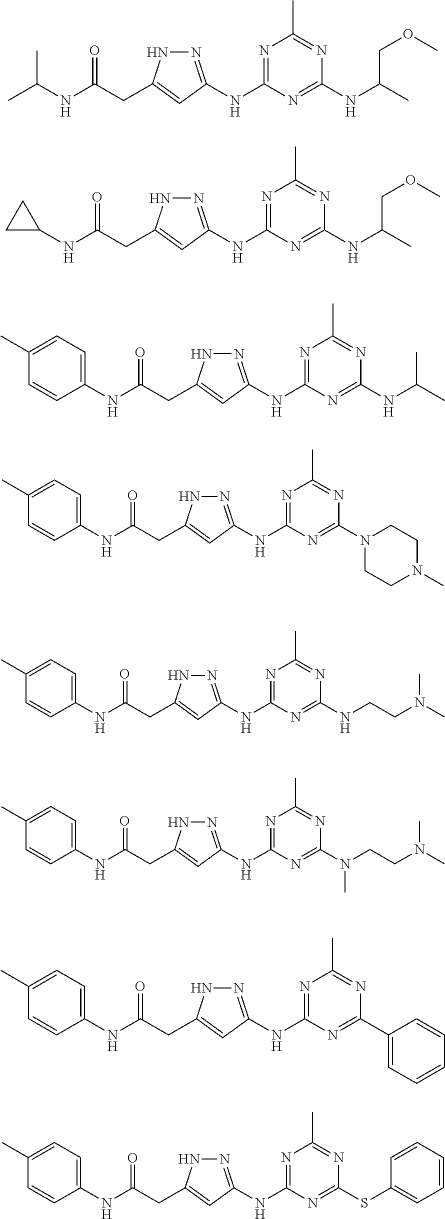

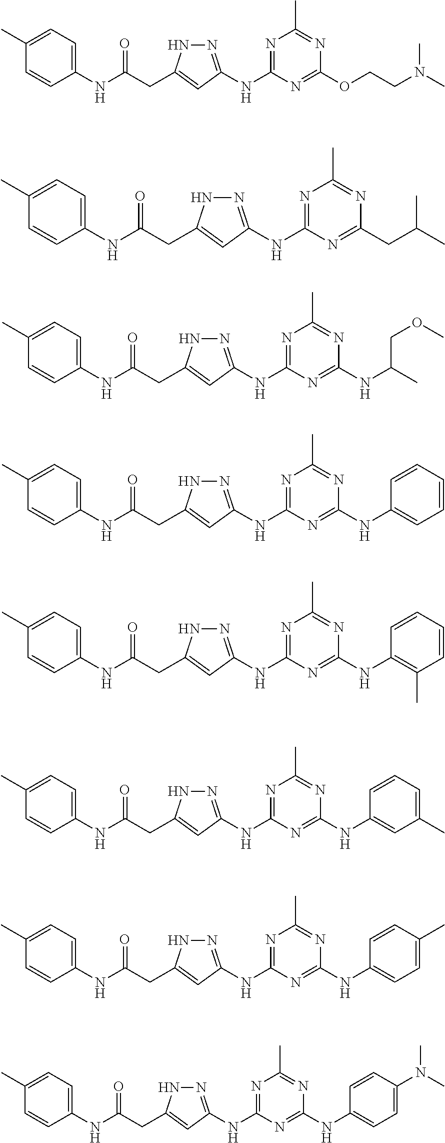

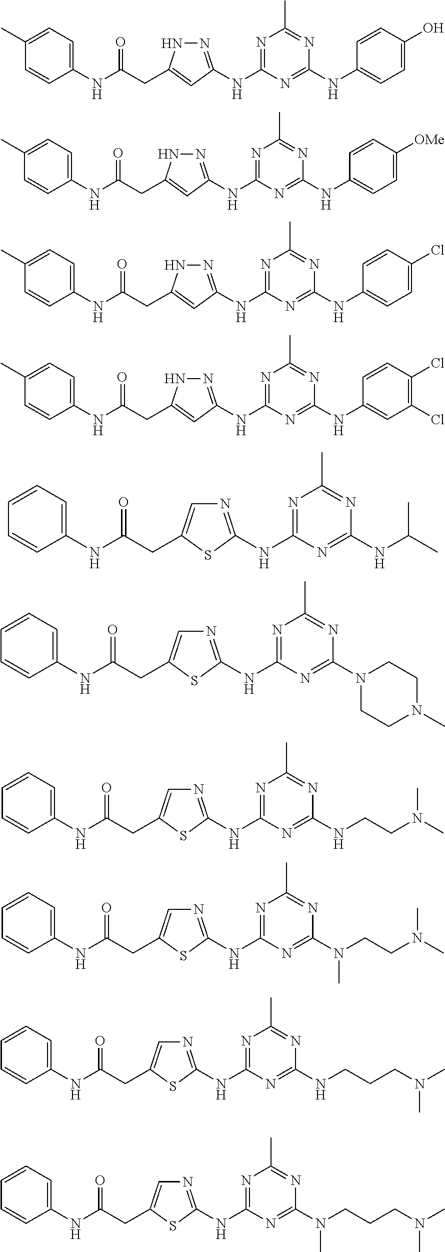

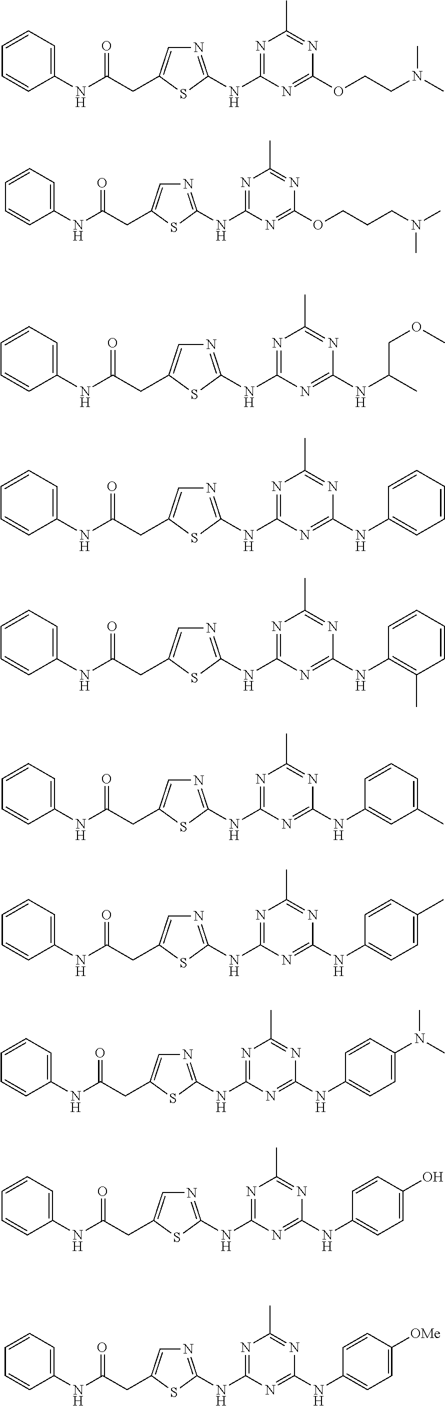

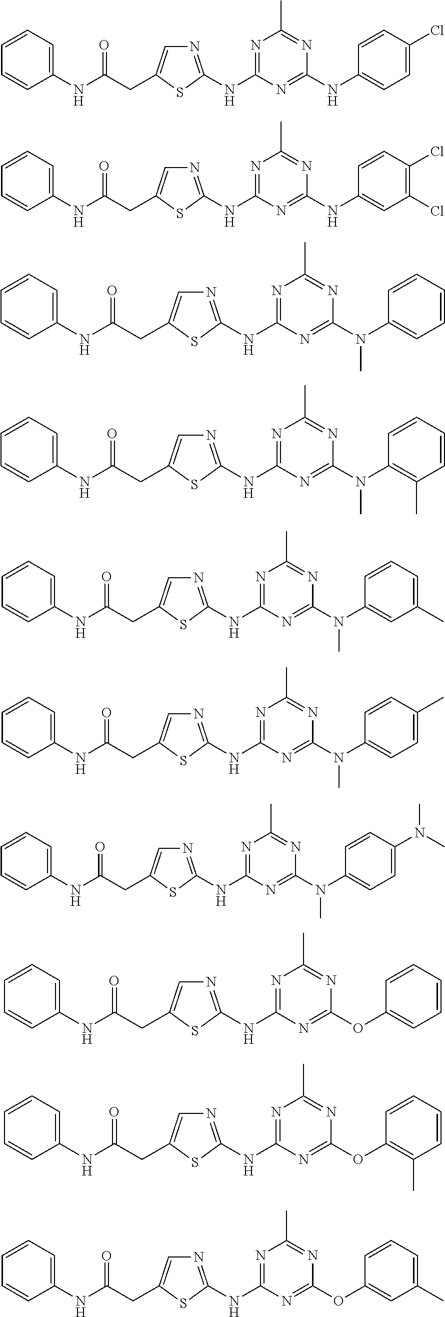

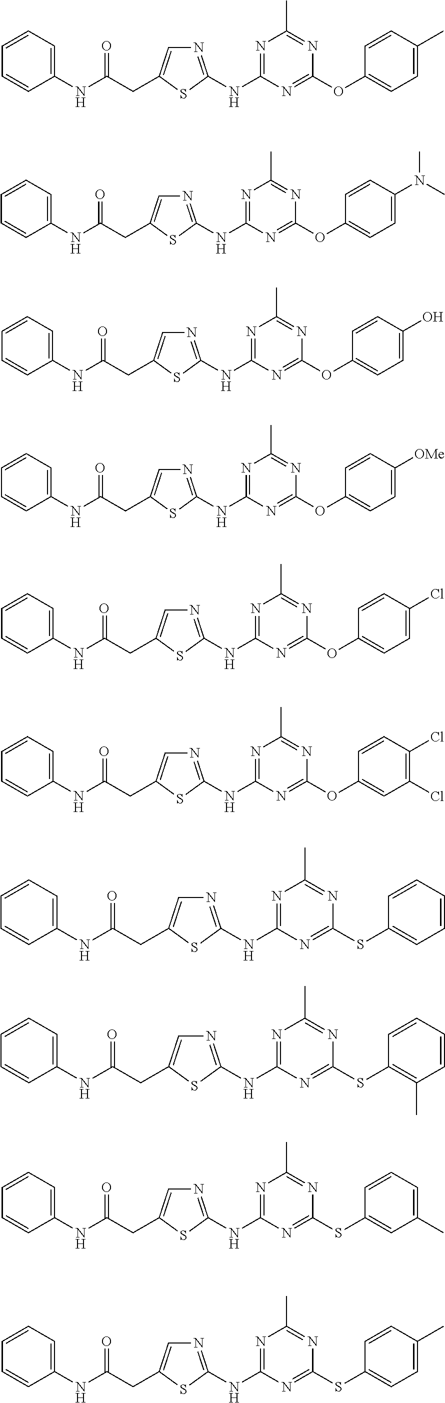

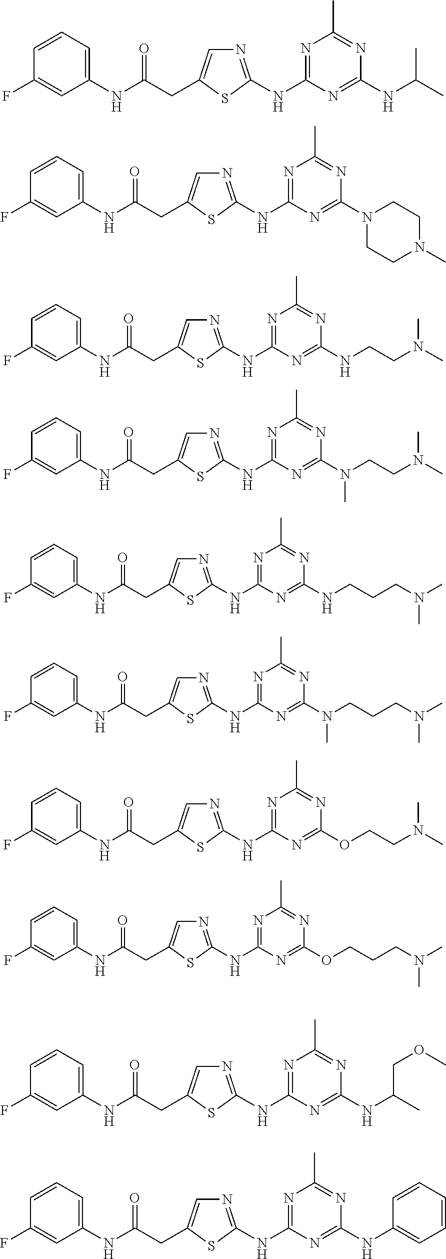

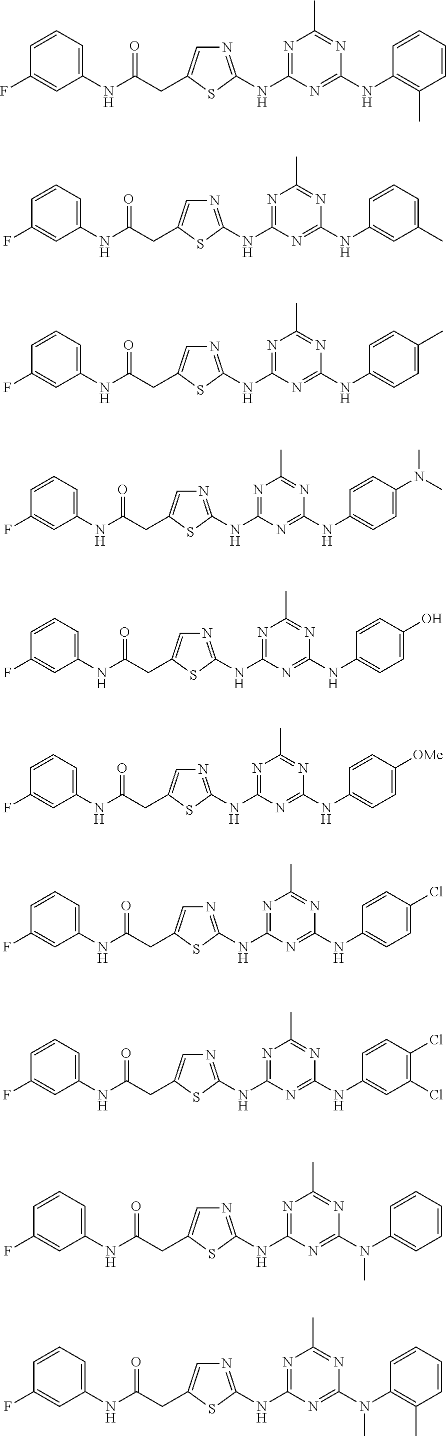

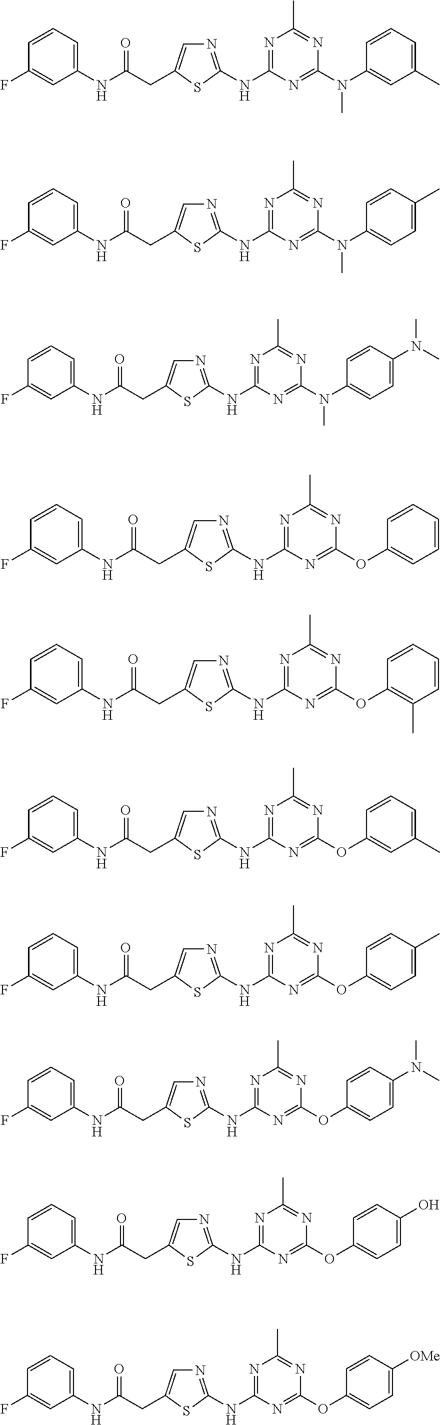

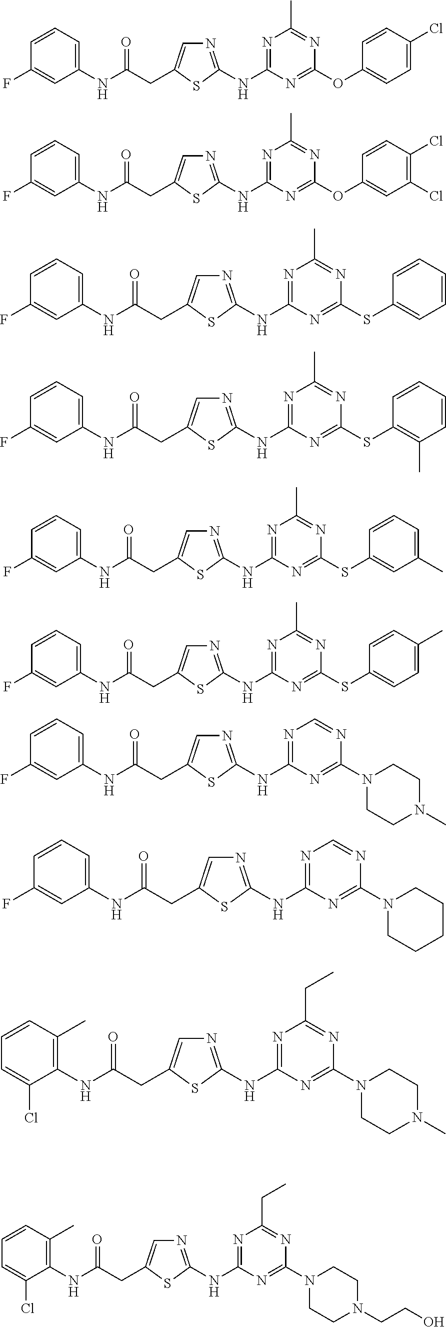

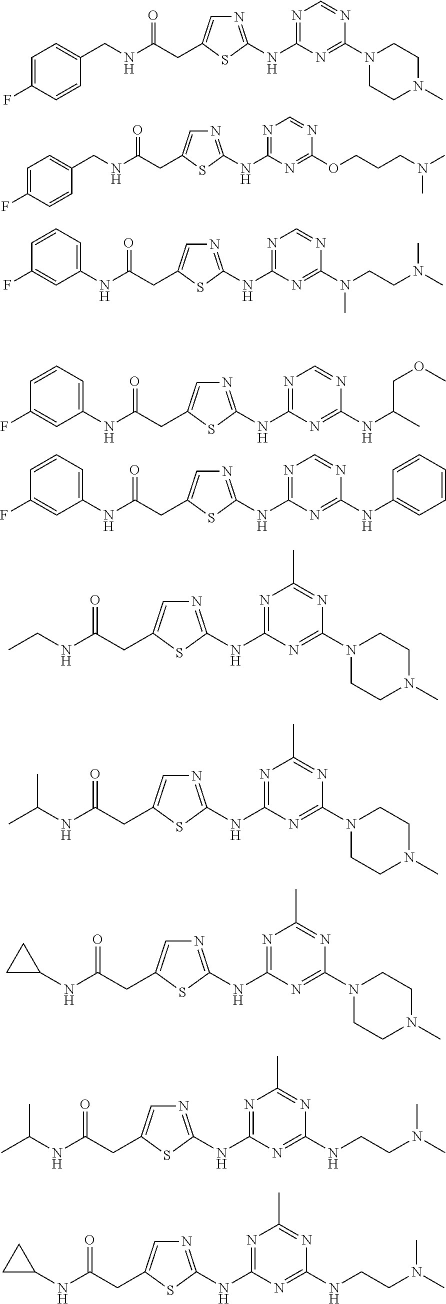

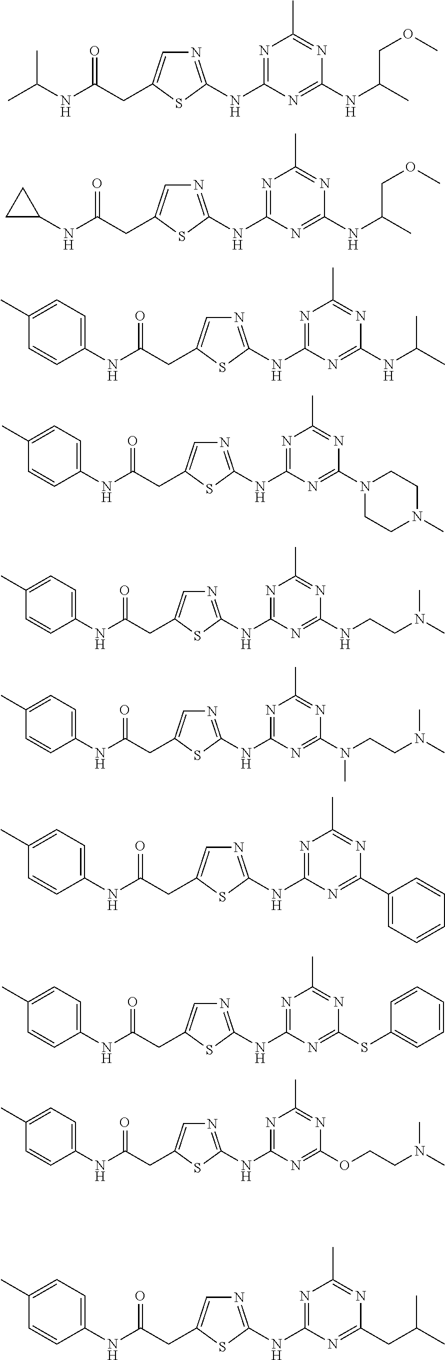

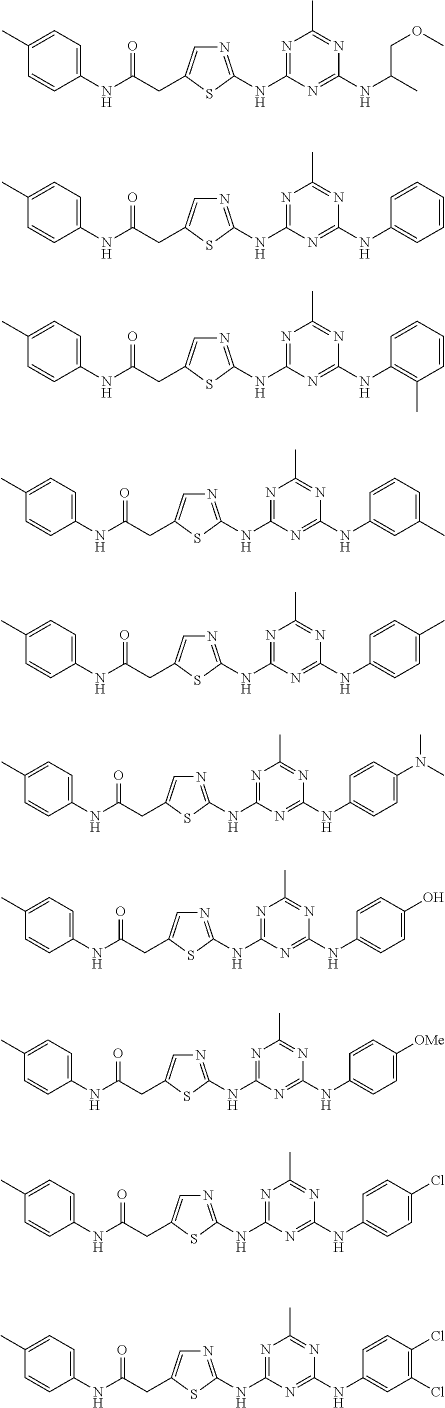

5. The composition according to claim 3, further comprising an additional therapeutic agent.

6. A method for treating a disease or condition in a mammal characterized by undesired cellular proliferation or hyperproliferation comprising identifying the mammal afflicted with said disease or condition and administering to said afflicted mammal a composition comprising the compound of claim 1.

7. The method of claim 6, wherein the disease or condition is cancer, stroke, congestive heart failure, an ischemia or reperfusion injury, arthritis or other arthropathy, retinopathy or vitreoretinal disease, macular degeneration, autoimmune disease, vascular leakage syndrome, inflammatory disease, edema, transplant rejection, burn, or acute or adult respiratory distress syndrome.

8. The method of claim 7, wherein the disease or condition is cancer.

9. The method of claim 7, wherein the disease or condition is autoimmune disease.

10. The method of claim 7, wherein the disease or condition is stroke.

11. The method of claim 7, wherein the disease or condition is arthritis.

12. The method of claim 7, wherein the disease or condition is inflammatory disease.

13. The method of claim 7, wherein the disease or condition is associated with a kinase.

14. The method according to claim 7, wherein said method further comprises administering an additional therapeutic agent.

15. The method according to claim 7, wherein said additional therapeutic agent is a chemotherapeutic agent.

16. The method of claim 13, wherein the kinase is a tyrosine kinase.

17. The method of claim 13, wherein the kinase is a serine kinase or a threonine kinase.

18. The method of claim 16, wherein the kinase is a Src family kinase.

19. The method of claim 16, wherein the kinase is a Abl family kinase.

20. The method of claim 8, wherein said cancer is selected from the group consisting of cancers of the liver and biliary tree, intestinal cancers, colorectal cancer, ovarian cancer, small cell and non-small cell lung cancer, breast cancer, sarcomas, fibrosarcoma, malignant fibrous histiocytoma, embryonal rhabdomysocarcoma, leiomysosarcoma, neuro-fibrosarcoma, osteosarcoma, synovial sarcoma, liposarcoma, alveolar soft part sarcoma, neoplasms of the central nervous systems, brain cancer, and lymphomas, including Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma, and combinations thereof.

21. A compound of the formula 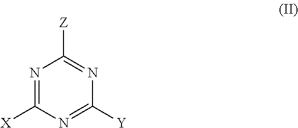
or a pharmaceutically acceptable salt thereof, wherein:
Y is selected from —OR⁴, —NR⁴R⁵, and -Q-R³;
Q is selected from cycloalkyl and heterocycloalkyl, each of which is optionally substituted with C₁-C₆ alkyl or oxo;
R³ is selected from H, C₁-C₆ alkyl, C₁-C₆ alkyl-R⁶, aryl, and heteroaryl, each of which is optionally substituted with C₁-C₆ alkyl, halo, trifluoromethyl, or oxo;
R⁴ and R⁵ are each independently selected from H, C₁-C₆ alkyl-R⁶, aryl, and heteroaryl;
R⁶ is selected from hydroxy, cyano, aryl, heteroaryl, cycloalkyl, heterocycloalkyl, —NH₂, mono(C₁-C₆)alkylamino, di(C₁-C₆)alkylamino, and C₁-C₆ alkoxy;
X is —NH—Ar¹—R¹;
Ar¹ is selected from aryl and heteroaryl, each of which is optionally substituted with C₁-C₆ alkyl or halo;
R¹ is selected from —(CH₂)ₙC(O)NHW, —CH₂C(O)NHAr¹, and —NH₂;
n=0, 1;
W is selected from C₁-C₆ alkyl, cycloalkyl, and —(CH₂)Ar¹;
Z is selected from H, C₁-C₆ alkyl, aryl, and heteroaryl.

22. A compound of the formula 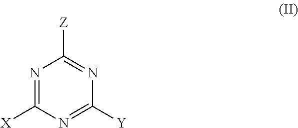
or a pharmaceutically acceptable salt thereof, wherein:
Y is selected from —OR⁴, —NR⁴R⁵, and -Q-R³;
Q is selected from morpholinyl, piperazinyl and piperidinyl;
R³ is selected from H, C₁-C₆ alkyl, hydroxy(C₁-C₆)alkyl, cyano(C₁-C₆)alkyl, pyridinylmethyl, pyridinyl, phenyl, trifluoromethylphenyl, and oxo;
R⁴ and R⁵ are each independently selected from H, C₁-C₆ alkyl-R⁶, and phenyl;
R⁶ is selected from hydroxy, morpholinyl, di(C₁-C₆)alkylamino, imidazolyl, and C₁-C₆ alkoxy;
X is —NH—Ar¹—R¹;
Ar¹ is selected from thiazolyl, oxazolyl, oxadiazolyl, methyl-imidazolyl, pyrazolyl;
R¹ is selected from —(CH₂)ₙC(O)NHW and —NH₂;
n=0, 1;
W is selected from C₁-C₆ alkyl and —(CH₂)ₙPh optionally substituted with C₁-C₆ alkyl or halo;
Z is selected from H, C₁-C₆ alkyl, and phenyl.

23. A compound of the formula 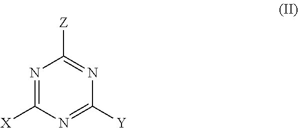
or a pharmaceutically acceptable salt thereof, wherein:
Y is selected from —OR⁴, —NR⁴R⁵, and -Q-R³;
Q is selected from morpholinyl, piperazinyl and piperidinyl;
R³ is selected from H, C₁-C₆ alkyl, hydroxy(C₁-C₆)alkyl, cyano(C₁-C₆)alkyl, pyridinylmethyl, pyridinyl, phenyl, trifluoromethylphenyl, and oxo;
R⁴ and R⁵ are each independently selected from H, C₁-C₆ alkyl-R⁶, and phenyl;
R⁶ is selected from hydroxy, morpholinyl, di(C₁-C₆)alkylamino, imidazolyl, and C₁-C₆ alkoxy;
X is —NH—Ar¹—R′;
Ar¹ is selected from thiazolyl, oxazolyl, oxadiazolyl, methyl-imidazolyl, pyrazolyl;
R¹ is selected from —(CH₂)ₙC(O)NHW, —CH₂C(O)NHAr², and —NH₂;
n=0, 1;
W is selected from C₁-C₆ alkyl, cycloalkyl, and —(CH₂)Ar²;
Ar² is phenyl, optionally substituted with C₁-C₆ alkyl or halo;
Z is selected from H, C₁-C₆ alkyl, and phenyl.

24. A process for making compound of claim 21 or its pharmaceutically acceptable salts, hydrates, solvates, crystal forms salts and individual diastereomers thereof.

25. A pharmaceutical composition comprising at least one compound of claim 21 or its pharmaceutically acceptable salts, hydrates, solvates, crystal forms salts and individual diastereomers thereof, and a pharmaceutically acceptable carrier.

26. A process for making compound of claim 22 or its pharmaceutically acceptable salts, hydrates, solvates, crystal forms salts and individual diastereomers thereof.

27. A pharmaceutical composition comprising at least one compound of claim 22 or its pharmaceutically acceptable salts, hydrates, solvates, crystal forms salts and individual diastereomers thereof, and a pharmaceutically acceptable carrier.

28. A process for making compound of claim 23 or its pharmaceutically acceptable salts, hydrates, solvates, crystal forms salts and individual diastereomers thereof.

29. A pharmaceutical composition comprising at least one compound of claim 23 or its pharmaceutically acceptable salts, hydrates, solvates, crystal forms salts and individual diastereomers thereof, and a pharmaceutically acceptable carrier.
